(19) United States
(12) Patent Application Publication
(10) Pub. No.: US 2024/0074306 A1
(43) Pub. Date: Feb. 29, 2024

(54) HETEROCYCLIC COMPOUND, ORGANIC LIGHT-EMITTING DEVICE COMPRISING SAME, MANUFACTURING METHOD THEREFOR, AND COMPOSITION FOR ORGANIC LAYER

(71) Applicant: LT MATERIALS CO., LTD., Yongin-si, Gyeonggi-do (KR)

(72) Inventors: Sol LEE, Yongin-si (KR); Jun Tae MO, Yongin-si (KR); Woo Jeong CHAE, Yongin-si (KR); Hyung Jin YANG, Yongin-si (KR); Dong Jun KIM, Yongin-si (KR)

(73) Assignee: LT MATERIALS CO., LTD., Yongin-si, Gyeonggi-do (KR)

(21) Appl. No.: 18/267,313

(22) PCT Filed: Nov. 5, 2021
(86) PCT No.: PCT/KR2021/016015

§ 371 Date: Jun. 14, 2023

(30) Foreign Application Priority Data
Dec. 15, 2020 (KR) 10-2020-0175130

(51) Int. Cl. H10K 85/60; C07D 209/96; C07D 405/04; C07D 405/12; C07D 409/04; C07D 409/12; C07D 513/04

(52) U.S. Cl. CPC H10K 85/636; C07D 209/96; C07D 405/04; C07D 405/12; C07D 409/04; C07D 409/12; C07D 513/04; H10K 85/633; H10K 85/654; H10K 85/657; H10K 85/6574; H10K 50/11

(57) ABSTRACT

The present specification relates to a heterocyclic compound represented by Formula 1, an organic light emitting device comprising the same, a method for preparing the same, and a composition for organic layer.

### TECHNICAL FIELD

The present application claims the benefit of priority based on Korean Patent Application No. 10-2020-0175130 filed on Dec. 15, 2020, the entire contents of which are incorporated herein by reference.

The present invention relates to a heterocyclic compound, an organic light emitting device comprising the same, a method for preparing the same, and a composition for organic layer.

### BACKGROUND ART

An organic light emitting device is a kind of self-emitting display device, which has the advantage of having a wide viewing angle, excellent contrast, and also fast response speed.

The organic light emitting device has a structure in which an organic thin film is disposed between two electrodes. When a voltage is applied to the organic light emitting device having such structure, electrons and holes injected from the two electrodes combine in the organic thin film to form a pair, and then emit light while disappearing. The organic thin film may be composed of a single layer or multiple layers, as required.

The material of the organic thin film may have a light emitting function, if necessary. For example, as a material for the organic thin film, a compound capable of forming a light emitting layer by itself alone may be used, or a compound capable of serving as a host or dopant of a host-dopant-based light emitting layer may also be used. In addition, as a material for the organic thin film, a compound capable of performing the roles of hole injection, hole transport, electron blocking, hole blocking, electron transport, electron injection, and the like may be used.

In order to improve the performance, lifetime or efficiency of an organic light emitting device, there is a continuous demand for the development of materials for the organic thin film.

### PRIOR ART DOCUMENT

[Patent Document]
(Patent Document 1) U.S. Pat. No. 4,356,429

### DISCLOSURE

### Technical Problem

It is an object of the present invention to provide a heterocyclic compound, an organic light emitting device comprising the same, a manufacturing method thereof, and a composition for organic layer.

### Technical Solution

The present invention provides a heterocyclic compound represented by Formula 1 below:

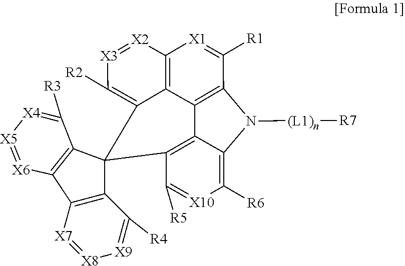

wherein,
X1 is N or CR11, X2 is N or CR12, X3 is N or CR13, X4 is N or CR14, X5 is N or CR15, X6 is N or CR16, X7 is N or CR17, X8 is N or CR18, X9 is N or CR19, and X10 is N or CR20,
R1 to R6 and R11 to R20 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other combine with each other to form a substituted or unsubstituted C6 to C60 aromatic hydrocarbon ring or a substituted or unsubstituted C2 to C60 heterocyclic ring, wherein R101, R102, and R103 are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
R7 is a substituted or unsubstituted C6 to C60 aryl group; or a substituted or unsubstituted C2 to C60 heteroaryl group,
L1 is a direct bond; a substituted or unsubstituted C6 to C60 arylene group; or a substituted or unsubstituted C2 to C60 heteroarylene group,
n is an integer from 0 to 5, and when n is 2 or more, each L1 is the same as or different from each other.

In addition, the present invention provides an organic light emitting device comprising a first electrode; a second electrode provided to face the first electrode; and one or more organic layers provided between the first electrode and the second electrode, wherein at least one of the one or more organic layers comprises a heterocyclic compound represented by Formula 1 above.

In addition, the present invention provides an organic light emitting device in which the organic layer further comprises a heterocyclic compound represented by Formula 2 below:

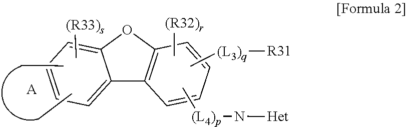

wherein,
N-Het is a substituted or unsubstituted, C2 to C60 monocyclic or polycyclic heterocyclic group containing one or more N,
L3 and L4 are the same as or different from each other, and are each independently a direct bond; a substituted or unsubstituted C6 to C60 arylene group; or a substituted or unsubstituted C6 to C60 heteroarylene group, p is an integer from 0 to 5, and when p is 2 or more, each L3 is the same as or different from each other, q is an integer from 0 to 5, and when q is 2 or more, each L4 is the same or different from each other,
A is a substituted or unsubstituted C6 to C60 aryl ring; or a substituted or unsubstituted C6 to C60 heteroaryl ring,
R31 to R33 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other combine with each other to form a substituted or unsubstituted C6 to C60 aromatic hydrocarbon ring or a substituted or unsubstituted C2 to C60 heterocyclic ring, wherein R201, R202, and R203 are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
r and s are each an integer from 0 to 2, and when r is 2 or more, each R32 is the same as or different from each other, and when s is 2 or more, each R33 is the same as or different from each other.

In addition, the present invention provides a composition for an organic layer of an organic light emitting device comprising the heterocyclic compound represented by Formula 1 above and the heterocyclic compound represented by Formula 2 above.

In addition, the present invention provides a method for manufacturing an organic light emitting device comprising the steps of preparing a substrate; forming a first electrode on the substrate; forming one or more organic layers on the first electrode; and forming a second electrode on the one or more organic layers, wherein the step of forming the one or more organic layers comprises a step of forming the one or more organic layers using the composition for organic layer of the organic light emitting device.

### Advantageous Effect

The compounds described herein can be used as a material for organic layer of an organic light emitting device. The compound may serve as a material for a hole injection layer, a material for a hole transport layer, a material for a light emitting layer, a material for an electron transport layer, a material for an electron injection layer or the like in an organic light emitting device. In particular, the compound can be used as a material for a light emitting layer of an organic light emitting device.

Specifically, the compound may be used alone or in combination with other compounds as a light emitting material, and may be used as a host material or a dopant material for a light emitting layer. If the compound represented by Formula 1 is used for an organic layer, it is possible to lower the operating voltage of the organic light emitting device, improve its luminous efficiency, and improve its lifetime characteristics.

In particular, the heterocyclic compound represented by Formula 1 of the present invention has a delocalized LUMO orbital, thereby exhibiting an effect of improving the lifetime of an organic electroluminescence device and thus improving electron stability and mobility.

In addition, the heterocyclic compound represented by Formula 1 of the present invention has a high triplet energy level (T1 level), thereby exhibiting an effect of preventing the reverse of energy transfer from the dopant to the host and well-preserving triplet excitons in the light emitting layer.

In addition, the heterocyclic compound represented by Formula 1 of the present invention exhibits an effect of well-preserving excitons by facilitating intramolecular charge transfer and reducing the energy gap between singlet energy level (Si) and triplet energy level (T1).

### DESCRIPTION OF DRAWING

FIGS. 1 to 4 are views schematically showing a stacked structure of an organic light emitting device according to an embodiment of the present invention, respectively.

### BEST MODE

Hereinafter, the present application will be described in detail.

In the present specification, the term “substituted” means that a hydrogen atom bonded to a carbon atom of a compound is replaced with another substituent, the position to be substituted is not limited as long as it is a position where a hydrogen atom may be substituted, that is, it is a position that can be substituted by a substituent, and when substituted by two or more substituents, two or more substituents may be the same or different from each other.

In the present specification, the term “substituted or unsubstituted” means that it is substituted or unsubstituted by one or more substituents selected from the group consisting of C1 to C60 straight or branched chain alkyl; C2 to C60 straight or branched alkenyl; C2 to C60 straight or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroaryl amine, or means that it is substituted or unsubstituted with a substituent formed by connecting two or more substituents selected from the above-exemplified substituents.

In the present specification, the halogen may be fluorine, chlorine, bromine or iodine.

In the present specification, the alkyl group comprises a straight or branched chain having 1 to 60 carbon atoms, and may be further substituted by other substituents. The number of carbon atoms in the alkyl group may be 1 to 60, specifically 1 to 40, more specifically, 1 to 20. Specific examples of the alkyl group may be, but is not limited to, a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like.

In the present specification, the alkenyl group comprises a straight or branched chain having 2 to 60 carbon atoms, and may be further substituted by other substituents. The carbon number of the alkenyl group may be 2 to 60, specifically 2 to 40, more specifically 2 to 20. Specific examples of the alkenyl group may be, but is not limited to, a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like.

In the present specification, the alkynyl group comprises a straight or branched chain having 2 to 60 carbon atoms, and may be further substituted by other substituents. The carbon number of the alkynyl group may be 2 to 60, specifically 2 to 40, more specifically 2 to 20.

In the present specification, the alkoxy group may be a straight-chain, a branched-chain or a cyclic chain. Although the number of carbon atoms in the alkoxy group is not particularly limited, it is preferable that the number of carbon atoms is 1-20. Specific examples of the alkoxy group may be, but is not limited to, methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like.

In the present specification, the cycloalkyl group comprises a monocyclic or polycyclic ring having 3 to 60 carbon atoms, and may be further substituted by other substituents. In this case, the polycyclic ring refers to a group formed by directly connecting or condensing a cycloalkyl group with another cyclic group. In this case, the other cyclic group may be a cycloalkyl group, but may be a different type of cyclic group, for example, a heterocycloalkyl group, an aryl group, a heteroaryl group, or the like. The number of carbon atoms in the cycloalkyl group may be 3 to 60, specifically 3 to 40, more specifically 5 to 20. Specific examples of the cycloalkyl group may be, but is not limited to, a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like.

In the present specification, the heterocycloalkyl group comprises a monocyclic or polycyclic ring containing 0, S, Se, N or Si as a hetero atom and having 2 to 60 carbon atoms, and may be further substituted by other substituents. In this case, the polycyclic ring refers to a group formed by directly connecting or condensing a heterocycloalkyl group with another cyclic group. In this case, the other cyclic group may be a heterocycloalkyl group, but may be a different type of cyclic group, for example, a cycloalkyl group, an aryl group, a heteroaryl group, or the like. The number of carbon atoms in the heterocycloalkyl group may be 2 to 60, specifically 2 to 40, more specifically 3 to 20.

In the present specification, the aryl group comprises a monocyclic or polycyclic ring having 6 to 60 carbon atoms, and may be further substituted by other substituents. In this case, the polycyclic ring means a group formed by directly connecting or condensing an aryl group with another cyclic group. In this case, the other cyclic group may be an aryl group, but may be a different type of cyclic group, for example, a cycloalkyl group, a heterocycloalkyl group, a heteroaryl group, or the like. The aryl group comprises a spiro group. The number of carbon atoms in the aryl group may be 6 to 60, specifically 6 to 40, more specifically 6 to 25. Specific examples of the aryl group may be, but is not limited to, a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenylenyl group, a pyrenyl group, a tetracenyl group, a pentacenyl group, a fluorenyl group, an indenyl group, an acenaphthyl group, a benzofluorenyl group, a spirobifluorenyl group, a 2,3-dihydro-1H-indenyl group, condensed ring groups thereof and the like.

In the present specification, the phosphine oxide group is represented by —P(═O)R101R102, wherein R101 and R102 are the same as or different from each other, and may each independently be a substituent consisting of at least one of hydrogen; deuterium; a halogen group; an alkyl group; an alkenyl group; an alkoxy group; a cycloalkyl group; an aryl group; and a heterocyclic group. Specifically, it may be substituted with an aryl group, and the above-described examples may be applied to the aryl group. For example, the phosphine oxide group is, but is not limited to, a diphenyl phosphine oxide group, dinaphthyl phosphine oxide, or the like.

In the present specification, the silyl group contains Si and is a substituent formed by directly connecting the Si atoms as a radical, and is represented by —SiR₁₀₄R₁₀₅R₁₀₆, wherein R₁₀₄ to R₁₀₆ are the same as or different from each other, and may each independently be a substituent consisting of at least one of hydrogen; deuterium; a halogen group; an alkyl group; an alkenyl group; an alkoxy group; a cycloalkyl group; an aryl group; and a heterocyclic group. Specific examples of the silyl group may be, but is not limited to, a trimethylsilyl group, a triethylsilyl group, a t-butyldimethylsilyl group, vinyldimethylsilyl group, a propyldimethylsilyl group, a triphenylsilyl group, a diphenylsilyl group, a phenylsilyl group and the like.

In the present specification, the fluorenyl group may be substituted, and adjacent substituents may be combined with each other to form a ring.

If the fluorenyl group is substituted, it may be

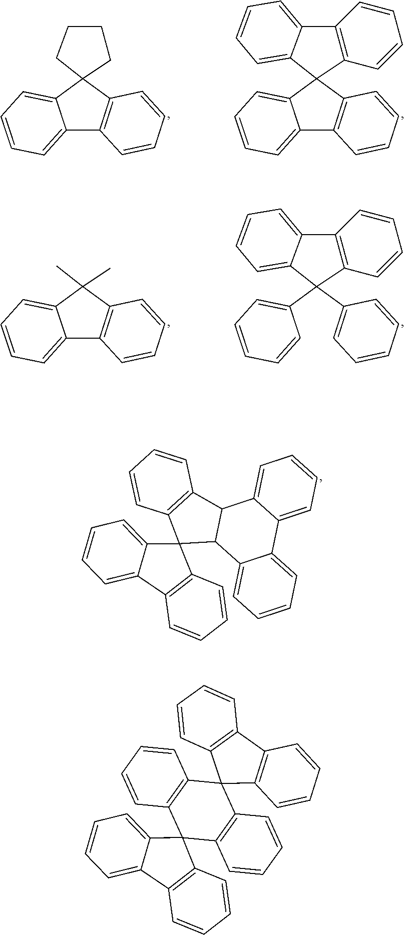

and the like, but is not limited thereto.

In the present specification, the heteroaryl group comprises a monocyclic or polycyclic ring containing S, 0, Se, N or Si as a hetero atom and having 2 to 60 carbon atoms, and may be further substituted by other substituents. In this case, the polycyclic ring refers to a group formed by directly connecting or condensing a heteroaryl group with another cyclic group. In this case, the other cyclic group may be a heteroaryl group, but may be a different type of cyclic group, for example, a cycloalkyl group, a heterocycloalkyl group, an aryl group, or the like. The number of carbon atoms in the heteroaryl group may be 2 to 60, specifically 2 to 40, more specifically 3 to 25. Specific examples of the heteroaryl group may be, but is not limited to, a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, tetrazinyl group, a quinolyl group, an isoquinolyl group, a quinazolinyl group, an isoquinazolinyl group, a quinazolylyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilolyl group, spirobi(dibenzosilolyl), a dihydrophenazinyl group, a phenoxazinyl group, a phenanthridyl group, an imidazopyridinyl group, a thienyl group, an indolo[2,3-a] carbazolyl group, an indolo[2,3-b] carbazolyl group, an indolinyl group, a 10,11-dihydro-dibenzo[b,f]azepinyl group, a 9,10-dihydroacridinyl group, a phenanthrazinyl group, a phenothiazinyl group, phthalazinyl group, a naphthylidinyl group, a phenanthrolinyl group, a benzo[c][1,2,5]thiadiazolyl group, 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms of the amine group is not particularly limited, but is preferably 1 to 30. Specific examples of the amine group may be, but is not limited to, a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like.

In the present specification, the arylene group means that the aryl group has two bonding positions, that is, a divalent group. These are the same as those described for the aryl group described above, except that each of them is a divalent group. In addition, the heteroarylene group means that the heteroaryl group has two bonding positions, that is, a divalent group. These are the same as those described for the heteroaryl group described above, except that each of them is a divalent group.

In the present specification, an “adjacent” group may mean a substituent substituted at the atom directly linked to the atom substituted by that substituent, a substituent that is sterically closest to that substituent, or another substituent substituted at the atom substituted by that substituent. For example, two substituents substituted at an ortho position in a benzene ring and two substituents substituted at the same carbon in an aliphatic ring may be interpreted as “adjacent” groups to each other.

In the present invention, “when a substituent is not indicated in the structure of the formula or the compound” means that a hydrogen atom is bonded to a carbon atom. However, since deuterium (2H) is an isotope of hydrogen, some hydrogen atoms may be deuterium.

In one embodiment of the present invention, “when a substituent is not indicated in the structure of the formula or the compound” may mean that all positions that may be substituted by the substituent are hydrogen or deuterium. That is, in the case of deuterium, some hydrogen atoms as an isotope of hydrogen may be isotopes of deuterium. In this case, the content of deuterium may be 0% to 100%.

In one embodiment of the present invention, in the case of “when a substituent is not indicated in the structure of the formula or the compound”, if deuterium is not explicitly excluded, except that “the content of deuterium is 0%”, “the content of hydrogen is 100%”, “all substituents are hydrogen” etc., thus hydrogen and deuterium may be used in admixture in the compound.

In one embodiment of the present invention, deuterium is one of the isotopes of hydrogen, and is an element having as an atomic nucleus a deuteron consisting of one proton and one neutron, and can be expressed as hydrogen-2, and the element symbol can also be written as D or ²H.

In one embodiment of the present invention, an isotope means an atom with the same atomic number (Z) but a different mass number (A), and can also be interpreted as an element that has the same number of protons but the different number of neutrons.

In one embodiment of the present invention, the meaning of content T % of a specific substituent may be defined as T2/T1X100=T % wherein T1 is defined as the total number of substituents that the basic compound can have, and T2 is defined as the number of specific substituents substituted among them.

That is, in one example, the 20% content of deuterium in the phenyl group represented by

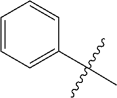 [SMILES: *c1ccccc1]

may mean that the total number of substituents that the phenyl group can have is 5 (T1 in the formula), and the number of deuterium among them is 1 (T2 in the formula). That is, in the phenyl group, that the content of deuterium is 20% may be represented by Structural Formulas below:

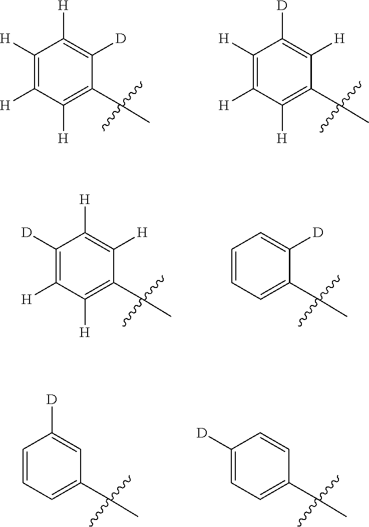 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Also, in one embodiment of the present invention, in the case of “a phenyl group having a deuterium content of 0%”, it means a phenyl group that does not contain a deuterium atom, i.e., has 5 hydrogen atoms.

In the present invention, the content of deuterium in the heterocyclic compound represented by Formula 1 may be 0 to 100%, more preferably 30 to 100%.

In the present invention, C6 to C60 aromatic hydrocarbon ring means a compound containing an aromatic ring consisting of C6 to C60 carbons and hydrogen, and for example, may be, but is not limited to, benzene, biphenyl, terphenyl, triphenylene, naphthalene, anthracene, phenalene, phenanthrene, fluorene, pyrene, chrysene, perylene, azulene and the like, and comprises all of the aromatic hydrocarbon ring compounds known in the art as satisfying the carbon number described above.

The present invention provides a heterocyclic compound represented by the following Formula 1:

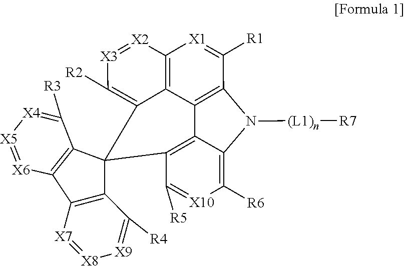

wherein,
X1 is N or CR11, X2 is N or CR12, X3 is N or CR13, X4 is N or CR14, X5 is N or CR15, X6 is N or CR16, X7 is N or CR17, X8 is N or CR18, X9 is N or CR19, and X10 is N or CR20,
R1 to R6 and R11 to R20 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other are combined with each other to form a substituted or unsubstituted C6 to C60 aromatic hydrocarbon ring or a substituted or unsubstituted C2 to C60 heterocyclic ring, wherein R101, R102, and R103 are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
L1 is a direct bond; a substituted or unsubstituted C6 to C60 arylene group; or a substituted or unsubstituted C2 to C60 heteroarylene group, and
n is an integer from 0 to 5, and when n is 2 or more, each L1 is the same as or different from each other.

The aliphatic or aromatic hydrocarbon ring or hetero ring that may be formed by the adjacent groups may be structures exemplified by the aforementioned cycloalkyl group, the cycloheteroalkyl group, the aryl group and the heteroaryl group, except that it is not a monovalent group.

In one embodiment of the present invention, Formula 1 may be a heterocyclic compound represented by any one of Formula 1-1 to Formula 1-3 below:

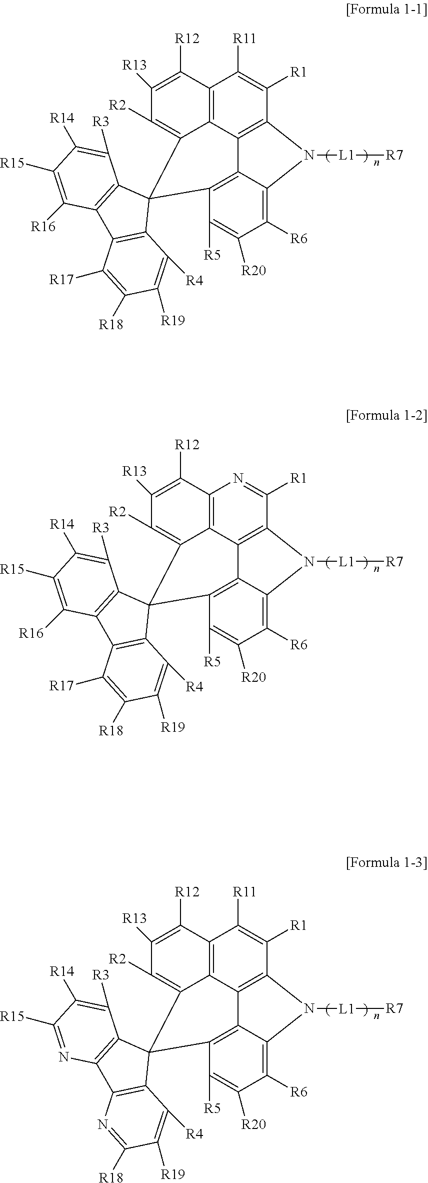

wherein the definitions of R1 to R7, R11 to R20, L1, and n are the same as in Formula 1 above.

In one embodiment of the present invention, at least one of R11 to R13 and R20 may be a group represented by Formula 1-4 below:

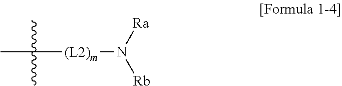

wherein,
Ra and Rb are the same as or different from each other, and are each independently a substituted or unsubstituted C6 to C60 aryl group; or a substituted or unsubstituted C2 to C60 heteroaryl group,
L2 is a direct bond; a substituted or unsubstituted C6 to C60 arylene group; or a substituted or unsubstituted C2 to C60 heteroarylene group, and
m is an integer from 0 to 5, and when m is 2 or more, each L2 is the same as or different from each other.

In other embodiment of the present invention, one of R11 to R13 and R20 may be a group represented by Formula 1-4.

In one embodiment of the present invention, Ra and Rb may be the same as or different from each other, and may each independently be a substituted or unsubstituted C6 to C30 aryl group; or a substituted or unsubstituted C2 to C30 heteroaryl group.

In other embodiment of the present invention, Ra and Rb may be the same as or different from each other, and may each independently be a substituted or unsubstituted C6 to C20 aryl group; or a substituted or unsubstituted C2 to C20 heteroaryl group.

In other embodiment of the present invention, Ra and Rb may be the same as or different from each other, and may each independently be a substituted or unsubstituted phenyl group, a naphthyl group, a phenanthrenyl group, a fluorenyl group; or a substituted or unsubstituted dibenzofuranyl group, a dibenzothiophenyl group.

In one embodiment of the present invention, L2 may be a direct bond; a substituted or unsubstituted C6 to C30 arylene group; or a substituted or unsubstituted C2 to C30 heteroarylene group.

In other embodiment of the present invention, L2 may be a direct bond; a substituted or unsubstituted C6 to C20 arylene group; or a substituted or unsubstituted C2 to C20 heteroarylene group.

In other embodiment of the present invention, L2 may be a direct bond; a substituted or unsubstituted phenylene group, a naphthylene group; or a substituted or unsubstituted dibenzofuranylene group, a dibenzothiophenylene group.

In one embodiment of the present invention, m may be an integer of 1 to 3.

In other embodiment of the present invention, m may be an integer of 1 to 2.

In one embodiment of the present invention, R7 may be a group represented by Formula 1-5 below:

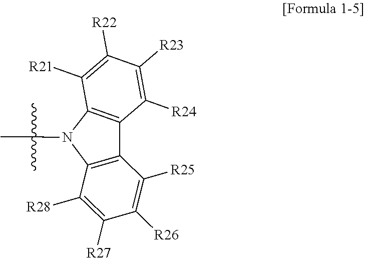

wherein,
R21 to R28 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other are combine with each other to form a substituted or unsubstituted C6 to C60 aromatic hydrocarbon ring or a substituted or unsubstituted C2 to C60 heterocyclic ring, wherein R101, R102, and R103 are the same as or different from each other, and are each independently a substituted or unsubstituted C1 to C60 alkyl group; a substituted or unsubstituted C6 to C60 aryl group; or a substituted or unsubstituted C2 to C60 heteroaryl group.

In other embodiment of the present invention, R21 to R28 may be the same as or different from each other, and may each independently be selected from the group consisting of hydrogen; deuterium; a substituted or unsubstituted C1 to C60 alkyl group; a substituted or unsubstituted C2 to C60 alkenyl group; a substituted or unsubstituted C6 to C60 aryl group; and a substituted or unsubstituted C2 to C60 heteroaryl group, or two or more groups adjacent to each other may be combined with each other to form a substituted or unsubstituted C6 to C60 aromatic hydrocarbon ring or a substituted or unsubstituted C2 to C60 heterocyclic ring.

In other embodiment of the present invention, R21 to R28 may be the same as or different from each other, and may each independently be selected from the group consisting of hydrogen; deuterium; substituted or unsubstituted C1 to C30 alkyl group; a substituted or unsubstituted C2 to C30 alkenyl group; a substituted or unsubstituted C6 to C30 aryl group; and substituted or unsubstituted C2 to C30 heteroaryl group, or two or more groups adjacent to each other may be combined with each other to form a substituted or unsubstituted C6 to C30 aromatic hydrocarbon ring or substituted or unsubstituted C2 to C30 heterocyclic ring.

In other embodiment of the present invention, R21 to R28 may be the same as or different from each other, and may each independently be selected from the group consisting of hydrogen; deuterium; a substituted or unsubstituted C1 to C20 alkyl group; a substituted or unsubstituted C2 to C20 alkenyl group; a substituted or unsubstituted C6 to C20 aryl group; and a substituted or unsubstituted C2 to C20 heteroaryl group, or two or more groups adjacent to each other may be combined with each other to form a substituted or unsubstituted C6 to C20 aromatic hydrocarbon ring or a substituted or unsubstituted C2 to C20 heterocyclic ring.

In other embodiment of the present invention, R21 to R28 may be the same as or different from each other, and may each independently be hydrogen; deuterium; a substituted or unsubstituted phenyl group, or two or more groups adjacent to each other may be combined with each other to form a substituted or unsubstituted benzene ring.

In one embodiment of the present invention, Formula 1 may be a heterocyclic compound represented by any one of Formula 1-6 to Formula 1-9 below:

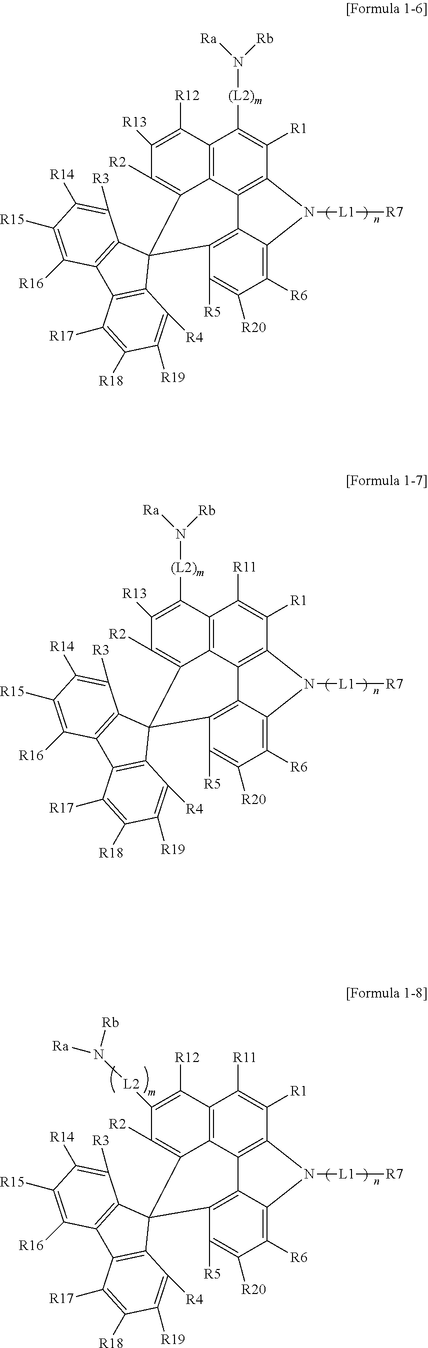

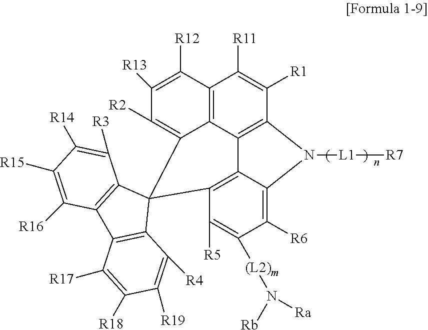

wherein,
R11 to R20 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other are combined with each other to form a substituted or unsubstituted C6 to C60 aromatic hydrocarbon ring or a substituted or unsubstituted C2 to C60 heterocyclic ring,
L2 is a direct bond; a substituted or unsubstituted C6 to C60 arylene group; or a substituted or unsubstituted C2 to C60 heteroarylene group, and
m is an integer of 0 to 5, and when m is 2 or more, each L2 is the same as or different from each other,

The definitions of R1 to R7, L1 and n are the same as in Formula 1 above.

In one embodiment of the present invention, R11 to R20 may be the same as or different from each other, and may each independently be selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; and substituted or unsubstituted C2 to C30 heteroaryl group, or two or more groups adjacent to each other may be combined with each other to form a substituted or unsubstituted C6 to C30 aromatic hydrocarbon ring or a substituted or unsubstituted C2 to C30 heterocyclic ring.

In other embodiment of the present invention, R11 to R20 may be the same as or different from each other, and may each independently be hydrogen; deuterium; a substituted or unsubstituted C1 to C30 alkyl group; or a substituted or unsubstituted C6 to C30 aryl group.

In other embodiment of the present invention, R11 to R20 may be the same as or different from each other, and may each independently be hydrogen; deuterium; a substituted or unsubstituted C1 to C20 alkyl group; or a substituted or unsubstituted C6 to C20 aryl group.

In other embodiment of the present invention, R11 to R20 may be the same as or different from each other, and may each independently be hydrogen; deuterium; a substituted or unsubstituted tert-butyl group; or a substituted or unsubstituted phenyl group.

In other embodiment of the present invention, at least one of R11 to R20 may be a substituted or unsubstituted tert-butyl group; or a substituted or unsubstituted phenyl group, and the other may be hydrogen; or deuterium.

In other embodiment of the present invention, one of R11 to R20 may be a substituted or unsubstituted tert-butyl group; or a substituted or unsubstituted phenyl group, and the remainder may be hydrogen; or deuterium.

In other embodiment of the present invention, R14 and R19 of R11 to R20 may be a substituted or unsubstituted tert-butyl group, and the remainder may be hydrogen; or deuterium.

In other embodiment of the present invention, R15 and R18 of R11 to R20 may be a substituted or unsubstituted phenyl group, and the remainder may be hydrogen; or deuterium.

In one embodiment of the present invention, R1 to R6 may be the same as or different from each other, and may each independently be selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; and a substituted or unsubstituted C2 to C30 heteroaryl group, or two or more groups adjacent to each other may be combined with each other to form a substituted or unsubstituted C6 to C30 aromatic hydrocarbon ring or a substituted or unsubstituted C2 to C30 heterocyclic ring.

In other embodiment of the present invention, R1 to R6 may be the same as or different from each other, and may each independently be hydrogen; deuterium; a substituted or unsubstituted C6 to C30 aryl group; or a substituted or unsubstituted C2 to C30 heteroaryl group.

In other embodiment of the present invention, R1 to R6 may be the same as or different from each other, and may each independently be hydrogen; or deuterium.

In one embodiment of the present invention, R7 may be a substituted or unsubstituted C6 to C30 aryl group; or a substituted or unsubstituted C2 to C30 heteroaryl group.

In other embodiment of the present invention, R7 may be a substituted or unsubstituted C6 to C20 aryl group; or a substituted or unsubstituted C2 to C20 heteroaryl group.

In other embodiment of the present invention, R7 may be a substituted or unsubstituted phenyl group, a naphthyl group, a fluorenyl group, a phenanthrenyl group; or a substituted or unsubstituted dibenzofuranyl group, a dibenzothiophenyl group.

In one embodiment of the present invention, L1 may be a direct bond; a substituted or unsubstituted C6 to C30 arylene group; or a substituted or unsubstituted C2 to C30 heteroarylene group.

In other embodiment of the present invention, L1 may be a direct bond; a substituted or unsubstituted C6 to C20 arylene group; or a substituted or unsubstituted C2 to C20 heteroarylene group.

In other embodiment of the present invention, L1 may be a direct bond; or a substituted or unsubstituted phenylene group, a naphthylene group.

In one embodiment of the present invention, L2 may be a direct bond; a substituted or unsubstituted C6 to C30 arylene group; or a substituted or unsubstituted C2 to C30 heteroarylene group.

In other embodiment of the present invention, L2 may be a direct bond; a substituted or unsubstituted C6 to C20 arylene group; or a substituted or unsubstituted C2 to C20 heteroarylene group.

In other embodiment of the present invention, L2 may be a direct bond; a substituted or unsubstituted phenylene group, a naphthylene group; or a substituted or unsubstituted dibenzofuranylene group, a dibenzothiophenylene group.

In one embodiment of the present invention, n and m may be the same as or different from each other, and may each independently be an integer from 1 to 3.

In other embodiment of the present invention, n and m may be the same as or different from each other, and may each independently be an integer from 1 to 2.

In other embodiment of the present invention, the ‘substitution’ of L1, L2, Ra, Rb, R1 to R7, and R11 to R20 may each independently be made by one or more substituents selected from the group consisting of a C1 to C10 alkyl group; a C2 to C10 alkenyl group; a C2 to C10 alkynyl group; a C3 to C15 cycloalkyl group; a C2 to C20 heterocycloalkyl group; a C6 to C30 aryl group; a C2 to C30 heteroaryl group; a C1 to C10 alkylamine group; a C6 to C30 arylamine group; and a C2 to C30 heteroarylamine group.

In other embodiment of the present invention, the ‘substitution’ of L1, L2, Ra, Rb, R1 to R7 and R11 to R20 may each independently be made by one or more substituents selected from the group consisting of a C1 to C10 alkyl group; a C6 to C30 aryl group; a C2 to C30 heteroaryl group.

In other embodiment of the present invention, the ‘substitution’ of L1, L2, Ra, Rb, R1 to R7 and R11 to R20 may each independently be made by one or more substituents selected from the group consisting of a C1 to C5 alkyl group; a C6 to C20 aryl group; and a C2 to C20 heteroaryl group.

In other embodiment of the present invention, the ‘substitution’ of L1, L2, Ra, Rb, R1 to R7, and R11 to R20 may each independently be made by one or more substituents selected from the group consisting of a methyl group, an ethyl group, a straight or branched propyl group, a straight or branched butyl group, a straight or branched pentyl group, a phenyl group, a naphthyl group, a pyridinyl group, an anthracenyl group, a carbazolyl group, a dibenzothiophenyl group, a dibenzofuranyl group, and a phenanthrenyl group.

In other embodiment of the present invention, the ‘substitution’ of L1, L2, Ra, Rb, R1 to R7, and R11 to R20 may each independently be made by a methyl group, an ethyl group, a straight or branched propyl group, a straight or branched butyl group, and a straight or branched pentyl group.

In one embodiment of the present invention, Formula 1 may be a heterocyclic compound represented by Formula 1-10 below:

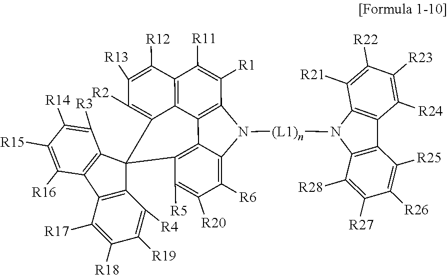

wherein,
R11 to R20 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other are combined with each other to form a substituted or unsubstituted C6 to C60 aromatic hydrocarbon ring or a substituted or unsubstituted C2 to C60 heterocyclic ring,
R21 to R28 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other are combined with each other to form a substituted or unsubstituted C6 to C60 aromatic hydrocarbon ring or a substituted or unsubstituted C2 to C60 heterocyclic ring, wherein R101, R102, and R103 are the same as or different from each other, and are each independently a substituted or unsubstituted C1 to C60 alkyl group; a substituted or unsubstituted C6 to C60 aryl group; or a substituted or unsubstituted C2 to C60 heteroaryl group, and
the definitions of R1 to R6, L1, and n are the same as in Formula 1 above.

In one embodiment of the present invention, R11 to R20 and R1 to R6 may be the same as or different from each other, and may each independently be selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; and substituted or unsubstituted C2 to C30 heteroaryl group, or two or more groups adjacent to each other may be combined with each other to form a substituted or unsubstituted C6 to C30 aromatic hydrocarbon ring or a substituted or unsubstituted C2 to C30 heterocyclic ring.

In other embodiment of the present invention, R11 to R20 may be the same as or different from each other, and may each independently be hydrogen; deuterium; a substituted or unsubstituted C1 to C30 alkyl group; or a substituted or unsubstituted C6 to C30 aryl group.

In other embodiment of the present invention, R11 to R20 may be the same as or different from each other, and may each independently be hydrogen; deuterium; a substituted or unsubstituted C1 to C20 alkyl group; or a substituted or unsubstituted C6 to C20 aryl group.

In other embodiment of the present invention, R11 to R20 may be the same as or different from each other, and may each independently be hydrogen; or deuterium.

In one embodiment of the present invention, L1 may be a direct bond; a substituted or unsubstituted C6 to C30 arylene group; or a substituted or unsubstituted C2 to C30 heteroarylene group.

In other embodiment of the present invention, L1 may be a direct bond; a substituted or unsubstituted C6 to C20 arylene group; or a substituted or unsubstituted C2 to C20 heteroarylene group.

In other embodiment of the present invention, L1 may be a direct bond; or a substituted or unsubstituted phenylene group.

In one embodiment of the present invention, n may be an integer of 1 to 3.

In other embodiment of the present invention, n may be an integer from 1 to 2.

In other embodiment of the present invention, the ‘substitution’ of R1 to R7, R11 to R20, R21 to R28, Ra, Rb, L1, and L2 may each independently be made by one or more substituents selected from the group consisting of a C1 to C10 alkyl group; a C2 to C10 alkenyl group; a C2 to C10 alkynyl group; a C6 to C30 aryl group; and a C2 to C30 heteroaryl group.

In other embodiment of the present invention, the ‘substitution’ of R1 to R7, R11 to R20, R21 to R28, Ra, Rb, L1, and L2 may each independently be made by one or more substituents selected from the group consisting of a C1 to C5 alkyl group; a C6 to C20 aryl group; and a C2 to C20 heteroaryl group.

In other embodiment of the present invention, the ‘substitution’ of R1 to R7, R11 to R20, R21 to R28, Ra, Rb, L1, and L2 may each independently be made by one or more substituents selected from the group consisting of C1 to C5 alkyl; C6 to C12 aryl; and C2 to C12 heteroaryl.

In other embodiment of the present invention, the ‘substitution’ of R1 to R7, R11 to R20, R21 to R28, Ra, Rb, L1 and L2 may each independently be made by one or more substituents selected from the group consisting of methyl, ethyl, straight or branched propyl, straight or branched butyl, straight or branched pentyl, phenyl, and naphthyl.

In other embodiment of the present invention, the ‘substitution’ of R1 to R7, R11 to R20, R21 to R28, Ra, Rb, L1, and L2 may each independently be made by one or more substituents selected from the group consisting of methyl, ethyl, straight or branched propyl, straight or branched butyl, and straight or branched pentyl.

In one embodiment of the present invention, the heterocyclic compound represented by Formula 1 may be at least one selected from the compounds below:

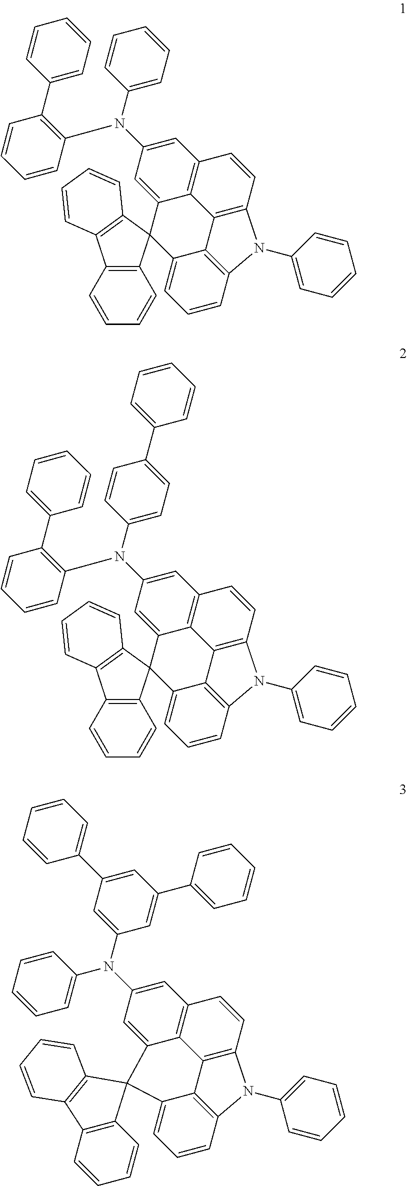 [SMILES: c1ccc(-c2ccccc2N(c2ccccc2)c2cc3c4c(ccc5c4c4c(cccc4n5-c4ccccc4)C34c3ccccc3-c3ccccc34)c2)cc1 ; c1ccc(-c2ccc(N(c3cc4c5c(ccc6c5c5c(cccc5n6-c5ccccc5)C45c4ccccc4-c4ccccc45)c3)c3ccccc3-c3ccccc3)cc2)cc1 ; c1ccc(-c2cc(-c3ccccc3)cc(N(c3ccccc3)c3cc4c5c(ccc6c5c5c(cccc5n6-c5ccccc5)C45c4ccccc4-c4ccccc45)c3)c2)cc1]

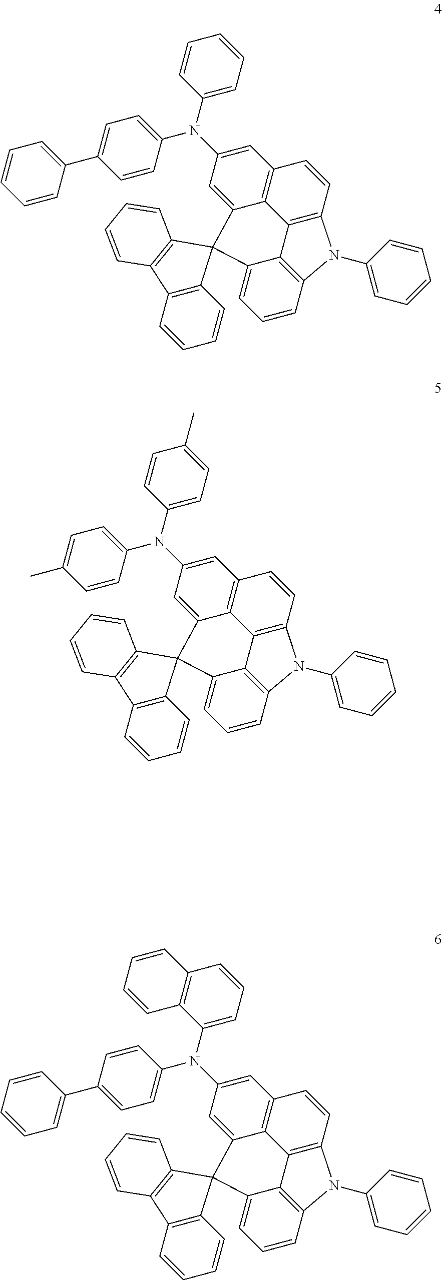 [SMILES: c1ccc(-c2ccc(N(c3ccccc3)c3cc4c5c(ccc6c5c5c(cccc5n6-c5ccccc5)C45c4ccccc4-c4ccccc45)c3)cc2)cc1 ; Cc1ccc(N(c2ccc(C)cc2)c2cc3c4c(ccc5c4c4c(cccc4n5-c4ccccc4)C34c3ccccc3-c3ccccc34)c2)cc1 ; c1ccc(-c2ccc(N(c3cc4c5c(ccc6c5c5c(cccc5n6-c5ccccc5)C45c4ccccc4-c4ccccc45)c3)c3cccc4ccccc34)cc2)cc1]

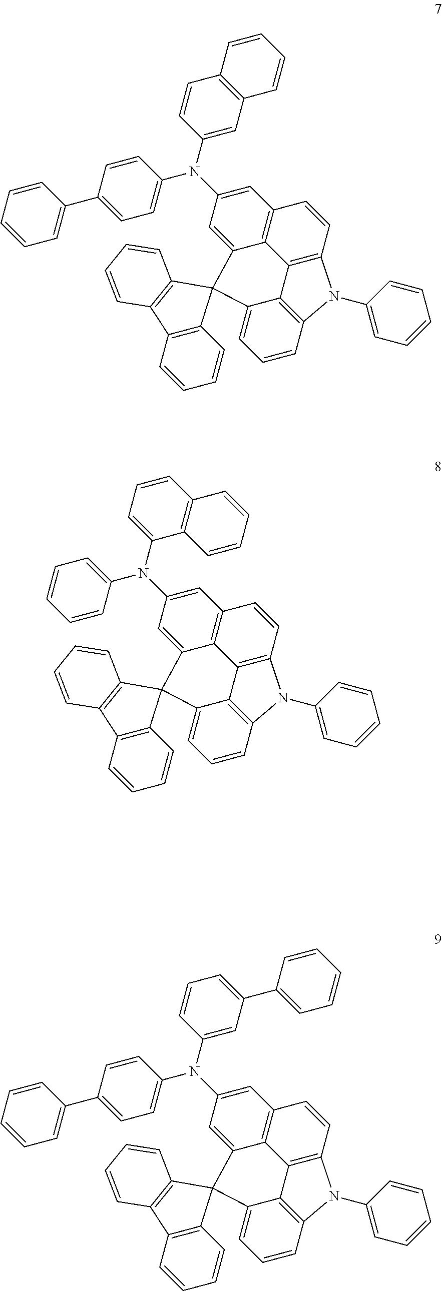 [SMILES: c1ccc(-c2ccc(N(c3ccc4ccccc4c3)c3cc4c5c(ccc6c5c5c(cccc5n6-c5ccccc5)C45c4ccccc4-c4ccccc45)c3)cc2)cc1 ; c1ccc(N(c2cc3c4c(ccc5c4c4c(cccc4n5-c4ccccc4)C34c3ccccc3-c3ccccc34)c2)c2cccc3ccccc23)cc1 ; c1ccc(-c2ccc(N(c3cccc(-c4ccccc4)c3)c3cc4c5c(ccc6c5c5c(cccc5n6-c5ccccc5)C45c4ccccc4-c4ccccc45)c3)cc2)cc1]

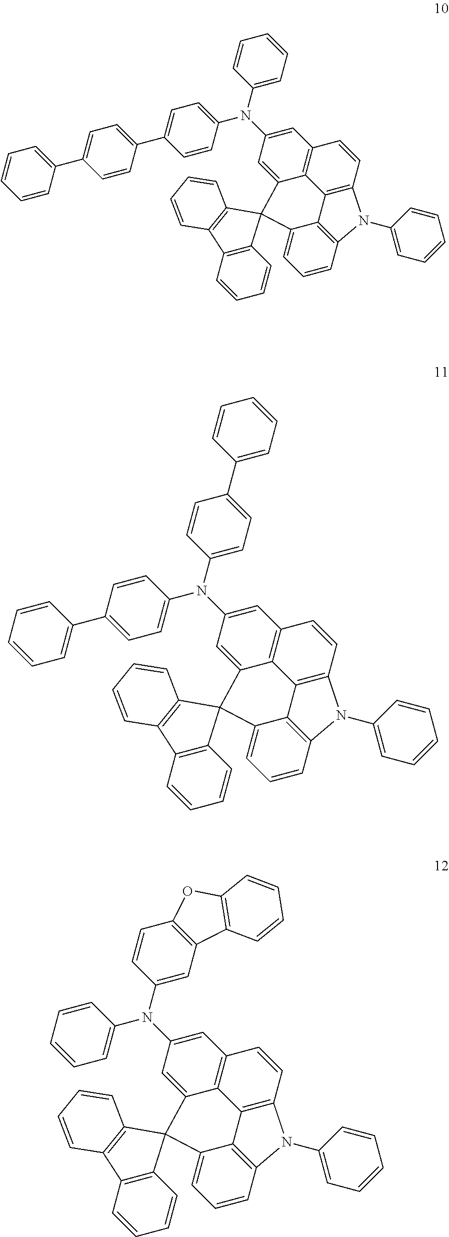 [SMILES: c1ccc(-c2ccc(-c3ccc(N(c4ccccc4)c4cc5c6c(ccc7c6c6c(cccc6n7-c6ccccc6)C56c5ccccc5-c5ccccc56)c4)cc3)cc2)cc1 ; c1ccc(-c2ccc(N(c3ccc(-c4ccccc4)cc3)c3cc4c5c(ccc6c5c5c(cccc5n6-c5ccccc5)C45c4ccccc4-c4ccccc45)c3)cc2)cc1 ; c1ccc(N(c2cc3c4c(ccc5c4c4c(cccc4n5-c4ccccc4)C34c3ccccc3-c3ccccc34)c2)c2ccc3oc4ccccc4c3c2)cc1]

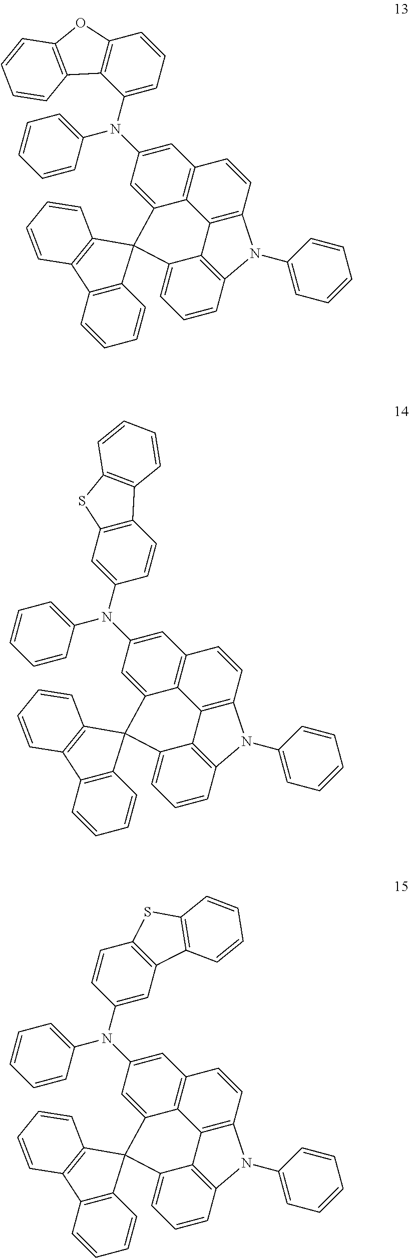 [SMILES: c1ccc(N(c2cc3c4c(ccc5c4c4c(cccc4n5-c4ccccc4)C34c3ccccc3-c3ccccc34)c2)c2cccc3oc4ccccc4c23)cc1 ; c1ccc(N(c2cc3c4c(ccc5c4c4c(cccc4n5-c4ccccc4)C34c3ccccc3-c3ccccc34)c2)c2ccc3c(c2)sc2ccccc23)cc1 ; c1ccc(N(c2cc3c4c(ccc5c4c4c(cccc4n5-c4ccccc4)C34c3ccccc3-c3ccccc34)c2)c2ccc3sc4ccccc4c3c2)cc1]

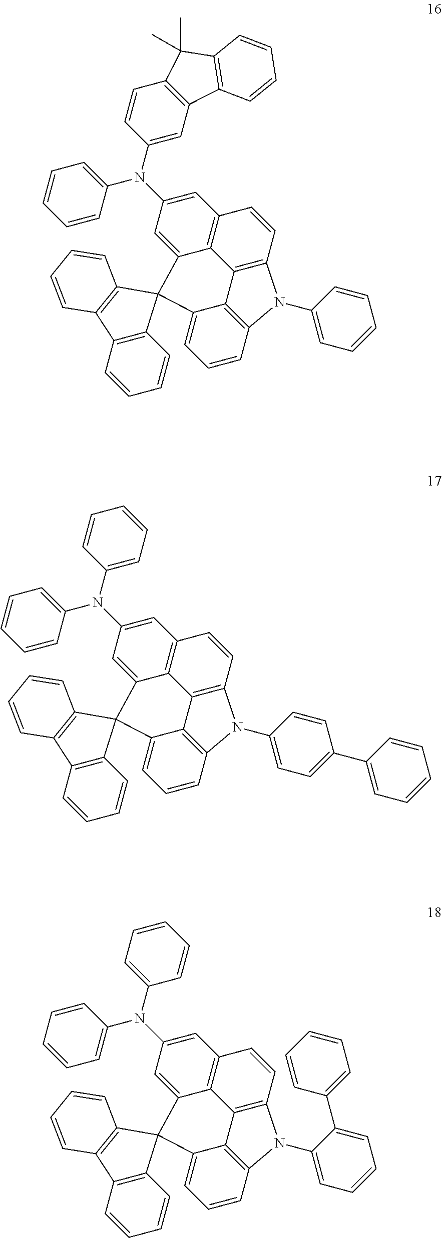 [SMILES: CC1(C)c2ccccc2-c2cc(N(c3ccccc3)c3cc4c5c(ccc6c5c5c(cccc5n6-c5ccccc5)C45c4ccccc4-c4ccccc45)c3)ccc21 ; c1ccc(-c2ccc(-n3c4cccc5c4c4c6c(cc(N(c7ccccc7)c7ccccc7)cc6ccc43)C53c4ccccc4-c4ccccc43)cc2)cc1 ; c1ccc(-c2ccccc2-n2c3cccc4c3c3c5c(cc(N(c6ccccc6)c6ccccc6)cc5ccc32)C42c3ccccc3-c3ccccc32)cc1]

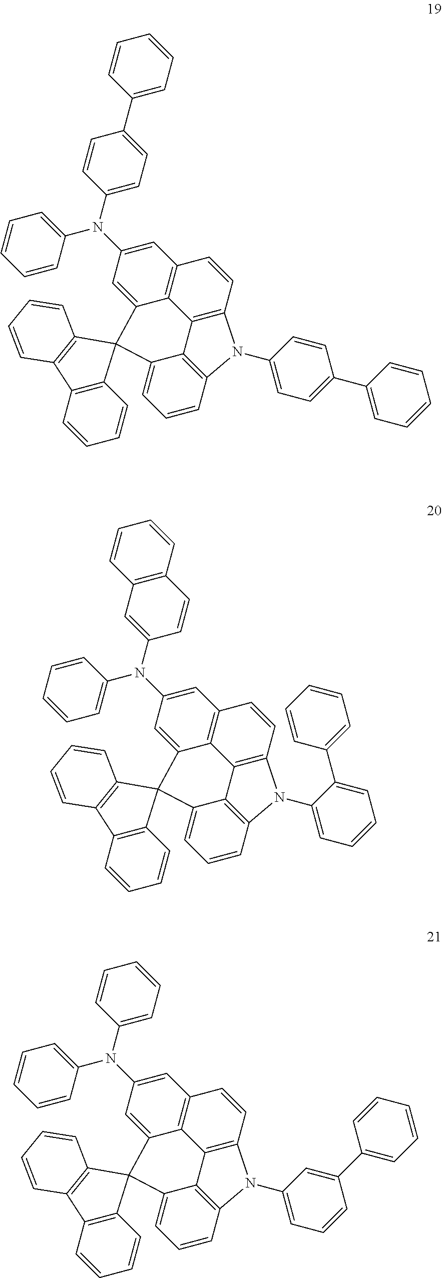 [SMILES: c1ccc(-c2ccc(N(c3ccccc3)c3cc4c5c(ccc6c5c5c(cccc5n6-c5ccc(-c6ccccc6)cc5)C45c4ccccc4-c4ccccc45)c3)cc2)cc1 ; c1ccc(-c2ccccc2-n2c3cccc4c3c3c5c(cc(N(c6ccccc6)c6ccc7ccccc7c6)cc5ccc32)C42c3ccccc3-c3ccccc32)cc1 ; c1ccc(-c2cccc(-n3c4cccc5c4c4c6c(cc(N(c7ccccc7)c7ccccc7)cc6ccc43)C53c4ccccc4-c4ccccc43)c2)cc1]

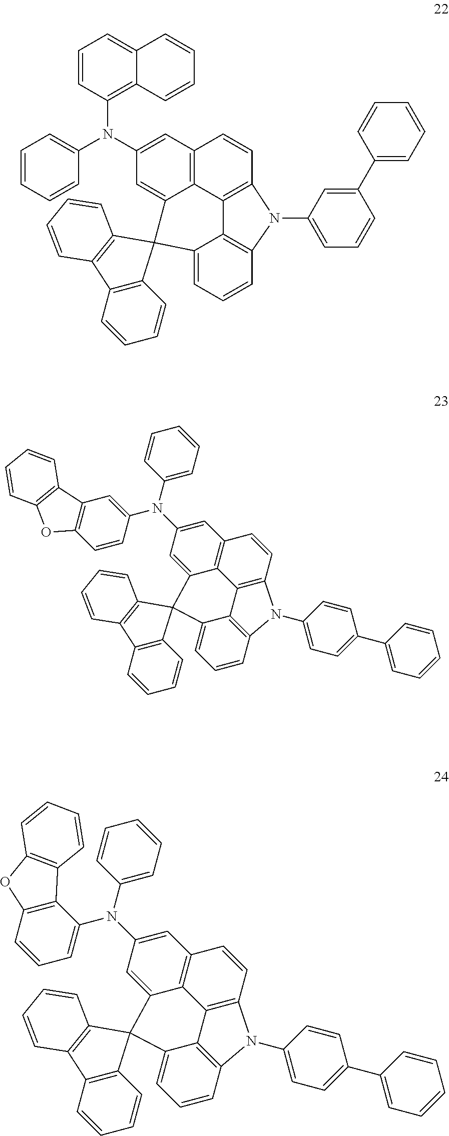 [SMILES: c1ccc(-c2cccc(-n3c4cccc5c4c4c6c(cc(N(c7ccccc7)c7cccc8ccccc78)cc6ccc43)C53c4ccccc4-c4ccccc43)c2)cc1 ; c1ccc(-c2ccc(-n3c4cccc5c4c4c6c(cc(N(c7ccccc7)c7ccc8oc9ccccc9c8c7)cc6ccc43)C53c4ccccc4-c4ccccc43)cc2)cc1 ; c1ccc(-c2ccc(-n3c4cccc5c4c4c6c(cc(N(c7ccccc7)c7cccc8oc9ccccc9c78)cc6ccc43)C53c4ccccc4-c4ccccc43)cc2)cc1]

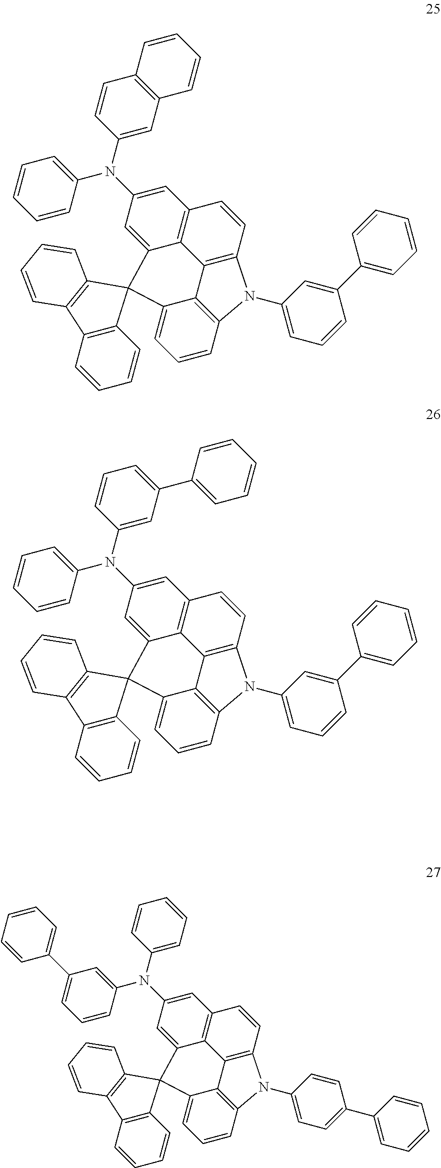 [SMILES: c1ccc(-c2cccc(-n3c4cccc5c4c4c6c(cc(N(c7ccccc7)c7ccc8ccccc8c7)cc6ccc43)C53c4ccccc4-c4ccccc43)c2)cc1 ; c1ccc(-c2cccc(N(c3ccccc3)c3cc4c5c(ccc6c5c5c(cccc5n6-c5cccc(-c6ccccc6)c5)C45c4ccccc4-c4ccccc45)c3)c2)cc1 ; c1ccc(-c2ccc(-n3c4cccc5c4c4c6c(cc(N(c7ccccc7)c7cccc(-c8ccccc8)c7)cc6ccc43)C53c4ccccc4-c4ccccc43)cc2)cc1]

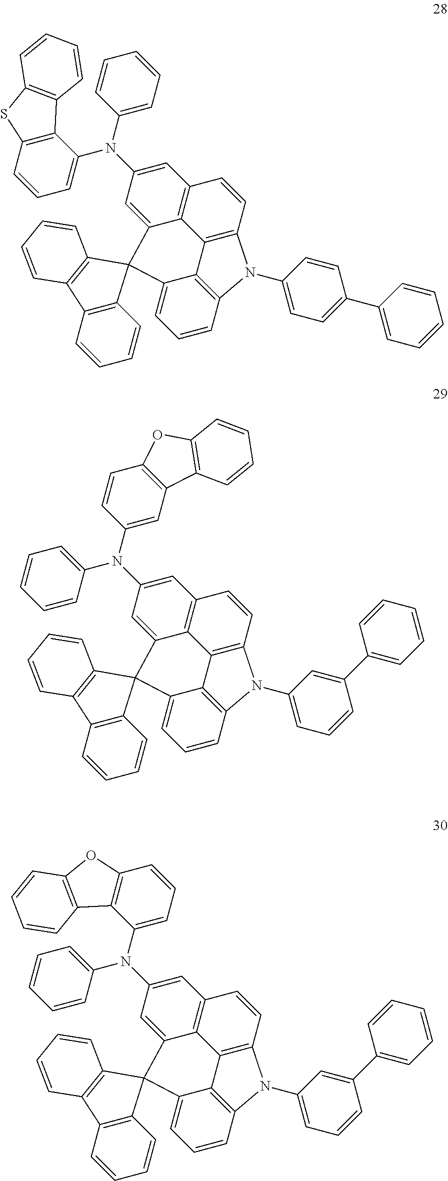 [SMILES: c1ccc(-c2ccc(-n3c4cccc5c4c4c6c(cc(N(c7ccccc7)c7cccc8sc9ccccc9c78)cc6ccc43)C53c4ccccc4-c4ccccc43)cc2)cc1 ; c1ccc(-c2cccc(-n3c4cccc5c4c4c6c(cc(N(c7ccccc7)c7ccc8oc9ccccc9c8c7)cc6ccc43)C53c4ccccc4-c4ccccc43)c2)cc1 ; c1ccc(-c2cccc(-n3c4cccc5c4c4c6c(cc(N(c7ccccc7)c7cccc8oc9ccccc9c78)cc6ccc43)C53c4ccccc4-c4ccccc43)c2)cc1]

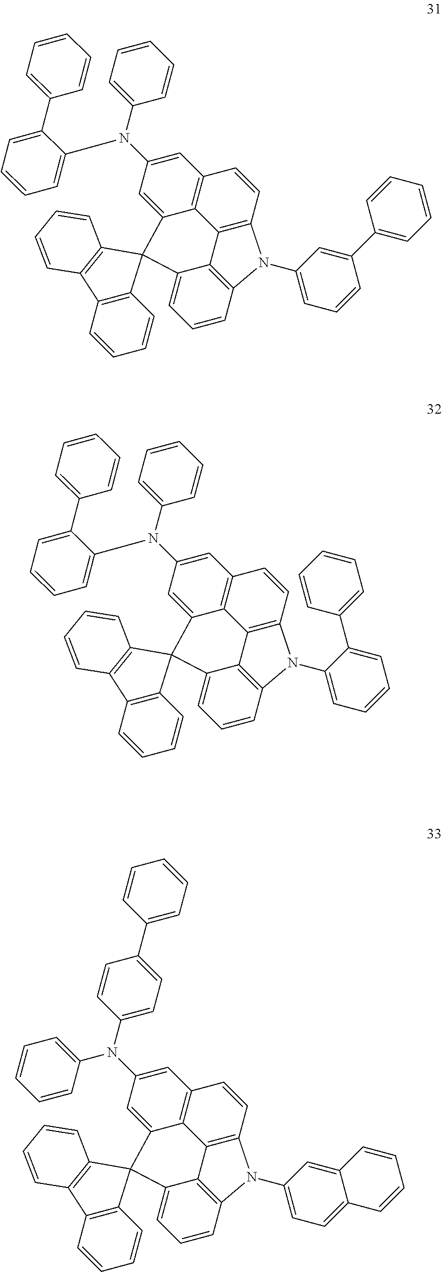 [SMILES: c1ccc(-c2cccc(-n3c4cccc5c4c4c6c(cc(N(c7ccccc7)c7ccccc7-c7ccccc7)cc6ccc43)C53c4ccccc4-c4ccccc43)c2)cc1 ; c1ccc(-c2ccccc2N(c2ccccc2)c2cc3c4c(ccc5c4c4c(cccc4n5-c4ccccc4-c4ccccc4)C34c3ccccc3-c3ccccc34)c2)cc1 ; c1ccc(-c2ccc(N(c3ccccc3)c3cc4c5c(ccc6c5c5c(cccc5n6-c5ccc6ccccc6c5)C45c4ccccc4-c4ccccc45)c3)cc2)cc1]

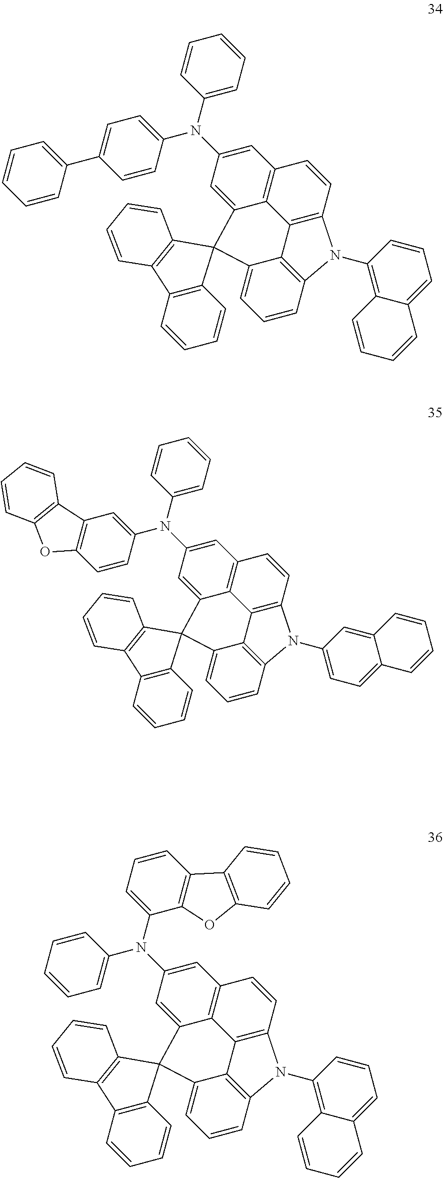 [SMILES: c1ccc(-c2ccc(N(c3ccccc3)c3cc4c5c(ccc6c5c5c(cccc5n6-c5cccc6ccccc56)C45c4ccccc4-c4ccccc45)c3)cc2)cc1 ; c1ccc(N(c2cc3c4c(ccc5c4c4c(cccc4n5-c4ccc5ccccc5c4)C34c3ccccc3-c3ccccc34)c2)c2ccc3oc4ccccc4c3c2)cc1 ; c1ccc(N(c2cc3c4c(ccc5c4c4c(cccc4n5-c4cccc5ccccc45)C34c3ccccc3-c3ccccc34)c2)c2cccc3c2oc2ccccc23)cc1]

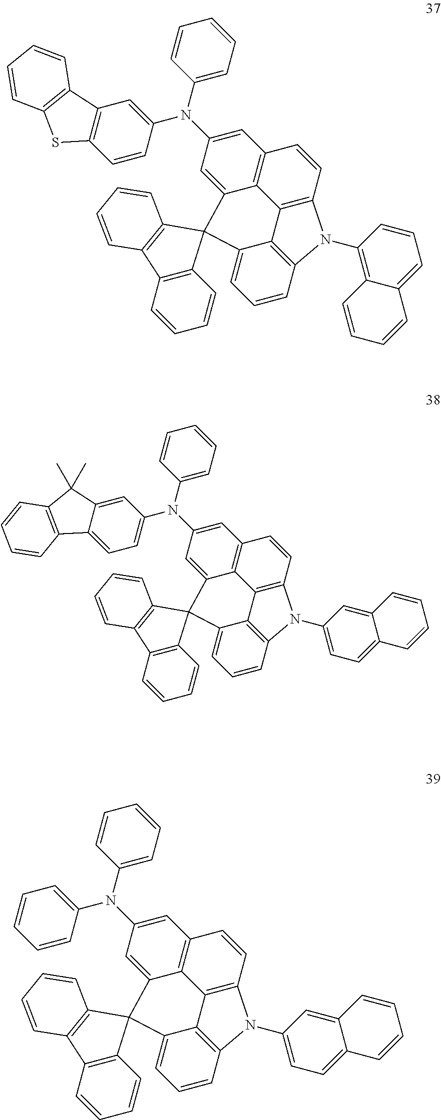 [SMILES: c1ccc(N(c2cc3c4c(ccc5c4c4c(cccc4n5-c4cccc5ccccc45)C34c3ccccc3-c3ccccc34)c2)c2ccc3sc4ccccc4c3c2)cc1 ; CC1(C)c2ccccc2-c2ccc(N(c3ccccc3)c3cc4c5c(ccc6c5c5c(cccc5n6-c5ccc6ccccc6c5)C45c4ccccc4-c4ccccc45)c3)cc21 ; c1ccc(N(c2ccccc2)c2cc3c4c(ccc5c4c4c(cccc4n5-c4ccc5ccccc5c4)C34c3ccccc3-c3ccccc34)c2)cc1]

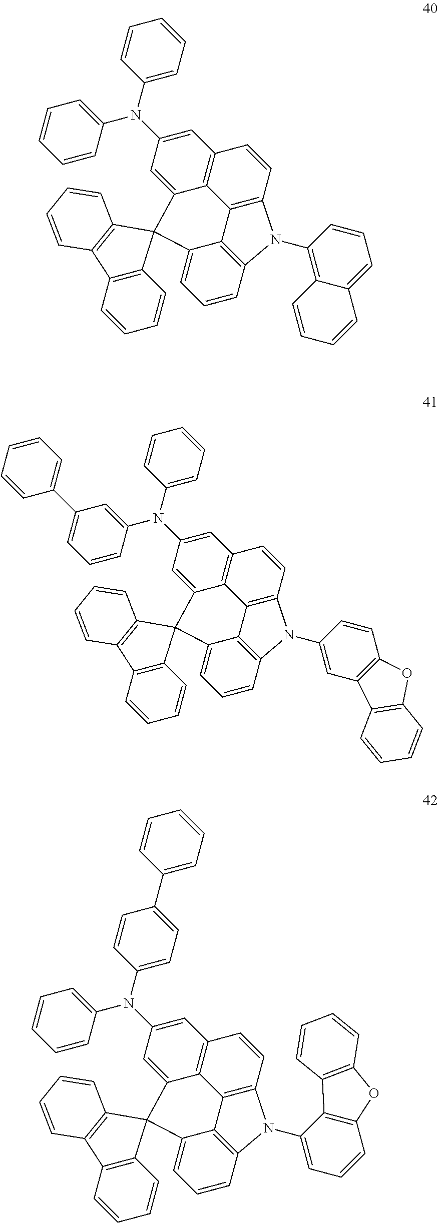 [SMILES: c1ccc(N(c2ccccc2)c2cc3c4c(ccc5c4c4c(cccc4n5-c4cccc5ccccc45)C34c3ccccc3-c3ccccc34)c2)cc1 ; c1ccc(-c2cccc(N(c3ccccc3)c3cc4c5c(ccc6c5c5c(cccc5n6-c5ccc6oc7ccccc7c6c5)C45c4ccccc4-c4ccccc45)c3)c2)cc1 ; c1ccc(-c2ccc(N(c3ccccc3)c3cc4c5c(ccc6c5c5c(cccc5n6-c5cccc6oc7ccccc7c56)C45c4ccccc4-c4ccccc45)c3)cc2)cc1]

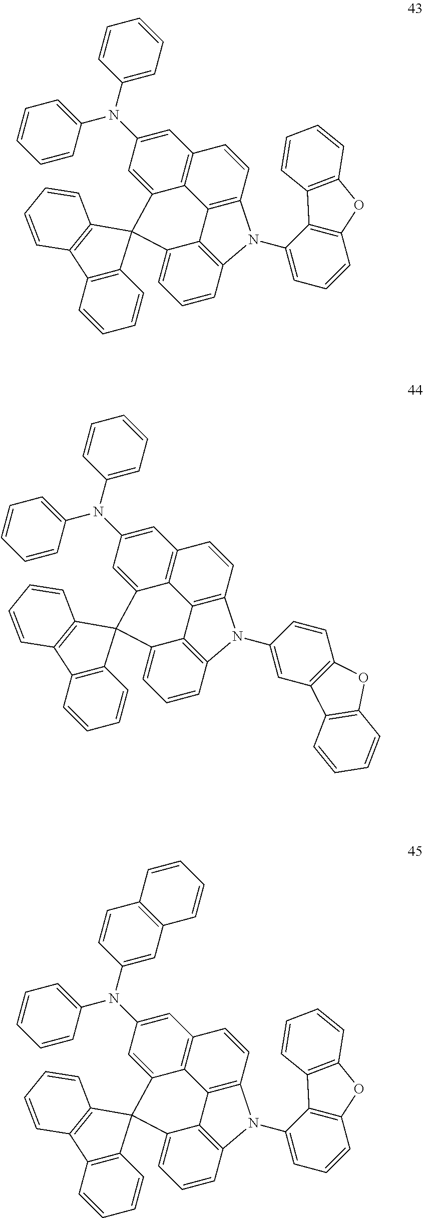 [SMILES: c1ccc(N(c2ccccc2)c2cc3c4c(ccc5c4c4c(cccc4n5-c4cccc5oc6ccccc6c45)C34c3ccccc3-c3ccccc34)c2)cc1 ; c1ccc(N(c2ccccc2)c2cc3c4c(ccc5c4c4c(cccc4n5-c4ccc5oc6ccccc6c5c4)C34c3ccccc3-c3ccccc34)c2)cc1 ; c1ccc(N(c2ccc3ccccc3c2)c2cc3c4c(ccc5c4c4c(cccc4n5-c4cccc5oc6ccccc6c45)C34c3ccccc3-c3ccccc34)c2)cc1]

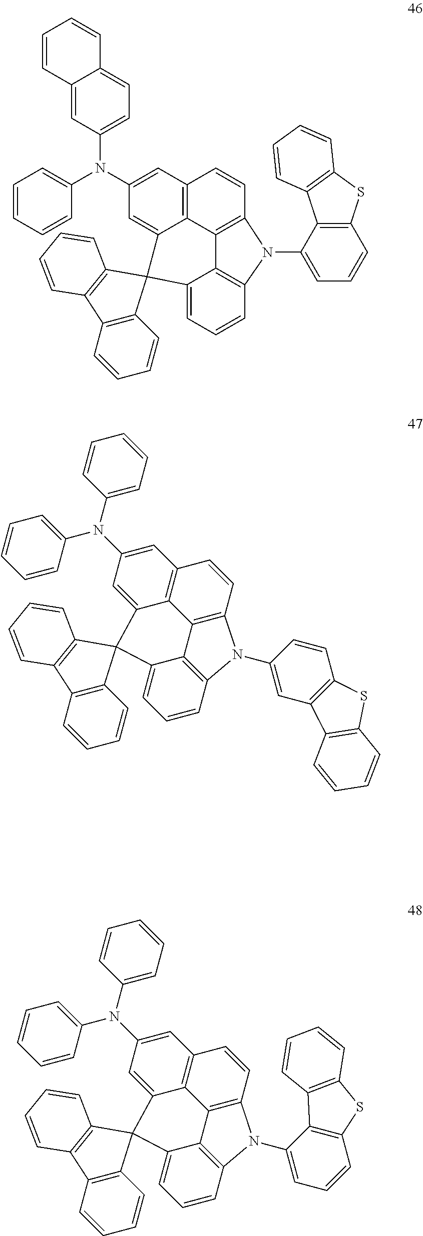 [SMILES: c1ccc(N(c2ccc3ccccc3c2)c2cc3c4c(ccc5c4c4c(cccc4n5-c4cccc5sc6ccccc6c45)C34c3ccccc3-c3ccccc34)c2)cc1 ; c1ccc(N(c2ccccc2)c2cc3c4c(ccc5c4c4c(cccc4n5-c4ccc5sc6ccccc6c5c4)C34c3ccccc3-c3ccccc34)c2)cc1 ; c1ccc(N(c2ccccc2)c2cc3c4c(ccc5c4c4c(cccc4n5-c4cccc5sc6ccccc6c45)C34c3ccccc3-c3ccccc34)c2)cc1]

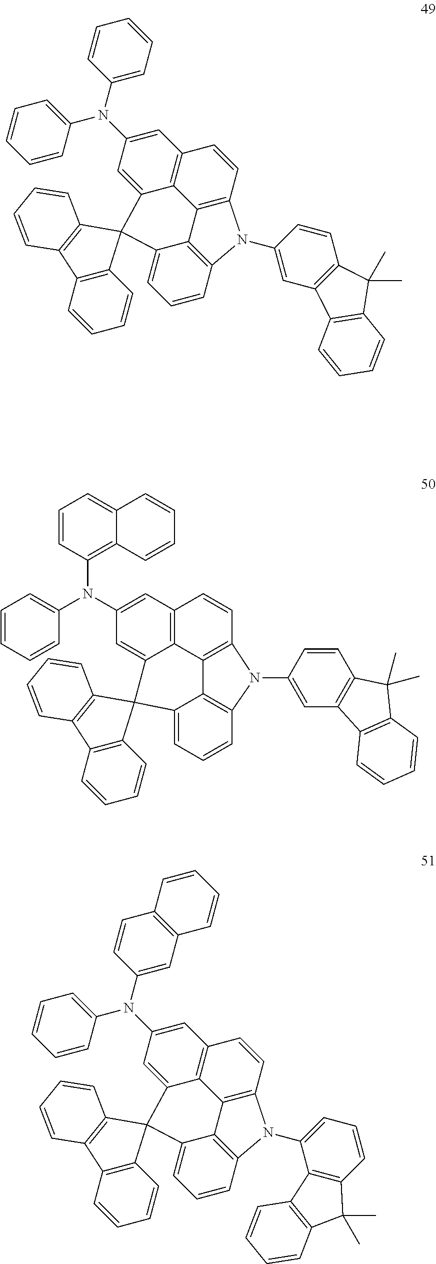 [SMILES: CC1(C)c2ccccc2-c2cc(-n3c4cccc5c4c4c6c(cc(N(c7ccccc7)c7ccccc7)cc6ccc43)C53c4ccccc4-c4ccccc43)ccc21 ; CC1(C)c2ccccc2-c2cc(-n3c4cccc5c4c4c6c(cc(N(c7ccccc7)c7cccc8ccccc78)cc6ccc43)C53c4ccccc4-c4ccccc43)ccc21 ; CC1(C)c2ccccc2-c2c(-n3c4cccc5c4c4c6c(cc(N(c7ccccc7)c7ccc8ccccc8c7)cc6ccc43)C53c4ccccc4-c4ccccc43)cccc21]

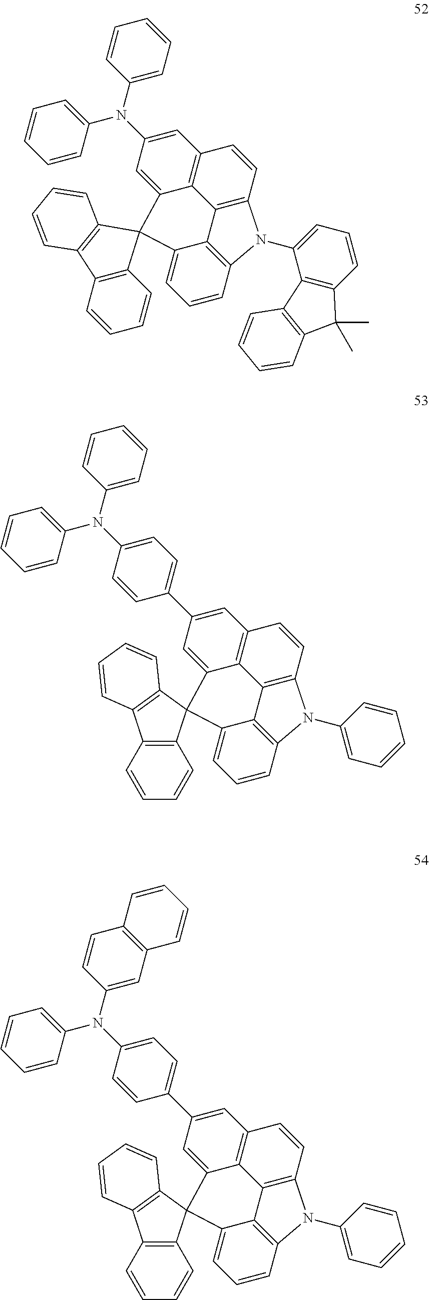 [SMILES: CC1(C)c2ccccc2-c2c(-n3c4cccc5c4c4c6c(cc(N(c7ccccc7)c7ccccc7)cc6ccc43)C53c4ccccc4-c4ccccc43)cccc21 ; c1ccc(N(c2ccccc2)c2ccc(-c3cc4c5c(ccc6c5c5c(cccc5n6-c5ccccc5)C45c4ccccc4-c4ccccc45)c3)cc2)cc1 ; c1ccc(N(c2ccc(-c3cc4c5c(ccc6c5c5c(cccc5n6-c5ccccc5)C45c4ccccc4-c4ccccc45)c3)cc2)c2ccc3ccccc3c2)cc1]

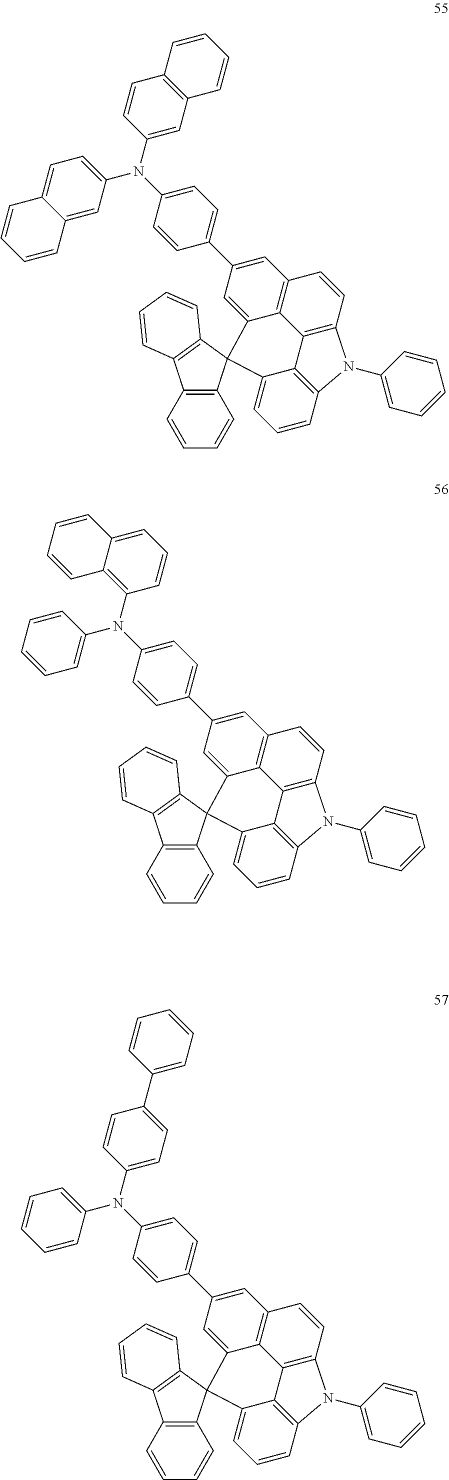 [SMILES: c1ccc(-n2c3cccc4c3c3c5c(cc(-c6ccc(N(c7ccc8ccccc8c7)c7ccc8ccccc8c7)cc6)cc5ccc32)C42c3ccccc3-c3ccccc32)cc1 ; c1ccc(N(c2ccc(-c3cc4c5c(ccc6c5c5c(cccc5n6-c5ccccc5)C45c4ccccc4-c4ccccc45)c3)cc2)c2cccc3ccccc23)cc1 ; c1ccc(-c2ccc(N(c3ccccc3)c3ccc(-c4cc5c6c(ccc7c6c6c(cccc6n7-c6ccccc6)C56c5ccccc5-c5ccccc56)c4)cc3)cc2)cc1]

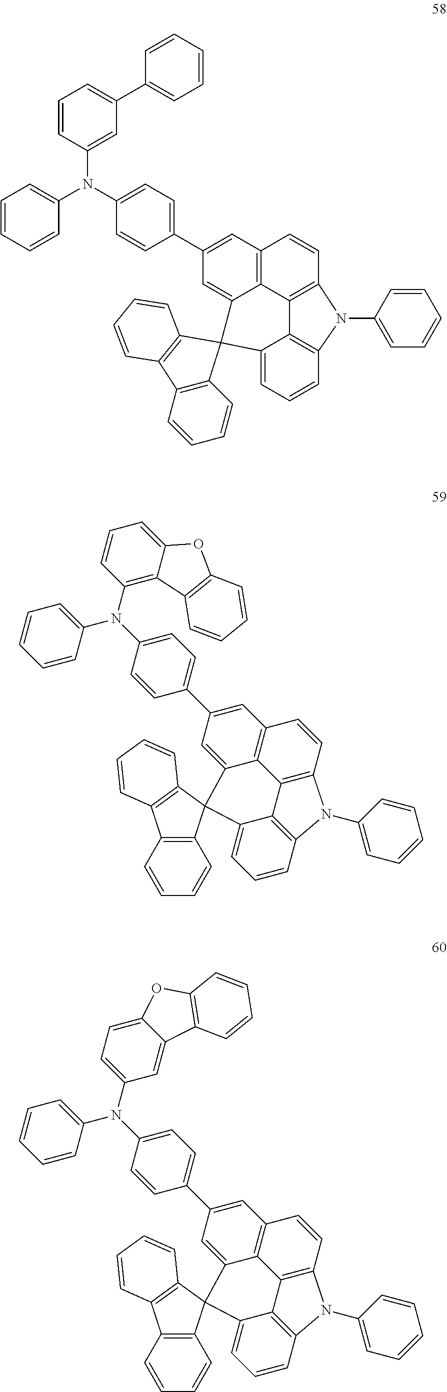 [SMILES: c1ccc(-c2cccc(N(c3ccccc3)c3ccc(-c4cc5c6c(ccc7c6c6c(cccc6n7-c6ccccc6)C56c5ccccc5-c5ccccc56)c4)cc3)c2)cc1 ; c1ccc(N(c2ccc(-c3cc4c5c(ccc6c5c5c(cccc5n6-c5ccccc5)C45c4ccccc4-c4ccccc45)c3)cc2)c2cccc3oc4ccccc4c23)cc1 ; c1ccc(N(c2ccc(-c3cc4c5c(ccc6c5c5c(cccc5n6-c5ccccc5)C45c4ccccc4-c4ccccc45)c3)cc2)c2ccc3oc4ccccc4c3c2)cc1]

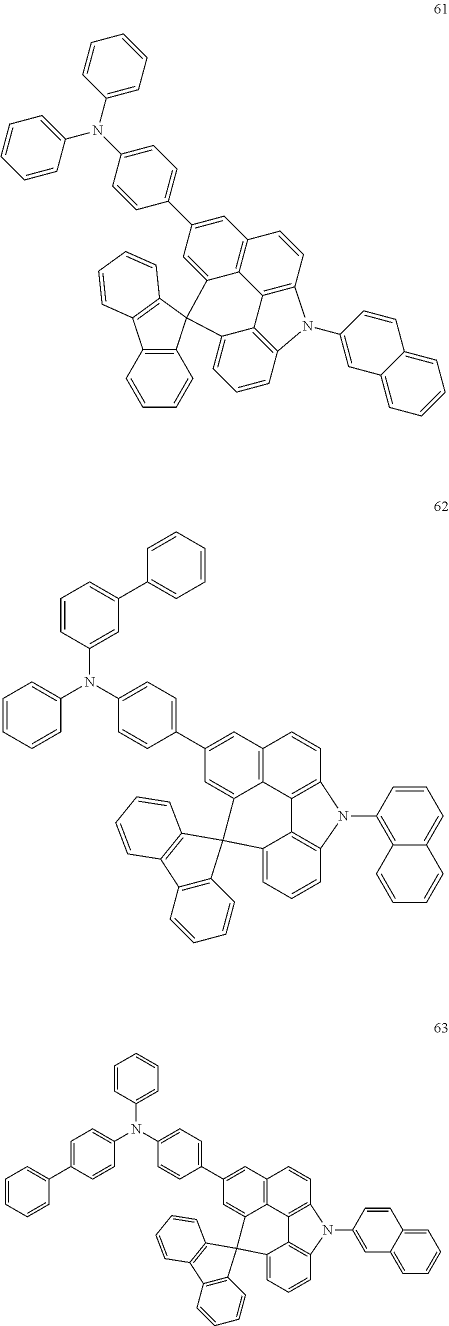 [SMILES: c1ccc(N(c2ccccc2)c2ccc(-c3cc4c5c(ccc6c5c5c(cccc5n6-c5ccc6ccccc6c5)C45c4ccccc4-c4ccccc45)c3)cc2)cc1 ; c1ccc(-c2cccc(N(c3ccccc3)c3ccc(-c4cc5c6c(ccc7c6c6c(cccc6n7-c6cccc7ccccc67)C56c5ccccc5-c5ccccc56)c4)cc3)c2)cc1 ; c1ccc(-c2ccc(N(c3ccccc3)c3ccc(-c4cc5c6c(ccc7c6c6c(cccc6n7-c6ccc7ccccc7c6)C56c5ccccc5-c5ccccc56)c4)cc3)cc2)cc1]

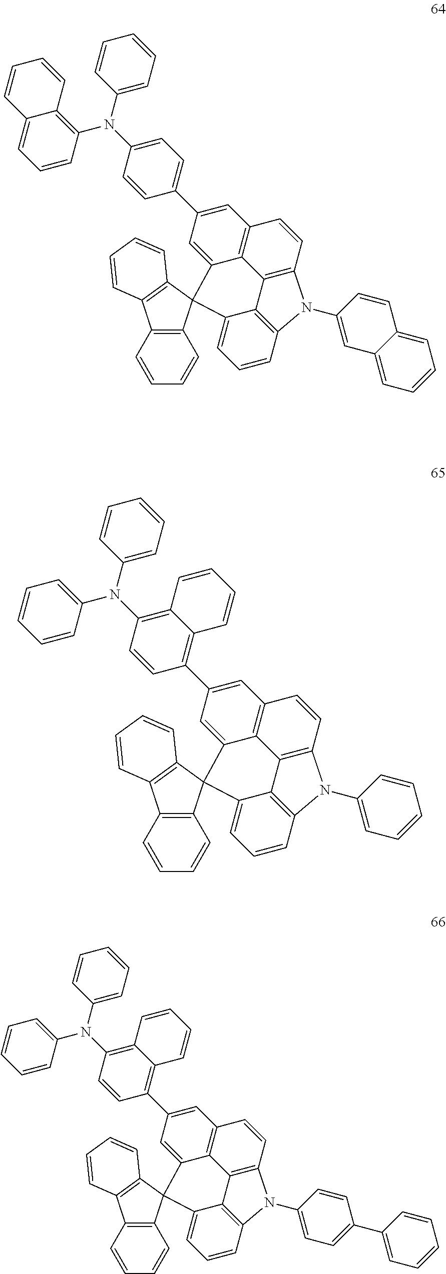 [SMILES: c1ccc(N(c2ccc(-c3cc4c5c(ccc6c5c5c(cccc5n6-c5ccc6ccccc6c5)C45c4ccccc4-c4ccccc45)c3)cc2)c2cccc3ccccc23)cc1 ; c1ccc(N(c2ccccc2)c2ccc(-c3cc4c5c(ccc6c5c5c(cccc5n6-c5ccccc5)C45c4ccccc4-c4ccccc45)c3)c3ccccc23)cc1 ; c1ccc(-c2ccc(-n3c4cccc5c4c4c6c(cc(-c7ccc(N(c8ccccc8)c8ccccc8)c8ccccc78)cc6ccc43)C53c4ccccc4-c4ccccc43)cc2)cc1]

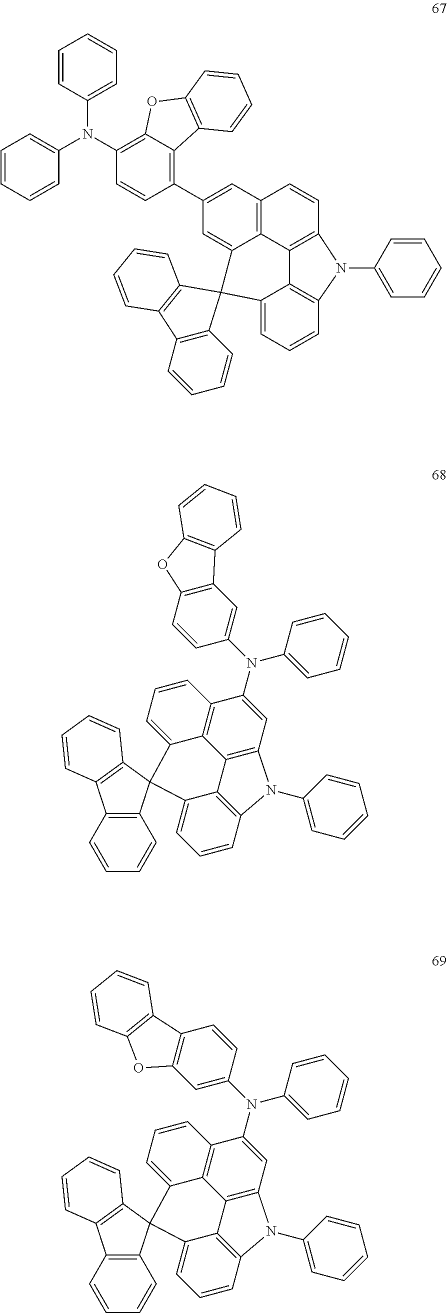 [SMILES: c1ccc(N(c2ccccc2)c2ccc(-c3cc4c5c(ccc6c5c5c(cccc5n6-c5ccccc5)C45c4ccccc4-c4ccccc45)c3)c3c2oc2ccccc23)cc1 ; c1ccc(N(c2ccc3oc4ccccc4c3c2)c2cc3c4c5c(cccc25)C2(c5ccccc5-c5ccccc52)c2cccc(c24)n3-c2ccccc2)cc1 ; c1ccc(N(c2ccc3c(c2)oc2ccccc23)c2cc3c4c5c(cccc25)C2(c5ccccc5-c5ccccc52)c2cccc(c24)n3-c2ccccc2)cc1]

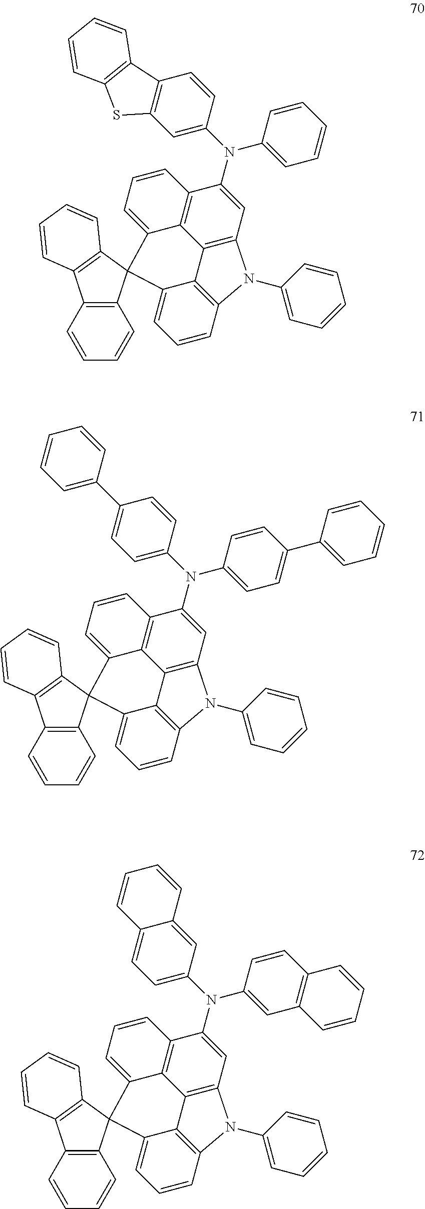 [SMILES: c1ccc(N(c2ccc3c(c2)sc2ccccc23)c2cc3c4c5c(cccc25)C2(c5ccccc5-c5ccccc52)c2cccc(c24)n3-c2ccccc2)cc1 ; c1ccc(-c2ccc(N(c3ccc(-c4ccccc4)cc3)c3cc4c5c6c(cccc36)C3(c6ccccc6-c6ccccc63)c3cccc(c35)n4-c3ccccc3)cc2)cc1 ; c1ccc(-n2c3cccc4c3c3c5c(cccc5c(N(c5ccc6ccccc6c5)c5ccc6ccccc6c5)cc32)C42c3ccccc3-c3ccccc32)cc1]

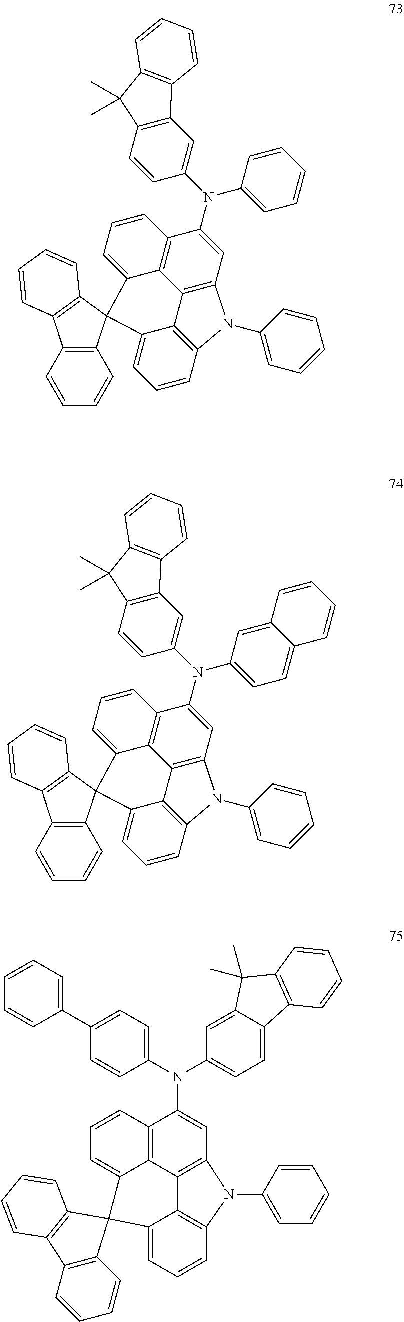 [SMILES: CC1(C)c2ccccc2-c2cc(N(c3ccccc3)c3cc4c5c6c(cccc36)C3(c6ccccc6-c6ccccc63)c3cccc(c35)n4-c3ccccc3)ccc21 ; CC1(C)c2ccccc2-c2cc(N(c3ccc4ccccc4c3)c3cc4c5c6c(cccc36)C3(c6ccccc6-c6ccccc63)c3cccc(c35)n4-c3ccccc3)ccc21 ; CC1(C)c2ccccc2-c2ccc(N(c3ccc(-c4ccccc4)cc3)c3cc4c5c6c(cccc36)C3(c6ccccc6-c6ccccc63)c3cccc(c35)n4-c3ccccc3)cc21]

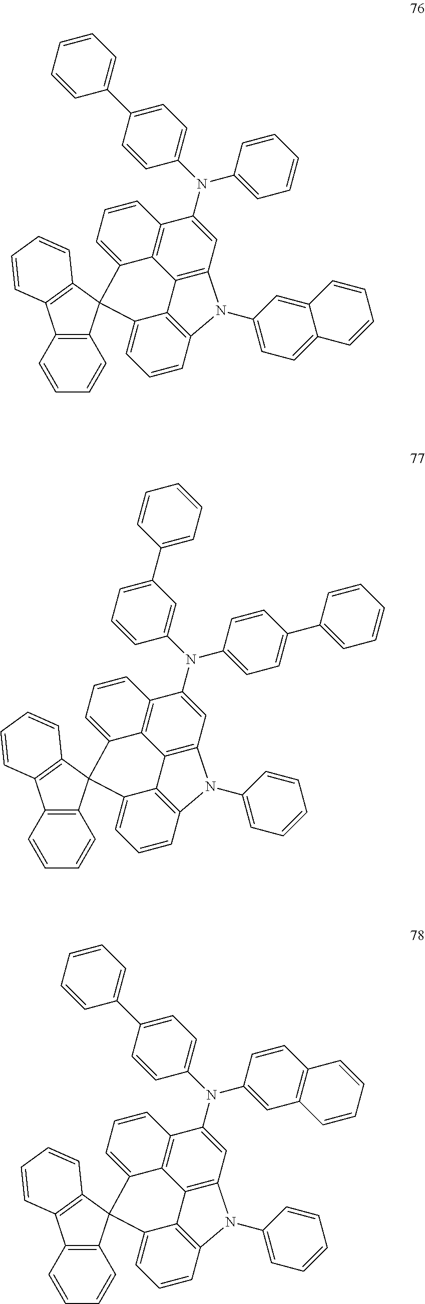 [SMILES: c1ccc(-c2ccc(N(c3ccccc3)c3cc4c5c6c(cccc36)C3(c6ccccc6-c6ccccc63)c3cccc(c35)n4-c3ccc4ccccc4c3)cc2)cc1 ; c1ccc(-c2ccc(N(c3cccc(-c4ccccc4)c3)c3cc4c5c6c(cccc36)C3(c6ccccc6-c6ccccc63)c3cccc(c35)n4-c3ccccc3)cc2)cc1 ; c1ccc(-c2ccc(N(c3ccc4ccccc4c3)c3cc4c5c6c(cccc36)C3(c6ccccc6-c6ccccc63)c3cccc(c35)n4-c3ccccc3)cc2)cc1]

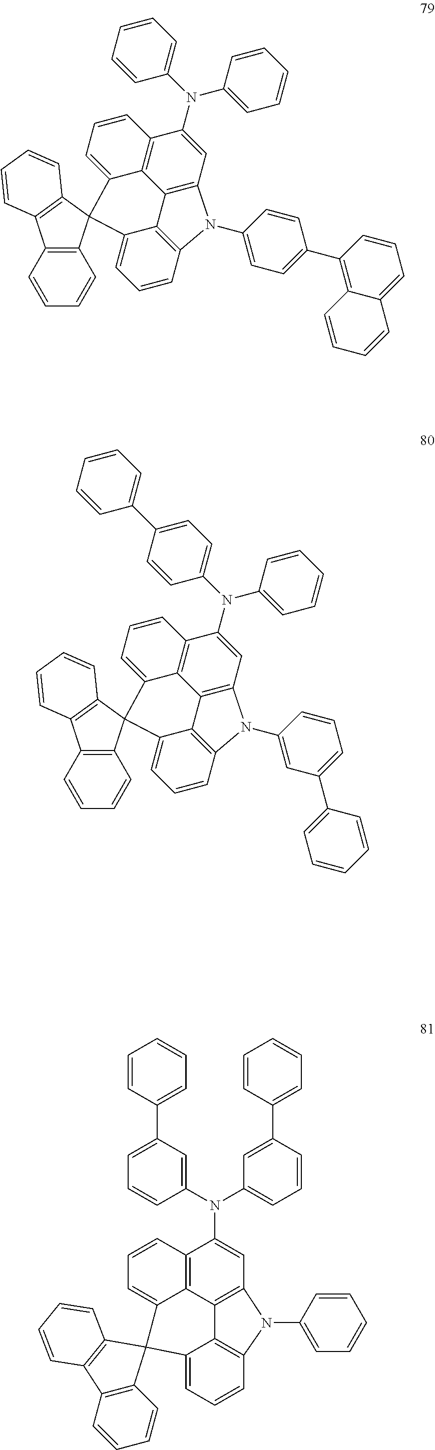 [SMILES: c1ccc(N(c2ccccc2)c2cc3c4c5c(cccc25)C2(c5ccccc5-c5ccccc52)c2cccc(c24)n3-c2ccc(-c3cccc4ccccc34)cc2)cc1 ; c1ccc(-c2ccc(N(c3ccccc3)c3cc4c5c6c(cccc36)C3(c6ccccc6-c6ccccc63)c3cccc(c35)n4-c3cccc(-c4ccccc4)c3)cc2)cc1 ; c1ccc(-c2cccc(N(c3cccc(-c4ccccc4)c3)c3cc4c5c6c(cccc36)C3(c6ccccc6-c6ccccc63)c3cccc(c35)n4-c3ccccc3)c2)cc1]

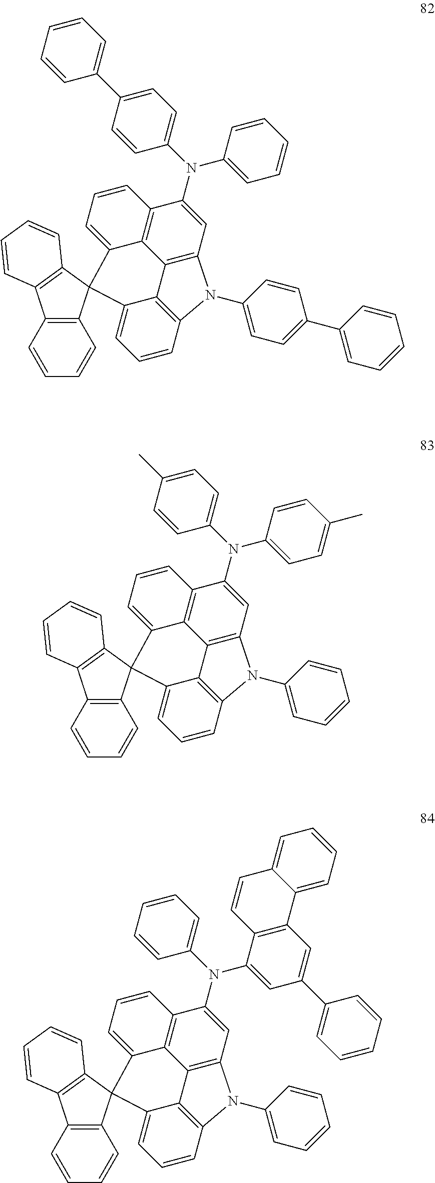 [SMILES: c1ccc(-c2ccc(N(c3ccccc3)c3cc4c5c6c(cccc36)C3(c6ccccc6-c6ccccc63)c3cccc(c35)n4-c3ccc(-c4ccccc4)cc3)cc2)cc1 ; Cc1ccc(N(c2ccc(C)cc2)c2cc3c4c5c(cccc25)C2(c5ccccc5-c5ccccc52)c2cccc(c24)n3-c2ccccc2)cc1 ; c1ccc(-c2cc(N(c3ccccc3)c3cc4c5c6c(cccc36)C3(c6ccccc6-c6ccccc63)c3cccc(c35)n4-c3ccccc3)c3ccc4ccccc4c3c2)cc1]

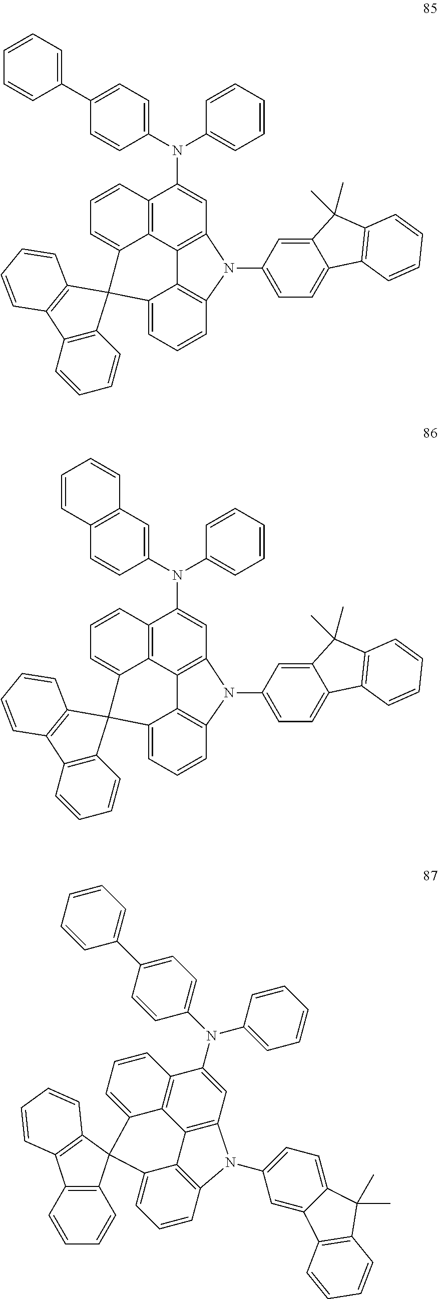 [SMILES: CC1(C)c2ccccc2-c2ccc(-n3c4cccc5c4c4c6c(cccc6c(N(c6ccccc6)c6ccc(-c7ccccc7)cc6)cc43)C53c4ccccc4-c4ccccc43)cc21 ; CC1(C)c2ccccc2-c2ccc(-n3c4cccc5c4c4c6c(cccc6c(N(c6ccccc6)c6ccc7ccccc7c6)cc43)C53c4ccccc4-c4ccccc43)cc21 ; CC1(C)c2ccccc2-c2cc(-n3c4cccc5c4c4c6c(cccc6c(N(c6ccccc6)c6ccc(-c7ccccc7)cc6)cc43)C53c4ccccc4-c4ccccc43)ccc21]

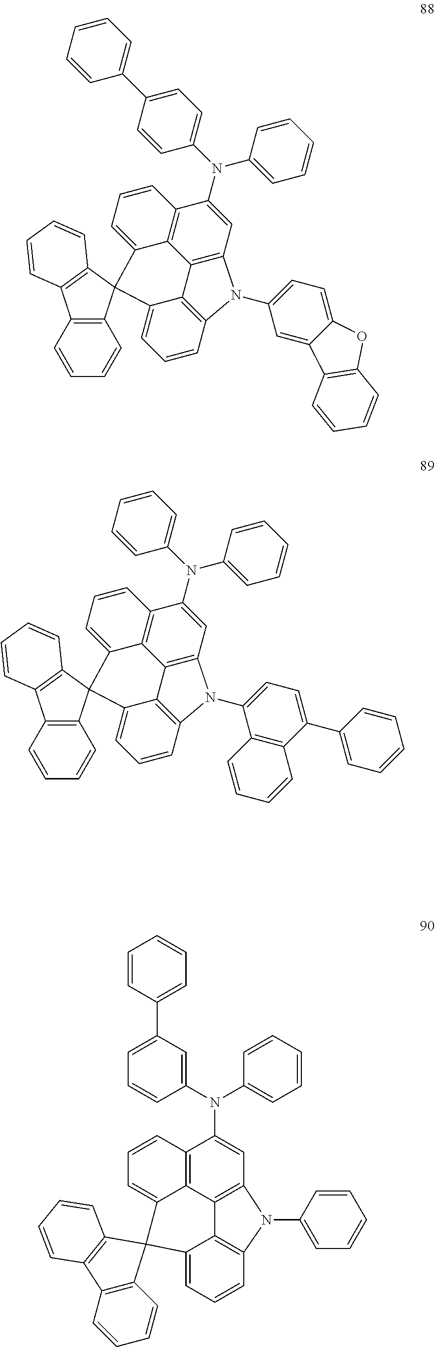 [SMILES: c1ccc(-c2ccc(N(c3ccccc3)c3cc4c5c6c(cccc36)C3(c6ccccc6-c6ccccc63)c3cccc(c35)n4-c3ccc4oc5ccccc5c4c3)cc2)cc1 ; c1ccc(-c2ccc(-n3c4cccc5c4c4c6c(cccc6c(N(c6ccccc6)c6ccccc6)cc43)C53c4ccccc4-c4ccccc43)c3ccccc23)cc1 ; c1ccc(-c2cccc(N(c3ccccc3)c3cc4c5c6c(cccc36)C3(c6ccccc6-c6ccccc63)c3cccc(c35)n4-c3ccccc3)c2)cc1]

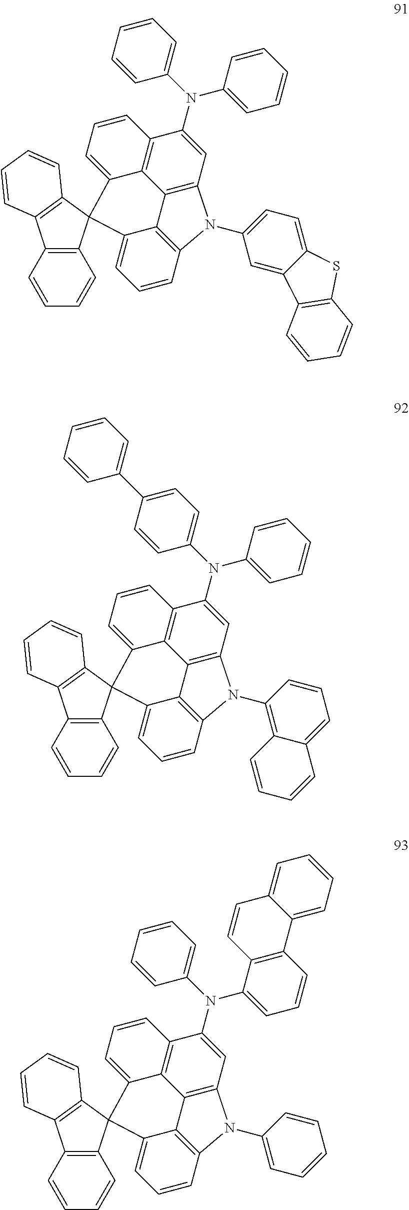 [SMILES: c1ccc(N(c2ccccc2)c2cc3c4c5c(cccc25)C2(c5ccccc5-c5ccccc52)c2cccc(c24)n3-c2ccc3sc4ccccc4c3c2)cc1 ; c1ccc(-c2ccc(N(c3ccccc3)c3cc4c5c6c(cccc36)C3(c6ccccc6-c6ccccc63)c3cccc(c35)n4-c3cccc4ccccc34)cc2)cc1 ; c1ccc(N(c2cccc3c2ccc2ccccc23)c2cc3c4c5c(cccc25)C2(c5ccccc5-c5ccccc52)c2cccc(c24)n3-c2ccccc2)cc1]

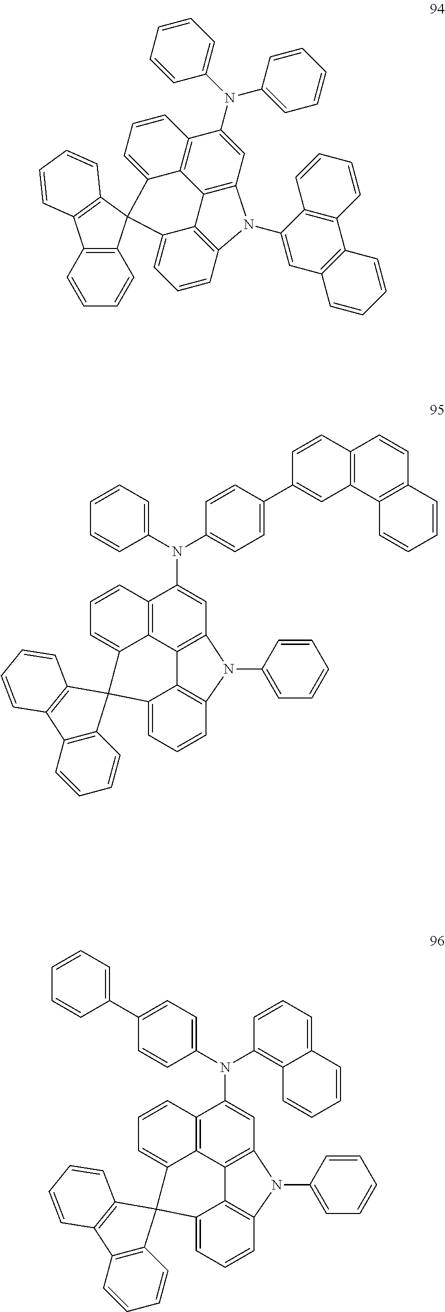 [SMILES: c1ccc(N(c2ccccc2)c2cc3c4c5c(cccc25)C2(c5ccccc5-c5ccccc52)c2cccc(c24)n3-c2cc3ccccc3c3ccccc23)cc1 ; c1ccc(N(c2ccc(-c3ccc4ccc5ccccc5c4c3)cc2)c2cc3c4c5c(cccc25)C2(c5ccccc5-c5ccccc52)c2cccc(c24)n3-c2ccccc2)cc1 ; c1ccc(-c2ccc(N(c3cccc4ccccc34)c3cc4c5c6c(cccc36)C3(c6ccccc6-c6ccccc63)c3cccc(c35)n4-c3ccccc3)cc2)cc1]

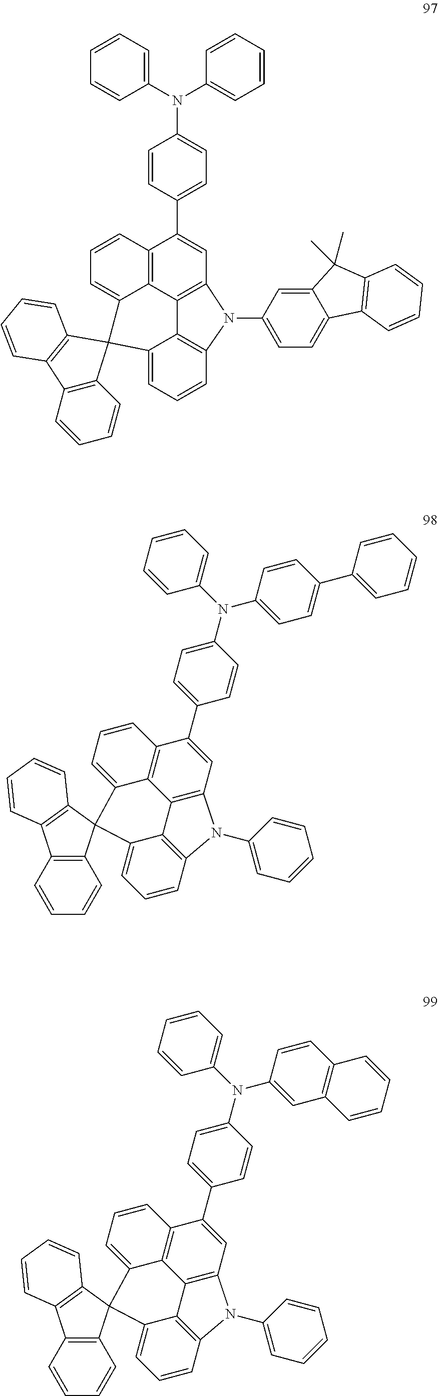 [SMILES: CC1(C)c2ccccc2-c2ccc(-n3c4cccc5c4c4c6c(cccc6c(-c6ccc(N(c7ccccc7)c7ccccc7)cc6)cc43)C53c4ccccc4-c4ccccc43)cc21 ; c1ccc(-c2ccc(N(c3ccccc3)c3ccc(-c4cc5c6c7c(cccc47)C4(c7ccccc7-c7ccccc74)c4cccc(c46)n5-c4ccccc4)cc3)cc2)cc1 ; c1ccc(N(c2ccc(-c3cc4c5c6c(cccc36)C3(c6ccccc6-c6ccccc63)c3cccc(c35)n4-c3ccccc3)cc2)c2ccc3ccccc3c2)cc1]

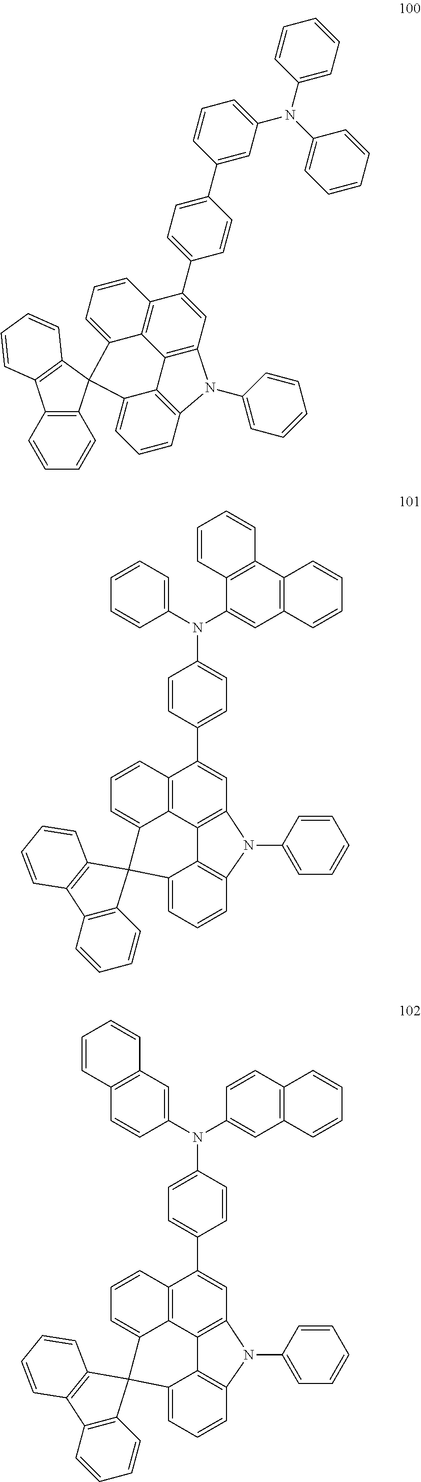 [SMILES: c1ccc(N(c2ccccc2)c2cccc(-c3ccc(-c4cc5c6c7c(cccc47)C4(c7ccccc7-c7ccccc74)c4cccc(c46)n5-c4ccccc4)cc3)c2)cc1 ; c1ccc(N(c2ccc(-c3cc4c5c6c(cccc36)C3(c6ccccc6-c6ccccc63)c3cccc(c35)n4-c3ccccc3)cc2)c2cc3ccccc3c3ccccc23)cc1 ; c1ccc(-n2c3cccc4c3c3c5c(cccc5c(-c5ccc(N(c6ccc7ccccc7c6)c6ccc7ccccc7c6)cc5)cc32)C42c3ccccc3-c3ccccc32)cc1]

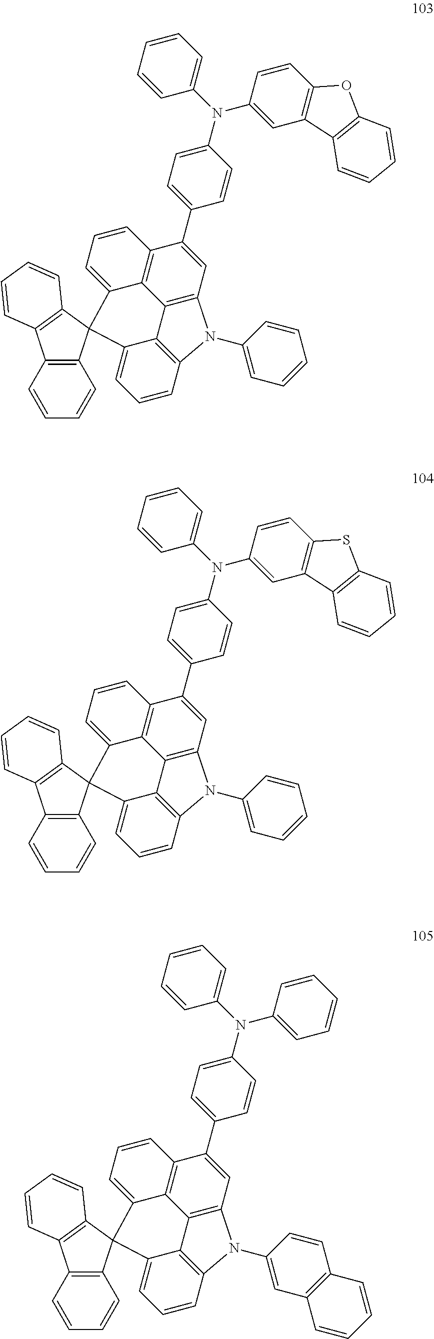 [SMILES: c1ccc(N(c2ccc(-c3cc4c5c6c(cccc36)C3(c6ccccc6-c6ccccc63)c3cccc(c35)n4-c3ccccc3)cc2)c2ccc3oc4ccccc4c3c2)cc1 ; c1ccc(N(c2ccc(-c3cc4c5c6c(cccc36)C3(c6ccccc6-c6ccccc63)c3cccc(c35)n4-c3ccccc3)cc2)c2ccc3sc4ccccc4c3c2)cc1 ; c1ccc(N(c2ccccc2)c2ccc(-c3cc4c5c6c(cccc36)C3(c6ccccc6-c6ccccc63)c3cccc(c35)n4-c3ccc4ccccc4c3)cc2)cc1]

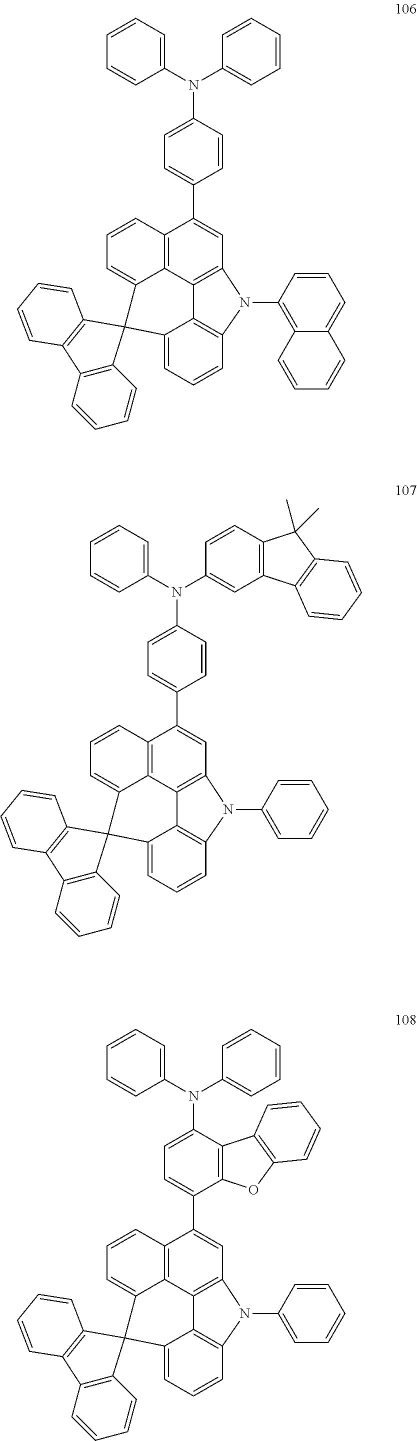 [SMILES: c1ccc(N(c2ccccc2)c2ccc(-c3cc4c5c6c(cccc36)C3(c6ccccc6-c6ccccc63)c3cccc(c35)n4-c3cccc4ccccc34)cc2)cc1 ; CC1(C)c2ccccc2-c2cc(N(c3ccccc3)c3ccc(-c4cc5c6c7c(cccc47)C4(c7ccccc7-c7ccccc74)c4cccc(c46)n5-c4ccccc4)cc3)ccc21 ; c1ccc(N(c2ccccc2)c2ccc(-c3cc4c5c6c(cccc36)C3(c6ccccc6-c6ccccc63)c3cccc(c35)n4-c3ccccc3)c3oc4ccccc4c23)cc1]

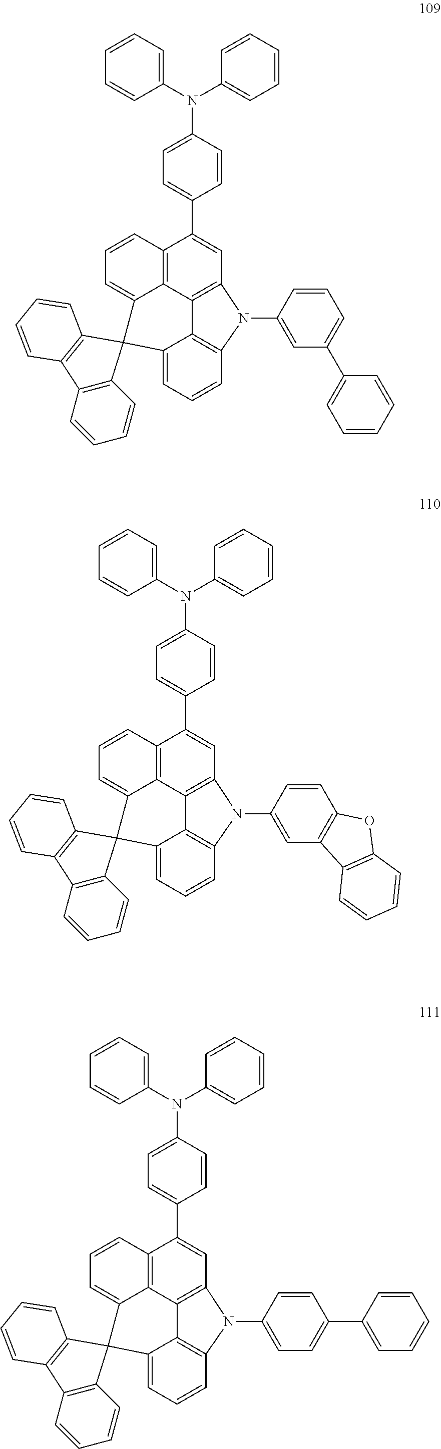 [SMILES: c1ccc(-c2cccc(-n3c4cccc5c4c4c6c(cccc6c(-c6ccc(N(c7ccccc7)c7ccccc7)cc6)cc43)C53c4ccccc4-c4ccccc43)c2)cc1 ; c1ccc(N(c2ccccc2)c2ccc(-c3cc4c5c6c(cccc36)C3(c6ccccc6-c6ccccc63)c3cccc(c35)n4-c3ccc4oc5ccccc5c4c3)cc2)cc1 ; c1ccc(-c2ccc(-n3c4cccc5c4c4c6c(cccc6c(-c6ccc(N(c7ccccc7)c7ccccc7)cc6)cc43)C53c4ccccc4-c4ccccc43)cc2)cc1]

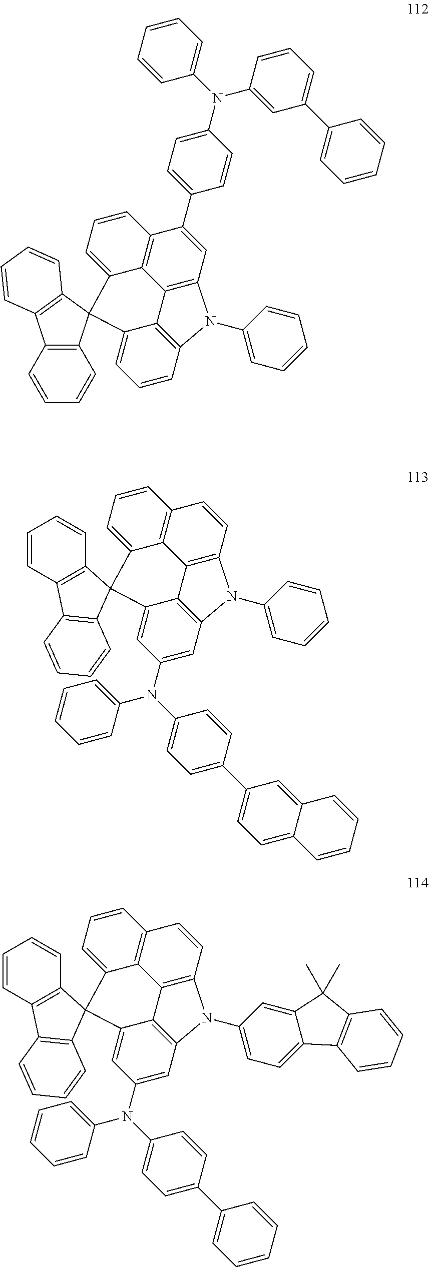 [SMILES: c1ccc(-c2cccc(N(c3ccccc3)c3ccc(-c4cc5c6c7c(cccc47)C4(c7ccccc7-c7ccccc74)c4cccc(c46)n5-c4ccccc4)cc3)c2)cc1 ; c1ccc(N(c2ccc(-c3ccc4ccccc4c3)cc2)c2cc3c4c5c6c(cccc6ccc5n(-c5ccccc5)c4c2)C32c3ccccc3-c3ccccc32)cc1 ; CC1(C)c2ccccc2-c2ccc(-n3c4cc(N(c5ccccc5)c5ccc(-c6ccccc6)cc5)cc5c4c4c6c(cccc6ccc43)C53c4ccccc4-c4ccccc43)cc21]

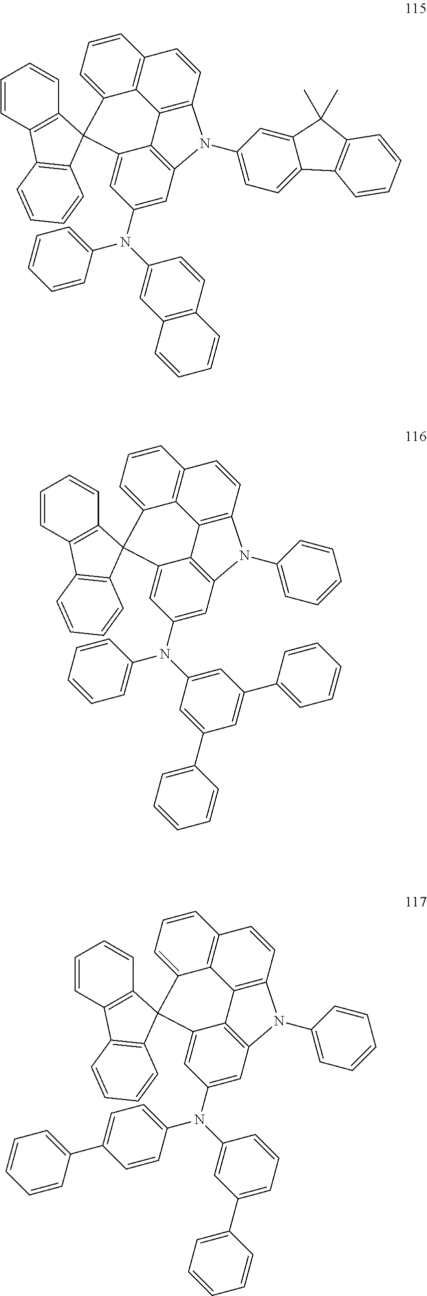 [SMILES: CC1(C)c2ccccc2-c2ccc(-n3c4cc(N(c5ccccc5)c5ccc6ccccc6c5)cc5c4c4c6c(cccc6ccc43)C53c4ccccc4-c4ccccc43)cc21 ; c1ccc(-c2cc(-c3ccccc3)cc(N(c3ccccc3)c3cc4c5c6c7c(cccc7ccc6n(-c6ccccc6)c5c3)C43c4ccccc4-c4ccccc43)c2)cc1 ; c1ccc(-c2ccc(N(c3cccc(-c4ccccc4)c3)c3cc4c5c6c7c(cccc7ccc6n(-c6ccccc6)c5c3)C43c4ccccc4-c4ccccc43)cc2)cc1]

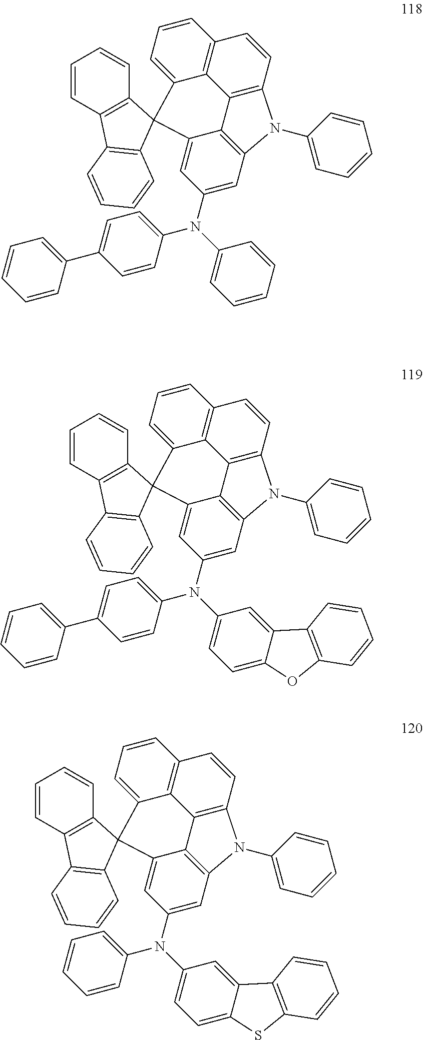 [SMILES: c1ccc(-c2ccc(N(c3ccccc3)c3cc4c5c6c7c(cccc7ccc6n(-c6ccccc6)c5c3)C43c4ccccc4-c4ccccc43)cc2)cc1 ; c1ccc(-c2ccc(N(c3ccc4oc5ccccc5c4c3)c3cc4c5c6c7c(cccc7ccc6n(-c6ccccc6)c5c3)C43c4ccccc4-c4ccccc43)cc2)cc1 ; c1ccc(N(c2ccc3sc4ccccc4c3c2)c2cc3c4c5c6c(cccc6ccc5n(-c5ccccc5)c4c2)C32c3ccccc3-c3ccccc32)cc1]

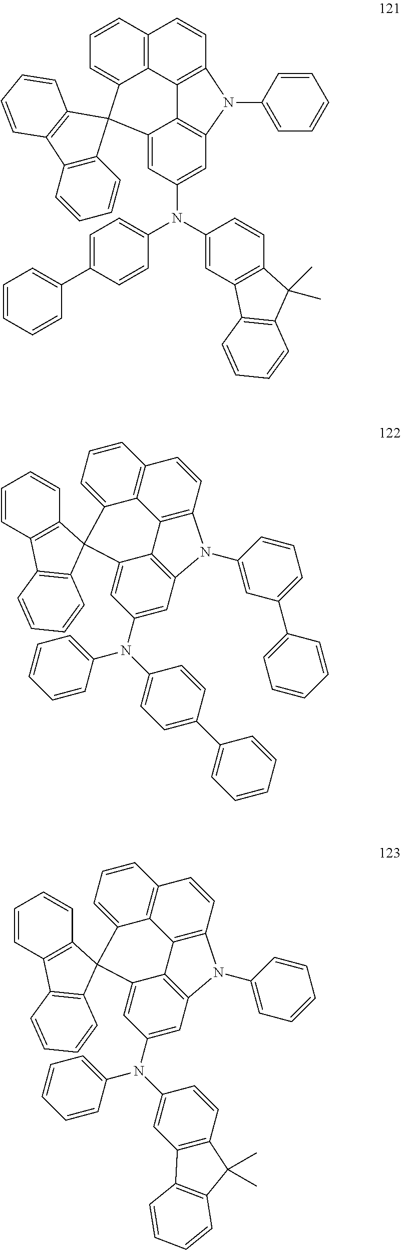 [SMILES: CC1(C)c2ccccc2-c2cc(N(c3ccc(-c4ccccc4)cc3)c3cc4c5c6c7c(cccc7ccc6n(-c6ccccc6)c5c3)C43c4ccccc4-c4ccccc43)ccc21 ; c1ccc(-c2ccc(N(c3ccccc3)c3cc4c5c6c7c(cccc7ccc6n(-c6cccc(-c7ccccc7)c6)c5c3)C43c4ccccc4-c4ccccc43)cc2)cc1 ; CC1(C)c2ccccc2-c2cc(N(c3ccccc3)c3cc4c5c6c7c(cccc7ccc6n(-c6ccccc6)c5c3)C43c4ccccc4-c4ccccc43)ccc21]

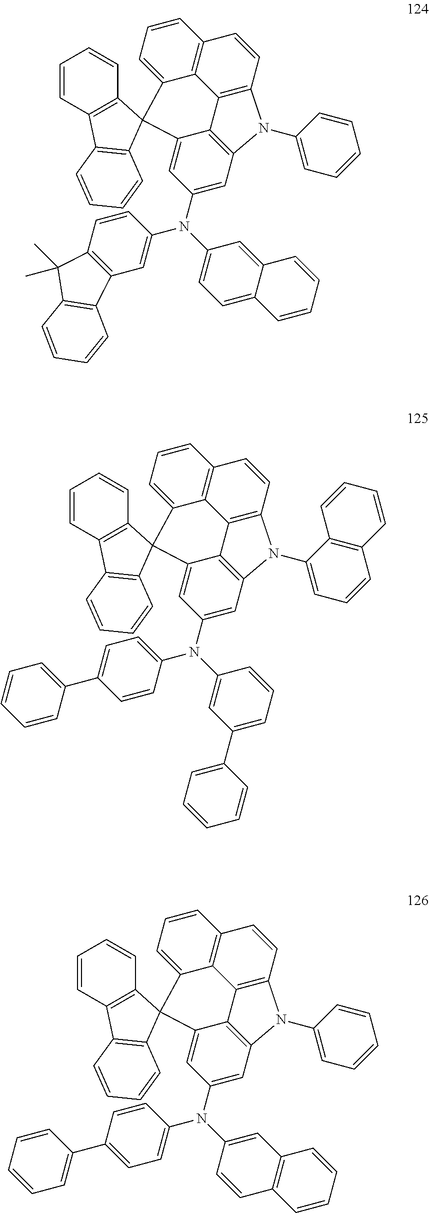 [SMILES: CC1(C)c2ccccc2-c2cc(N(c3ccc4ccccc4c3)c3cc4c5c6c7c(cccc7ccc6n(-c6ccccc6)c5c3)C43c4ccccc4-c4ccccc43)ccc21 ; c1ccc(-c2ccc(N(c3cccc(-c4ccccc4)c3)c3cc4c5c6c7c(cccc7ccc6n(-c6cccc7ccccc67)c5c3)C43c4ccccc4-c4ccccc43)cc2)cc1 ; c1ccc(-c2ccc(N(c3ccc4ccccc4c3)c3cc4c5c6c7c(cccc7ccc6n(-c6ccccc6)c5c3)C43c4ccccc4-c4ccccc43)cc2)cc1]

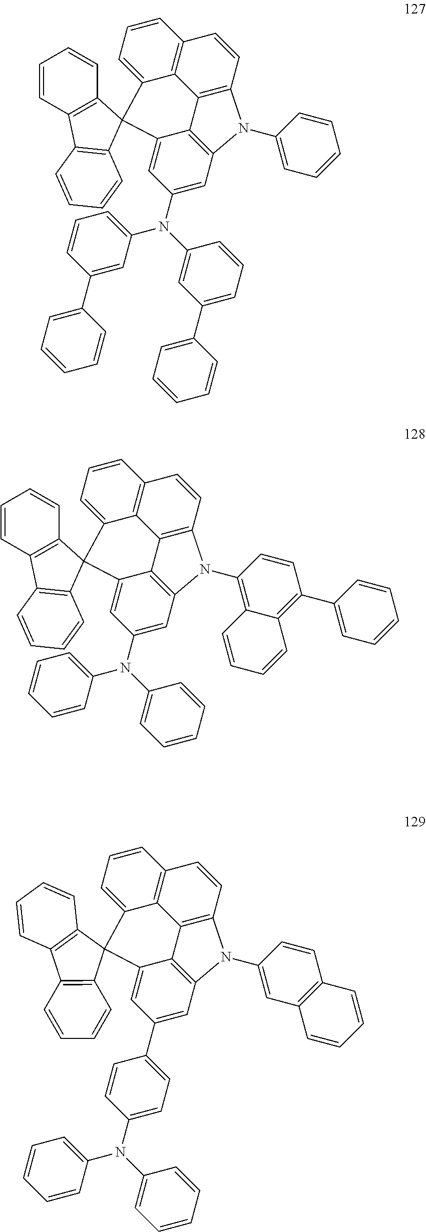 [SMILES: c1ccc(-c2cccc(N(c3cccc(-c4ccccc4)c3)c3cc4c5c6c7c(cccc7ccc6n(-c6ccccc6)c5c3)C43c4ccccc4-c4ccccc43)c2)cc1 ; c1ccc(-c2ccc(-n3c4cc(N(c5ccccc5)c5ccccc5)cc5c4c4c6c(cccc6ccc43)C53c4ccccc4-c4ccccc43)c3ccccc23)cc1 ; c1ccc(N(c2ccccc2)c2ccc(-c3cc4c5c6c7c(cccc7ccc6n(-c6ccc7ccccc7c6)c5c3)C43c4ccccc4-c4ccccc43)cc2)cc1]

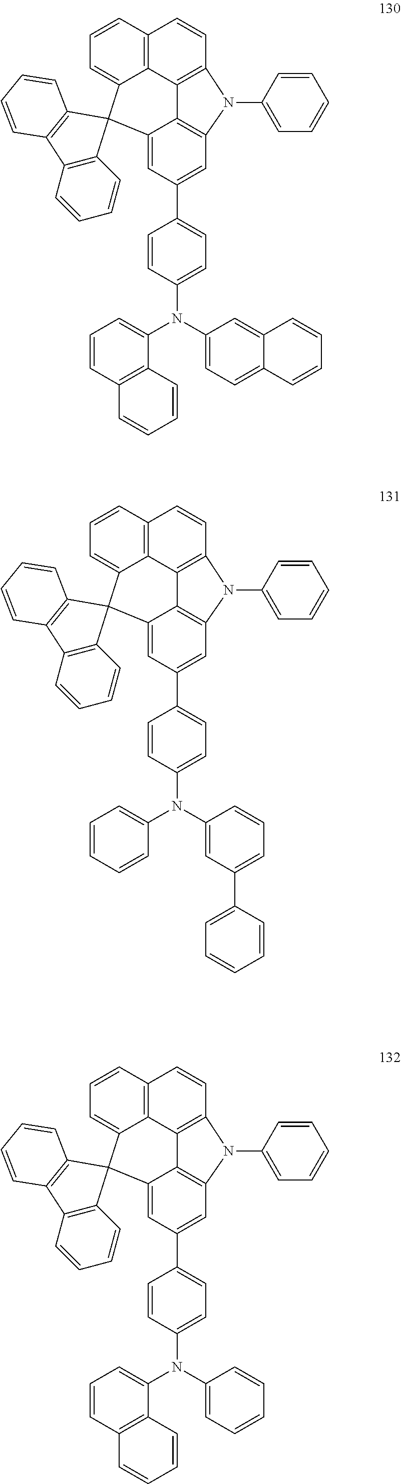 [SMILES: c1ccc(-n2c3cc(-c4ccc(N(c5ccc6ccccc6c5)c5cccc6ccccc56)cc4)cc4c3c3c5c(cccc5ccc32)C42c3ccccc3-c3ccccc32)cc1 ; c1ccc(-c2cccc(N(c3ccccc3)c3ccc(-c4cc5c6c7c8c(cccc8ccc7n(-c7ccccc7)c6c4)C54c5ccccc5-c5ccccc54)cc3)c2)cc1 ; c1ccc(N(c2ccc(-c3cc4c5c6c7c(cccc7ccc6n(-c6ccccc6)c5c3)C43c4ccccc4-c4ccccc43)cc2)c2cccc3ccccc23)cc1]

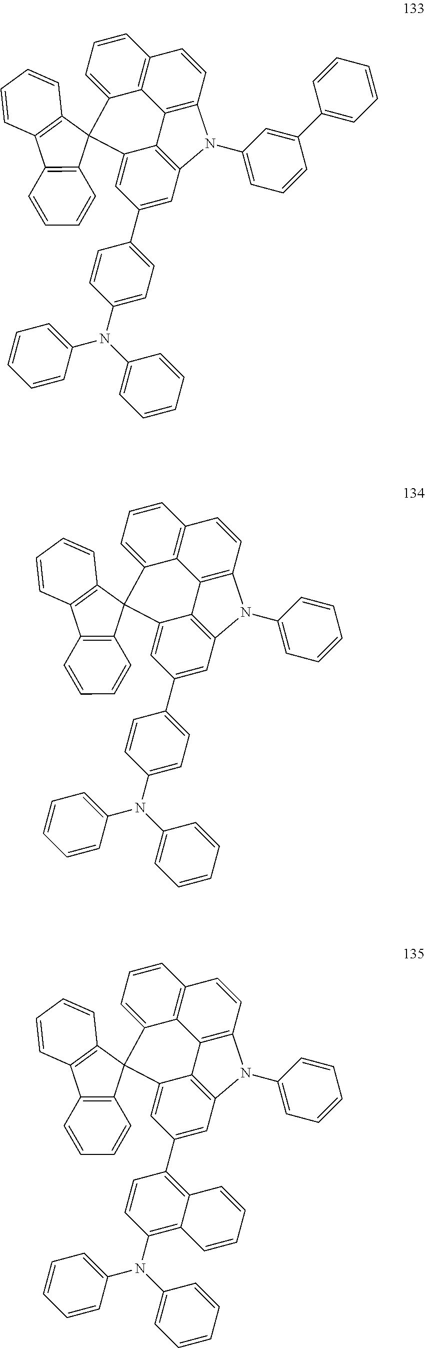 [SMILES: c1ccc(-c2cccc(-n3c4cc(-c5ccc(N(c6ccccc6)c6ccccc6)cc5)cc5c4c4c6c(cccc6ccc43)C53c4ccccc4-c4ccccc43)c2)cc1 ; c1ccc(N(c2ccccc2)c2ccc(-c3cc4c5c6c7c(cccc7ccc6n(-c6ccccc6)c5c3)C43c4ccccc4-c4ccccc43)cc2)cc1 ; c1ccc(N(c2ccccc2)c2ccc(-c3cc4c5c6c7c(cccc7ccc6n(-c6ccccc6)c5c3)C43c4ccccc4-c4ccccc43)c3ccccc23)cc1]

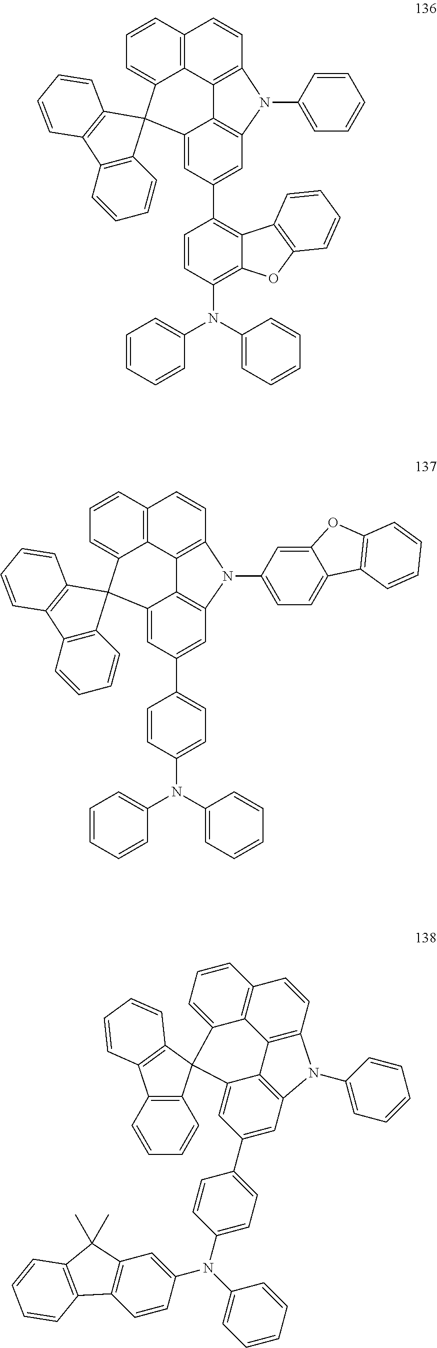 [SMILES: c1ccc(N(c2ccccc2)c2ccc(-c3cc4c5c6c7c(cccc7ccc6n(-c6ccccc6)c5c3)C43c4ccccc4-c4ccccc43)c3c2oc2ccccc23)cc1 ; c1ccc(N(c2ccccc2)c2ccc(-c3cc4c5c6c7c(cccc7ccc6n(-c6ccc7c(c6)oc6ccccc67)c5c3)C43c4ccccc4-c4ccccc43)cc2)cc1 ; CC1(C)c2ccccc2-c2ccc(N(c3ccccc3)c3ccc(-c4cc5c6c7c8c(cccc8ccc7n(-c7ccccc7)c6c4)C54c5ccccc5-c5ccccc54)cc3)cc21]

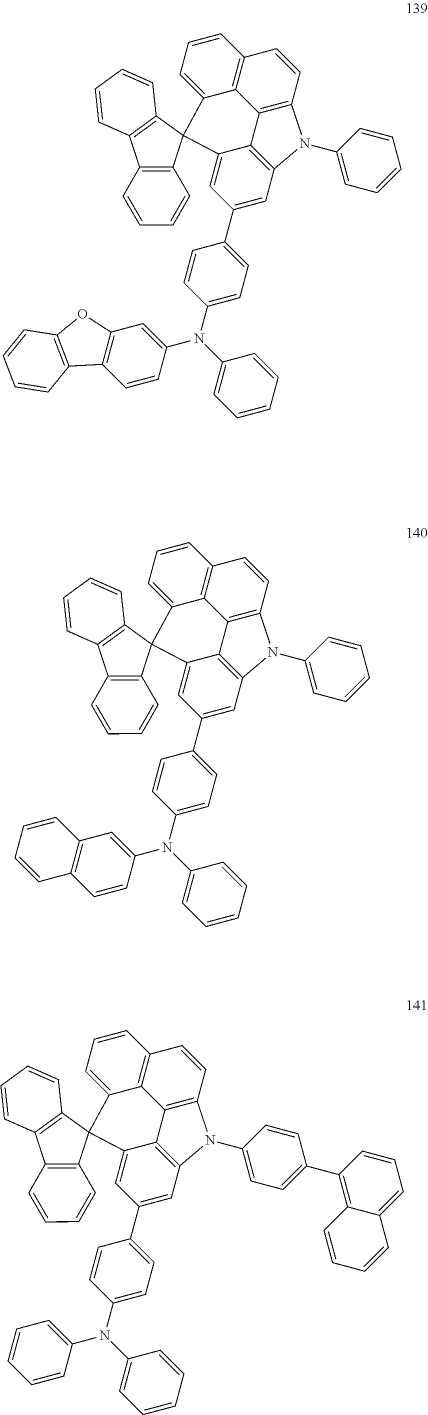 [SMILES: c1ccc(N(c2ccc(-c3cc4c5c6c7c(cccc7ccc6n(-c6ccccc6)c5c3)C43c4ccccc4-c4ccccc43)cc2)c2ccc3c(c2)oc2ccccc23)cc1 ; c1ccc(N(c2ccc(-c3cc4c5c6c7c(cccc7ccc6n(-c6ccccc6)c5c3)C43c4ccccc4-c4ccccc43)cc2)c2ccc3ccccc3c2)cc1 ; c1ccc(N(c2ccccc2)c2ccc(-c3cc4c5c6c7c(cccc7ccc6n(-c6ccc(-c7cccc8ccccc78)cc6)c5c3)C43c4ccccc4-c4ccccc43)cc2)cc1]

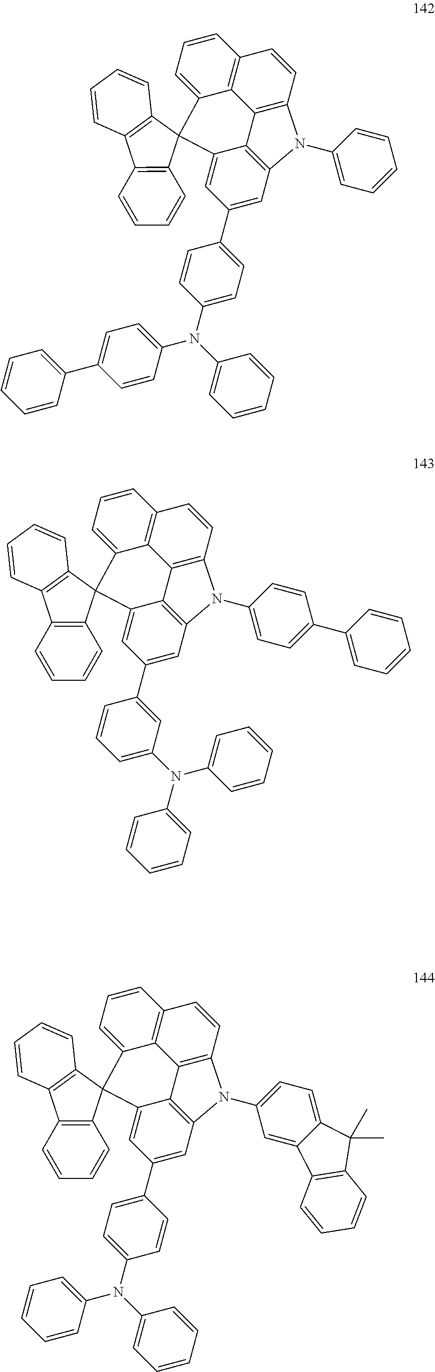 [SMILES: c1ccc(-c2ccc(N(c3ccccc3)c3ccc(-c4cc5c6c7c8c(cccc8ccc7n(-c7ccccc7)c6c4)C54c5ccccc5-c5ccccc54)cc3)cc2)cc1 ; c1ccc(-c2ccc(-n3c4cc(-c5cccc(N(c6ccccc6)c6ccccc6)c5)cc5c4c4c6c(cccc6ccc43)C53c4ccccc4-c4ccccc43)cc2)cc1 ; CC1(C)c2ccccc2-c2cc(-n3c4cc(-c5ccc(N(c6ccccc6)c6ccccc6)cc5)cc5c4c4c6c(cccc6ccc43)C53c4ccccc4-c4ccccc43)ccc21]

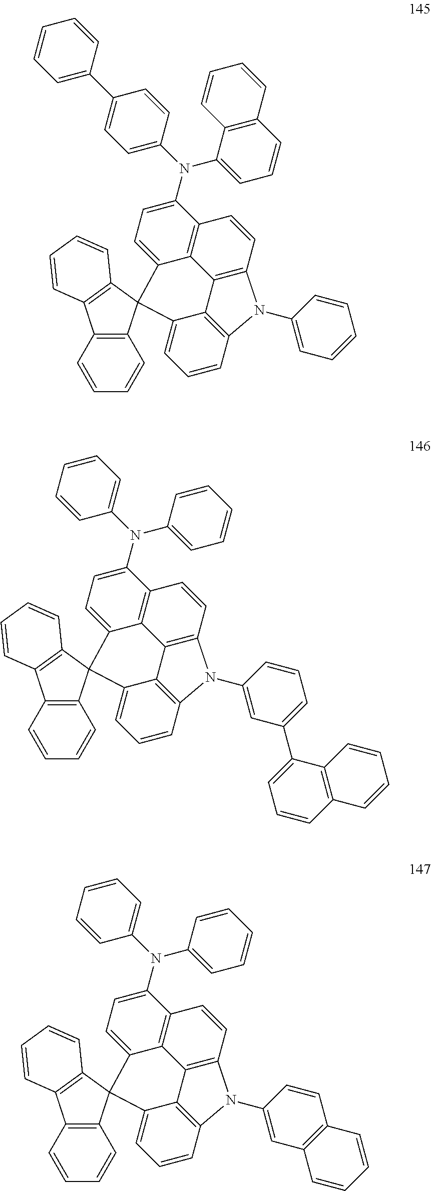 [SMILES: c1ccc(-c2ccc(N(c3cccc4ccccc34)c3ccc4c5c3ccc3c5c5c(cccc5n3-c3ccccc3)C43c4ccccc4-c4ccccc43)cc2)cc1 ; c1ccc(N(c2ccccc2)c2ccc3c4c2ccc2c4c4c(cccc4n2-c2cccc(-c4cccc5ccccc45)c2)C32c3ccccc3-c3ccccc32)cc1 ; c1ccc(N(c2ccccc2)c2ccc3c4c2ccc2c4c4c(cccc4n2-c2ccc4ccccc4c2)C32c3ccccc3-c3ccccc32)cc1]

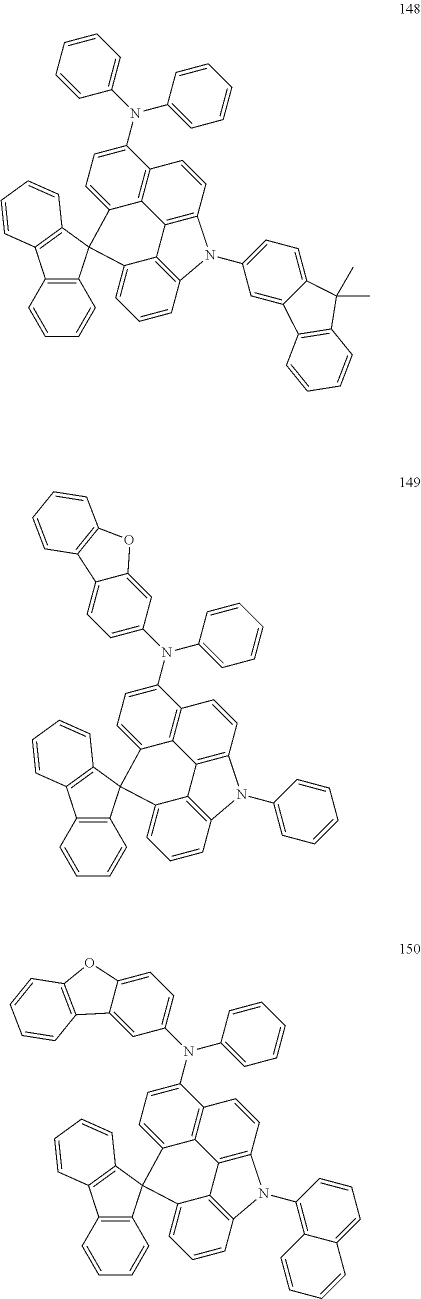 [SMILES: CC1(C)c2ccccc2-c2cc(-n3c4cccc5c4c4c6c(ccc(N(c7ccccc7)c7ccccc7)c6ccc43)C53c4ccccc4-c4ccccc43)ccc21 ; c1ccc(N(c2ccc3c(c2)oc2ccccc23)c2ccc3c4c2ccc2c4c4c(cccc4n2-c2ccccc2)C32c3ccccc3-c3ccccc32)cc1 ; c1ccc(N(c2ccc3oc4ccccc4c3c2)c2ccc3c4c2ccc2c4c4c(cccc4n2-c2cccc4ccccc24)C32c3ccccc3-c3ccccc32)cc1]

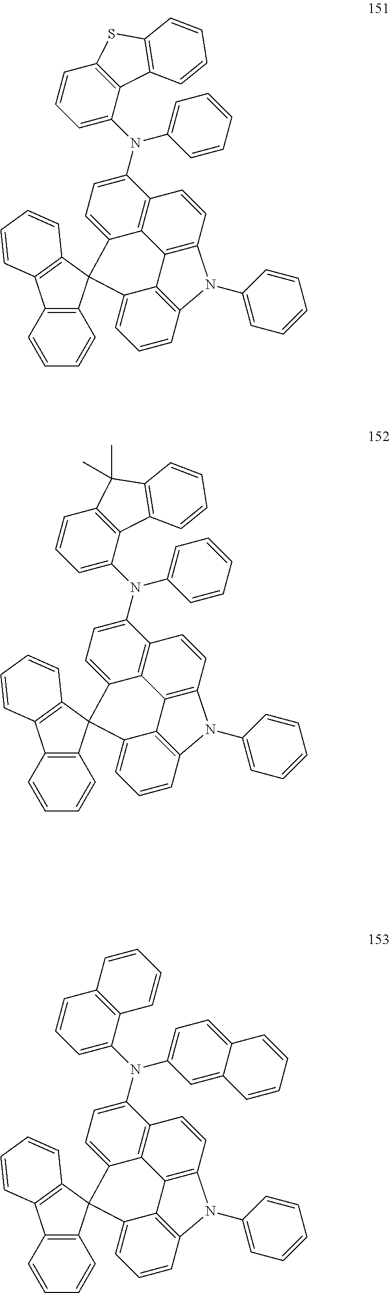 [SMILES: c1ccc(N(c2ccc3c4c2ccc2c4c4c(cccc4n2-c2ccccc2)C32c3ccccc3-c3ccccc32)c2cccc3sc4ccccc4c23)cc1 ; CC1(C)c2ccccc2-c2c(N(c3ccccc3)c3ccc4c5c3ccc3c5c5c(cccc5n3-c3ccccc3)C43c4ccccc4-c4ccccc43)cccc21 ; c1ccc(-n2c3cccc4c3c3c5c(ccc(N(c6ccc7ccccc7c6)c6cccc7ccccc67)c5ccc32)C42c3ccccc3-c3ccccc32)cc1]

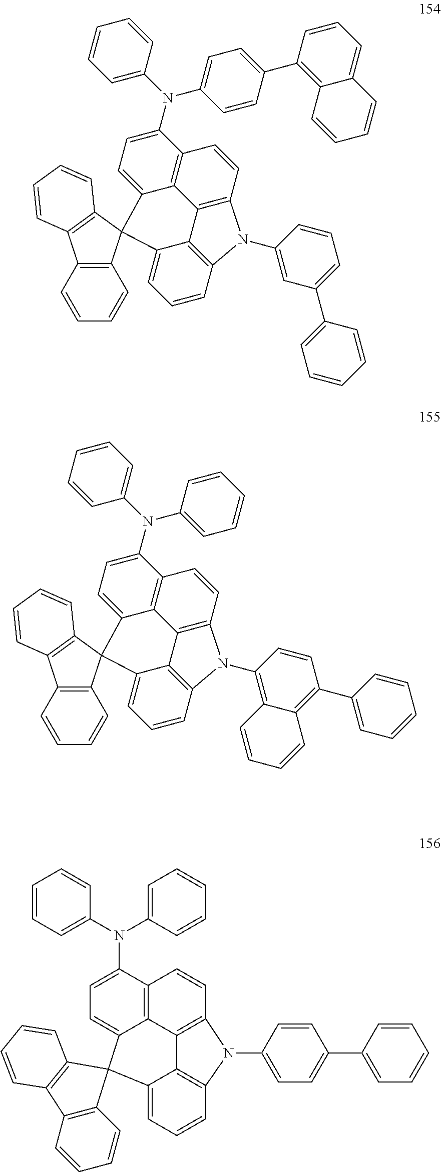 [SMILES: c1ccc(-c2cccc(-n3c4cccc5c4c4c6c(ccc(N(c7ccccc7)c7ccc(-c8cccc9ccccc89)cc7)c6ccc43)C53c4ccccc4-c4ccccc43)c2)cc1 ; c1ccc(-c2ccc(-n3c4cccc5c4c4c6c(ccc(N(c7ccccc7)c7ccccc7)c6ccc43)C53c4ccccc4-c4ccccc43)c3ccccc23)cc1 ; c1ccc(-c2ccc(-n3c4cccc5c4c4c6c(ccc(N(c7ccccc7)c7ccccc7)c6ccc43)C53c4ccccc4-c4ccccc43)cc2)cc1]

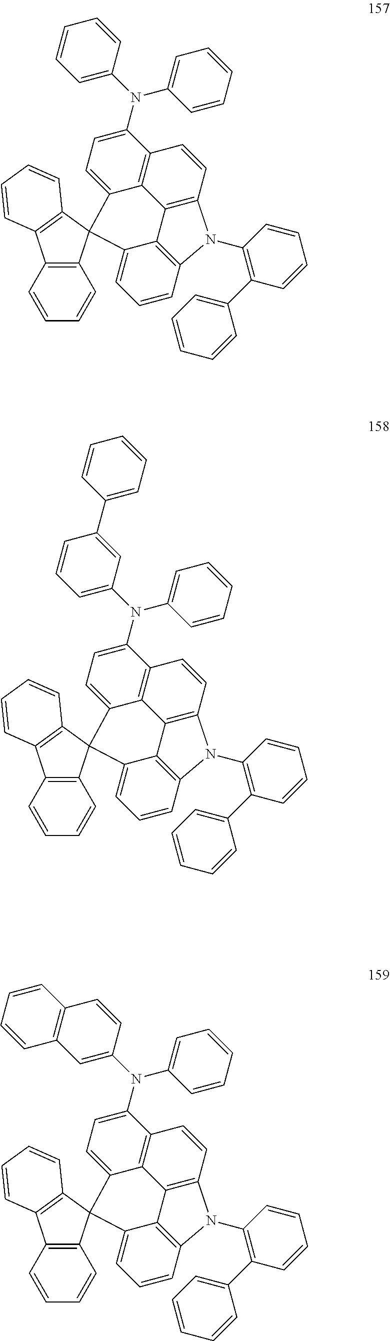 [SMILES: c1ccc(-c2ccccc2-n2c3cccc4c3c3c5c(ccc(N(c6ccccc6)c6ccccc6)c5ccc32)C42c3ccccc3-c3ccccc32)cc1 ; c1ccc(-c2cccc(N(c3ccccc3)c3ccc4c5c3ccc3c5c5c(cccc5n3-c3ccccc3-c3ccccc3)C43c4ccccc4-c4ccccc43)c2)cc1 ; c1ccc(-c2ccccc2-n2c3cccc4c3c3c5c(ccc(N(c6ccccc6)c6ccc7ccccc7c6)c5ccc32)C42c3ccccc3-c3ccccc32)cc1]

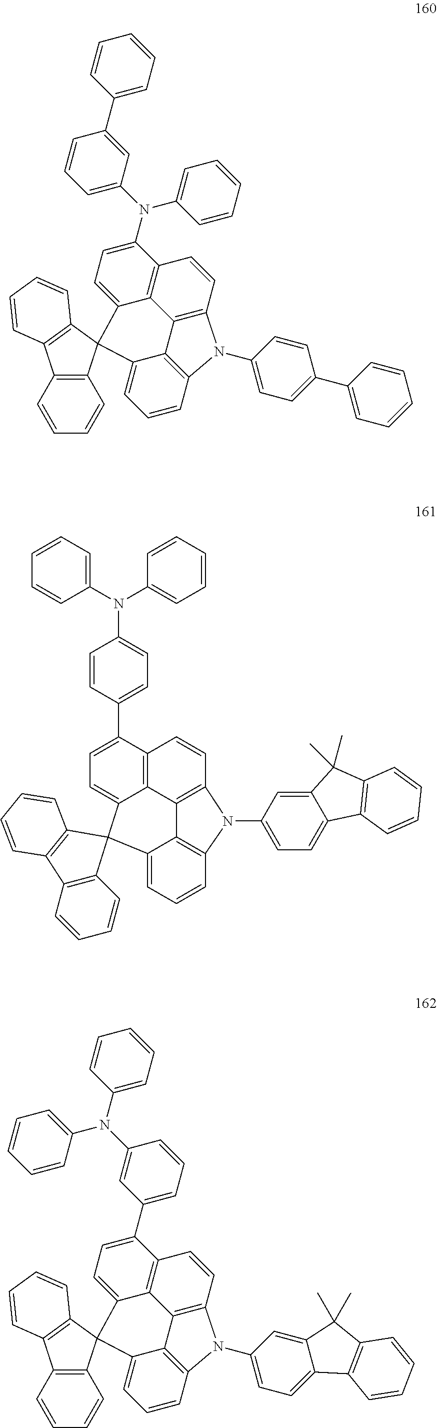 [SMILES: c1ccc(-c2ccc(-n3c4cccc5c4c4c6c(ccc(N(c7ccccc7)c7cccc(-c8ccccc8)c7)c6ccc43)C53c4ccccc4-c4ccccc43)cc2)cc1 ; CC1(C)c2ccccc2-c2ccc(-n3c4cccc5c4c4c6c(ccc(-c7ccc(N(c8ccccc8)c8ccccc8)cc7)c6ccc43)C53c4ccccc4-c4ccccc43)cc21 ; CC1(C)c2ccccc2-c2ccc(-n3c4cccc5c4c4c6c(ccc(-c7cccc(N(c8ccccc8)c8ccccc8)c7)c6ccc43)C53c4ccccc4-c4ccccc43)cc21]

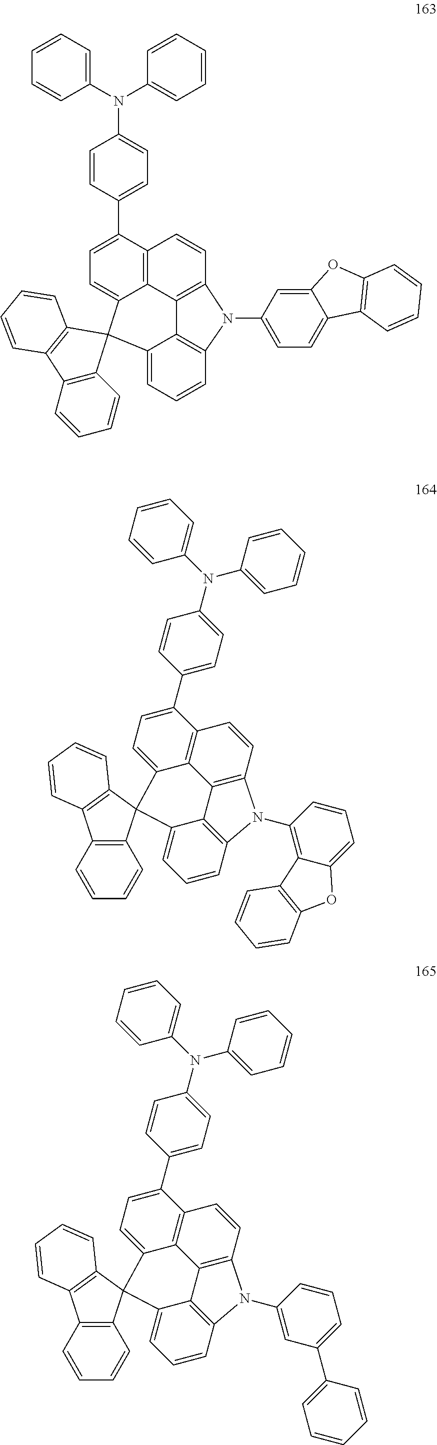 [SMILES: c1ccc(N(c2ccccc2)c2ccc(-c3ccc4c5c3ccc3c5c5c(cccc5n3-c3ccc5c(c3)oc3ccccc35)C43c4ccccc4-c4ccccc43)cc2)cc1 ; c1ccc(N(c2ccccc2)c2ccc(-c3ccc4c5c3ccc3c5c5c(cccc5n3-c3cccc5oc6ccccc6c35)C43c4ccccc4-c4ccccc43)cc2)cc1 ; c1ccc(-c2cccc(-n3c4cccc5c4c4c6c(ccc(-c7ccc(N(c8ccccc8)c8ccccc8)cc7)c6ccc43)C53c4ccccc4-c4ccccc43)c2)cc1]

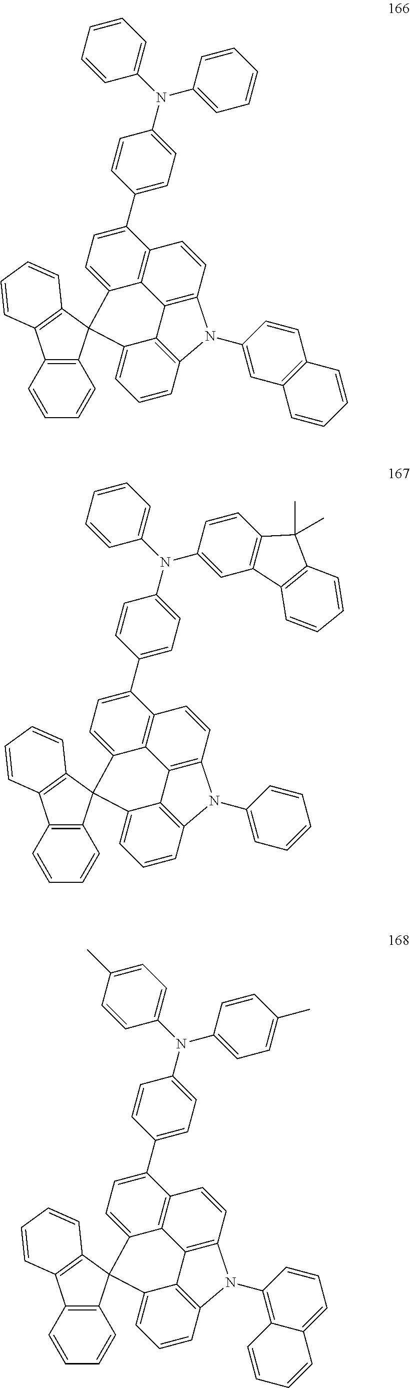 [SMILES: c1ccc(N(c2ccccc2)c2ccc(-c3ccc4c5c3ccc3c5c5c(cccc5n3-c3ccc5ccccc5c3)C43c4ccccc4-c4ccccc43)cc2)cc1 ; CC1(C)c2ccccc2-c2cc(N(c3ccccc3)c3ccc(-c4ccc5c6c4ccc4c6c6c(cccc6n4-c4ccccc4)C54c5ccccc5-c5ccccc54)cc3)ccc21 ; Cc1ccc(N(c2ccc(C)cc2)c2ccc(-c3ccc4c5c3ccc3c5c5c(cccc5n3-c3cccc5ccccc35)C43c4ccccc4-c4ccccc43)cc2)cc1]

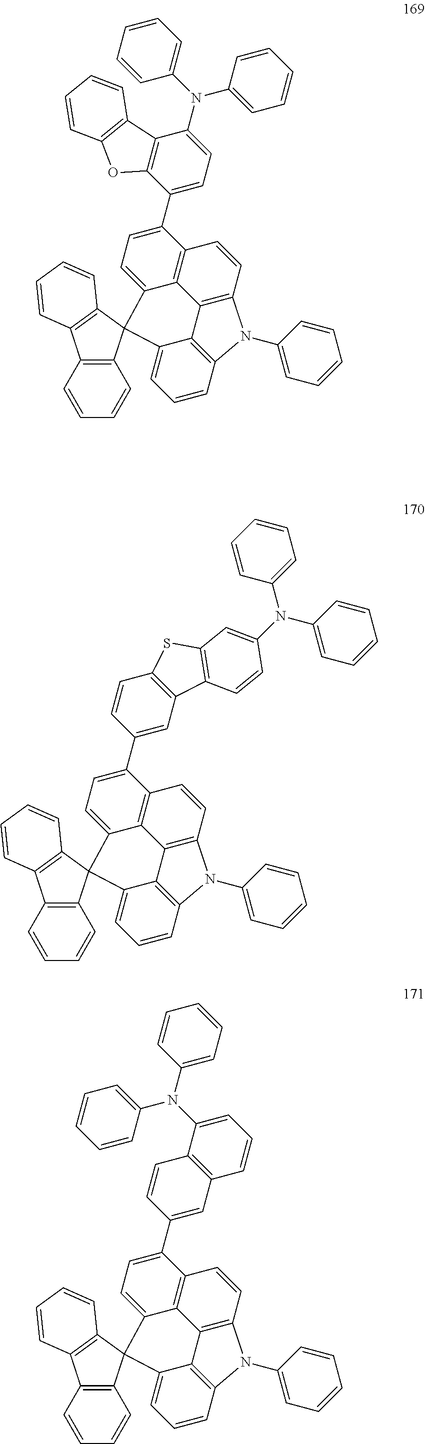 [SMILES: c1ccc(N(c2ccccc2)c2ccc(-c3ccc4c5c3ccc3c5c5c(cccc5n3-c3ccccc3)C43c4ccccc4-c4ccccc43)c3oc4ccccc4c23)cc1 ; c1ccc(N(c2ccccc2)c2ccc3c(c2)sc2ccc(-c4ccc5c6c4ccc4c6c6c(cccc6n4-c4ccccc4)C54c5ccccc5-c5ccccc54)cc23)cc1 ; c1ccc(N(c2ccccc2)c2cccc3cc(-c4ccc5c6c4ccc4c6c6c(cccc6n4-c4ccccc4)C54c5ccccc5-c5ccccc54)ccc23)cc1]

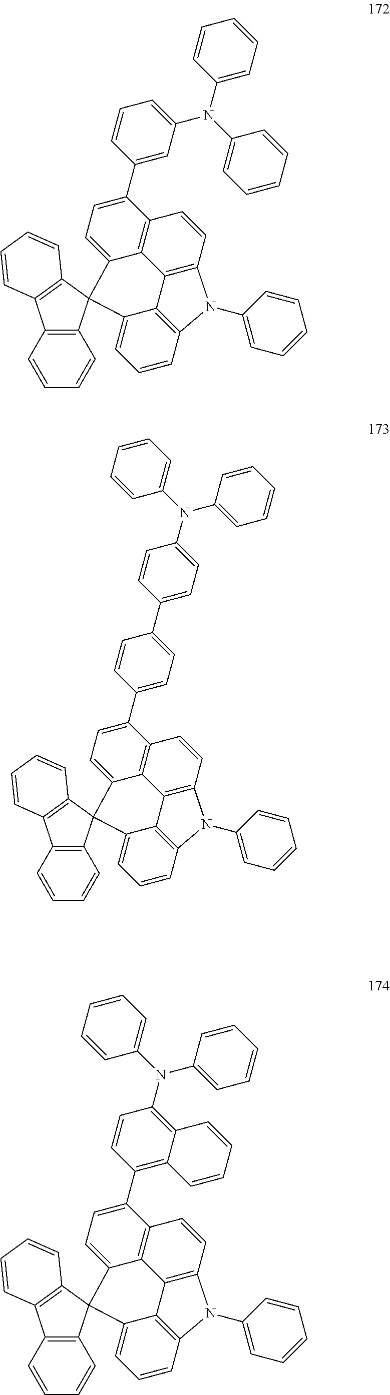 [SMILES: c1ccc(N(c2ccccc2)c2cccc(-c3ccc4c5c3ccc3c5c5c(cccc5n3-c3ccccc3)C43c4ccccc4-c4ccccc43)c2)cc1 ; c1ccc(N(c2ccccc2)c2ccc(-c3ccc(-c4ccc5c6c4ccc4c6c6c(cccc6n4-c4ccccc4)C54c5ccccc5-c5ccccc54)cc3)cc2)cc1 ; c1ccc(N(c2ccccc2)c2ccc(-c3ccc4c5c3ccc3c5c5c(cccc5n3-c3ccccc3)C43c4ccccc4-c4ccccc43)c3ccccc23)cc1]

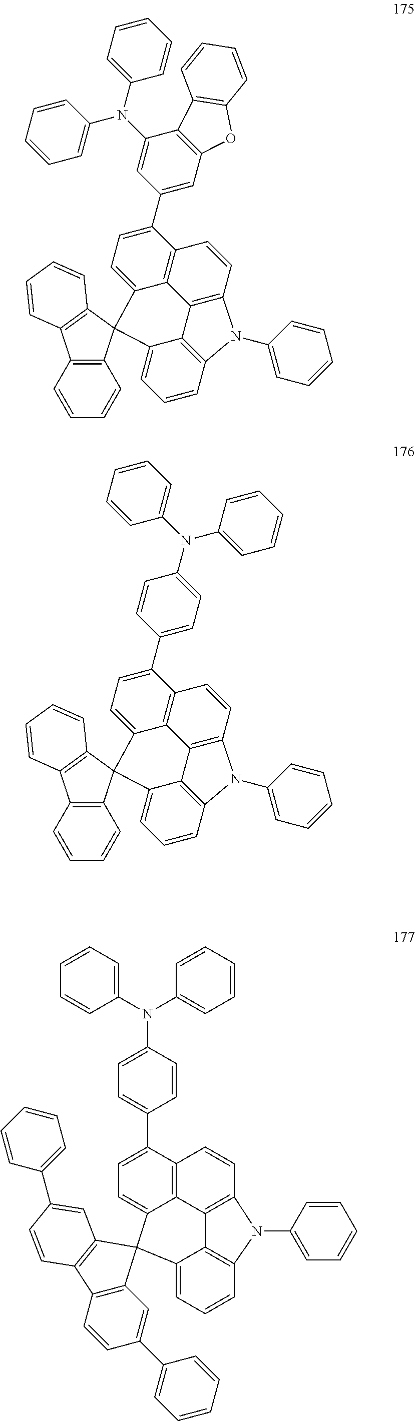 [SMILES: c1ccc(N(c2ccccc2)c2cc(-c3ccc4c5c3ccc3c5c5c(cccc5n3-c3ccccc3)C43c4ccccc4-c4ccccc43)cc3oc4ccccc4c23)cc1 ; c1ccc(N(c2ccccc2)c2ccc(-c3ccc4c5c3ccc3c5c5c(cccc5n3-c3ccccc3)C43c4ccccc4-c4ccccc43)cc2)cc1 ; c1ccc(-c2ccc3c(c2)C2(c4cc(-c5ccccc5)ccc4-3)c3ccc(-c4ccc(N(c5ccccc5)c5ccccc5)cc4)c4ccc5c(c34)c3c2cccc3n5-c2ccccc2)cc1]

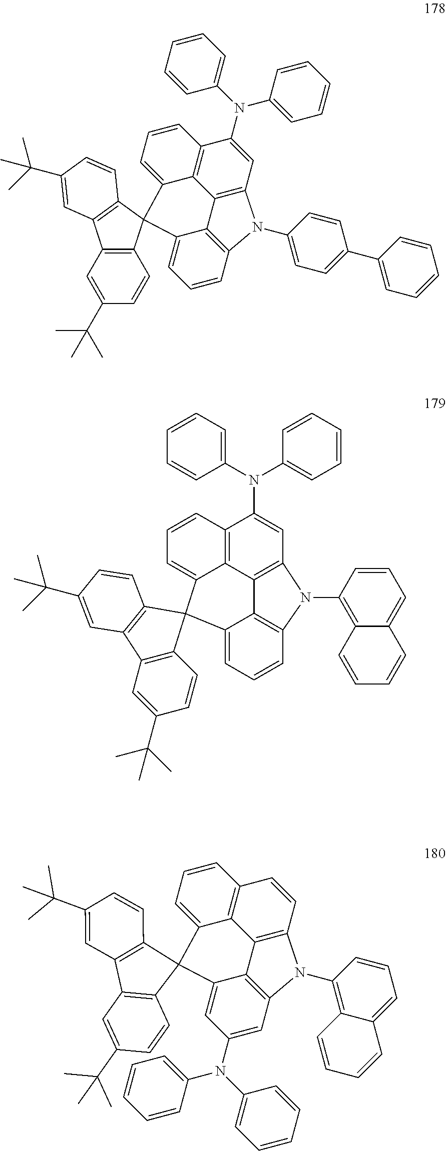 [SMILES: CC(C)(C)c1ccc2c(c1)-c1cc(C(C)(C)C)ccc1C21c2cccc3c(N(c4ccccc4)c4ccccc4)cc4c(c23)c2c1cccc2n4-c1ccc(-c2ccccc2)cc1 ; CC(C)(C)c1ccc2c(c1)-c1cc(C(C)(C)C)ccc1C21c2cccc3c(N(c4ccccc4)c4ccccc4)cc4c(c23)c2c1cccc2n4-c1cccc2ccccc12 ; CC(C)(C)c1ccc2c(c1)-c1cc(C(C)(C)C)ccc1C21c2cccc3ccc4c(c23)c2c1cc(N(c1ccccc1)c1ccccc1)cc2n4-c1cccc2ccccc12]

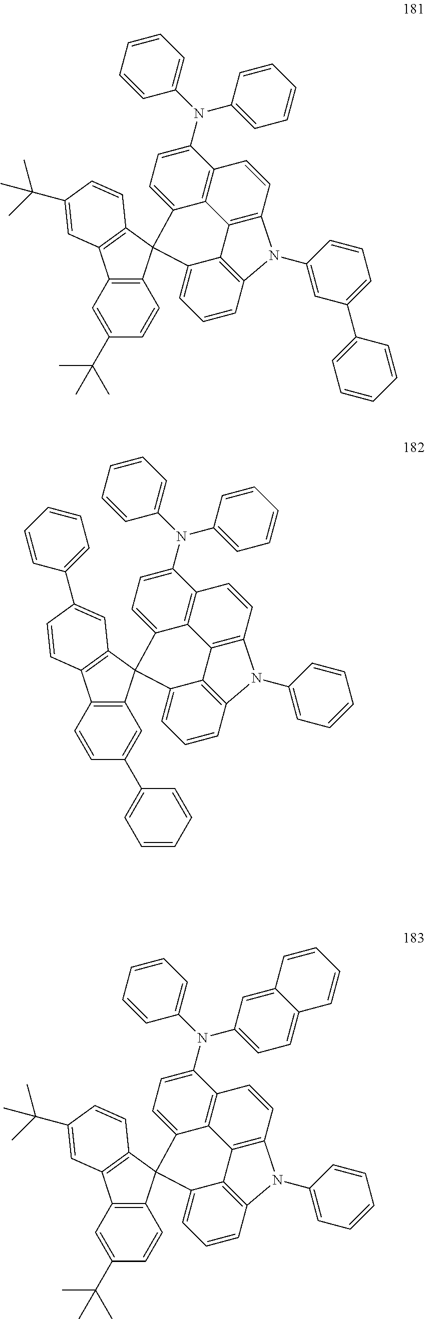 [SMILES: CC(C)(C)c1ccc2c(c1)-c1cc(C(C)(C)C)ccc1C21c2ccc(N(c3ccccc3)c3ccccc3)c3ccc4c(c23)c2c1cccc2n4-c1cccc(-c2ccccc2)c1 ; c1ccc(-c2ccc3c(c2)C2(c4cc(-c5ccccc5)ccc4-3)c3ccc(N(c4ccccc4)c4ccccc4)c4ccc5c(c34)c3c2cccc3n5-c2ccccc2)cc1 ; CC(C)(C)c1ccc2c(c1)-c1cc(C(C)(C)C)ccc1C21c2ccc(N(c3ccccc3)c3ccc4ccccc4c3)c3ccc4c(c23)c2c1cccc2n4-c1ccccc1]

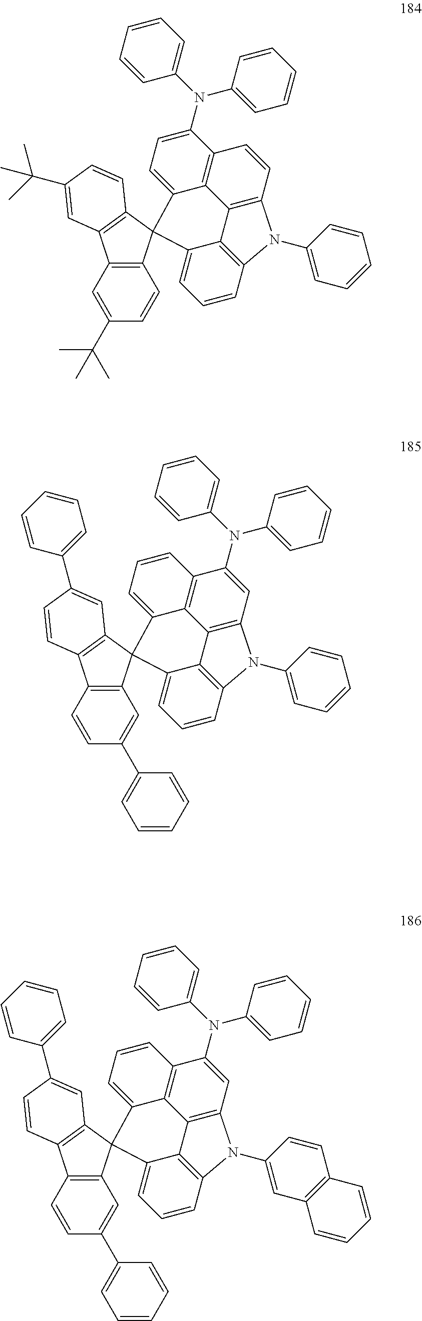 [SMILES: CC(C)(C)c1ccc2c(c1)-c1cc(C(C)(C)C)ccc1C21c2ccc(N(c3ccccc3)c3ccccc3)c3ccc4c(c23)c2c1cccc2n4-c1ccccc1 ; c1ccc(-c2ccc3c(c2)C2(c4cc(-c5ccccc5)ccc4-3)c3cccc4c(N(c5ccccc5)c5ccccc5)cc5c(c34)c3c2cccc3n5-c2ccccc2)cc1 ; c1ccc(-c2ccc3c(c2)C2(c4cc(-c5ccccc5)ccc4-3)c3cccc4c(N(c5ccccc5)c5ccccc5)cc5c(c34)c3c2cccc3n5-c2ccc3ccccc3c2)cc1]

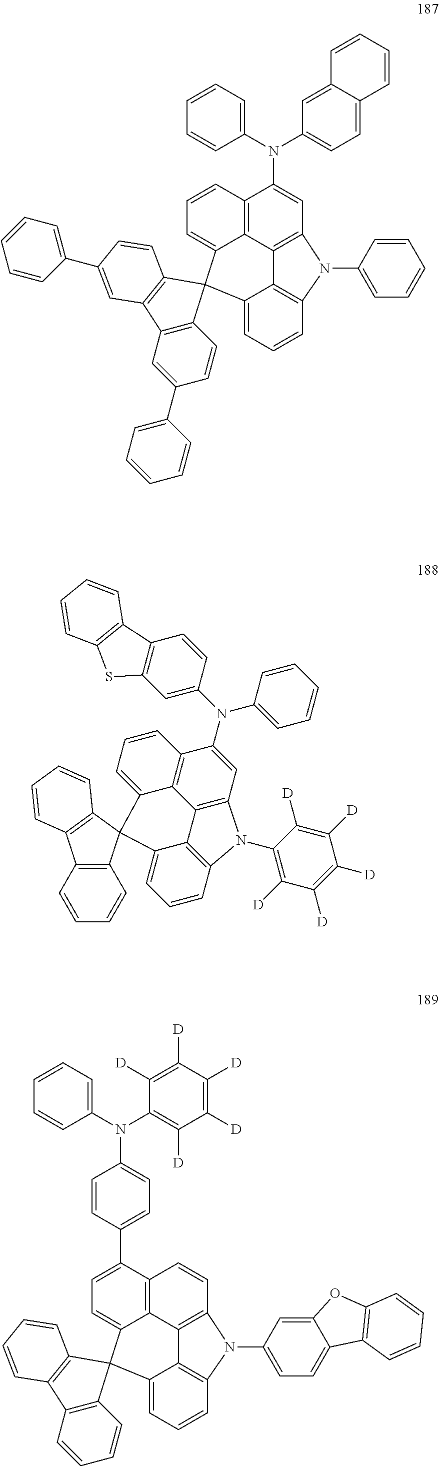 [SMILES: c1ccc(-c2ccc3c(c2)-c2cc(-c4ccccc4)ccc2C32c3cccc4c(N(c5ccccc5)c5ccc6ccccc6c5)cc5c(c34)c3c2cccc3n5-c2ccccc2)cc1 ; [2H]c1c([2H])c([2H])c(-n2c3cccc4c3c3c5c(cccc5c(N(c5ccccc5)c5ccc6c(c5)sc5ccccc56)cc32)C42c3ccccc3-c3ccccc32)c([2H])c1[2H] ; [2H]c1c([2H])c([2H])c(N(c2ccccc2)c2ccc(-c3ccc4c5c3ccc3c5c5c(cccc5n3-c3ccc5c(c3)oc3ccccc35)C43c4ccccc4-c4ccccc43)cc2)c([2H])c1[2H]]

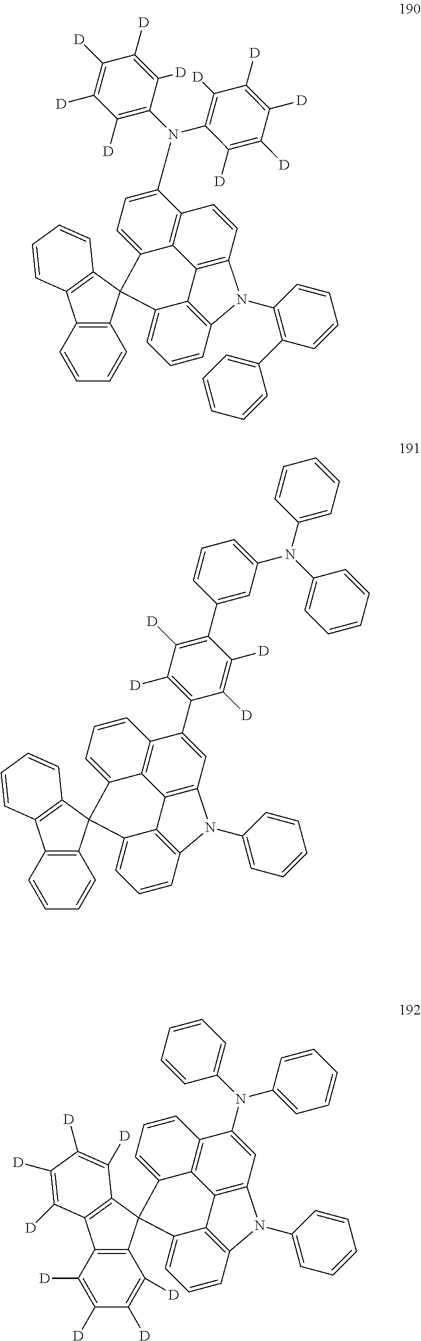 [SMILES: [2H]c1c([2H])c([2H])c(N(c2c([2H])c([2H])c([2H])c([2H])c2[2H])c2ccc3c4c2ccc2c4c4c(cccc4n2-c2ccccc2-c2ccccc2)C32c3ccccc3-c3ccccc32)c([2H])c1[2H] ; [2H]c1c([2H])c(-c2cc3c4c5c(cccc25)C2(c5ccccc5-c5ccccc52)c2cccc(c24)n3-c2ccccc2)c([2H])c([2H])c1-c1cccc(N(c2ccccc2)c2ccccc2)c1 ; [2H]c1c([2H])c([2H])c2c(c1[2H])-c1c([2H])c([2H])c([2H])c([2H])c1C21c2cccc3c(N(c4ccccc4)c4ccccc4)cc4c(c23)c2c1cccc2n4-c1ccccc1]

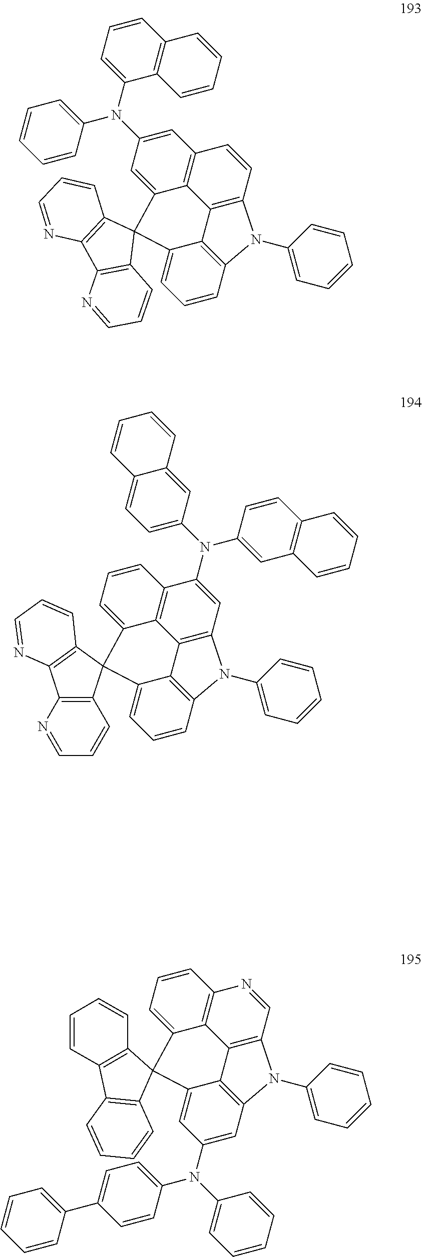 [SMILES: c1ccc(N(c2cc3c4c(ccc5c4c4c(cccc4n5-c4ccccc4)C34c3cccnc3-c3ncccc34)c2)c2cccc3ccccc23)cc1 ; c1ccc(-n2c3cccc4c3c3c5c(cccc5c(N(c5ccc6ccccc6c5)c5ccc6ccccc6c5)cc32)C42c3cccnc3-c3ncccc32)cc1 ; c1ccc(-c2ccc(N(c3ccccc3)c3cc4c5c6c7c(cccc7ncc6n(-c6ccccc6)c5c3)C43c4ccccc4-c4ccccc43)cc2)cc1]

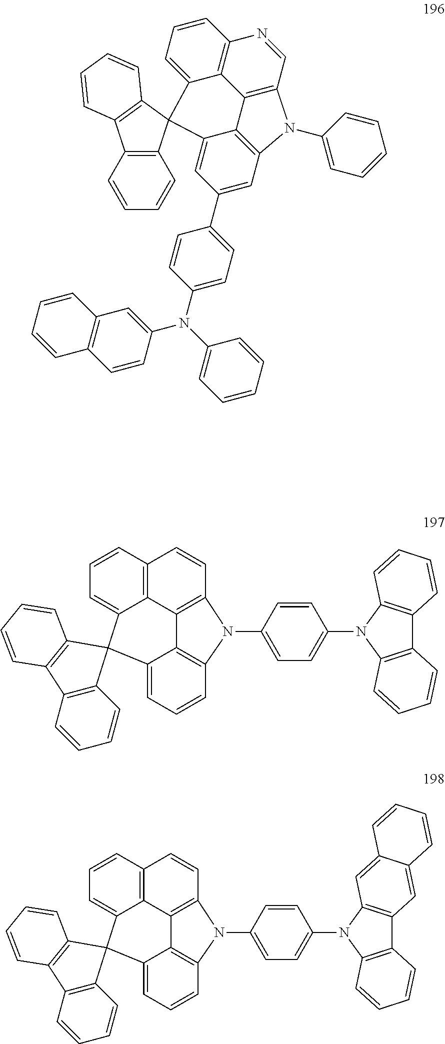 [SMILES: c1ccc(N(c2ccc(-c3cc4c5c6c7c(cccc7ncc6n(-c6ccccc6)c5c3)C43c4ccccc4-c4ccccc43)cc2)c2ccc3ccccc3c2)cc1 ; c1ccc2c(c1)-c1ccccc1C21c2cccc3ccc4c(c23)c2c1cccc2n4-c1ccc(-n2c3ccccc3c3ccccc32)cc1 ; c1ccc2c(c1)-c1ccccc1C21c2cccc3ccc4c(c23)c2c1cccc2n4-c1ccc(-n2c3ccccc3c3cc4ccccc4cc32)cc1]

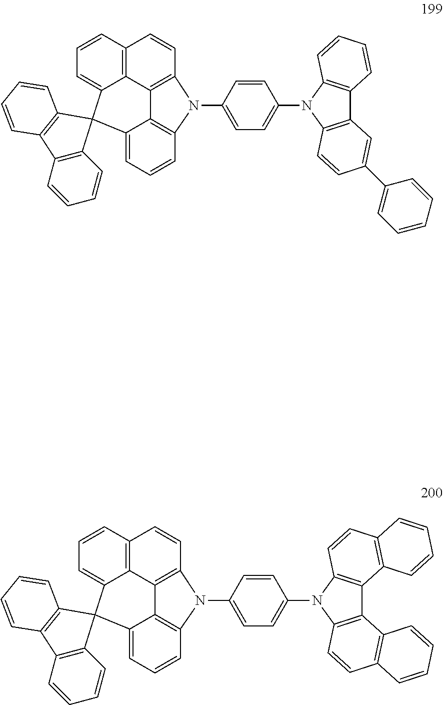 [SMILES: c1ccc(-c2ccc3c(c2)c2ccccc2n3-c2ccc(-n3c4cccc5c4c4c6c(cccc6ccc43)C53c4ccccc4-c4ccccc43)cc2)cc1 ; c1ccc2c(c1)-c1ccccc1C21c2cccc3ccc4c(c23)c2c1cccc2n4-c1ccc(-n2c3ccc4ccccc4c3c3c4ccccc4ccc32)cc1]

In addition, by introducing various substituents into the structure of Formula 1, compounds having intrinsic properties of the introduced substituents can be synthesized. For example, by introducing into the core structure a substituent mainly used in a material for a hole injection layer, a material for a hole transport layer, a material for a light emitting layer, a material for an electron transport layer and a material for a charge generating layer used in manufacturing an organic light emitting device, substances that satisfy the requirements of each organic layer can be synthesized.

Also, by introducing various substituents into the structure of Formula 1, it is possible to finely control the energy bandgap, and on the other hand, to improve the properties at the interface between organic substances, and to diversify the use of the substances.

Also, in one embodiment of the present invention, the present invention provides an organic light emitting device comprising a first electrode; a second electrode provided to face the first electrode; and one or more organic layers provided between the first electrode and the second electrode, wherein at least one of the one or more organic layers comprises a heterocyclic compound represented by Formula 1 above.

In one embodiment of the present invention, the first electrode may be a positive electrode, and the second electrode may be a negative electrode.

In another embodiment, the first electrode may be a negative electrode, and the second electrode may be a positive electrode.

In one embodiment of the present invention, the organic light emitting device may be a blue organic light emitting device, and the heterocyclic compound represented by Formula 1 may be used as a material of the blue organic light emitting device.

In one embodiment of the present invention, the organic light emitting device may be a green organic light emitting device, and the heterocyclic compound represented by Formula 1 may be used as a material of the green organic light emitting device.

In one embodiment of the present invention, the organic light emitting device may be a red organic light emitting device, and the heterocyclic compound represented by Formula 1 may be used as a material of the red organic light emitting device.

In one embodiment of the present invention, the organic light emitting device may be a blue organic light emitting device, and the heterocyclic compound represented by Formula 1 may be used as a material for a light emitting layer of the blue organic light emitting device.

In one embodiment of the present invention, the organic light emitting device may be a green organic light emitting device, and the heterocyclic compound represented by Formula 1 may be used as a material for a light emitting layer of the green organic light emitting device.

In one embodiment of the present invention, the organic light emitting device may be a red organic light emitting device, and the heterocyclic compound represented by Formula 1 may be used as a material for a light emitting layer of the red organic light emitting device.

Specific details of the heterocyclic compound represented by Formula 1 are the same as described above.

The organic light emitting device of the present invention may be manufactured by a conventional method and material for manufacturing an organic light emitting device, except that one or more organic layers are formed using the aforementioned heterocyclic compound.

The heterocyclic compound may be formed as an organic layer by a solution coating method as well as a vacuum deposition method when manufacturing an organic light emitting device. In this case, the solution coating method means, but is not limited to, spin coating, dip coating, inkjet printing, screen printing, spraying, roll coating, and the like.

The organic layer of the organic light emitting device of the present invention may have a single-layer structure, and may also have a multi-layer structure formed by stacking two or more organic layers. For example, the organic light emitting device of the present invention may have a structure comprising a hole injection layer, a hole transport layer, a light emitting layer, an electron transport layer, an electron injection layer and the like as an organic layer. However, the structure of the organic light emitting device is not limited thereto and may include a smaller number of organic layers.

In the organic light emitting device according to an embodiment of the present invention, the organic layer including the heterocyclic compound represented by Formula 1 further comprises a heterocyclic compound represented by Formula 2 below:

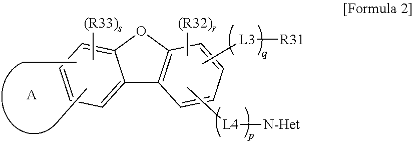

wherein,
N-Het is a substituted or unsubstituted, C2 to C60 monocyclic or polycyclic heterocyclic group containing one or more N,
L3 and L4 are the same as or different from each other, and are each independently a direct bond; a substituted or unsubstituted C6 to C60 arylene group; or a substituted or unsubstituted C6 to C60 heteroarylene group, p is an integer from 0 to 5, and when p is 2 or more, each L3 is the same as or different from each other, and q is an integer from 0 to 5, and when q is 2 or more, each L4 is the same as or different from each other,
A is a substituted or unsubstituted C6 to C60 aryl ring; or a substituted or unsubstituted C6 to C60 heteroaryl ring,
R31 to R33 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other are combined with each other to form a substituted or unsubstituted C6 to C60 aromatic hydrocarbon ring or a substituted or unsubstituted C2 to C60 heterocyclic ring, wherein R201, R202, and R203 are the same as or different from each other, and are each independently a substituted or unsubstituted C1 to C60 alkyl group; a substituted or unsubstituted C6 to C60 aryl group; or a substituted or unsubstituted C2 to C60 heteroaryl group, and
r and s are each an integer from 0 to 2, and when r is 2 or more, each R32 is the same as or different from each other, and when s is 2 or more, each R33 is the same as or different from each other.

In one embodiment of the present invention, the N-Het may be a substituted or unsubstituted, C2 to C30 monocyclic or polycyclic heterocyclic group containing one or more N.

In other embodiment of the present invention, the N-Het may be a substituted or unsubstituted, C3 to C30 monocyclic or polycyclic heterocyclic group containing 1 or more and 3 or less N.

In other embodiment of the present invention, he N-Het may be a substituted or unsubstituted, C3 to C10 monocyclic or polycyclic heterocyclic group containing 1 or more and 3 or less N.

In other embodiment of the present invention, the N-Het may be a monocyclic or polycyclic C2 to C30 heterocyclic group containing one or more N, which is unsubstituted or substituted with one or more substituents selected from the group consisting of a C6 to C60 aryl group and a C3 to C60 heteroaryl group.

In other embodiment of the present invention, the N-Het may be a triazine group; a pyrimidine group; a pyridine group; a quinoline group; a quinazoline group; a phenanthroline group; an imidazole group; a benzothiazole group; or a benzo[4,5]thieno[2,3-d]pyrimidine group which is unsubstituted or substituted with one or more substituents selected from the group consisting of a C6 to C60 aryl group and a C2 to C60 heteroaryl group.

In other embodiment of the present invention, the N-Het may be a triazine group; a pyrimidine group; a pyridine group; a quinoline group; a quinazoline group; a phenanthroline group; an imidazole group; a benzothiazole group; or a benzo[4,5]thieno[2,3-d]pyrimidine group substituted or unsubstituted with one or more substituents selected from the group consisting of a phenyl group, a biphenyl group, a naphthyl group, a triphenylenyl group, a dibenzofuranyl group, a dibenzothiophenyl group, a pyridinyl group, a dimethylfluorenyl group, a diphenylfluorenyl group, and a spirobifluorenyl group.

In other embodiment of the present invention, the N-Het may be a triazine group; a pyrimidine group; a quinoline group; or a benzo[4,5]thieno[2,3-d]pyrimidine group substituted or unsubstituted with one or more substituents selected from the group consisting of a phenyl group, a biphenyl group, a naphthyl group, a triphenylenyl group, a dibenzofuranyl group, a dibenzothiophenyl group, a pyridinyl group, a dimethylfluorenyl group, a diphenylfluorenyl group, and a spirobifluorenyl group.

In other embodiment of the present invention, the N-Het may be substituted again with —CN, a phenyl group, P(═O)RR′, or SiRR′R″. R, R′ and R″ are the same as or different from each other, and may each independently be a substituted or unsubstituted C1 to C60 alkyl group; a substituted or unsubstituted C6 to C60 aryl group; or a substituted or unsubstituted C2 to C60 heteroaryl group.

In one embodiment of the present invention, L3 and L4 may be the same as or different from each other, and may each independently be a direct bond; a substituted or unsubstituted C6 to C40 arylene group; or a substituted or unsubstituted C2 to C40 heteroarylene group.

In other embodiment of the present invention, L3 and L4 may be the same as or different from each other, and may each independently be a direct bond; a substituted or unsubstituted C6 to C10 arylene group; or a substituted or unsubstituted C2 to C10 heteroarylene group.

In other embodiment of the present invention, L3 and L4 may be the same as or different from each other, and may each independently be a direct bond; or a substituted or unsubstituted C6 to C10 arylene group.

In other embodiment of the present invention, L3 and L4 may be the same as or different from each other, and may each independently be a direct bond; a phenylene group; or a naphthylene group.

In one embodiment of the present invention, p and q may be the same as or different from each other, and may each independently be an integer of 1 to 3, and when p is 2 or more, each L4 may be the same as or different from each other, and when q is 2 or more, each L3 may be the same as or different from each other.

In one embodiment of the present invention, A may be a substituted or unsubstituted C6 to C40 aryl ring; or a substituted or unsubstituted C6 to C40 heteroaryl ring.

In other embodiment of the present invention, A may be a substituted or unsubstituted C6 to C40 aryl ring.

In other embodiment of the present invention, A may be a substituted or unsubstituted benzene ring; or a substituted or unsubstituted naphthyl ring.

In other embodiment of the present invention, A may be a benzene ring.

In one embodiment of the present application, the statement that A has a substituted or unsubstituted C6 to C40 aryl ring means that it comprises an unsubstituted C6 to C40 aryl ring or a substituted C6 to C40 aryl ring, and the substituent in the substituted C6 to C40 aryl ring comprises a condensed form by bonding with an adjacent group.

In one embodiment of the present invention, R31 may be a substituted or unsubstituted C6 to C60 aryl group; or a substituted or unsubstituted C2 to C60 heteroaryl group.

In other embodiment of the present invention, R31 may be a substituted or unsubstituted C6 to C30 aryl group; or a substituted or unsubstituted C2 to C30 heteroaryl group.

In other embodiment of the present invention, R31 may be a substituted or unsubstituted C6 to C20 aryl group; or a substituted or unsubstituted C2 to C20 heteroaryl group.

In other embodiment of the present invention, R31 may be a substituted or unsubstituted phenyl group.

In one embodiment of the present invention, R32 and R33 may be the same as or different from each other, and may each independently be selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C20 cycloalkyl group; a substituted or unsubstituted C2 to C20 heterocycloalkyl group; a substituted or unsubstituted C6 to C30 aryl group; a substituted or unsubstituted C2 to C30 heteroaryl group; —P(═O)R201R202; —SiR201R202R203; and —NR201R202, or two or more groups adjacent to each other may be combined with each other to form a substituted or unsubstituted C6 to C30 aromatic hydrocarbon ring or a substituted or unsubstituted C2 to C30 heterocycle, wherein R201, R202, and R203 above may be the same as or different from each other, and may each independently be a substituted or unsubstituted C1 to C10 alkyl group; a substituted or unsubstituted C6 to C30 aryl group; or a substituted or unsubstituted C2 to C30 heteroaryl group, and when R32 and R33 are plural, each of R32 and R33 may be the same as or different from each other.

In other embodiment of the present invention, R32 and R33 may be the same as or different from each other, and may each independently be hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C6 to C30 aryl group; or a substituted or unsubstituted C2 to C30 heteroaryl group, and when R32 and R33 are plural, each of R32 and R33 may be the same as or different from each other.

In other embodiment of the present invention, R32 and R33 may be the same as or different from each other, and may each independently be hydrogen; deuterium; a substituted or unsubstituted C1 to C5 alkyl group; a substituted or unsubstituted C2 to C5 alkenyl group; a substituted or unsubstituted C2 to C5 alkynyl group; a substituted or unsubstituted C6 to C20 aryl group; or a substituted or unsubstituted C2 to C20 heteroaryl group, and when R32 and R33 are plural, each of R32 and R33 may be the same as or different from each other.

In other embodiment of the present invention, R32 and R33 may be the same as or different from each other, and may each independently be hydrogen; deuterium; a substituted or unsubstituted C1 to C5 alkyl group; a substituted or unsubstituted C6 to C20 aryl group; or a substituted or unsubstituted C2 to C20 heteroaryl group, and when R32 and R33 are plural, each of R32 and R33 may be the same as or different from each other.

In other embodiment of the present invention, R32 and R33 may be the same as or different from each other, and may each independently be hydrogen, deuterium, a substituted or unsubstituted C1 to C5 alkyl group, a substituted or unsubstituted phenyl group, a naphthalenyl group, a pyridinyl group, an anthracenyl group, a carbazole group, a dibenzothiophene group, a dibenzofuran group, or a phenanthrenyl group, and when R32 and R33 are plural, each of R32 and R33 may be the same as or different from each other.

In other embodiment of the present invention, R32 and R33 may be the same as or different from each other, and may each independently be hydrogen, deuterium, and a substituted or unsubstituted C1 to C5 alkyl group. In this case, the C1 to C5 alkyl group may be a methyl group, an ethyl group, a straight or branched propyl group, a straight or branched butyl group, or a straight or branched pentyl group, and when R32 and R33 are plural, each of R32 and R33 may be the same as or different from each other.

In other embodiment of the present invention, R32 and R33 may be the same as or different from each other, and may each independently be hydrogen or deuterium, and when R32 and R33 are plural, each of R32 and R33 may be the same as or different from each other.

In one embodiment of the present invention, r and s may be the same as or different from each other, and may each independently be an integer of 1 to 2, and when r is 2, each R32 may be the same as or different from each other, and when s is 2, each R33 may be the same as or different from each other.

In other embodiment of the present invention, the ‘substitution’ of N-Het, L3, L4, A, and R31 to R33 may each independently be made by one or more substituents selected from the group consisting of a C1 to C10 alkyl group; a C2 to C10 alkenyl group; a C2 to C10 alkynyl group; a C3 to C15 cycloalkyl group; a C2 to C20 heterocycloalkyl group; a C6 to C30 aryl group; a C2 to C30 heteroaryl group; a C1 to C10 alkylamine group; a C6 to C30 arylamine group; and a C2 to C30 heteroarylamine group.

In other embodiment of the present invention, the ‘substitution’ of N-Het, L3, L4, A, and R31 to R33 may each independently be made by one or more substituents selected from the group consisting of a C1 to C10 alkyl group; a C6 to C30 aryl group; and a C2 to C30 heteroaryl group.

In other embodiment of the present invention, the ‘substitution’ of N-Het, L3, L4, A, and R31 to R33 may each independently be made by one or more substituents selected from the group consisting of a C1 to C5 alkyl group; a C6 to C20 aryl group; and a C2 to C20 heteroaryl group.

In other embodiment of the present invention, the ‘substitution’ of N-Het, L3, L4, A, and R31 to R33 may each independently be made by one or more substituents selected from the group consisting of a methyl group, an ethyl group, a straight or branched propyl group, a straight or branched butyl group, a straight or branched pentyl group, a phenyl group, a naphthyl group, a pyridinyl group, an anthracenyl group, a carbazolyl group, a dibenzothiophenyl group, a dibenzofuranyl group, and a phenanthrenyl group.

In other embodiment of the present invention, the ‘substitution’ of N-Het, L3, L4, A, and R31 to R33 may each independently be made by a methyl group, an ethyl group, a straight or branched propyl group, a straight or branched butyl group, and a straight or branched pentyl group.

In one embodiment of the present invention, Formula 2 may be a heterocyclic compound represented by any one of Formula 2-1 to Formula 2-3 below:

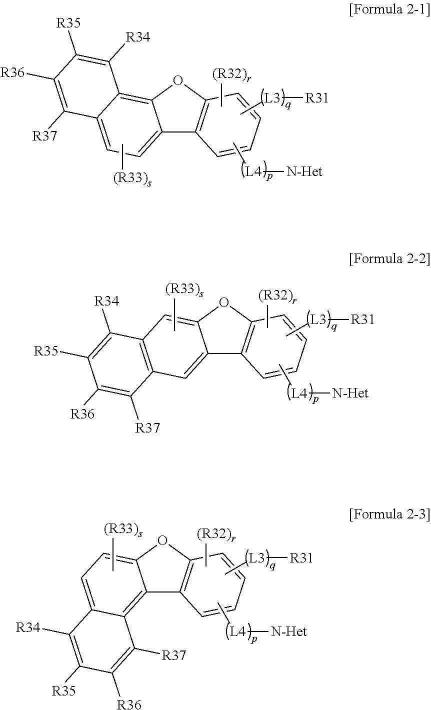

wherein,
R34 to R37 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other are combined with each other to form a substituted or unsubstituted C6 to C60 aromatic hydrocarbon ring or a substituted or unsubstituted C2 to C60 heterocyclic ring, wherein R201, R202, and R203 are the same as or different from each other, and are each independently a substituted or unsubstituted C1 to C60 alkyl group; a substituted or unsubstituted C6 to C60 aryl group; or a substituted or unsubstituted C2 to C60 heteroaryl group, and
the definitions of N-Het, L3, L4, R31 to R33, p, q, r, and s are the same as those in Formula 2 above.

In one embodiment of the present invention, R34 to R37 may be the same as or different from each other, and may each independently be selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other may be combined with each other to form a substituted or unsubstituted C6 to C60 aromatic hydrocarbon ring or a substituted or unsubstituted C2 to C60 heterocyclic ring, wherein R201, R202, and R203 may be the same as or different from each other, and may each independently be a substituted or unsubstituted C1 to C60 alkyl group; a substituted or unsubstituted C6 to C60 aryl group; or a substituted or unsubstituted C2 to C60 heteroaryl group.

In other embodiment of the present invention, R34 to R37 may be the same as or different from each other, and may each independently be hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C6 to C30 aryl group; or a substituted or unsubstituted C2 to C30 heteroaryl group.

In other embodiment of the present invention, R34 to R37 may be the same as or different from each other, and may each independently be hydrogen; deuterium; a substituted or unsubstituted C1 to C5 alkyl group; a substituted or unsubstituted C2 to C5 alkenyl group; a substituted or unsubstituted C2 to C5 alkynyl group; a substituted or unsubstituted C6 to C20 aryl group; or a substituted or unsubstituted C2 to C20 heteroaryl group.

In other embodiment of the present invention, R34 to R37 may be the same as or different from each other, and may each independently be hydrogen; deuterium; a substituted or unsubstituted C1 to C5 alkyl group; a substituted or unsubstituted C6 to C20 aryl group; or a substituted or unsubstituted C2 to C20 heteroaryl group.

In other embodiment of the present invention, R34 to R37 may be the same as or different from each other, and may each independently be hydrogen, deuterium, a substituted or unsubstituted C1 to C5 alkyl group, a substituted or unsubstituted phenyl group, naphthyl group, pyridinyl group, anthracenyl, carbazolyl, dibenzothiophenyl, dibenzofuranyl, or phenanthrenyl group.

In other embodiment of the present invention, R34 to R37 may be the same as or different from each other, and may each independently be hydrogen, deuterium, and a substituted or unsubstituted C1 to C5 alkyl group. In this case, the C1 to C5 alkyl group may be a methyl, ethyl, straight or branched propyl, straight or branched butyl, or straight or branched pentyl group.

In other embodiment of the present invention, R34 to R37 may be the same as or different from each other, and may each independently be hydrogen or deuterium.

In other embodiment of the present invention, the ‘substitution’ of R34 to R37 may each independently be made by one or more substituents selected from the group consisting of a C1 to C10 alkyl group; a C2 to C10 alkenyl group; a C2 to C10 alkynyl group; a C3 to C15 cycloalkyl group; a C2 to C20 heterocycloalkyl group; a C6 to C30 aryl group; a C2 to C30 heteroaryl group; a C1 to C10 alkylamine group; a C6 to C30 arylamine group; and a C2 to C30 heteroarylamine group.

In other embodiment of the present invention, the ‘substitution’ of R34 to R37 may each independently be made by one or more substituents selected from the group consisting of a C1 to C10 alkyl group; a C6 to C30 aryl group; and a C2 to C30 heteroaryl group.

In other embodiment of the present invention, the ‘substitution’ of R34 to R37 may each independently be made by one or more substituents selected from the group consisting of a C1 to C5 alkyl group; a C6 to C20 aryl group; and a C2 to C20 heteroaryl group.

In other embodiment of the present invention, the ‘substitution’ of R34 to R37 may each independently be made by one or more substituents selected from the group consisting of a methyl group, an ethyl group, a straight or branched propyl group, a straight or branched butyl group, a straight or branched pentyl group, a phenyl group, a naphthyl group, a pyridinyl group, an anthracenyl group, a carbazolyl group, a dibenzothiophenyl group, a dibenzofuranyl group, and a phenanthrenyl group.

In other embodiment of the present invention, the ‘substitution’ of R34 to R37 may each independently be made by a methyl group, an ethyl group, a straight or branched propyl group, a straight or branched butyl group, and a straight or branched pentyl group.

In one embodiment of the present invention, the N-Het may be a heterocyclic compound represented by any one of Formula 3-1 to Formula 3-4 below:

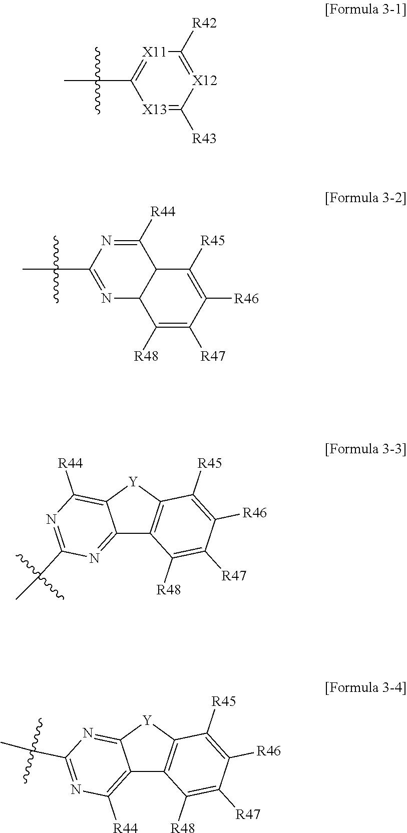

wherein,
X11 to X13 are the same as or different from each other, and each independently are N or CR41, and at least two of X11 to X13 are N,
Y is O; or S,
R42 to R44 are the same as or different from each other, and are each independently a substituted or unsubstituted C6 to C60 aryl group; a substituted or unsubstituted C2 to C60 heteroaryl group, and
R41 and R45 to R48 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other are combined with each other to form a substituted or unsubstituted C6 to C60 aromatic hydrocarbon ring or a substituted or unsubstituted C2 to C60 heterocyclic ring, wherein R301, R302, and R303 are the same as or different from each other, and are each independently a substituted or unsubstituted C1 to C60 alkyl group; a substituted or unsubstituted C6 to C60 aryl group; or a substituted or unsubstituted C2 to C60 heteroaryl group.

In one embodiment of the present invention, X11 to X13 may be the same as or different from each other, and may each independently be N or CR41, and Two or all of X11 to X13 may be N.

In one embodiment of the present invention, Y may be O or S.

In other embodiment of the present invention, Y may be O.

In other embodiment of the present invention, Y may be S.

In one embodiment of the present invention, R42 to R44 may be the same as or different from each other, and may each independently be a substituted or unsubstituted C6 to C30 aryl group; a substituted or unsubstituted C2 to C30 heteroaryl group.

In other embodiment of the present invention, R42 to R44 may be the same as or different from each other, and may each independently be a substituted or unsubstituted C6 to C20 aryl group; a substituted or unsubstituted C2 to C20 heteroaryl group.

In other embodiment of the present invention, R42 to R44 may be the same as or different from each other, and may each independently be a substituted or unsubstituted phenyl group, a naphthyl group; or a substituted or unsubstituted dibenzofuranyl group.

In one embodiment of the present invention, R41 and R45 to R48 may be the same as or different from each other, and may each independently be selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R301R302; —SiR301R302R303; and —NR301R302, or two or more groups adjacent to each other may be combined with each other to form a substituted or unsubstituted C6 to C30 aromatic hydrocarbon ring or substituted or unsubstituted C2 to C30 heterocyclic ring, wherein R301, R302, and R303 may be the same as or different from each other, and may each independently be a substituted or unsubstituted C1 to C30 alkyl group; a substituted or unsubstituted C6 to C30 aryl group; or a substituted or unsubstituted C2 to C30 heteroaryl group.

In other embodiment of the present invention, R41 and R45 to R48 may be the same as or different from each other, and may each independently be hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C6 to C20 aryl group; or a substituted or unsubstituted C2 to C20 heteroaryl group.

In other embodiment of the present invention, R41 and R45 to R48 may be the same as or different from each other, and may each independently be hydrogen; deuterium; substituted or unsubstituted C1 to C5 alkyl group; a substituted or unsubstituted C2 to C5 alkenyl group; a substituted or unsubstituted C2 to C5 alkynyl group; a substituted or unsubstituted C6 to C10 aryl group; or a substituted or unsubstituted C2 to C10 heteroaryl group.

In other embodiment of the present invention, R41 and R45 to R48 may be the same as or different from each other, and may each independently be hydrogen; deuterium; a substituted or unsubstituted C1 to C5 alkyl group; a substituted or unsubstituted C6 to C10 aryl group; or a substituted or unsubstituted C2 to C10 heteroaryl group.

In other embodiment of the present invention, R41 and R45 to R48 may be the same as or different from each other, and may each independently be hydrogen, deuterium, a substituted or unsubstituted C1 to C5 alkyl group, a substituted or unsubstituted phenyl group, a naphthyl group, a pyridinyl group, an anthracenyl group, a carbazolyl group, a dibenzothiophenyl group, a dibenzofuranyl group, or a phenanthrenyl group.

In other embodiment of the present invention, R41 and R45 to R48 may be the same as or different from each other, and may each independently be hydrogen, deuterium, and a substituted or unsubstituted C1 to C5 alkyl group. In this case, the C1 to C5 alkyl group may be a methyl group, an ethyl group, a straight or branched propyl group, a straight or branched butyl group, or a straight or branched pentyl group.

In other embodiment of the present invention, R41 and R45 to R48 may be the same as or different from each other, and may each independently be hydrogen or deuterium.

In other embodiment of the present invention, the ‘substitution’ of R41 to R48 may each independently be made by one or more substituents selected from the group consisting of a C1 to C10 alkyl group; a C2 to C10 alkenyl group; a C2 to C10 alkynyl group; a C3 to C15 cycloalkyl group; a C2 to C20 heterocycloalkyl group; a C6 to C30 aryl group; a C2 to C30 heteroaryl group; a C1 to C10 alkylamine group; a C6 to C30 arylamine group; and a C2 to C30 heteroarylamine group.

In other embodiment of the present invention, the ‘substitution’ of R41 to R48 may each independently be made by one or more substituents selected from the group consisting of a C1 to C10 alkyl group; and a C6 to C30 aryl group; a C2 to C30 heteroaryl group.

In other embodiment of the present invention, the ‘substitution’ of R41 to R48 may each independently be made by one or more substituents selected from the group consisting of a C1 to C5 alkyl group; a C6 to C20 aryl group; and a C2 to C20 heteroaryl group.

In other embodiment of the present invention, the ‘substitution’ of R41 to R48 may each independently be made by one or more substituents selected from the group consisting of a methyl group, an ethyl group, a straight or branched propyl group, a straight or branched butyl group, a straight or branched pentyl group, a phenyl group, a naphthyl group, a pyridinyl group, an anthracenyl group, a carbazolyl group, a dibenzothiophenyl group, a dibenzofuranyl group, and a phenanthrenyl group.

In other embodiment of the present invention, the ‘substitution’ of R41 to R48 may each independently be made by a methyl group, an ethyl group, a straight or branched propyl group, a straight or branched butyl group, and a straight or branched pentyl group.

In one embodiment of the present invention, the heterocyclic compound represented by Formula 2 may be at least one selected from the compounds below:

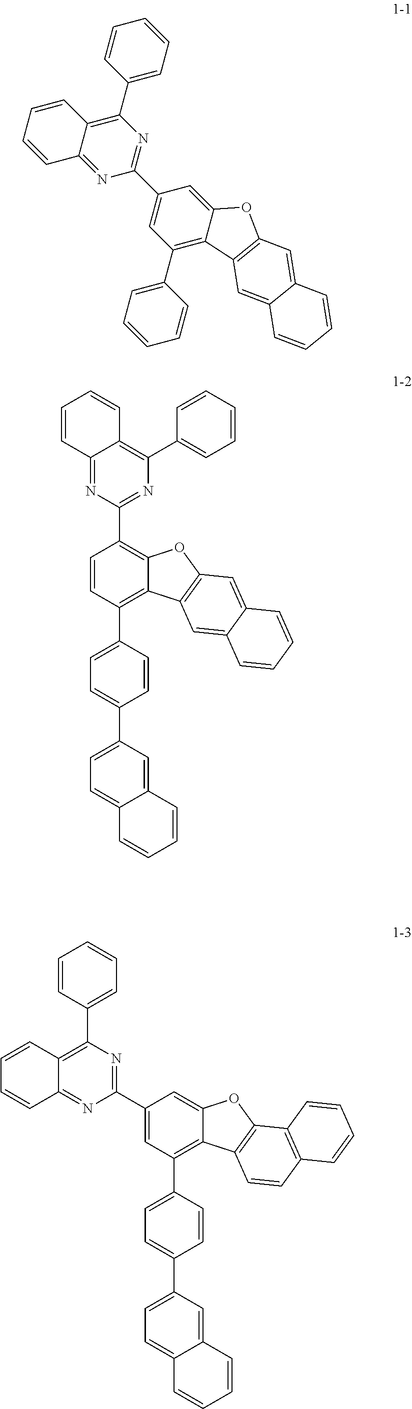 [SMILES: c1ccc(-c2nc(-c3cc(-c4ccccc4)c4c(c3)oc3cc5ccccc5cc34)nc3ccccc23)cc1 ; c1ccc(-c2nc(-c3ccc(-c4ccc(-c5ccc6ccccc6c5)cc4)c4c3oc3cc5ccccc5cc34)nc3ccccc23)cc1 ; c1ccc(-c2nc(-c3cc(-c4ccc(-c5ccc6ccccc6c5)cc4)c4c(c3)oc3c5ccccc5ccc34)nc3ccccc23)cc1]

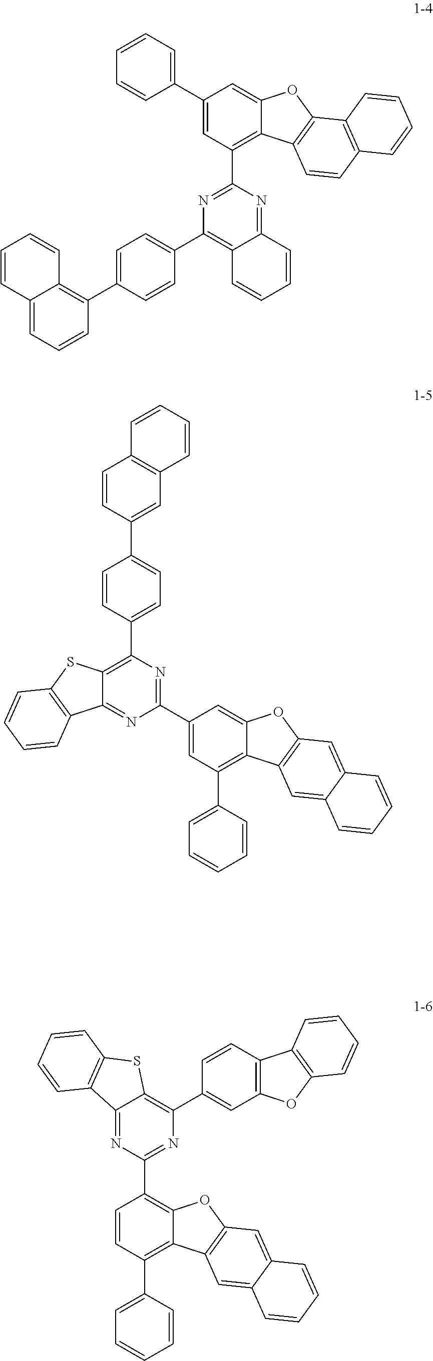 [SMILES: c1ccc(-c2cc(-c3nc(-c4ccc(-c5cccc6ccccc56)cc4)c4ccccc4n3)c3c(c2)oc2c4ccccc4ccc23)cc1 ; c1ccc(-c2cc(-c3nc(-c4ccc(-c5ccc6ccccc6c5)cc4)c4sc5ccccc5c4n3)cc3oc4cc5ccccc5cc4c23)cc1 ; c1ccc(-c2ccc(-c3nc(-c4ccc5c(c4)oc4ccccc45)c4sc5ccccc5c4n3)c3oc4cc5ccccc5cc4c23)cc1]

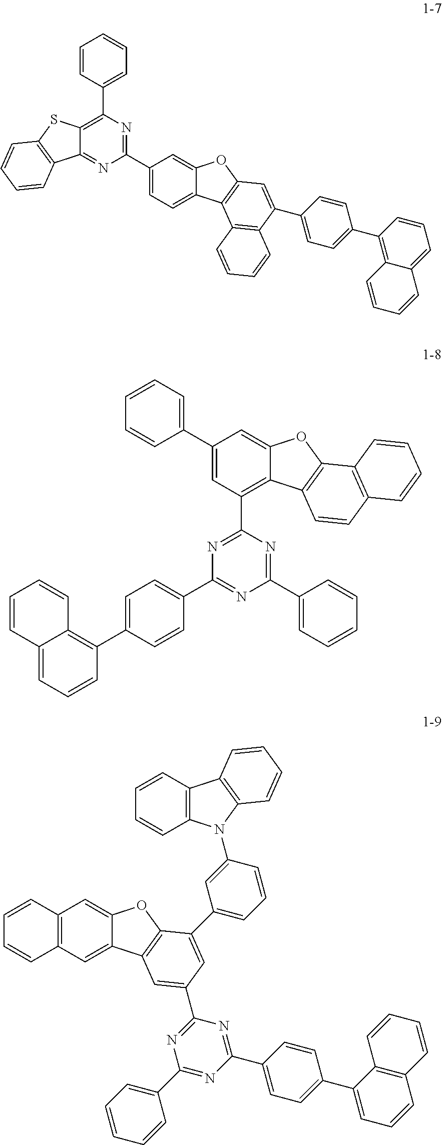 [SMILES: c1ccc(-c2nc(-c3ccc4c(c3)oc3cc(-c5ccc(-c6cccc7ccccc67)cc5)c5ccccc5c34)nc3c2sc2ccccc23)cc1 ; c1ccc(-c2cc(-c3nc(-c4ccccc4)nc(-c4ccc(-c5cccc6ccccc56)cc4)n3)c3c(c2)oc2c4ccccc4ccc23)cc1 ; c1ccc(-c2nc(-c3ccc(-c4cccc5ccccc45)cc3)nc(-c3cc(-c4cccc(-n5c6ccccc6c6ccccc65)c4)c4oc5cc6ccccc6cc5c4c3)n2)cc1]

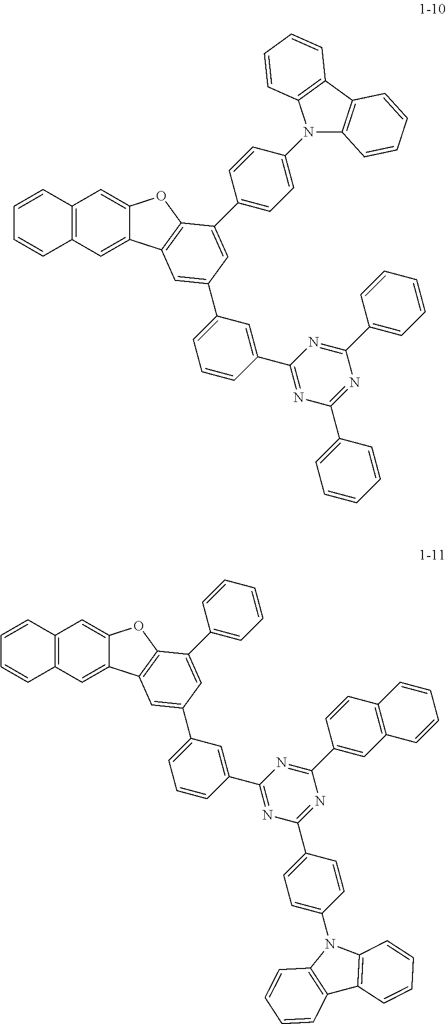 [SMILES: c1ccc(-c2nc(-c3ccccc3)nc(-c3cccc(-c4cc(-c5ccc(-n6c7ccccc7c7ccccc76)cc5)c5oc6cc7ccccc7cc6c5c4)c3)n2)cc1 ; c1ccc(-c2cc(-c3cccc(-c4nc(-c5ccc(-n6c7ccccc7c7ccccc76)cc5)nc(-c5ccc6ccccc6c5)n4)c3)cc3c2oc2cc4ccccc4cc23)cc1]

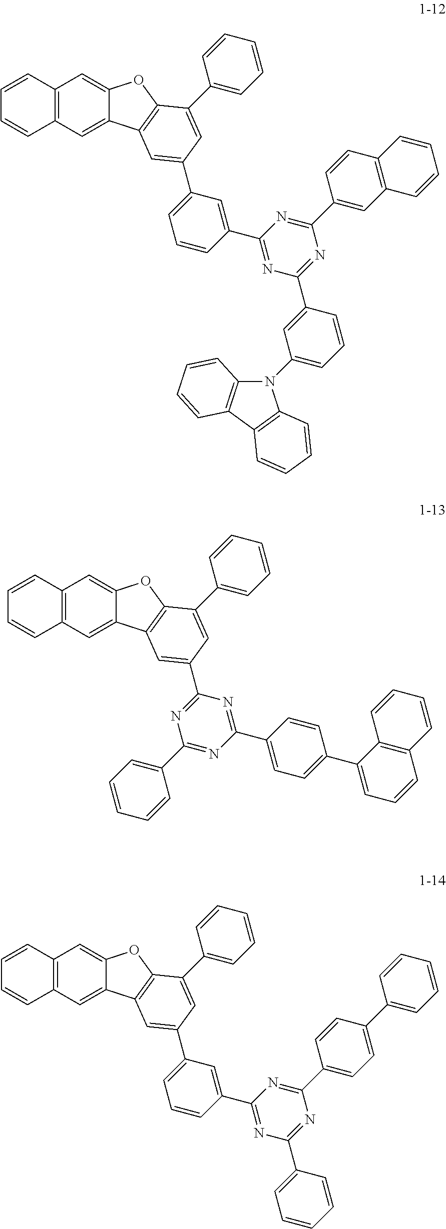 [SMILES: c1ccc(-c2cc(-c3cccc(-c4nc(-c5cccc(-n6c7ccccc7c7ccccc76)c5)nc(-c5ccc6ccccc6c5)n4)c3)cc3c2oc2cc4ccccc4cc23)cc1 ; c1ccc(-c2nc(-c3ccc(-c4cccc5ccccc45)cc3)nc(-c3cc(-c4ccccc4)c4oc5cc6ccccc6cc5c4c3)n2)cc1 ; c1ccc(-c2ccc(-c3nc(-c4ccccc4)nc(-c4cccc(-c5cc(-c6ccccc6)c6oc7cc8ccccc8cc7c6c5)c4)n3)cc2)cc1]

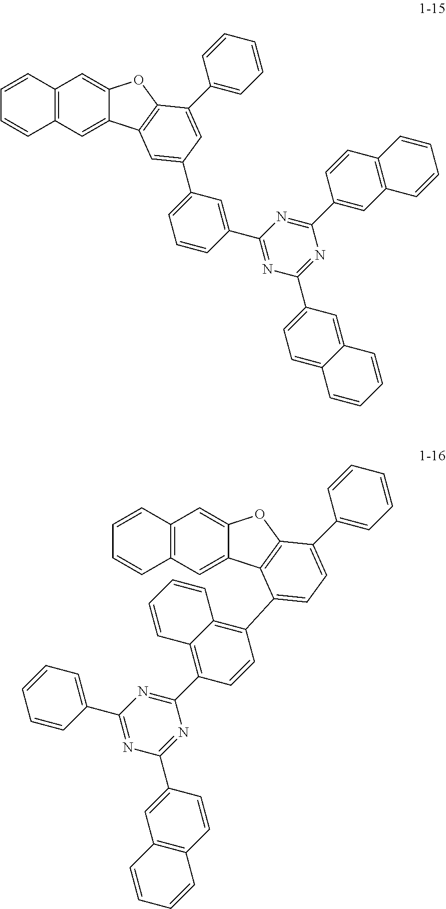 [SMILES: c1ccc(-c2cc(-c3cccc(-c4nc(-c5ccc6ccccc6c5)nc(-c5ccc6ccccc6c5)n4)c3)cc3c2oc2cc4ccccc4cc23)cc1 ; c1ccc(-c2nc(-c3ccc4ccccc4c3)nc(-c3ccc(-c4ccc(-c5ccccc5)c5oc6cc7ccccc7cc6c45)c4ccccc34)n2)cc1]

In addition, one embodiment of the present invention provides a composition for organic layer of an organic light emitting device including the heterocyclic compound represented by Formula 1 and the heterocyclic compound represented by Formula 2.

Specific details of the heterocyclic compound represented by Formula 1 and the heterocyclic compound represented by Formula 2 are the same as described above.

In one embodiment of the present invention, a weight ratio of the heterocyclic compound represented by Formula 1 above and the heterocyclic compound represented by Formula 2 above in the composition for organic layer of the organic light emitting device may be 1:10 to 10:1, 1:8 to 8:1, 1:5 to 5:1, and 1:2 to 2:1, but is not limited thereto.

The composition for organic layer of the organic light emitting device may be used when forming an organic material of the organic light emitting device, and may be in particular used when forming a host of the light emitting layer.

In one embodiment of the present invention, the organic layer includes the heterocyclic compound represented by Formula 1 and the heterocyclic compound represented by Formula 2, and may be used together with a phosphorescent dopant.

The phosphorescent dopant material may be those known in the art. For example, phosphorescent dopant materials represented by LL′MX′, LL′L″M, LMX′X″, L₂MX′ and L₃M may be used, but the scope of the present invention is not limited to these examples.

M may be iridium, platinum, osmium, or the like.

L is an anionic bidentate ligand coordinated to M by sp² carbon and a hetero atom, and X may function to trap electrons or holes. Non-limiting examples of L comprise 2-(1-naphthyl)benzoxazole, (2-phenylbenzoxazole), (2-phenylbenzothiazole), (2-phenylbenzothiazole), (7,8-benzoquinoline), (thiophenyl pyridine), phenylpyridine, benzothiophenyl pyridine, 3-methoxy-2-phenylpyridine, thiophene group pyrizine, tolyl pyridine and the like. Non-limiting examples of X′ and X″cetylacetonate (acac), hexafluoroacetylacetonate, salicylidene, picolinate, 8-hydroxyquinolinate, and the like.

Specific examples of the phosphorescent dopant are shown below, but are not limited thereto:

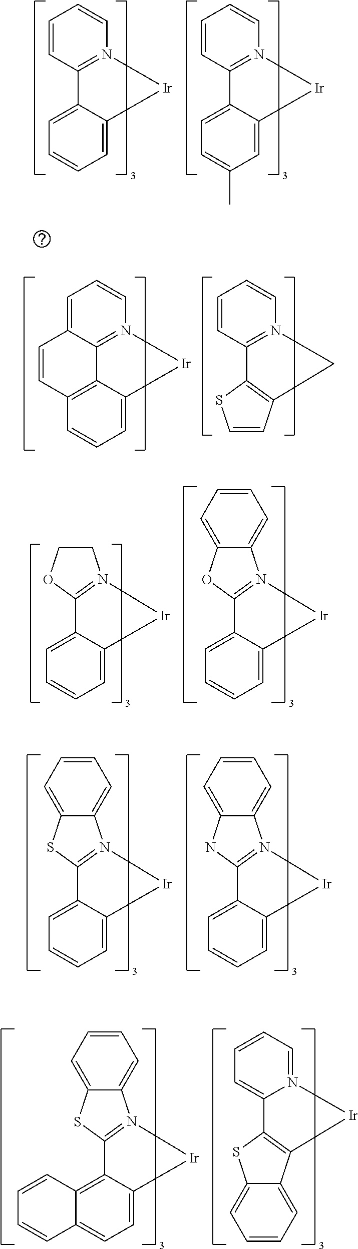

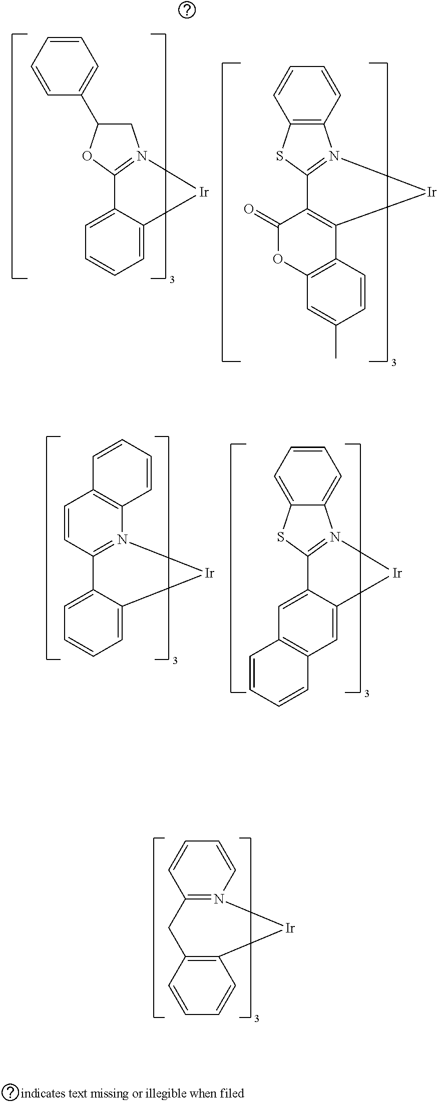

In one embodiment of the present invention, the organic layer comprises the heterocyclic compound represented by Formula 1 and the heterocyclic compound represented by Formula 2, and may be used together with an iridium-based dopant.

In one embodiment of the present invention, (piq)₂(Ir)(acac) which is a red phosphorescent dopant may be used as the iridium-based dopant.

In one embodiment of the present invention, the content of the dopant may be 1 wt. % to 15 wt. %, preferably 2 wt. % to 10 wt. %, based on the total of the light emitting layer.

In the organic light emitting device according to an embodiment of the present invention, the organic layer may comprise an electron injection layer or an electron transport layer, and the electron injection layer or the electron transport layer may comprise the heterocyclic compound.

In the organic light emitting device according to another embodiment, the organic layer may comprise an electron blocking layer or a hole blocking layer, and the electron blocking layer or the hole blocking layer may comprise the heterocyclic compound.

In the organic light emitting device according to another embodiment, the organic layer may comprise an electron transport layer, a light emitting layer, or a hole blocking layer, and the electron transport layer, the light emitting layer, or the hole blocking layer may comprise the heterocyclic compound.

The organic light emitting device according to an embodiment of the present invention may further comprise one layer or more layers selected from the group consisting of a light emitting layer, a hole injection layer, a hole transport layer, an electron injection layer, an electron transport layer, an electron blocking layer, and a hole blocking layer.

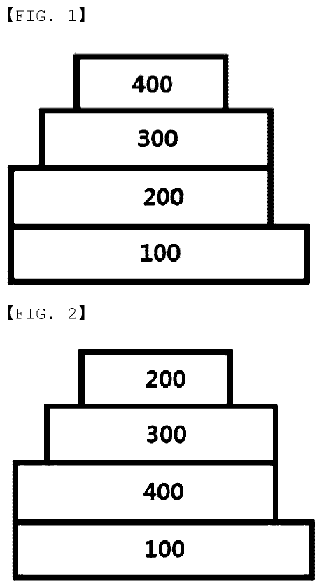

FIGS. 1 to 3 illustrate the stacking order of the electrode and the organic layer of the organic light emitting device according to an embodiment of the present invention. However, it is not intended that the scope of the present application be limited by these drawings, and the structure of the organic light emitting device known in the art may also be applied to the present application.

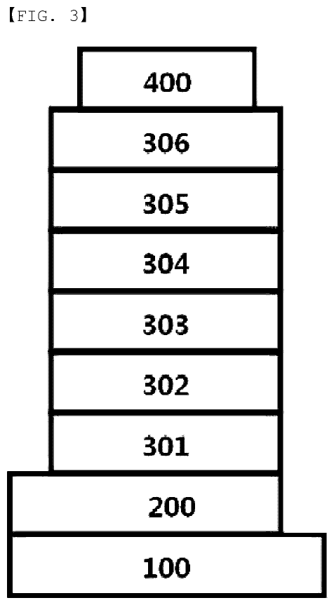

According to FIG. 1, an organic light emitting device formed by sequentially stacking a positive electrode 200, an organic layer 300, and a negative electrode 400 on a substrate 100 is shown. However, it is not limited to such a structure, and an organic light emitting device formed by sequentially stacking a negative electrode, an organic layer, and a positive electrode on a substrate may be implemented, as shown in FIG. 2.

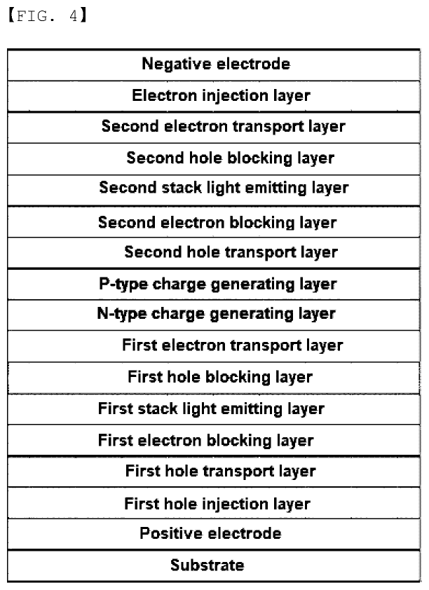

FIG. 3 illustrates a case in which the organic layer is composed of multiple layers. The organic light emitting device according to FIG. 3 comprises a hole injection layer 301, a hole transport layer 302, a light emitting layer 303, a hole blocking layer 304, an electron transport layer 305, and an electron injection layer 306. However, the scope of the present application is not limited by the stacked structure as described above, and if necessary, the remaining layers except for the light emitting layer may be omitted, and other necessary functional layers may be further added.

According to an embodiment of the present invention, the organic light emitting device may have a tandem structure in which two or more independent devices are connected in series. In one embodiment, the tandem structure may be in a form in which each organic light emitting device is bonded through a charge generating layer. Since the device with the tandem structure can operate at a lower current than each unit device based on the same luminance, there is an advantage that the lifetime characteristics of the device are greatly improved.

According to one embodiment of the present invention, the organic layer comprises the first stack comprising one or more light emitting layers; the second stack comprising one or more light emitting layers; and one or more charge generating layers provided between the first stack and the second stack.

According to another embodiment of the present invention, the organic layer comprises the first stack comprising one or more light emitting layers; the second stack comprising one or more light emitting layers; and the third stack comprising one or more light emitting layers, and comprises one or more charge generating layers between the first stack and the second stack and between the second stack and the third stack, respectively.

The term charge generating layer may mean a layer that generates holes and electrons when a voltage is applied thereto. The charge generating layer may be an N-type charge generating layer or a P-type charge generating layer. In the present invention, the term N-type charge generating layer means a charge generating layer located closer to the positive electrode than the P-type charge generating layer, and the term P-type charge generating layer means a charge generating layer located closer to the negative electrode than the N-type charge generating layer.

The N-type charge generating layer and the P-type charge generating layer may be provided in contact, and in this case, an N+P junction is formed. By the N+P junction, holes are easily formed in the P-type charge generating layer and electrons are easily formed in the N-type charge generating layer. The electrons are transported in the direction of the positive electrode through the LUMO level of the N-type charge generating layer, and holes are transported in the direction of the negative electrode through the HOMO level of the P-type charge generating layer.

The first stack, the second stack and the third stack each independently comprise one or more light emitting layers, and may additionally comprise a hole injection layer, a hole transport layer, an electron blocking layer, an electron injection layer, an electron transport layer, a hole blocking layer, and one or more layers among a layer that transports and injects holes at the same time (hole injection and transport layer), and a layer that simultaneously transports and injects electrons (electron injection and transport layer).

An organic light emitting device comprising the first stack and the second stack is illustrated in FIG. 4. However, it is not intended that the scope of the present invention be limited by these drawings, and the structure of an organic light emitting device known in the art may also be applied to the present invention.

The first electron blocking layer, the first hole blocking layer, and the second hole blocking layer illustrated in FIG. 4 may be omitted in some cases.

In one embodiment of the present invention, the present invention provides a method of manufacturing an organic light emitting device comprising the steps of preparing a substrate; forming a first electrode on the substrate; forming one or more organic layers on the first electrode; and forming a second electrode on the one or more organic layers, wherein the step of forming the one or more organic layers comprises a step of forming the one or more organic layers using the composition for organic layer according to an embodiment of the present invention.

The pre-mixing refers to first mixing the heterocyclic compound represented by Formula 1 and the heterocyclic compound represented by Formula 2 and putting them in one source to mix them, before deposition on the organic layer.

The pre-mixed material may be referred to as a composition for organic layer according to one embodiment of the present application.

The organic layer comprising the heterocyclic compound represented by Formula 1 may further comprise another material, if necessary.

The organic layer simultaneously comprising the heterocyclic compounds represented by Formula 1 and 2 may further comprise another material, if necessary.

In the organic light emitting device according to an embodiment of the present invention, the materials other than the heterocyclic compounds represented by Formula 1 or Formula 2 are exemplified below, but these are for illustrative purposes only and are not intended to limit the scope of the present application, and may be replaced by materials known in the art.

As the positive electrode material, materials having a relatively large work function can be used, and a transparent conductive oxide, metal, a conductive polymer or the like may be used. Specific examples of the positive electrode material may be, but is not limited to, metals such as vanadium, chromium, copper, zinc, and gold or alloys thereof; metal oxides such as zinc oxide, indium oxide, indium tin oxide (ITO), and indium zinc oxide (IZO); a combination of metal and oxide such as ZnO:Al or SnO₂:Sb; conductive polymers such as poly(3-methylthiophene), poly[3,4-(ethylene-1,2-dioxy)thiophene](PEDOT), polypyrrole and polyaniline; and the like.

As the negative electrode material, materials having a relatively low work function may be used, and metal, metal oxide, a conductive polymer or the like may be used. Specific examples of the negative electrode material may be, but is not limited to, metals such as magnesium, calcium, sodium, potassium, titanium, indium, yttrium, lithium, gadolinium, aluminum, silver, tin and lead or alloys thereof; multilayer-structured materials such as LiF/Al or LiO₂/Al; and the like.

As the hole injection layer material, a known material for the hole injection layer may be used, and for example, phthalocyanine compounds such as copper phthalocyanine disclosed in U.S. Pat. No. 4,356,429, or starburst-type amine derivatives described in [Advanced Material, 6, p. 677 (1994)], such as tris(4-carbazoyl-9-ylphenyl)amine(TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid which is a soluble conductive polymer, or poly(3,4-ethylenedioxythiophene)/poly(4-styrenesulfonate), polyaniline/camphor sulfonic acid, polyaniline/poly(4-styrene-sulfonate), or the like may be used.

As a material for the hole transport layer, a pyrazoline derivative, an arylamine-based derivative, a stilbene derivative, a triphenyldiamine derivative, or the like may be used, and a low-molecular or high-molecular material may be used.

As a material for the electron transport layer, metal complexes of oxadiazole derivatives, anthraquinodimethane and derivatives thereof, benzoquinone and derivatives thereof, naphthoquinone and its derivatives, anthraquinone and its derivatives, tetracyanoanthraquinodimethane and its derivatives, fluorenone derivatives, diphenyldicyanoethylene and its derivatives, diphenoquinone derivatives, 8-hydroxyquinoline and its derivatives, and the like may be used, and high-molecular materials as well as low-molecular materials may be also used.

As the electron injection layer material, for example, LiF is typically used in the art, but the present application is not limited thereto.

As a material for the light emitting layer, a red, green or blue light emitting material may be used, and if necessary, two or more light emitting materials may be mixed and used. In this case, 2 or more luminescent materials may be used by deposition from separate sources, or may be pre-mixed and deposited from one source. In addition, as a material for the light emitting layer, a fluorescent material may be used, and a phosphorescent material may also be used. As a material for the light emitting layer, a material that emits light by combining holes and electrons respectively injected from the positive electrode and the negative electrode alone may be used, and materials in which the host material and the dopant material together participate in light emission may be also used.

If the hosts of the material for the light emitting layer are mixed and used, hosts of the same type may be mixed and used, or hosts of different types may be mixed and used. For example, any two or more types of n-type host material or p-type host material may be selected and used as a host material for the light emitting layer.

The organic light emitting device according to an embodiment of the present invention may be a top emission type, a bottom emission type, or a double side emission type depending on the material used.

The heterocyclic compound according to an embodiment of the present invention may also act in organic electronic devices including an organic solar cell, an organic photoreceptor, an organic transistor, and the like through the principle similar to that applied to the organic light emitting device.

Hereinafter, preferred examples are presented to help the understanding of the present invention. However, the following examples are provided only to make the present invention easier to understand, and the present invention is not limited thereto.

### PREPARATION EXAMPLE

### <Preparation Example 1-1> Preparation of Compound A7(71) and Compound A′ (98)

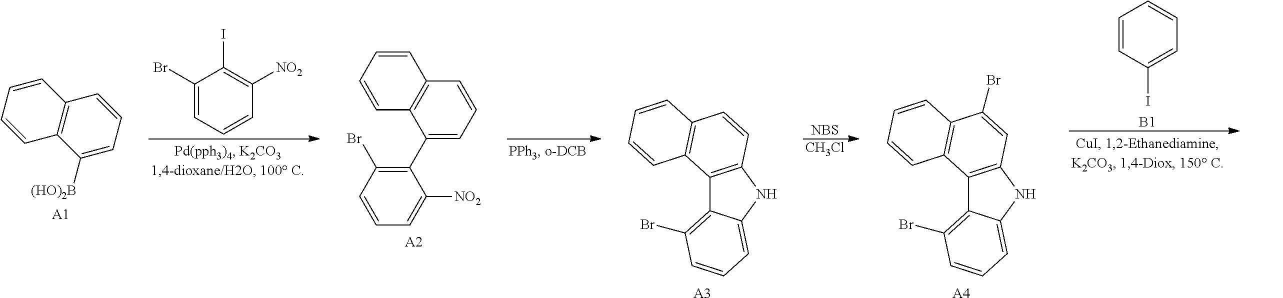

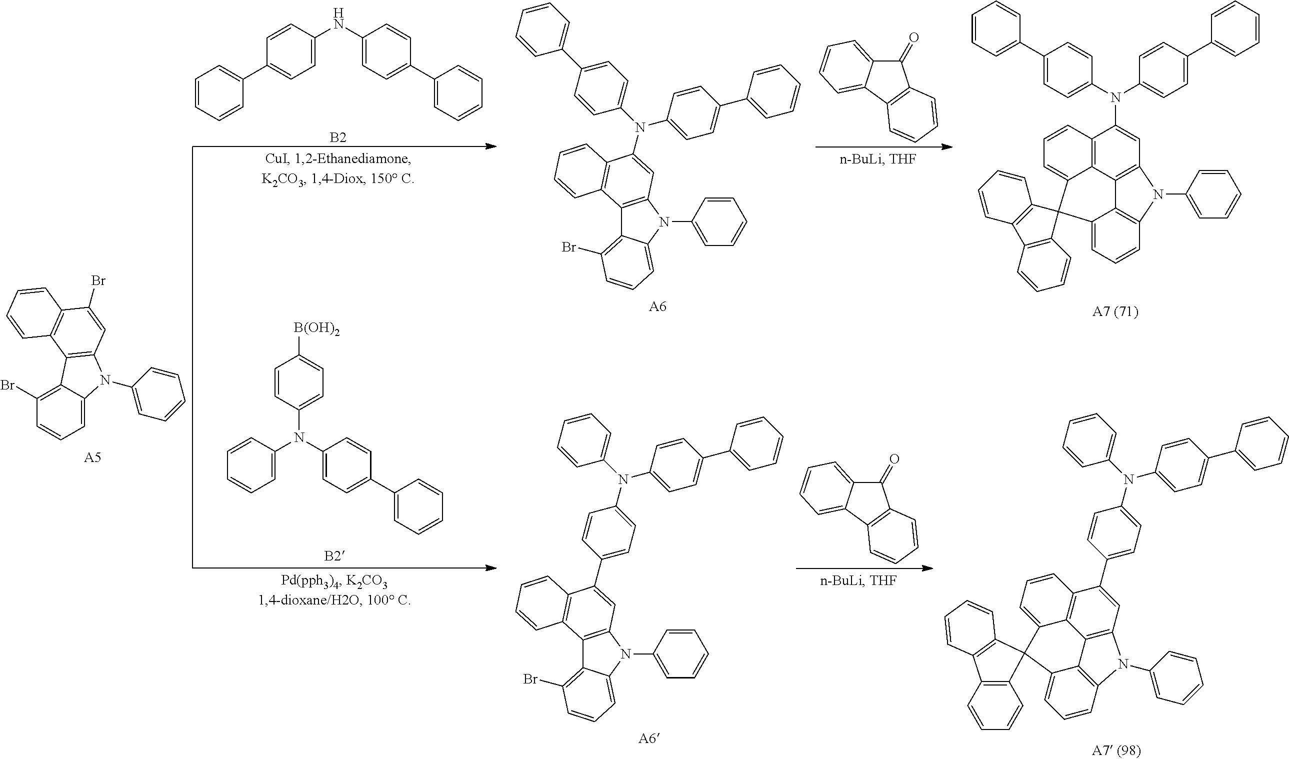

1) Preparation of Compound A2

20.0 g of naphthalen-1-ylboronic acid (116.28 mmol), 38.13 g of 1-bromo-2-iodo-3-nitrobenzene (116.28 mmol), 6.72 g of tetrakis(triphenylhosphine)palladium(0) (Pd(PPh₃)₄) (5.814 mmol), and 32.14 g of K₂CO₃ (232.56 mmol) were dissolved in 200 ml/40 ml of 1,4-dioxane/H₂O, and then refluxed for 3 hours. After the reaction was completed, the reactant was extracted by adding distilled water and dichloromethane (DCM) at room temperature, and then the organic layer was dried over MgSO₄, and the solvent was removed by a rotary evaporator. The reactant was purified by column chromatography (DCM:Hex=1:5) to obtain 30.0 g (78.6%) of compound A2.

2) Preparation of compound A3

30.0 g of the compound A2 (91.41 mmol) and 59.94 g of triphenylphosphine (PPh3) (228.54 mmol) were dissolved in 300 ml of 1,2-dichlorobenzene (1,2-DCB) and refluxed at 200° C. for 5 hours. After the reaction was completed, the solvent was removed using a rotary evaporator. The reactant was purified by column chromatography (DCM:Hex=1:4) to obtain 19.0 g (70.37%) of compound A3.

3) Preparation of Compound A4

19.0 g of the compound A3 (64.15 mmol) and 11.41 g of N-bromosuccinimide (NBS) (64.15 mmol) were dissolved in 200 ml of chloroform, and then stirred at room temperature for 7 hours. After the reaction was completed, the reactant was extracted with distilled water and DCM at room temperature, and then the organic layer was dried over MgSO₄, and then the solvent was removed by a rotary evaporator. The reactant was purified by column chromatography (DCM:Hex=1:5) to obtain 17.8 g (74.01%) of compound A4.

4) Preparation of Compound A5

17.8 g of the compound A4 (47.46 mmol), 9.68 g of iodobenzene (47.46 mmol), 10.85 g of CuI (56.95 mmol), 3.42 g of 1,2-ethanediamine (56.95 mmol), and 19.68 g of K₂CO₃ (142.38 mmol) were dissolved in 180 ml of 1,4-dioxane, and then refluxed at 200° C. for 12 hours. After the reaction was completed, the reactant was extracted with distilled water and DCM at room temperature, and then the organic layer was dried over MgSO₄ and the solvent was removed by a rotary evaporator. The reactant was purified by column chromatography (DCM:Hex=1:7) to obtain 18.62 g (87%) of compound A5.

5) Preparation of Compound A6

18.62 g of the compound A5 (21.83 mmol), 7.02 g of di([1,1′-biphenyl]-4-yl)amine (21.83 mmol), 4.99 g of CuI (26.19 mmol), 1.31 g of 1,2-ethanediamine (21.83 mmol), and 9.05 g of K₂CO₃ (65.49 mmol) were dissolved in 180 ml of 1,4-dioxane, and then refluxed at 200° C. for 12 hours. After the reaction was completed, the reactant was extracted with distilled water and DCM at room temperature, and then the organic layer was dried over MgSO₄ and the solvent was removed by a rotary evaporator. The reactant was purified by column chromatography (DCM:Hex=1:4) to obtain 7.40 g (49%) of compound A6.

6) Preparation of Compound A6′ 18.62 g of the compound A5 (21.83 mmol), 7.97 g of (4-([1,1′-biphenyl]-4-yl(phenyl)amino)phenyl)boronic acid (21.83 mmol), 1.3 g of tetrakis(triphenylhosphine)palladium(0) (Pd(PPh₃)₄) (1.0915 mmol), and 6.03 g of K₂CO₃ (46.33 mmol) were dissolved in 1,4-dioxane/H₂O 200 ml/40 ml, and then refluxed for 3 hours. After the reaction was completed, the reactant was extracted with distilled water and DCM at room temperature, and then the organic layer was dried over MgSO₄ and the solvent was removed by a rotary evaporator. The reactant was purified by column chromatography (DCM:Hex=1:4) to obtain 7.85 g (52%) of compound A6′.

7) Preparation of compound A7

7.40 g of the compound A6 (10.70 mmol) was placed in a 250 mL round-bottom flask, and 80 mL of tetrahydrofuran (THF) was added under a nitrogen atmosphere, and the internal temperature of the reaction vessel was lowered to −78° C. 4.71 mL of 2.5M n-BuLi/hexane (Hx) was slowly added dropwise and stirred for 2 hours, and then 2.9 g of 9H-fluoren-9-one (16.05 mmol) was slowly added dropwise at room temperature. After the reaction was completed after 12 hours, the reaction was completely terminated using 10 ml of Ac₂O:HCl (1:1), and then the reactant was extracted with distilled water and DCM at room temperature, and the organic layer was dried over MgSO₄, and the solvent was removed using a rotary evaporator. The reactant was purified by column chromatography (DCM:Hex=1:5) to obtain 3.56 g (43%) of the target compound A7(71).

The following target compounds were synthesized and prepared in the same manner as in Preparation Example 1-1, except that in Preparation Example 1-1, B1 and B2/B2′ of Table 1 below were used as intermediates.

TABLE 1

| Compound | Intermediate | Intermediate B2/ | | |
|---|---|---|---|---|
| no. | B1 | B2′ | Target compound | Yield |
| 71 | 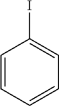 | 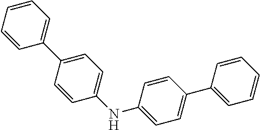 | 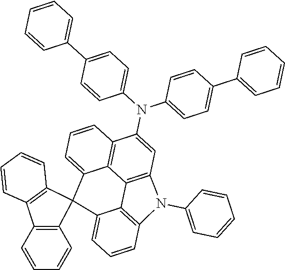 [SMILES: c1ccc(-c2ccc(N(c3ccc(-c4ccccc4)cc3)c3cc4c5c6c(cccc36)C3(c6ccccc6-c6ccccc63)c3cccc(c35)n4-c3ccccc3)cc2)cc1] | 50% |
| 73 | 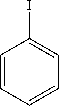 | 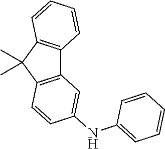 | 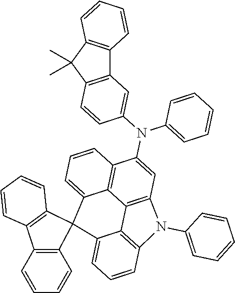 [SMILES: CC1(C)c2ccccc2-c2cc(N(c3ccccc3)c3cc4c5c6c(cccc36)C3(c6ccccc6-c6ccccc63)c3cccc(c35)n4-c3ccccc3)ccc21] | 46% |
| 79 | 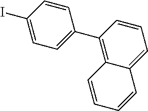 | 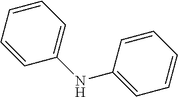 | 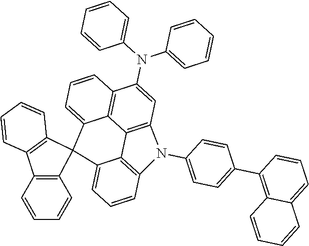 [SMILES: c1ccc(N(c2ccccc2)c2cc3c4c5c(cccc25)C2(c5ccccc5-c5ccccc52)c2cccc(c24)n3-c2ccc(-c3cccc4ccccc34)cc2)cc1] | 43% |
| 87 | 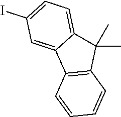 | 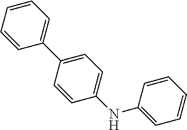 | 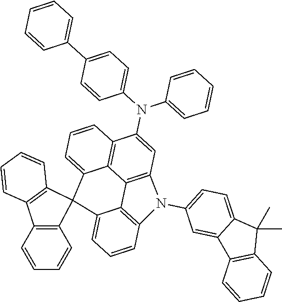 [SMILES: CC1(C)c2ccccc2-c2cc(-n3c4cccc5c4c4c6c(cccc6c(N(c6ccccc6)c6ccc(-c7ccccc7)cc6)cc43)C53c4ccccc4-c4ccccc43)ccc21] | 38% |
| 95 | 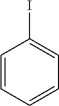 | 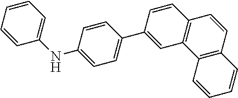 | 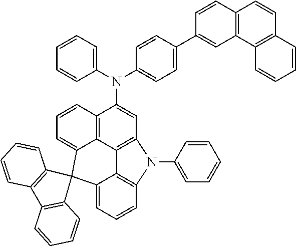 [SMILES: c1ccc(N(c2ccc(-c3ccc4ccc5ccccc5c4c3)cc2)c2cc3c4c5c(cccc25)C2(c5ccccc5-c5ccccc52)c2cccc(c24)n3-c2ccccc2)cc1] | 44% |
| 98 | 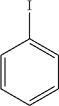 | 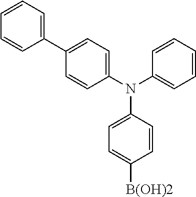 | 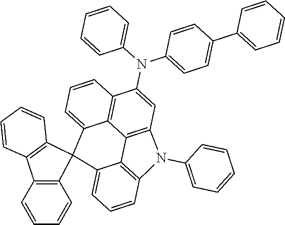 [SMILES: c1ccc(-c2ccc(N(c3ccccc3)c3cc4c5c6c(cccc36)C3(c6ccccc6-c6ccccc63)c3cccc(c35)n4-c3ccccc3)cc2)cc1] | 51% |
| 101 | 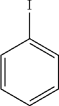 | 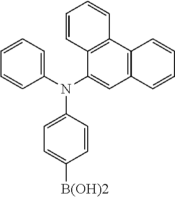 | 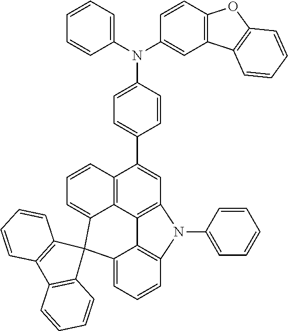 [SMILES: c1ccc(N(c2ccc(-c3cc4c5c6c(cccc36)C3(c6ccccc6-c6ccccc63)c3cccc(c35)n4-c3ccccc3)cc2)c2ccc3oc4ccccc4c3c2)cc1] | 50% |
| 103 | 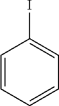 | 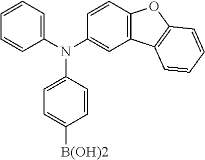 | 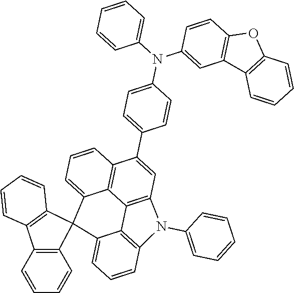 [SMILES: c1ccc(N(c2ccc(-c3cc4c5c6c(cccc36)C3(c6ccccc6-c6ccccc63)c3cccc(c35)n4-c3ccccc3)cc2)c2ccc3oc4ccccc4c3c2)cc1] | 48% |
| 108 | 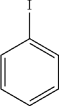 | 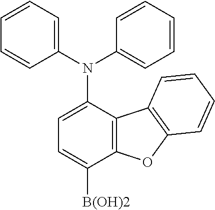 | 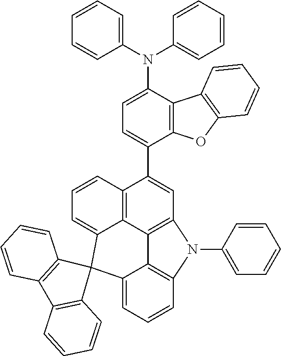 [SMILES: c1ccc(N(c2ccccc2)c2ccc(-c3cc4c5c6c(cccc36)C3(c6ccccc6-c6ccccc63)c3cccc(c35)n4-c3ccccc3)c3oc4ccccc4c23)cc1] | 45% |
| 110 | 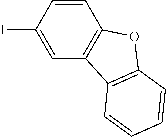 | 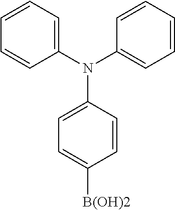 | 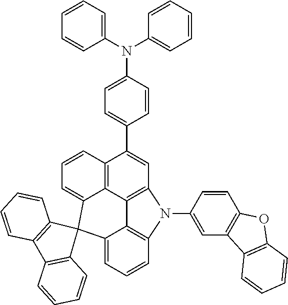 [SMILES: c1ccc(N(c2ccccc2)c2ccc(-c3cc4c5c6c(cccc36)C3(c6ccccc6-c6ccccc63)c3cccc(c35)n4-c3ccc4oc5ccccc5c4c3)cc2)cc1] | 49% |

### <Preparation Example 1-2> Preparation of Compound C6(153) and Compound C6′ (171)

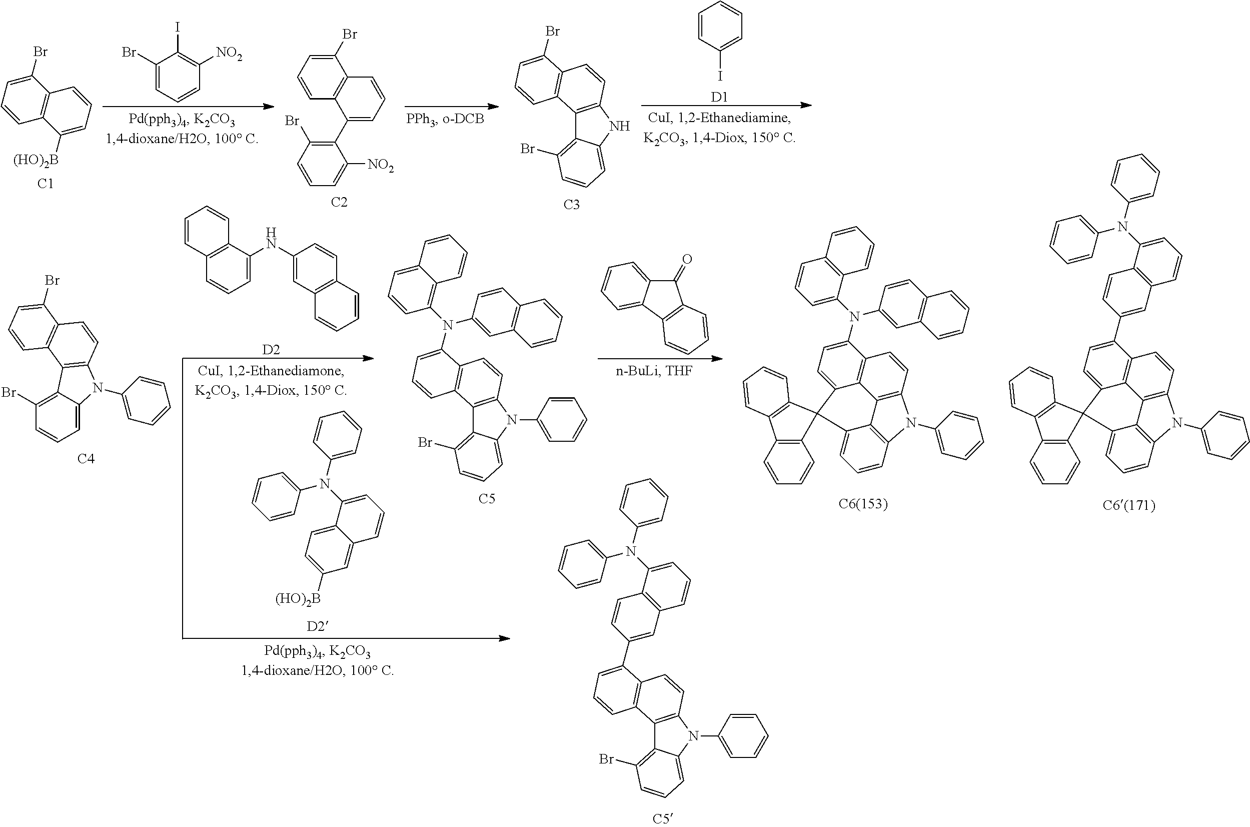

The following target compounds were synthesized and prepared in the same manner as in Preparation Example 1-1, except that in Preparation Example 1-2, D1 and D2/D2′ of Table 2 below were used as intermediates.

TABLE 2

| Compound | Intermediate | Intermediate D2/ | | |
|---|---|---|---|---|
| no. | D1 | D2′ | Compound | Yield |
| 146 | 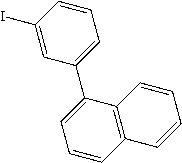 | 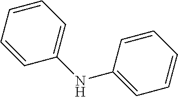 | 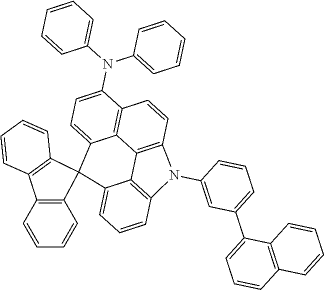 [SMILES: c1ccc(N(c2ccccc2)c2ccc3c4c2ccc2c4c4c(cccc4n2-c2cccc(-c4cccc5ccccc45)c2)C32c3ccccc3-c3ccccc32)cc1] | 57% |
| 148 | 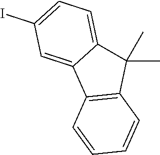 | 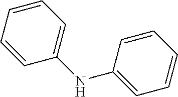 | 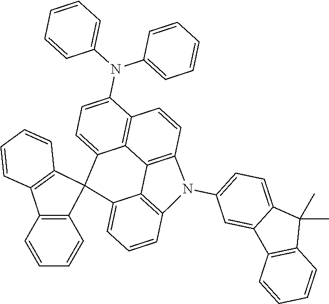 [SMILES: CC1(C)c2ccccc2-c2cc(-n3c4cccc5c4c4c6c(ccc(N(c7ccccc7)c7ccccc7)c6ccc43)C53c4ccccc4-c4ccccc43)ccc21] | 51% |
| 153 | 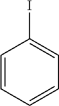 | 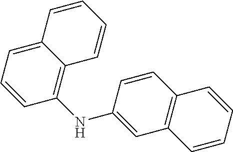 | 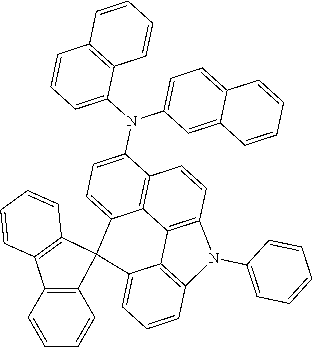 [SMILES: c1ccc(-n2c3cccc4c3c3c5c(ccc(N(c6ccc7ccccc7c6)c6cccc7ccccc67)c5ccc32)C42c3ccccc3-c3ccccc32)cc1] | 55% |
| 154 | 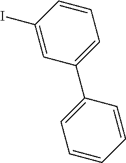 | 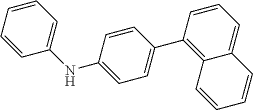 | 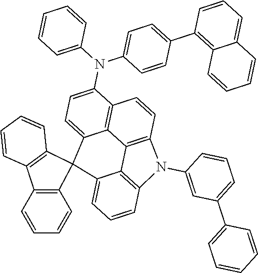 [SMILES: c1ccc(-c2cccc(-n3c4cccc5c4c4c6c(ccc(N(c7ccccc7)c7ccc(-c8cccc9ccccc89)cc7)c6ccc43)C53c4ccccc4-c4ccccc43)c2)cc1] | 48% |
| 159 | 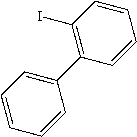 | 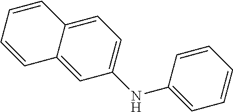 | 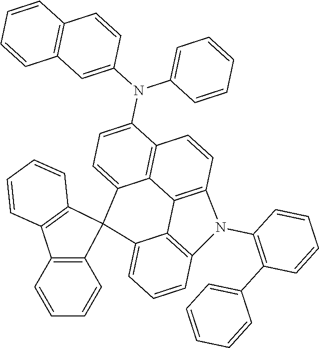 [SMILES: c1ccc(-c2ccccc2-n2c3cccc4c3c3c5c(ccc(N(c6ccccc6)c6ccc7ccccc7c6)c5ccc32)C42c3ccccc3-c3ccccc32)cc1] | 42% |
| 162 | 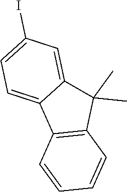 | 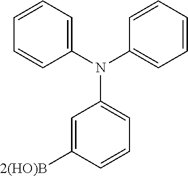 | 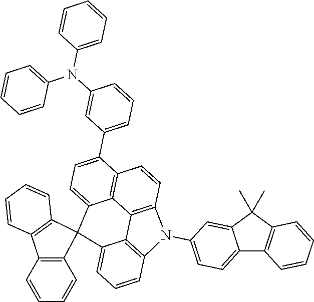 [SMILES: CC1(C)c2ccccc2-c2ccc(-n3c4cccc5c4c4c6c(ccc(-c7cccc(N(c8ccccc8)c8ccccc8)c7)c6ccc43)C53c4ccccc4-c4ccccc43)cc21] | 39% |
| 168 | 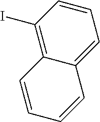 | 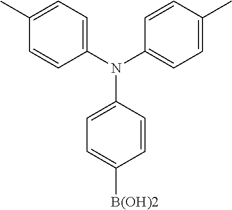 | 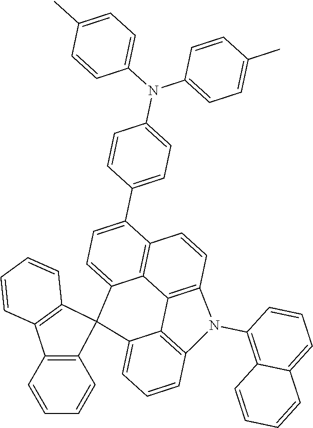 [SMILES: Cc1ccc(N(c2ccc(C)cc2)c2ccc(-c3ccc4c5c3ccc3c5c5c(cccc5n3-c3cccc5ccccc35)C43c4ccccc4-c4ccccc43)cc2)cc1] | 44% |
| 170 | 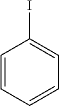 | 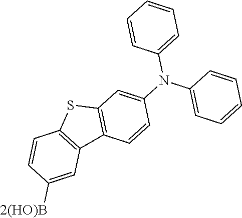 | 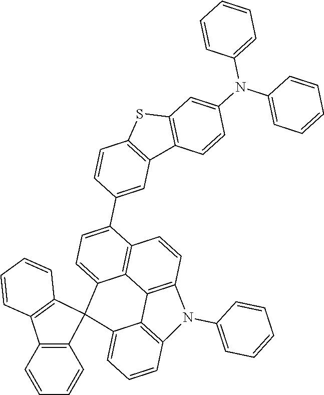 [SMILES: c1ccc(N(c2ccccc2)c2ccc3c(c2)sc2ccc(-c4ccc5c6c4ccc4c6c6c(cccc6n4-c4ccccc4)C54c5ccccc5-c5ccccc54)cc23)cc1] | 45% |
| 171 | 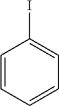 | 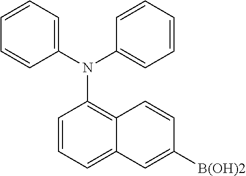 | 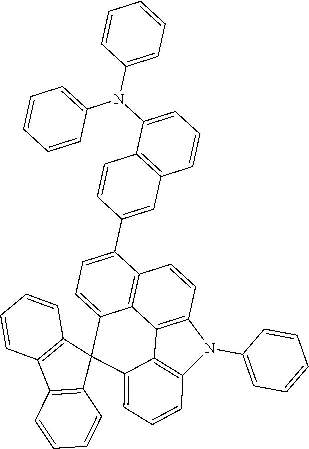 [SMILES: c1ccc(N(c2ccccc2)c2cccc3cc(-c4ccc5c6c4ccc4c6c6c(cccc6n4-c4ccccc4)C54c5ccccc5-c5ccccc54)ccc23)cc1] | 50% |
| 173 | 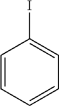 | 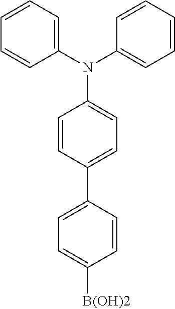 | 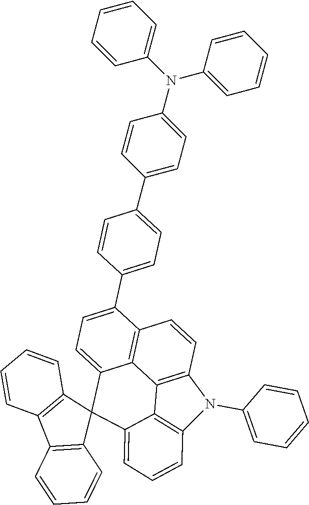 [SMILES: c1ccc(N(c2ccccc2)c2ccc(-c3ccc(-c4ccc5c6c4ccc4c6c6c(cccc6n4-c4ccccc4)C54c5ccccc5-c5ccccc54)cc3)cc2)cc1] | 45% |

### <Preparation Example 1-3> Preparation of Compound E6(118) and Compound E6′ (140)

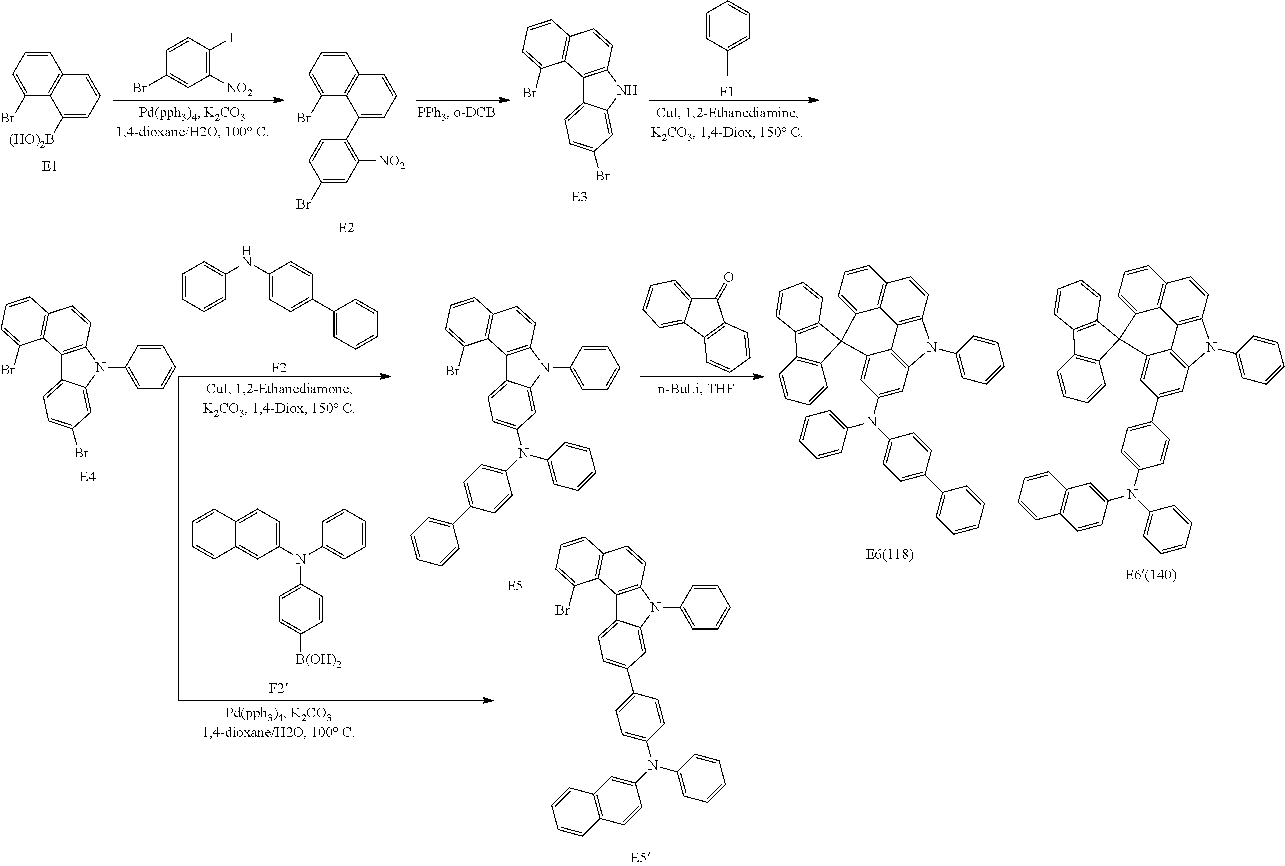

The following target compounds were synthesized and prepared in the same manner as in Preparation Example 1-1, except that in Preparation Example 1-3, F1 and F2/F2′ of Table 3 below were used as intermediates.

TABLE 3

| Compound | Intermediate | Intermediate F2/ | | |
|---|---|---|---|---|
| no. | F1 | F2′ | Target compound | Yield |
| 113 | 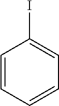 | 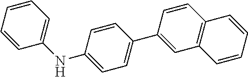 | 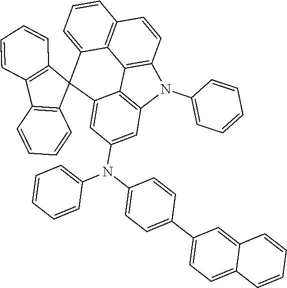 [SMILES: c1ccc(N(c2ccc(-c3ccc4ccccc4c3)cc2)c2cc3c4c5c6c(cccc6ccc5n(-c5ccccc5)c4c2)C32c3ccccc3-c3ccccc32)cc1] | 45% |
| 118 | 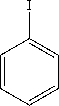 | 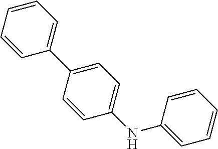 | 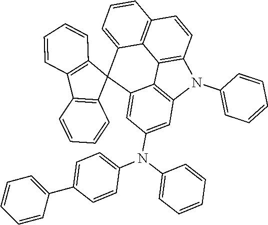 [SMILES: c1ccc(-c2ccc(N(c3ccccc3)c3cc4c5c6c7c(cccc7ccc6n(-c6ccccc6)c5c3)C43c4ccccc4-c4ccccc43)cc2)cc1] | 43% |
| 120 | 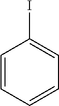 | 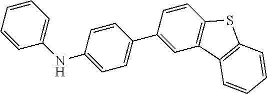 | 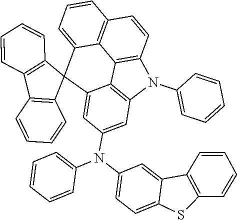 [SMILES: c1ccc(N(c2ccc3sc4ccccc4c3c2)c2cc3c4c5c6c(cccc6ccc5n(-c5ccccc5)c4c2)C32c3ccccc3-c3ccccc32)cc1] | 52% |
| 121 | 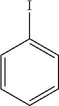 | 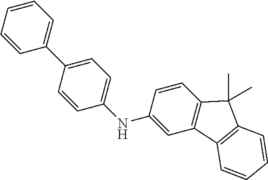 | 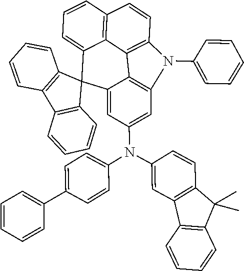 [SMILES: CC1(C)c2ccccc2-c2cc(N(c3ccc(-c4ccccc4)cc3)c3cc4c5c6c7c(cccc7ccc6n(-c6ccccc6)c5c3)C43c4ccccc4-c4ccccc43)ccc21] | 56% |
| 127 | 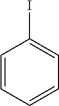 | 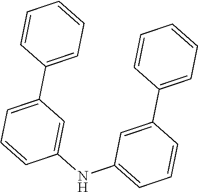 | 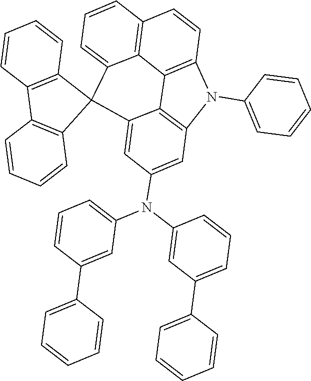 [SMILES: c1ccc(-c2cccc(N(c3cccc(-c4ccccc4)c3)c3cc4c5c6c7c(cccc7ccc6n(-c6ccccc6)c5c3)C43c4ccccc4-c4ccccc43)c2)cc1] | 55% |
| 130 | 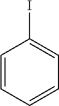 | 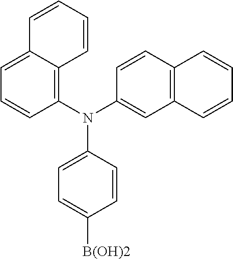 | 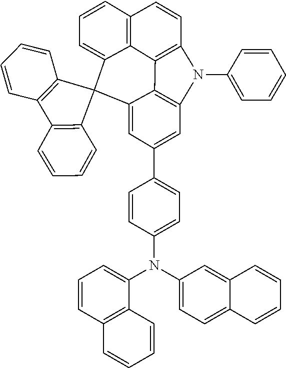 [SMILES: c1ccc(-n2c3cc(-c4ccc(N(c5ccc6ccccc6c5)c5cccc6ccccc56)cc4)cc4c3c3c5c(cccc5ccc32)C42c3ccccc3-c3ccccc32)cc1] | 48% |
| 133 | 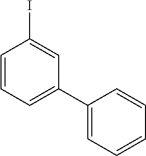 | 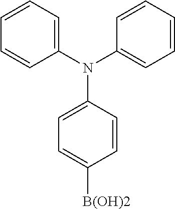 | 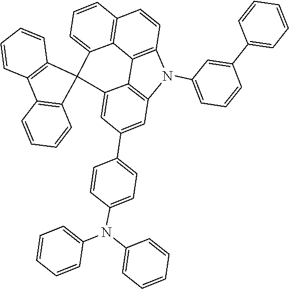 [SMILES: c1ccc(-c2cccc(-n3c4cc(-c5ccc(N(c6ccccc6)c6ccccc6)cc5)cc5c4c4c6c(cccc6ccc43)C53c4ccccc4-c4ccccc43)c2)cc1] | 52% |
| 137 | 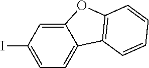 | 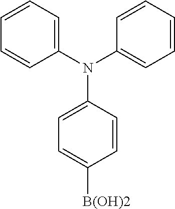 | 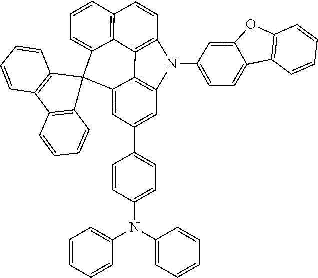 [SMILES: c1ccc(N(c2ccccc2)c2ccc(-c3cc4c5c6c7c(cccc7ccc6n(-c6ccc7c(c6)oc6ccccc67)c5c3)C43c4ccccc4-c4ccccc43)cc2)cc1] | 45% |
| 140 | 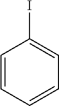 | 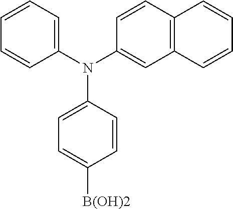 | 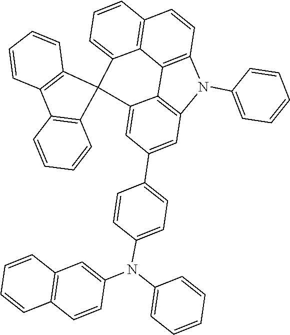 [SMILES: c1ccc(N(c2ccc(-c3cc4c5c6c7c(cccc7ccc6n(-c6ccccc6)c5c3)C43c4ccccc4-c4ccccc43)cc2)c2ccc3ccccc3c2)cc1] | 51% |
| 141 | 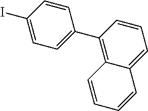 | 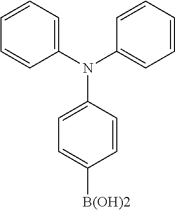 | 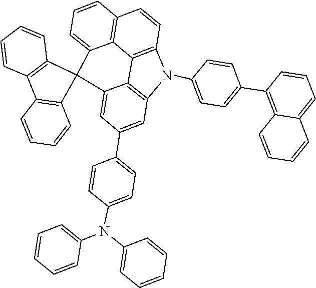 [SMILES: c1ccc(N(c2ccccc2)c2ccc(-c3cc4c5c6c7c(cccc7ccc6n(-c6ccc(-c7cccc8ccccc78)cc6)c5c3)C43c4ccccc4-c4ccccc43)cc2)cc1] | 44% |

### <Preparation Example 1-4> Preparation of Compound G5(16) and Compound G5′ (60)

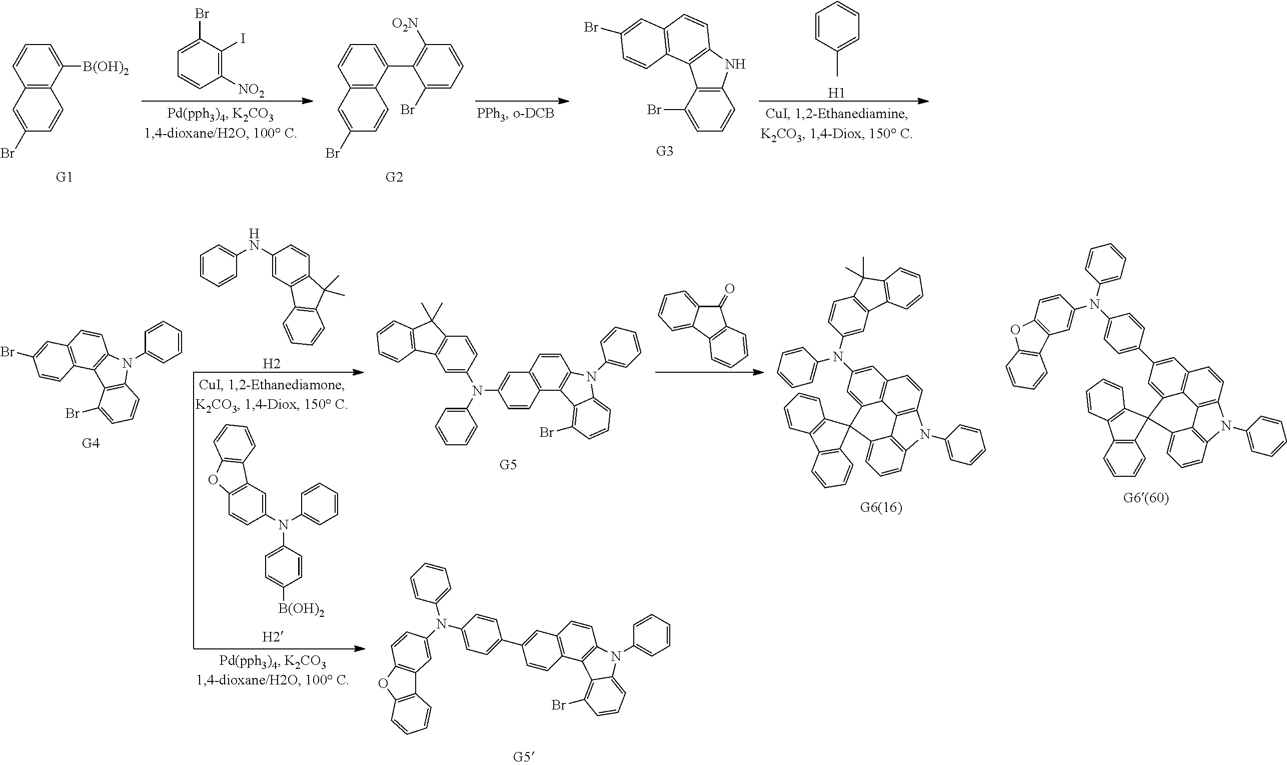

The following target compounds were synthesized and prepared in the same manner as in Preparation Example 1-1, except that in Preparation Example 1-4, H1 and H2/H2′ of Table 4 below were used as intermediates.

TABLE 4

| Compound | Intermediate | Intermediate H2/ | | |
|---|---|---|---|---|
| no. | H1 | H2′ | Target compound | Yield |
| 3 | 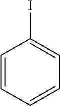 | 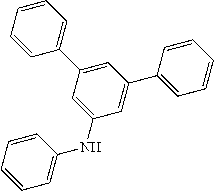 | 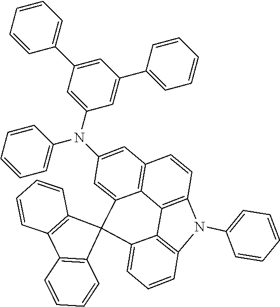 [SMILES: c1ccc(-c2cc(-c3ccccc3)cc(N(c3ccccc3)c3cc4c5c(ccc6c5c5c(cccc5n6-c5ccccc5)C45c4ccccc4-c4ccccc45)c3)c2)cc1] | 39% |
| 4 | 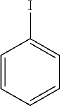 | 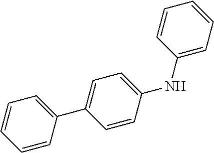 | 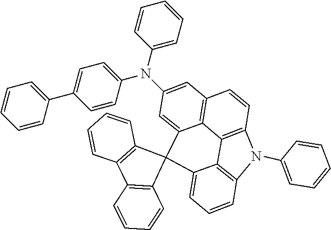 [SMILES: c1ccc(-c2ccc(N(c3ccccc3)c3cc4c5c(ccc6c5c5c(cccc5n6-c5ccccc5)C45c4ccccc4-c4ccccc45)c3)cc2)cc1] | 57% |
| 16 | 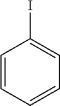 | 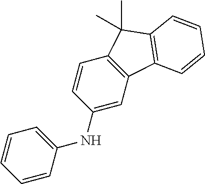 | 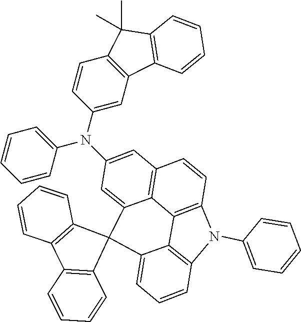 [SMILES: CC1(C)c2ccccc2-c2cc(N(c3ccccc3)c3cc4c5c(ccc6c5c5c(cccc5n6-c5ccccc5)C45c4ccccc4-c4ccccc45)c3)ccc21] | 47% |
| 30 | 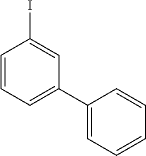 | 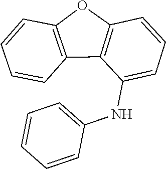 | 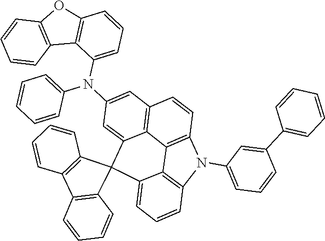 [SMILES: c1ccc(-c2cccc(-n3c4cccc5c4c4c6c(cc(N(c7ccccc7)c7cccc8oc9ccccc9c78)cc6ccc43)C53c4ccccc4-c4ccccc43)c2)cc1] | 52% |
| 42 | 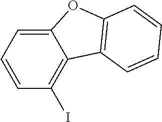 | 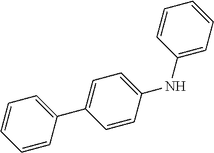 | 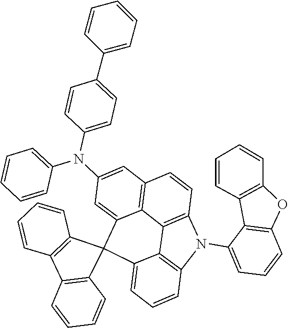 [SMILES: c1ccc(-c2ccc(N(c3ccccc3)c3cc4c5c(ccc6c5c5c(cccc5n6-c5cccc6oc7ccccc7c56)C45c4ccccc4-c4ccccc45)c3)cc2)cc1] | 56% |
| 55 | 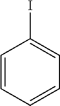 | 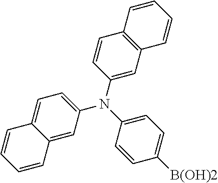 | 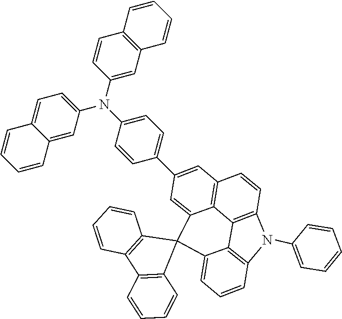 [SMILES: c1ccc(-n2c3cccc4c3c3c5c(cc(-c6ccc(N(c7ccc8ccccc8c7)c7ccc8ccccc8c7)cc6)cc5ccc32)C42c3ccccc3-c3ccccc32)cc1] | 32% |
| 60 | 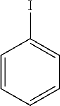 | 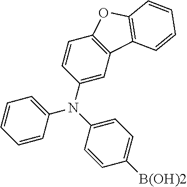 | 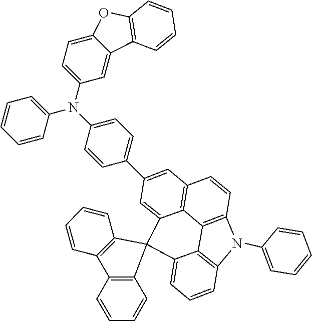 [SMILES: c1ccc(N(c2ccc(-c3cc4c5c(ccc6c5c5c(cccc5n6-c5ccccc5)C45c4ccccc4-c4ccccc45)c3)cc2)c2ccc3oc4ccccc4c3c2)cc1] | 42% |
| 62 | 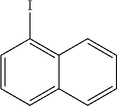 | 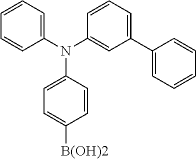 | 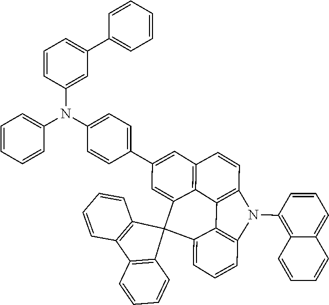 [SMILES: c1ccc(-c2cccc(N(c3ccccc3)c3ccc(-c4cc5c6c(ccc7c6c6c(cccc6n7-c6cccc7ccccc67)C56c5ccccc5-c5ccccc56)c4)cc3)c2)cc1] | 44% |
| 63 | 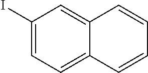 | 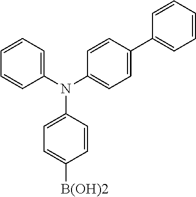 | 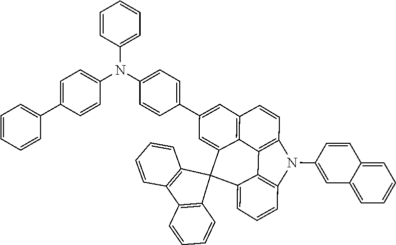 [SMILES: c1ccc(-c2ccc(N(c3ccccc3)c3ccc(-c4cc5c6c(ccc7c6c6c(cccc6n7-c6ccc7ccccc7c6)C56c5ccccc5-c5ccccc56)c4)cc3)cc2)cc1] | 48% |
| 67 | 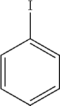 | 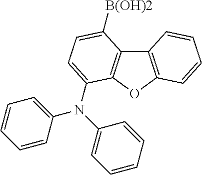 | 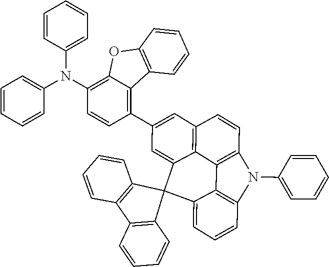 [SMILES: c1ccc(N(c2ccccc2)c2ccc(-c3cc4c5c(ccc6c5c5c(cccc5n6-c5ccccc5)C45c4ccccc4-c4ccccc45)c3)c3c2oc2ccccc23)cc1] | 50% |

The remaining compounds other than the compounds described in Preparation Examples 1-1 to 1-4 and Tables 1 to 4 were prepared in the same manner as in Preparation Examples described above, and the synthesis results are shown in Tables 5 and 6 below. Table 5 below is a measurement value of ¹H NMR (CDCl₃, 300 Mz), and Table 6 below is a measurement value of the FD-mass spectrometer (FD-MS: Field desorption mass spectrometry).

TABLE 5

| NO | ¹H NMR (CDC1₃, 300 Mz) |
|---|---|
| 3 | 7.90 (d, 2H), 7.75 (d, 4H), 7.75-7.24 (m, 27H), 7.08-7.00 (m, 4H), |
| | 6.66 (d, 1H) |
| 4 | 7.90 (d, 2H), 7.75 (d, 2H), 7.62-7.24 (m, 25H), 7.08-6.93 (m, 4H), |
| | 6.66 (d, 1H) |
| 16 | 7.90 (d, 3H), 7.62-7.50 (m, 12H), 7.38-7.17 (m, 11H), 7.08-6.93 |
| | (m, 5H), 6.66 (d, 1H) |
| 30 | 8.21 (d, 1H), 7.98 (d, 1H), 7.90 (d, 2H), 7.75-7.08 (m, 31H), 6.66 |
| | (d, 1H) |
| 42 | 7.98 (d, 1H), 7.90 (d, 2H), 7.75 (d, 2H), 7.55-7.17 (m, 27H), |
| | 7.08-7.00 (dd, 3H), 6.66 (d, 1H) |
| 55 | 7.90-7.87 (d, 3H), 7.62-7.27 (m, 31H), 7.17-7.11 (m, 3H), 6.93 |
| | (d, 1H) |
| 60 | 8.22 (s, 1H), 7.98 (d, 1H), 7.90-7.87 (d, 3H), 7.55-7.17 (m, 26H), |
| | 7.08-6.98 (m, 5H), |
| 62 | 7.90-7.75 (m, 8H), 7.62-7.17 (m, 28H), 7.08-6.98 (m, 4H) |
| 63 | 8.03 (dd, 3H), 7.90-7.83 (m, 4H), 7.75 (d, 2H), 7.58-7.27 (m, 27H), |
| | 7.08-6.98 (m, 4H) |
| 67 | 7.98 (d, 1H), 7.90-7.87 (d, 3H), 7.62-7.52 (m, 12H), 7.39-6.98 (m, |
| | 20H) |
| 71 | 7.95 (d, 1H), 7.90 (d, 2H), 7.75 (d, 4H), 7.49-7.27 (m, 28H), 7.18- |
| | 7.17 (m, 2H), 6.93 (d, 1H) |
| 73 | 7.95 (d, 1H), 7.90 (d, 2H), 7.62-7.50 (m, 10H), 7.38-7.17 (m, 12H), |
| | 7.03-6.93 (m, 5H) |
| 79 | 8.95 (d, 1H), 8.50 (d, 1H), 8.20 (d, 2H), 7.95-7.89 (m, 6H), 7.78 (d, |
| | 1H), 7.77 (t, 1H), 7.55 (d 2H), 7.41-6.93 (m, 22H) |
| 87 | 7.96-7.90 (m, 5H), 7.75 (d, 1H), 7.69 (d, 1H), 7.55-7.18 (m, 31H), |
| | 7.08-6.93 (m, 4H) |
| 95 | 9.27 (s, 1H), 8.85 (d, 1H), 8.37-8.35 (dd, 2H), 7.90 (d, 3H), 7.75- |
| | 7.50 (m, 13H), 7.38-6.98 (m, 17H) |
| 98 | 8.80 (d, 1H), 7.90 (d, 2H), 7.75 (d, 2H), 7.62-7.24 (m, 29H), 7.24- |
| | 6.93 (m, 4H) |
| 101 | 8.98 (d, 1H), 8.84-8.80 (m, 2H), 8.11 (d, 1H), 7.90 (d, 3H), 7.68- |
| | 7.49 (m, 14H), 7.38-7.17 (m, 12H), 7.08-6.93 (m, 5H) |
| 103 | 8.80 (d, 1H), 8.22 (d, 1H), 7.98 (d, 1H), 7.90 (d, 2H), 7.62-7.17 (m, |
| | 24H), 7.08-6.93 (m, 6H) |
| 108 | 8.80 (d, 1H), 7.98 (d, 1H), 7.90 (d, 1H), 7.62-7.50 (m, 9H), 7.39- |
| | 6.93 (m, 24H) |
| 110 | 8.80 (d, 1H), 7.98 (d, 1H), 7.90 (d, 2H), 7.64-7.54 (m, 7H), 7.39- |
| | 6.93 (m, 25H) |
| 113 | 8.09-7.99 (d, 3H), 7.90 (d, 2H), 7.67-7.50 (m, 15H), 7.38-7.27 (m, |
| | 10H), 7.08-6.93 (m, 4H), 6.68 (s, 1H) |
| 118 | 7.90 (d, 2H), 7.75 (d, 1H), 7.67-7.24 (m, 26H), 7.08-6.93 (m, 4H), |
| | 6.68 (s, 1H) |
| 120 | 8.45 (d, 1H), 7.95-7.85 (m, 5H), 7.97-7.50 (m, 11H), 7.41-7.24 (m, |
| | 5H), 6.68 (s, 1H) |
| 121 | 7.90 (d, 3H), 7.75 (d, 2H), 7.67-7.28 (m, 26H), 7.15 (d, 1H), 7.06 |
| | (d, 1H), 6.93 (d, 1H), 6.68 (s, 1H) |
| 127 | 7.90 (d, 2H), 7.75-7.41 (m, 28H), 7.18-7.15 (m, 5H), 6.93 (d, 2H), |
| | 6.68 (s, 1H) |
| 130 | 8.22 (d, 1H), 7.90-7.84 (m, 4H), 7.71-7.27 (m, 31H), 7.11 (s, 1H), |
| | 6.93 (d, 1H) |
| 133 | 8.21 (s, 1H), 7.90-7. 84 (m, 3H), 7. 75-7. 24 (m, 27H), 7.08-6.93 (m, 7H) |
| 137 | 7.98 (d, 1H), 7.90-7.84 (m, 3H), 7.73-7.67 (d, 2H), 7.64 (s, 1H), |
| | 7.55-7.52 (m, 8H), 7.39-7.24 (m, 14H), 7.24-6.93 (m, 7H) |
| 140 | 7.90 (d, 2H), 7.84 (s, 1H), 7.71-7.24 (m, 28H), 7.11-6.93 (m, 5H) |
| 141 | 8.95 (d, 1H), 8.50 (d, 1H), 8.20 (d, 2H), 7.92-7.77 (m, 8H), 7.67- |
| | 7.64 (d, 2H), 7.55-7.52 (m, 6H), 7.41-7.24 (m, 13H), 7.24-7.08 (m, 6H) |
| 146 | 8.95 (d, 1H), 8.50 (d, 1H), 8.21 (s, 1H), 7.90-7.89 (d, 3H), 7.78- |
| | 7.77 (m, 2H), 7.66-6.89 (m, 28H) |
| 148 | 7.96 (s, 1H), 7.90 (d, 3H), 7.69 (d, 1H), 7.55-7.52 (m, 5H), 7.38- |
| | 7.17 (m, 13H), 7.08-6.93 (m, 8H) |
| 153 | 8.22 (d, 1H), 7.90-7.84 (d, 3H), 7.63-7.38 (m, 26H), 7.17 (t, 1H), |
| | 7.11 (s, 1H), 6.93 (d, 2H) |
| 154 | 8.95 (d, 1H), 8.50 (d, 1H), 8.21 (s, 1H), 7.90 (d, 2H), 7.89 (d, 1H), |
| | 7.78-7.24 (m, 30H), 7.08-6.93 (m, 4H) |
| 159 | 8.20 (d, 1H), 7.92 (d, 1H), 7.90 (d, 2H), 7.80 (t, 1H), 7.71 (d, 1H), |
| | 7.49-6.93 (m, 30H) |
| 162 | 8.04 (d, 1H), 7.90 (d, 3H), 7.67 (s, 1H), 7.55-7.50 (m, 7H), 7.38- |
| | 7.17 (m, 16H), 7.08-6.93 (m, 8H) |
| 168 | 7.90 (d, 2H), 7.84 (d, 2H), 7.80 (d, 1H), 7.62-7.48 (m, 10H), 7.38- |
| | 7.15 (m, 13H), 7.03 (d, 4H), 6.93 (d, 2H) |
| 170 | 8.12 (d, 2H), 7.99 (d, 1H), 7.90 (d, 2H), 7.74 (d, 1H), 7.62-6.93 (m, 30H) |
| 171 | 8.19 (d, 1H), 8.04 (s, 1H), 7.90 (d, 2H), 7.65-7.48 (m, 13H), 7.38- |
| | 6.39 (m, 19H) |
| 173 | 7.90 (d, 2H), 7.62-7.55 (m, 11H), 7.38-7.17 (m, 17H), 7.08-6.93 (m, 8H) |

TABLE 6

| Compound | FD-MS | Compound | FD-MS |
|---|---|---|---|
| 1 | m/z = 698.27 (C₅₃H₃₄N₂ = 698.87) | 2 | m/z = 774.30 |
| | | | (C₅₉H₃₈N₂ = 774.97) |
| 3 | m/z = 774.30 (C₅₉H₃₈N₂ = 774.97) | 4 | m/z = 698.27 |
| | | | (C₅₃H₃₄N₂ = 698.87) |
| 5 | m/z = 650.27 (C₄₉H₃₄N₂ = 650.83) | 6 | m/z = 748.29 |
| | | | (C₅₇H₃₆N₂ = 748.93) |
| 7 | m/z = 748.29 (C₅₇H₃₆N₂ = 748.93) | 8 | m/z = 672.26 |
| | | | (C₅₁H₃₂N₂ = 672.83) |
| 9 | m/z = 774.30 (C₅₉H₃₈N₂ = 774.97) | 10 | m/z = 774.30 |
| | | | (C₅₉H₃₈N₂ = 774.97) |
| 11 | m/z = 774.30 (C₅₉H₃₈N₂ = 774.97) | 12 | m/z = 712.25 |
| | | | (C₅₃H₃₂N₂O = 712.85) |
| 13 | m/z = 712.25 (C₅₃H₃₂N₂O = 712.85) | 14 | m/z = 728.23 |
| | | | (C₅₃H₃₂N₂S = 728.91) |
| 15 | m/z = 728.23 (C₅₃H₃₂N₂S = 728.91) | 16 | m/z = 738.30 |
| | | | (C₅₆H₃₈N₂ = 738.93) |
| 17 | m/z = 698.27 (C₅₃H₃₄N₂ = 698.87) | 18 | m/z = 698.27 |
| | | | (C₅₃H₃₄N₂ = 698.87) |
| 19 | m/z = 774.30 (C₅₉H₃₈N₂ = 774.97) | 20 | m/z = 748.29 |
| | | | (C₅₇H₃₆N₂ = 748.93) |
| 21 | m/z = 698.27 (C₅₃H₃₄N₂ = 698.87) | 22 | m/z = 788.28 |
| | | | (C₅₉H₃₆N₂O = 788.95) |
| 23 | m/z = 788.28 (C₅₉H₃₆N₂O = 788.95) | 24 | m/z = 788.28 |
| | | | (C₅₉H₃₆N₂O = 788.95) |
| 25 | m/z = 788.28 (C₅₉H₃₆N₂O = 788.95) | 26 | m/z = 774.30 |
| | | | (C₅₉H₃₈N₂ = 774.97) |
| 27 | m/z = 774.30 (C₅₉H₃₈N₂ = 774.97) | 28 | m/z = 804.26 |
| | | | (C₅₉H₃₆N₂S = 805.01) |
| 29 | m/z = 788.28 (C₅₉H₃₆N₂O = 788.95) | 30 | m/z = 788.28 |
| | | | (C₅₉H₃₆N₂O = 788.95) |
| 31 | m/z = 774.30 (C₅₉H₃₈N₂ = 774.97) | 32 | m/z = 774.30 |
| | | | (C₅₉H₃₈N₂ = 774.97) |
| 33 | m/z = 748.29 (C₅₇H₃₆N₂ = 748.93) | 34 | m/z = 748.29 |
| | | | (C₅₇H₃₆N₂ = 748.93) |
| 35 | m/z = 762.27 (C₅₇H₃₄N₂O = 762.91) | 36 | m/z = 762.27 |
| | | | (C₅₇H₃₄N₂O = 762.91) |
| 37 | m/z = 778.24 (C₅₇H₃₄N₂S = 778.97) | 38 | m/z = 788.32 |
| | | | (C₆₀H₄₀N₂ = 788.99) |
| 39 | m/z = 672.26 (C₅₁H₃₂N₂ = 672.83) | 40 | m/z = 672.26 |
| | | | (C₅₁H₃₂N₂ = 672.83) |
| 41 | m/z = 788.28 (C₅₉H₃₆N₂O = 788.95) | 42 | m/z = 788.28 |
| | | | (C₅₉H₃₆N₂O = 788.95) |
| 43 | m/z = 712.25 (C₅₃H₃₂N₂O = 712.85) | 44 | m/z = 712.25 |
| | | | (C₅₃H₃₂N₂O = 712.85) |
| 45 | m/z = 762.27 (C₅₇H₃₄N₂O = 762.91) | 46 | m/z = 778.24 |
| | | | (C₅₇H₃₄N₂S = 778.97) |
| 47 | m/z = 728.23 (C₅₃H₃₂N₂S = 728.91) | 48 | m/z = 728.23 |
| | | | (C₅₃H₃₂N₂S = 728.91) |
| 49 | m/z = 738.30 (C₅₆H₃₈N₂ = 738.93) | 50 | m/z = 788.32 |
| | | | (C₆₀H₄₀N₂ = 788.99) |
| 51 | m/z = 788.32 (C₆₀H₄₀N₂ = 788.99) | 52 | m/z = 738.30 |
| | | | (C₅₆H₃₈N₂ = 738.93) |
| 53 | m/z = 698.27 (C₅₃H₃₄N₂ = 698.87) | 54 | m/z = 748.29 |
| | | | (C₅₇H₃₆N₂ = 748.93) |
| 55 | m/z = 798.30 (C₆₁H₃₈N₂ = 798.99) | 56 | m/z = 748.29 |
| | | | (C₅₇H₃₆N₂ = 748.93) |
| 57 | m/z = 774.30 (C₅₉H₃₈N₂ = 774.97) | 58 | m/z = 774.30 |
| | | | (C₅₉H₃₈N₂ = 774.97) |
| 59 | m/z = 788.28 (C₅₉H₃₆N₂O = 788.95) | 60 | m/z = 788.28 |
| | | | (C₅₉H₃₆N₂O = 788.95) |
| 61 | m/z = 748.29 (C₅₇H₃₆N₂ = 748.93) | 62 | m/z = 824.32 |
| | | | (C₆₃H₄₀N₂ = 825.03) |
| 63 | m/z = 824.32 (C₆₃H₄₀N₂ = 825.03) | 64 | m/z = 798.30 |
| | | | (C₆₁H₃₈N₂ = 798.99) |
| 65 | m/z = 748.29 (C₅₇H₃₆N₂ = 748.93) | 66 | m/z = 824.32 |
| | | | (C₆₃H₄₀N₂ = 825.03) |
| 67 | m/z = 788.28 (C₅₉H₃₆N₂O = 788.95) | 68 | m/z = 712.25 |
| | | | (C₅₃H₃₂N₂O = 712.85) |
| 69 | m/z = 712.25 (C₅₃H₃₂N₂O = 712.85) | 70 | m/z = 728.23 |
| | | | (C₅₃H₃₂N₂S = 728.91) |
| 71 | m/z = 774.30 (C₅₉H₃₈N₂ = 774.97) | 72 | m/z = 722.27 |
| | | | (C₅₅H₃₄N₂ = 722.89) |
| 73 | m/z = 738.30 (C₅₆H₃₈N₂ = 738.93) | 74 | m/z = 788.32 |
| | | | (C₆₀H₄₀N₂ = 788.99) |
| 75 | m/z = 814.33 (C₆₂H₄₂N₂ = 815.03) | 76 | m/z = 748.29 |
| | | | (C₅₇H₃₆N₂ = 748.93) |
| 77 | m/z = 774.30 (C₅₉H₃₈N₂ = 774.97) | 78 | m/z = 748.29 |
| | | | (C₅₇H₃₆N₂ = 748.93) |
| 79 | m/z = 748.29 (C₅₇H₃₆N₂ = 748.93) | 80 | m/z = 774.30 |
| | | | (C₅₉H₃₈N₂ = 774.97) |
| 81 | m/z = 774.30 (C₅₉H₃₈N₂ = 774.97) | 82 | m/z = 774.30 |
| | | | (C₅₉H₃₈N₂ = 774.97) |
| 83 | m/z = 650.27 (C₄₉H₃₄N₂ = 650.83) | 84 | m/z = 798.30 |
| | | | (C₆₁H₃₈N₂ = 798.99) |
| 85 | m/z = 814.33 (C₆₂H₄₂N₂ = 815.03) | 86 | m/z = 788.32 |
| | | | (C₆₀H₄₀N₂ = 788.99) |
| 87 | m/z = 814.33 (C₆₂H₄₂N₂ = 815.03) | 88 | m/z = 788.28 |
| | | | (C₅₉H₃₆N₂O = 788.95) |
| 89 | m/z = 748.29 (C₅₇H₃₆N₂ = 748.93) | 90 | m/z = 698.27 |
| | | | (C₅₃H₃₄N₂ = 698.87) |
| 91 | m/z = 728.23 (C₅₃H₃₂N₂S = 728.91) | 92 | m/z = 748.29 |
| | | | (C₅₇H₃₆N₂ = 748.93) |
| 93 | m/z = 722.27 (C₅₅H₃₄N₂ = 722.89) | 94 | m/z = 722.27 |
| | | | (C₅₅H₃₄N₂ = 722.89) |
| 95 | m/z = 798.30 (C₆₁H₃₈N₂ = 798.99) | 96 | m/z = 748.29 |
| | | | (C₅₇H₃₆N₂ = 748.93) |
| 97 | m/z = 814.33 (C₆₂H₄₂N₂ = 815.03) | 98 | m/z = 774.30 |
| | | | (C₅₉H₃₈N₂ = 774.97) |
| 99 | m/z = 748.29 (C₅₇H₃₆N₂ = 748.93) | 100 | m/z = 774.30 |
| | | | (C₅₉H₃₈N₂ = 774.97) |
| 101 | m/z = 798.30 (C₆₁H₃₈N₂ = 798.99) | 102 | m/z = 798.30 |
| | | | (C₆₁H₃₈N₂ = 798.99) |
| 103 | m/z = 788.28 (C₅₉H₃₆N₂O = 788.95) | 104 | m/z = 804.26 |
| | | | (C₅₉H₃₆N₂S = 805.01) |
| 105 | m/z = 748.29 (C₅₇H₃₆N₂ = 748.93) | 106 | m/z = 748.29 |
| | | | (C₅₇H₃₆N₂ = 748.93) |
| 107 | m/z = 814.33 (C₆₂H₄₂N₂ = 815.03) | 108 | m/z = 788.28 |
| | | | (C₅₉H₃₆N₂O = 788.95) |
| 109 | m/z = 774.30 (C₅₉H₃₈N₂ = 774.97) | 110 | m/z = 788.28 |
| | | | (C₅₉H₃₆N₂O = 788.95) |
| 111 | m/z = 774.30 (C₅₉H₃₈N₂ = 774.97) | 112 | m/z = 774.30 |
| | | | (C₅₉H₃₈N₂ = 774.97) |
| 113 | m/z = 748.29 (C₅₇H₃₆N₂ = 748.93) | 114 | m/z = 814.33 |
| | | | (C₆₂H₄₂N₂ = 815.03) |
| 115 | m/z = 788.32 (C₆₀H₄₀N₂ = 788.99) | 116 | m/z = 774.30 |
| | | | (C₅₉H₃₈N₂ = 774.97) |
| 117 | m/z = 774.30 (C₅₉H₃₈N₂ = 774.97) | 118 | m/z = 698.27 |
| | | | (C₅₃H₃₄N₂ = 26698.87) |
| 119 | m/z = 788.28 (C₅₉H₃₆N₂O = 788.95) | 120 | m/z = 728.23 |
| | | | (C₅₃H₃₂N₂S = 728.91) |
| 121 | m/z = 814.33 (C₆₂H₄₂N₂ = 815.03) | 122 | m/z = 774.30 |
| | | | (C₅₉H₃₈N₂ = 774.97) |
| 123 | m/z = 738.30 (C₅₆H₃₈N₂ = 738.93) | 124 | m/z = 788.32 |
| | | | (C₆₀H₄₀N₂ = 788.99) |
| 125 | m/z = 824.32 (C₆₃H₄₀N₂ = 825.03) | 126 | m/z = 748.29 |
| | | | (C₅₇H₃₆N₂ = 748.93) |
| 127 | m/z = 774.30 (C₅₉H₃₈N₂ = 774.97) | 128 | m/z = 748.29 |
| | | | (C₅₇H₃₆N₂ = 748.93) |
| 129 | m/z = 748.29 (C₅₇H₃₆N₂ = 748.93) | 130 | m/z = 798.30 |
| | | | (C₆₁H₃₈N₂ = 798.99) |
| 131 | m/z = 774.30 (C₅₉H₃₈N₂ = 774.97) | 132 | m/z = 748.29 |
| | | | (C₅₇H₃₆N₂ = 748.93) |
| 133 | m/z = 774.30 (C₅₉H₃₈N₂ = 774.97) | 134 | m/z = 698.27 |
| | | | (C₅₃H₃₄N₂ = 698.87) |
| 135 | m/z = 748.29 (C₅₇H₃₆N₂ = 748.93) | 136 | m/z = 788.28 |
| | | | (C₅₉H₃₆N₂O = 788.95) |
| 137 | m/z = 788.28 (C₅₉H₃₆N₂O = 788.95) | 138 | m/z = 814.33 |
| | | | (C₆₂H₄₂N₂ = 815.03) |
| 139 | m/z = 788.28 (C₅₉H₃₆N₂O = 788.95) | 140 | m/z = 748.29 |
| | | | (C₅₇H₃₆N₂ = 748.93) |
| 141 | m/z = 824.32 (C₆₃H₄₀N₂ = 825.03) | 142 | m/z = 774.30 |
| | | | (C₅₉H₃₈N₂ = 774.97) |
| 143 | m/z = 774.30 (C₅₉H₃₈N₂ = 774.97) | 144 | m/z = 814.33 |
| | | | (C₆₂H₄₂N₂ = 815.03) |
| 145 | m/z = 748.29 (C₅₇H₃₆N₂ = 748.93) | 146 | m/z = 748.29 |
| | | | (C₅₇H₃₆N₂ = 748.93) |
| 147 | m/z = 672.26 (C₅₁H₃₂N₂ = 672.83) | 148 | m/z = 738.30 |
| | | | (C₅₆H₃₈N₂ = 738.93) |
| 149 | m/z = 712.25 (C₅₃H₃₂N₂O = 712.85) | 150 | m/z = 762.27 |
| | | | (C₅₇H₃₄N₂O = 762.91) |
| 151 | m/z = 728.23 (C₅₃H₃₂N₂S = 728.91) | 152 | m/z = 738.30 |
| | | | (C₅₆H₃₈N₂ = 738.93) |
| 153 | m/z = 722.27 (C₅₅H₃₄N₂ = 722.89) | 154 | m/z = 824.32 |
| | | | (C₆₃H₄₀N₂ = 825.03) |
| 155 | m/z = 748.29 (C₅₇H₃₆N₂ = 748.93) | 156 | m/z = 698.27 |
| | | | (C₅₃H₃₄N₂ = 698.87) |
| 157 | m/z = 698.27 (C₅₃H₃₄N₂ = 698.87) | 158 | m/z = 774.30 |
| | | | (C₅₉H₃₈N₂ = 774.97) |
| 159 | m/z = 748.29 (C₅₇H₃₆N₂ = 748.93) | 160 | m/z = 774.30 |
| | | | (C₅₉H₃₈N₂ = 774.97) |
| 161 | m/z = 814.33 (C₆₂H₄₂N₂ = 815.03) | 162 | m/z = 814.33 |
| | | | (C₆₂H₄₂N₂ = 815.03) |
| 163 | m/z = 788.28 (C₅₉H₃₆N₂O = 788.95) | 164 | m/z = 788.28 |
| | | | (C₅₉H₃₆N₂O = 788.95) |
| 165 | m/z = 774.30 (C₅₉H₃₈N₂ = 774.97) | 166 | m/z = 748.29 |
| | | | (C₅₇H₃₆N₂ = 748.93) |
| 167 | m/z = 814.33 (C₆₂H₄₂N₂ = 815.03) | 168 | m/z = 776.32 |
| | | | (C₅₉H₄₀N₂ = 776.98) |
| 169 | m/z = 788.28 (C₅₉H₃₆N₂O = 788.95) | 170 | m/z = 804.26 |
| | | | (C₅₉H₃₆N₂S = 805.01) |
| 171 | m/z = 748.29 (C₅₇H₃₆N₂ = 748.93) | 172 | m/z = 698.27 |
| | | | (C₅₃H₃₄N₂ = 698.87) |
| 173 | m/z = 774.30 (C₅₉H₃₈N₂ = 774.97) | 174 | m/z = 748.29 |
| | | | (C₅₇H₃₆N₂ = 748.93) |
| 175 | m/z = 788.28 (C₅₉H₃₆N₂O = 788.95) | 176 | m/z = 698.27 |
| | | | (C₅₃H₃₄N₂ = 698.87) |
| 177 | m/z = 850.33 (C₆₅H₄₂N₂ = 851.07) | 178 | m/z = 810.40 |
| | | | (C₆₁H₅₀N₂ = 811.09) |
| 179 | m/z = 784.38 (C₅₉H₄₈N₂ = 785.05) | 180 | m/z = 784.38 |
| | | | (C₅₉H₄₈N₂ = 785.05) |
| 181 | m/z = 810.40 (C₆₁H₅₀N₂ = 811.09) | 182 | m/z = 774.30 |
| | | | (C₅₉H₃₈N₂ = 774.97) |
| 183 | m/z = 784.38 (C₅₉H₄₈N₂ = 785.05) | 184 | m/z = 734.37 |
| | | | (C₅₅H₄₆N₂ = 734.99) |
| 185 | m/z = 774.30 (C₅₉H₃₈N₂ = 774.97) | 186 | m/z = 824.32 |
| | | | (C₆₃H₄₀N₂ = 825.03) |
| 187 | m/z = 824.32 (C₆₃H₄₀N₂ = 825.03) | 188 | m/z = 733.26 |
| | | | (C₅₃H₂₇D₅N₂S = 733.94) |
| 189 | m/z = 793.31 | 190 | m/z = 708.33 |
| | (C₅₉H₃₁D₅N₂O = 793.98) | | (C₅₃H₂₄D₁₀N₂ = 708.93) |
| 191 | m/z = 778.33 | 192 | m/z = 630.29 |
| | (C₅₉H₃₄D₄N₂ = 778.99) | | (C₄₇H₂₂D₈N₂ = 630.82) |
| 193 | m/z = 674.25 (C₄₉H₃₀N₄ = 674.81) | 194 | m/z = 724.26 |
| | | | (C₅₃H₃₂N₄ = 724.87) |
| 195 | m/z = 699.27 (C₅₂H₃₃N₃ = 699.86) | 196 | m/z = 749.28 |
| | | | (C₅₆H₃₅N₃ = 749.92) |
| 197 | m/z = 620.23 (C₄₇H₂₈N₂ = 620.76) | 198 | m/z = 670.24 |
| | | | (C₅₁H₃₀N₂ = 670.82) |
| 199 | m/z = 699.26 (C₅₃H₃₂N₂ = 699.85) | 200 | m/z = 720.26 |
| | | | (C₅₅H₃₂N₂ = 720.88) |

### <Preparation Example 2> Preparation of Compound 10

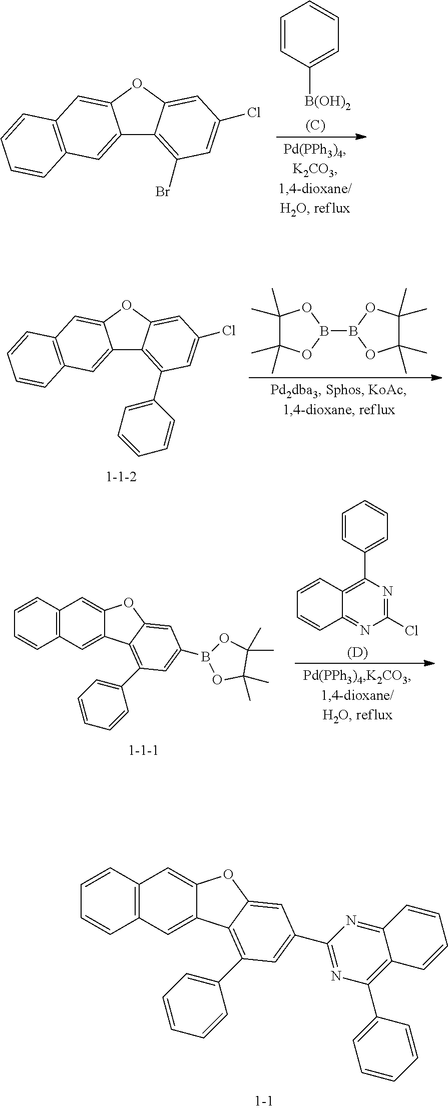

1) Preparation of Compound 1-1-2

6.0 g of 1-bromo-3-chloronaphtho[2,3-b]benzofuran (18.09 mmol), 2.65 g of phenylboronic acid (C) (21.71 mmol), 1.23 g of Pd(PPh₃)₄ (1.07 mmol), and 5.89 g of K₂CO₃ (42.62 mmol) were dissolved in 30 mL/6 mL of 1,4-dioxane/H₂O and refluxed for 24 hours. After the reaction was completed, the reactant was extracted with distilled water and dichloromethane (DCM) at room temperature, and the organic layer was dried over MgSO₄, and the solvent was removed by a rotary evaporator. The solvent-removed reactant was purified using silica, and the reactant was purified by column chromatography (DCM:Hex=1:5) to obtain 5 g (45%) of compound 1-1-2. The Hex means hexane.

2) Preparation of Compound 1-1-1

5.0 g of the compound 1-1-2 (15.21 mmol), 4.3 g of bis(pinacolato)diboron (16.73 mmol), 0.44 g of tris(dibenzylideneacetone)dipalladium (0) (Pd₂(dba)₃) (1.07 mmol), 0.4 g of dicyclohexyl-(2′,6′-dimethoxybiphenyl-2-yl) phosphine (Sphos) (0.958 mmol), and 1.9 g of KOAc (19.16 mmol) were dissolved in 50 mL of 1,4-dioxane and refluxed for 5 hours. After the reaction was completed, the reactant was extracted with distilled water and dichloromethane (DCM) at room temperature, and the organic layer was dried over MgSO₄, and the solvent was removed by a rotary evaporator. The solvent-removed reactant was purified using silica, and then recrystallized from methanol to obtain 3.27 g (56%) of compound 1-1-1.

3) Preparation of target compound 1-1

3.27 g of the compound 1-1-1 (7.8 mmol), 1.9 g of 2-chloro-4-phenylquinazoline (D) (7.8 mmol), 0.31 g of Pd(PPh₃)₄ (0.27 mmol), and 1.47 g of K₂CO₃ (10.66 mmol) were dissolved in 25 mL/5 mL of 1,4-dioxane/H₂O, and then refluxed for 3 hours. After the reaction was completed, the resulting solid was filtered, washed with distilled water, and dried. The dried solid was dissolved in chloroform and purified using silica, and then the solvent was removed using a rotary evaporator. By recrystallization with acetone, 3.1 g (80%) of the target compound 1-1 was obtained.

The target compounds of Table 7 below were additionally prepared in the same manner as in Preparation Example 2, except that in Preparation Example 2, C and D of Table 7 below were used as intermediates, instead of phenylboronic acid (C) and 2-chloro-4-phenylquinazoline (D).

TABLE 7

| Compound | | | | |
|---|---|---|---|---|
| no. | Intermediate C | Intermediate D | Target compound | Yield |
| 1-2 | 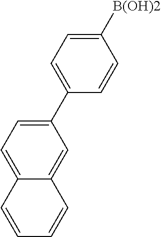 [SMILES: OB(O)c1ccc(-c2ccc3ccccc3c2)cc1] | 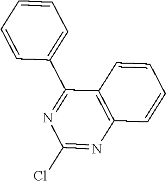 [SMILES: Clc1nc(-c2ccccc2)c2ccccc2n1] | 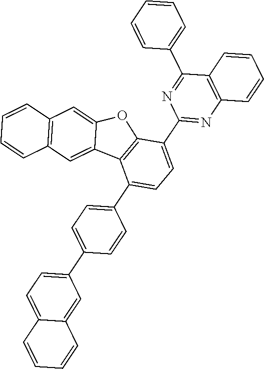 [SMILES: c1ccc(-c2nc(-c3ccc(-c4ccc(-c5ccc6ccccc6c5)cc4)c4c3oc3cc5ccccc5cc34)nc3ccccc23)cc1] | 63% |
| 1-3 | 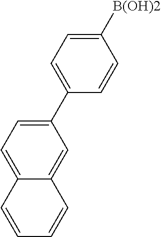 [SMILES: OB(O)c1ccc(-c2ccc3ccccc3c2)cc1] | 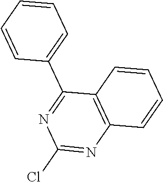 [SMILES: Clc1nc(-c2ccccc2)c2ccccc2n1] | 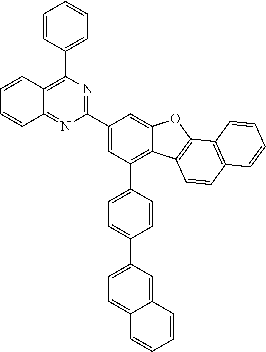 [SMILES: c1ccc(-c2nc(-c3cc(-c4ccc(-c5ccc6ccccc6c5)cc4)c4c(c3)oc3c5ccccc5ccc34)nc3ccccc23)cc1] | 59% |
| 1-5 | 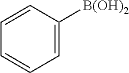 | 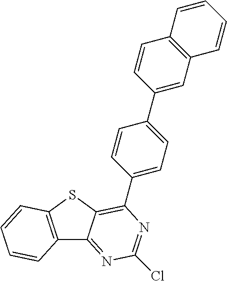 [SMILES: Clc1nc(-c2ccc(-c3ccc4ccccc4c3)cc2)c2sc3ccccc3c2n1] | 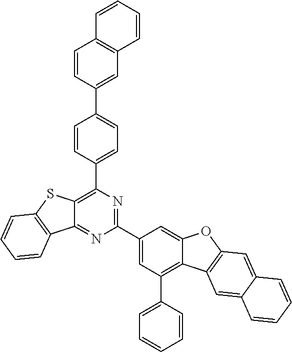 [SMILES: c1ccc(-c2cc(-c3nc(-c4ccc(-c5ccc6ccccc6c5)cc4)c4sc5ccccc5c4n3)cc3oc4cc5ccccc5cc4c23)cc1] | 69% |
| 1-6 | 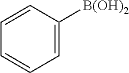 | 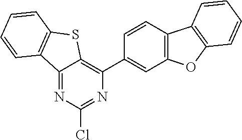 [SMILES: Clc1nc(-c2ccc3c(c2)oc2ccccc23)c2sc3ccccc3c2n1] | 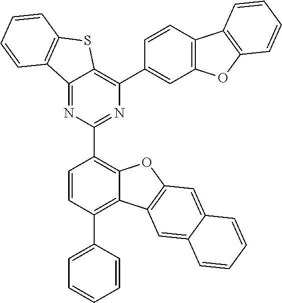 [SMILES: c1ccc(-c2ccc(-c3nc(-c4ccc5c(c4)oc4ccccc45)c4sc5ccccc5c4n3)c3oc4cc5ccccc5cc4c23)cc1] | 48% |

The remaining compounds other than the compounds described in Preparation Example 2 and Table 7 were prepared in the same manner as in Preparation Examples described above, and the synthesis results are shown in Tables 8 and 9 below. Table 8 below is a measurement value of 1H NMR (CDCl₃, 300 Mz), and Table 9 below is a measurement value of the FD-mass spectrometer (FD-MS: Field desorption mass spectrometry).

TABLE 8

| NO | ¹H NMR (CDC1₃, 300 Mz) |
|---|---|
| 1-1 | 8.28 (d, 1H), 8.13 (d, 1H), 8.86-7.75 (m, 10H), 7.55-7.39 (m, 24H), |
| | 7.65-7.41 (m, 10H) |
| 1-5 | 8.30 (d, 2H), 8.09 (d, 1H), 8.06 (d, 1H), 7.99 (d, 1H), 7.86-7.75 |
| | (m, 10H), 7.63-7.31 (m, 12H), |
| 1-6 | 8.28 (d, 1H), 7.98 (d, 1H), 7.86-7.75 (m, 10H), 7.54-7.31 (m, 12H) |
| 1-7 | 8.97 (d, 2H), 8.95 (d, 1H), 8.50 (dd, 1H), 7.86-7.76 (m, 9H), 7.53- |
| | 7.25 (m, 15H) |
| 1-8 | 8.95 (dd, 1H), 8.50 (dd, 1H), 8.36 (d, 2H), 8.25 (d, 2H), 8.19 (d, |
| | 1H), 7.89-7.75 (m, 8H), 7.50-7.35 (m, 10H), 7.25 (d, 2H), 7.23 (d, 1H), |
| 1-10 | 8.55 (d, 1H), 8.38 (d, 1H), 8.36 (dd, 4H), 8.28 (d, 1H), 8.20-8.19 |
| | (m, 3H), 7.94-7.84 (m, 6H), 7.75-7.71 (t, 2H), 7.52-7.40 (m, 11H), |
| | 7.20-7.11 (m, 4H) |
| 1-12 | 9.09 (s, 1H), 8.55-8.49 (d, 2H), 8.38 (d, 1H), 8.28-8.16 (m, 5H), |
| | 8.00-7.84 (m, 5H), 7.75-7.40 (m, 16H), 7.20-7.11 (m, 4H) |
| 1-13 | 8.95 (d, 1H), 8.50 (d, 1H), 8.36-8.25 (dd, 5H), 7.93-7.75 (m, 6H), |
| | 7.51-7.35 (m, 12H), 7.25 (d, 2H) |
| 1-15 | 9.09 (d, 2H), 8.49 (d, 2H), 8.38 (d, 1H), 8.28 (d, 1H), 8.16-7.41 |
| | (m, 25H) |
| 1-16 | 9.09 (d, 1H), 9.02-8.95 (dd, 2H), 8.49 (d, 1H), 8.36-8.28 (d, 3H), |
| | 8.08 (d, 1H), 8.00 (d, 1H), 7.84-7.75 (m, 5H), 7.51-7.35 (m, 15H) |

TABLE 9

| Compound | FD-MS | Compound | FD-MS |
|---|---|---|---|
| 1-1 | m/z = 498.17 | 1-5 | m/z = 680.19 |
| | (C₃₆H₂₂N₂O = 498.59) | | (C₄₈H₂₈N₂OS = 680.83) |
| 1-6 | m/z = 644.16 | 1-7 | m/z = 680.19 |
| | (C₄₄H₂₄N₂O₂S = 644.75) | | (C₄₈H₂₈N₂OS = 680.83) |
| 1-8 | m/z = 651.23 | 1-10 | m/z = 766.27 |
| | (C₄₇H₂₉N₃O = 651.77) | | (C₅₅H₃₄N₄O = 766.90) |
| 1-12 | m/z = 816.29 | 1-13 | m/z = 651.23 |
| | (C₅₉H₃₆N₄O = 816.96) | | (C ₄₇H₂₉N₃O = 651.77) |
| 1-15 | m/z = 701.25 | 1-16 | m/z = 701.25 |
| | (C₅₁H₃₁N₃O = 701.83) | | (C₅₁H₃₁N₃O = 701.83) |

### Experimental Example 1

(1) Manufacturing of Organic Light Emitting Device

A glass substrate coated with a thin film of ITO/Ag/ITO (indium tin oxide) to thicknesses of 115 Å/100 Å/15 Å was ultrasonically washed with distilled water. After washing with distilled water, the substrate was ultrasonically washed with a solvent such as acetone, methanol, isopropyl alcohol, etc., dried, and then treated with ultraviolet ozone (UVO) for 5 minutes using ultraviolet (UV) in a UV cleaner. Thereafter, the substrate was transferred to a plasma cleaner (PT), and then plasma-treated in a vacuum to increase the work function of ITO and remove the remaining film, and transferred to a thermal deposition equipment for organic deposition.

A hole injection layer of HAT-CN(Hexaazatriphenylenehexacarbonitrile), a hole transport layer of α-NPB(N,N′-Di(1-naphthyl)-N,N′-diphenyl-(1,1′-biphenyl)-4,4′-diamine), a light auxiliary layer of TPD(N-([1,1′-biphenyl]-4-yl)-N,1′-diphenyl-1′H-spiro[fluorene-9,5′-naphtho[8,1,2,3-cdef]carbazol]-7′-amine) 700˜900 Å, and an electron blocking layer of TAPC(N-([1,1′-biphenyl]-4-yl)-N,1′-diphenyl-1′H-spiro[fluorene-9,5′-naphtho[8,1,2,3-cdef]carbazol]-7′-amine) 100˜150 Å, or a exciton blocking layer of TCTA(tris(4-carbazoyl-9-ylphenyl)amine) as common layers were formed on the ITO electrode (positive electrode).

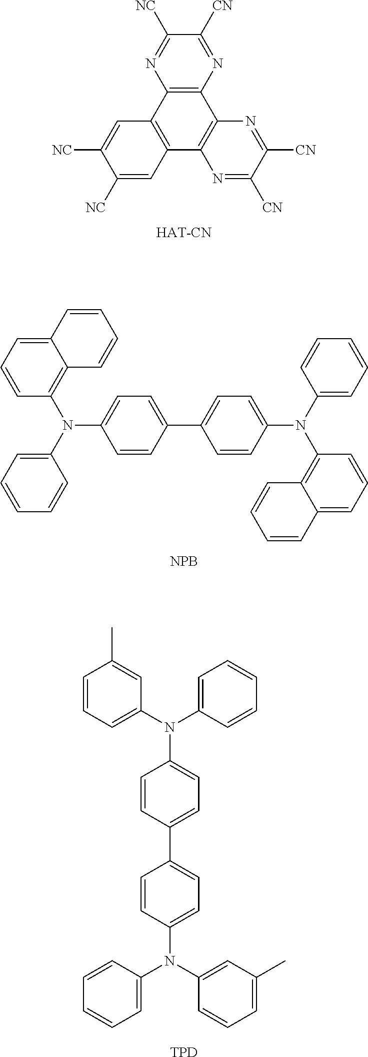

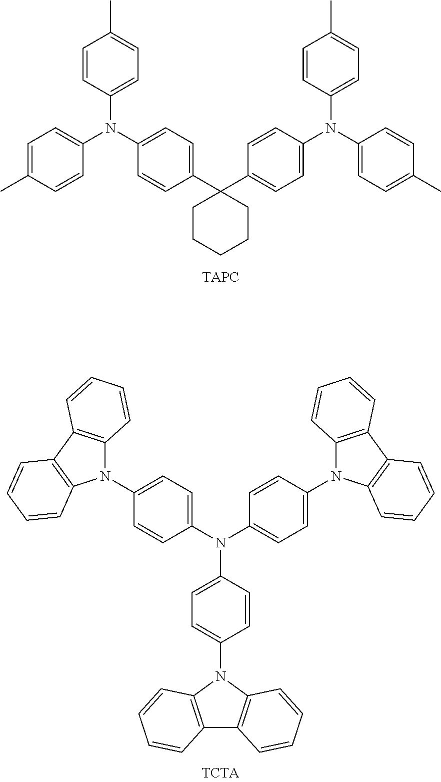

On top of that, a light emitting layer was thermally deposited in the vacuum as follows. The light emitting layer was formed by depositing a single or two types of compounds listed in Tables 10 and 11 as a red host through a single source, and doping (piq)₂(Ir) (acac) to a host at 3% while using (piq)₂(Ir) (acac) below as a red phosphorescent dopant, thereby depositing with a thickness of 400 Å. Thereafter, Bphen below was deposited with a thickness of 60 Å as a hole blocking layer, and TPBI was deposited with a thickness of 200 Å as an electron transport layer thereon. Finally, lithium fluoride (LiF) was deposited with a thickness of 10 Å on the electron transport layer to form an electron injection layer, and then a negative electrode of silver (Ag) was deposited with a thickness of 200 Å on the electron injection layer to form a negative electrode, thereby manufacturing an organic electroluminescence device (Example 1 to 40 and Comparative Examples 1 to 6).

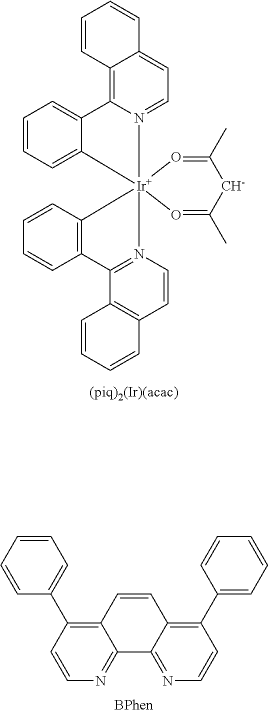

On the other hand, all organic compounds required for the manufacture of OLED devices were purified by vacuum sublimation under 10⁻⁶ to 10⁻⁸ torr for each material, and used for the manufacture of OLED (Organic Light Emitting Device).

(2) Operating Voltage and Luminous Efficiency of Organic Electroluminescence Device

For the organic electroluminescence devices manufactured as described above, electroluminescence (EL) characteristics were measured with M7000 from McScience Co. With the measurement results, T₉₀ was measured at a reference luminance of 6,000 cd/m² through the lifetime measuring device (M6000) manufactured by McScience Co.

The characteristics of the organic electroluminescence device of the present invention are shown in Tables 10 and 11 below. In the above, T₉₀ means a lifetime (unit: hour (h)) that is a time at which the luminance becomes 90% compared to the initial luminance.

The results of measuring the operating voltage, luminous efficiency, color coordinate (CIE), and lifetime of the organic light emitting devices manufactured according to the present invention are shown in Tables 10 and 11 below. In addition, the results of measuring the operating voltage, luminous efficiency, color coordinate (CIE), and lifetime of the organic light emitting devices manufactured using the following comparative compound as a compound for a light emitting layer are shown in Tables 10 and 11 below.

In this case, Table 10 below corresponds to the case where a single host material is applied, and Table 11 corresponds to a case where two host compounds were deposited through a single source by using a heterocyclic compound (donor (p-Host)) represented by Formula 1 of the present invention having excellent hole transport ability as the first host and a compound (acceptor (n-host)) corresponding to any one of the heterocyclic compounds (n-host) represented by Formula 2 of the present invention having excellent electron transport ability as the second host.

TABLE 10

| | | Operating | | Color | |
|---|---|---|---|---|---|
| | | voltage | Efficiency | coordinate | Lifetime |
| | Compound | (V) | (cd/A) | (x, y) | (T₉₀) |
| Example 1 | 3 | 3.84 | 21.7 | (0.682, | 98 |
| | | | | 0.317) | |
| Example 2 | 4 | 3.75 | 26.4 | (0.675, | 120 |
| | | | | 0.325) | |
| Example 3 | 16 | 3.78 | 26.6 | (0.682, | 115 |
| | | | | 0.317) | |
| Example 4 | 30 | 3.86 | 20.0 | (0.691, | 95 |
| | | | | 0.309) | |
| Example 5 | 42 | 4.03 | 20.2 | (0.691, | 96 |
| | | | | 0.309) | |
| Example 6 | 55 | 4.01 | 20.8 | (0.675, | 100 |
| | | | | 0.325) | |
| Example 7 | 60 | 3.91 | 20.8 | (0.681, | 102 |
| | | | | 0.319) | |
| Example 8 | 62 | 3.96 | 19.5 | (0.682, | 97 |
| | | | | 0.317) | |
| Example 9 | 63 | 4.07 | 20.0 | (0.675, | 105 |
| | | | | 0.325) | |
| Example 10 | 67 | 4.11 | 18.3 | (0.669, | 80 |
| | | | | 0.321) | |
| Example 11 | 71 | 3.90 | 21.1 | (0.681, | 106 |
| | | | | 0.319) | |
| Example 12 | 73 | 3.78 | 22.5 | (0.691, | 113 |
| | | | | 0.309) | |
| Example 13 | 79 | 3.92 | 18.9 | (0.681, | 95 |
| | | | | 0.319) | |
| Example 14 | 87 | 4.00 | 20.5 | (0.678, | 98 |
| | | | | 0.322) | |
| Example 15 | 95 | 4.01 | 19.8 | (0.674, | 85 |
| | | | | 0.326) | |
| Example 16 | 98 | 3.98 | 18.5 | (0.682, | 101 |
| | | | | 0.317) | |
| Example 17 | 101 | 4.04 | 18.9 | (0.691, | 87 |
| | | | | 0.309) | |
| Example 18 | 103 | 4.06 | 19.9 | (0.675, | 88 |
| | | | | 0.325) | |
| Example 19 | 108 | 4.10 | 18.5 | (0.681, | 85 |
| | | | | 0.319) | |
| Example 20 | 110 | 4.08 | 19.9 | (0.675, | 90 |
| | | | | 0.325) | |
| Example 21 | 113 | 4.03 | 19.5 | (0.669, | 92 |
| | | | | 0.321) | |
| Example 22 | 118 | 3.76 | 26.7 | (0.691, | 120 |
| | | | | 0.309) | |
| Example 23 | 120 | 4.12 | 19.2 | (0.681, | 86 |
| | | | | 0.319) | |
| Example 24 | 121 | 3.85 | 21.8 | (0.678, | 109 |
| | | | | 0.322) | |
| Example 25 | 127 | 3.86 | 20.0 | (0.685, | 92 |
| | | | | 0.315) | |
| Example 26 | 130 | 3.99 | 19.3 | (0.668, | 95 |
| | | | | 0.351) | |
| Example 27 | 133 | 3.98 | 19.6 | (0.685, | 92 |
| | | | | 0.315) | |
| Example 28 | 137 | 4.00 | 19.0 | (0.691, | 90 |
| | | | | 0.309) | |
| Example 29 | 140 | 3.92 | 20.6 | (0.681, | 96 |
| | | | | 0.319) | |
| Example 30 | 141 | 4.02 | 19.4 | (0.678, | 99 |
| | | | | 0.322) | |
| Example 31 | 146 | 4.08 | 18.9 | (0.674, | 89 |
| | | | | 0.326) | |
| Example 32 | 148 | 3.98 | 20.5 | (0.683, | 99 |
| | | | | 0.317) | |
| Example 33 | 153 | 3.95 | 20.6 | (0.681, | 101 |
| | | | | 0.319) | |
| Example 34 | 154 | 3.98 | 20.5 | (0.685, | 100 |
| | | | | 0.315) | |
| Example 35 | 159 | 4.00 | 20.1 | (0.676, | 98 |
| | | | | 0.324) | |
| Example 36 | 162 | 3.99 | 20.6 | (0.674, | 106 |
| | | | | 0.326) | |
| Example 37 | 168 | 4.15 | 18.2 | (0.691, | 83 |
| | | | | 0.309) | |
| Example 38 | 170 | 4.20 | 17.9 | (0.681, | 80 |
| | | | | 0.319) | |
| Example 39 | 171 | 4.11 | 18.6 | (0.685, | 87 |
| | | | | 0.315) | |
| Example 40 | 173 | 4.17 | 18.1 | (0.691, | 88 |
| | | | | 0.309) | |
| Comparative | A | 4.77 | 7.9 | (0.682, | 50 |
| Example 1 | | | | 0.317) | |
| Comparative | B | 4.67 | 7.1 | (0.691, | 45 |
| Example 2 | | | | 0.309) | |
| Comparative | C | 4.67 | 9.9 | (0.675, | 55 |
| Example 3 | | | | 0.325) | |
| Comparative | D | 4.96 | 5.5 | (0.681, | 40 |
| Example 4 | | | | 0.319) | |
| Comparative | E | 4.64 | 5.2 | (0.675, | 55 |
| Example 5 | | | | 0.325) | |
| Comparative | F | 4.42 | 7.5 | (0.684, | 42 |
| Example 6 | | | | 0.316) | |

TABLE 11

| | | | | Oper- | | Color | |
|---|---|---|---|---|---|---|---|
| | | | | ating | Effi- | coordi- | |
| | First | Second | Ratio | voltage | ciency | nate | Yield |
| | host | host | (N:P) | (V) | (cd/A) | (x, y) | (T₉₀) |
| Comparative | A | 1-8 | 1:1 | 3.99 | 12.6 | (0.669, | 70 |
| Example 10 | | | | | | 0.321) | |
| Comparative | D | | | 4.32 | 12.2 | (0.691, | 40 |
| Example 11 | | | | | | 0.309) | |
| Comparative | F | | | 3.98 | 13.1 | (0.681, | 55 |
| Example 12 | | | | | | 0.319) | |
| Example 44 | 4 | | | 3.64 | 38.9 | (0.678, | 250 |
| | | | | | | 0.322) | |
| Example 45 | 67 | 1-3 | 1:2 | 3.96 | 25.1 | (0.685, | 179 |
| | | | | | | 0.315) | |
| Example 46 | | 1-5 | | 3.89 | 26.5 | (0.668, | 186 |
| | | | | | | 0.351) | |
| Example 47 | | 1-8 | | 3.85 | 28.0 | (0.681, | 200 |
| | | | | | | 0.319) | |
| Example 48 | 79 | 1-1 | 1:3 | 3.83 | 22.4 | (0.683, | 199 |
| | | | | | | 0.317) | |
| Example 49 | | 1-6 | | 3.75 | 26.7 | (0.675, | 208 |
| | | | | | | 0.325) | |
| Example 50 | | 1-13 | | 3.69 | 35.5 | (0.684, | 227 |
| | | | | | | 0.316) | |
| Example 51 | 101 | 1-7 | | 3.92 | 24.3 | (0.682, | 191 |
| | | | | | | 0.317) | |
| Example 52 | 118 | 1-16 | 1:2 | 3.64 | 36.8 | (0.675, | 243 |
| | | | | | | 0.325) | |
| Example 53 | 154 | | | 3.86 | 30.0 | (0.682, | 209 |
| | | | | | | 0.317) | |
| Example 54 | 162 | 1-14 | 1:1 | 3.80 | 34.5 | (0.691, | 236 |
| | | | | | | 0.309) | |
| Example 55 | 171 | | | 3.91 | 28.6 | (0.681, | 189 |
| | | | | | | 0.319) | |
| Example 56 | 16 | | | 3.67 | 37.9 | (0.682, | 246 |
| | | | | | | 0.317) | |
| Example 57 | 137 | | | 3.88 | 29.9 | (0.691, | 199 |
| | | | | | | 0.309) | |
| Example 58 | 127 | 1-2 | 2:1 | 3.80 | 24.8 | (0.684, | 207 |
| | | | | | | 0.316) | |
| Example 59 | | 1-7 | 2:1 | 3.72 | 29.7 | (0.683, | 216 |
| | | | | | | 0.317) | |
| Example 60 | | 1-10 | 1:3 | 3.70 | 30.4 | (0.669, | 224 |
| | | | | | | 0.321) | |

In this case, the comparative compounds A to F used in Comparative Examples 1 to 6 are as follows:

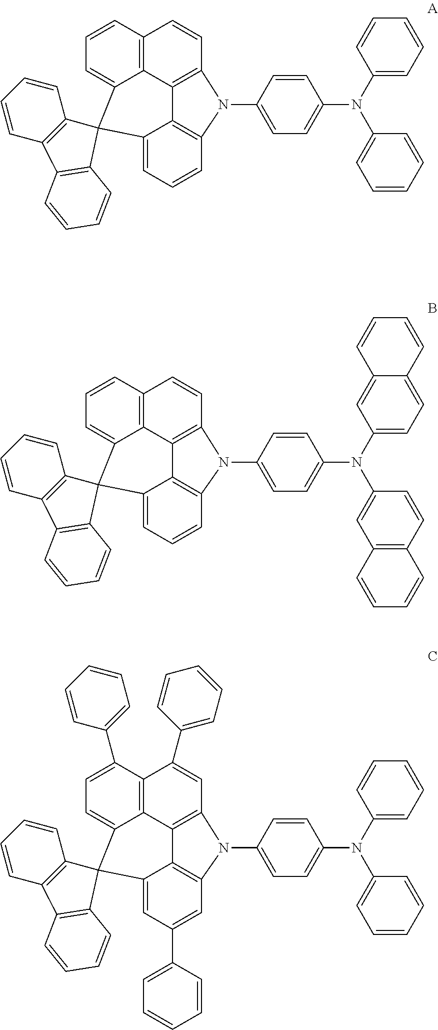 [SMILES: c1ccc(N(c2ccccc2)c2ccc(-n3c4cccc5c4c4c6c(cccc6ccc43)C53c4ccccc4-c4ccccc43)cc2)cc1 ; c1ccc2c(c1)-c1ccccc1C21c2cccc3ccc4c(c23)c2c1cccc2n4-c1ccc(N(c2ccc3ccccc3c2)c2ccc3ccccc3c2)cc1 ; c1ccc(-c2cc3c4c5c6c(ccc(-c7ccccc7)c6c(-c6ccccc6)cc5n(-c5ccc(N(c6ccccc6)c6ccccc6)cc5)c4c2)C32c3ccccc3-c3ccccc32)cc1]

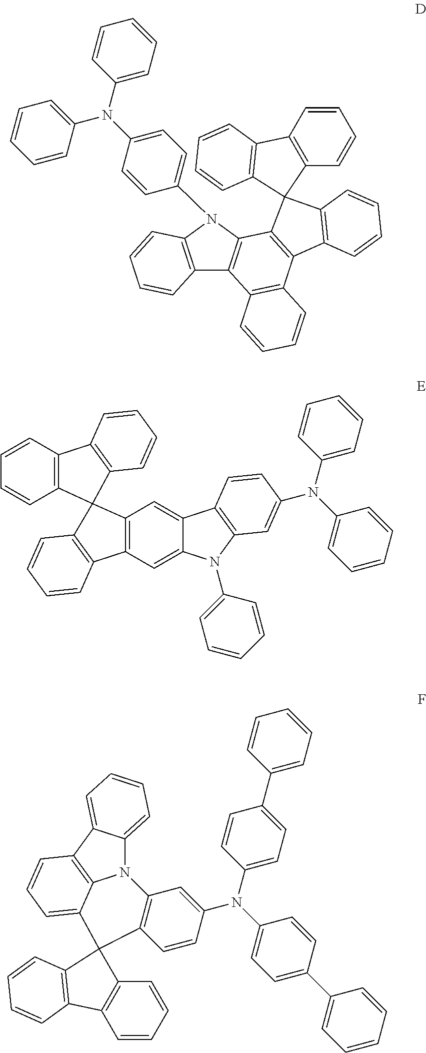 [SMILES: c1ccc(N(c2ccccc2)c2ccc(-n3c4ccccc4c4c5ccccc5c5c(c43)C3(c4ccccc4-c4ccccc43)c3ccccc3-5)cc2)cc1 ; c1ccc(N(c2ccccc2)c2ccc3c4cc5c(cc4n(-c4ccccc4)c3c2)-c2ccccc2C52c3ccccc3-c3ccccc32)cc1 ; c1ccc(-c2ccc(N(c3ccc(-c4ccccc4)cc3)c3ccc4c(c3)-n3c5ccccc5c5cccc(c53)C43c4ccccc4-c4ccccc43)cc2)cc1]

According to Table 10, it was confirmed that if the compound represented by Formula 1 is incorporated into the organic layer of the organic light emitting device, the operating voltage can be remarkably improved due to an increase in hole mobility. In addition, the efficiency is also improved due to the reduction of current leakage and the electron confinement through the electron blocking.

Specifically, if arylamine is linearly bonded as in comparative compounds A and B, the T1 level is increased, and thus energy transfer to the red dopant is not easy, and the band gap is increased to increase resistance, and thus the stability is lowered and the lifetime characteristics are lowered. On the other hand, it was confirmed that if arylamine is bonded to the positions of R11, R12, R13, and R17 as in the compound represented by Formula 1 of the present invention and is bent, the band gap is reduced and the T1 level is lowered, it is possible to significantly improve lifetime and efficiency.

In addition, it was confirmed from Table 11 that if the heterocyclic compound represented by Formula 1 and the heterocyclic compound represented by Formula 2 are simultaneously incorporated into the organic layer of an organic light emitting device, it is possible to improve operating voltage, efficiency and lifetime. From this, if the two compounds are incorporated at the same time, it can be expected that an exciplex phenomenon occurs.

The exciplex phenomenon is a phenomenon in which energy having the size of the HOMO energy level of the donor (p-host) and the LUMO energy level of the acceptor (n-host) is released due to electron exchange between two molecules. If an exciplex phenomenon occurs between two molecules, new singlet energy level (Si) and triplet energy level (T1) are formed, and thus photoluminescence (PL) that is red-shifted compared to each molecule can be observed. In particular, it was confirmed that if triazine or benzo thieno pyrimidine, which is a strong acceptor for n-host, exists, the effect of improving lifetime is greater than that of quinazoline.

In addition, if an exciplex phenomenon occurs between two molecules, a reverse intersystem crossing (RISC) may occur, thereby increasing the internal quantum efficiency of fluorescence to 100%. If a donor (p-host) with good hole transport ability and an acceptor (n-host) with good electron transport ability are used as the host of the light emitting layer, since holes are injected into the p-host and electrons are injected into the n-host, the operating voltage can be lowered, thereby helping to improve the lifetime.

In particular, it was confirmed that in the case of the heterocyclic compound represented by Formula 1, if an acceptor (n-host), which is a heterocyclic compound represented by Formula 2 having good electron transport ability, is injected, since a change of red-shifted PL is shown, an exiplex can be formed, thereby helping to improve luminescent properties. In addition, it was confirmed that if the acceptor (n-host), which is a heterocyclic compound represented by Formula 2 having good electron transport ability, is injected, the lifetime is significantly improved due to an appropriate shift of the light emitting region in the light emitting layer.

Meanwhile, in Table 12 below, the HOMO energy level, LUMO energy level, band gap and triplet energy level (T1 level) of the compound represented by Formula 1 according to the present invention and the comparative compounds A to F are shown.

As shown in Table 12 below, it was confirmed that in the case of the heterocyclic compound represented by Formula 1 according to the present invention, a donor with good hole transport ability is bonded to the spiro-naphtho carbazole core, thereby showing a high HOMO energy level, a reduced band gap, and a reduced T1 level, and thus making it suitable as a red host for an organic light emitting device.

TABLE 12

| | HOMO | LUMO | Band | T1 |
|---|---|---|---|---|
| Compound | (eV) | (eV) | Gap | (eV) |
| Comparative | −5.26 | −1.47 | 3.78 | 2.40 |
| compound A | | | | |
| Comparative | −5.22 | −1.49 | 3.73 | 2.40 |
| compound B | | | | |
| Comparative | −5.24 | −1.69 | 3.55 | 2.50 |
| compound C | | | | |
| Comparative | −5.34 | −1.72 | 3.62 | 2.41 |
| compound D | | | | |
| Comparative | −5.53 | −1.60 | 3.92 | 2.40 |
| compound E | | | | |
| Comparative | −5.47 | −1.65 | 3.81 | 2.41 |
| compound F | | | | |
| Invention | −5.01 | −1.57 | 3.44 | 2.24 |
| compound 4 | | | | |
| Invention | −5.15 | −1.70 | 3.45 | 2.20 |
| compound 71 | | | | |
| Invention | −5.01 | −1.56 | 3.45 | 2.22 |
| compound 118 | | | | |

### Experimental Example 2

(1) Manufacturing of Organic Light Emitting Device (Organic Light Emitting Device Comprising Two Stacks)

A glass substrate coated with a thin film of ITO (indium tin oxide) to thicknesses of 1,500 Å was ultrasonically washed with distilled water. After washing with distilled water, the substrate was ultrasonically washed with a solvent such as acetone, methanol, isopropyl alcohol, etc., dried, and then treated with ultraviolet ozone (UVO) for 5 minutes using ultraviolet (UV) in a UV cleaner. Thereafter, the substrate was transferred to a plasma cleaner (PT), and then plasma-treated in a vacuum to increase the work function of ITO and remove the remaining film, and transferred to a thermal deposition equipment for organic deposition.

A hole injection layer of 4,4′,4″-tris[2-naphthyl (phenyl) amino]triphenylamine (2-TNATA) and a hole transport layer of N,N′-Di(1-naphthyl)-N,N′-diphenyl-(1,1′-biphenyl)-4,4′-diamine (NPB) as common layers were formed on the ITO transparent electrode (positive electrode).

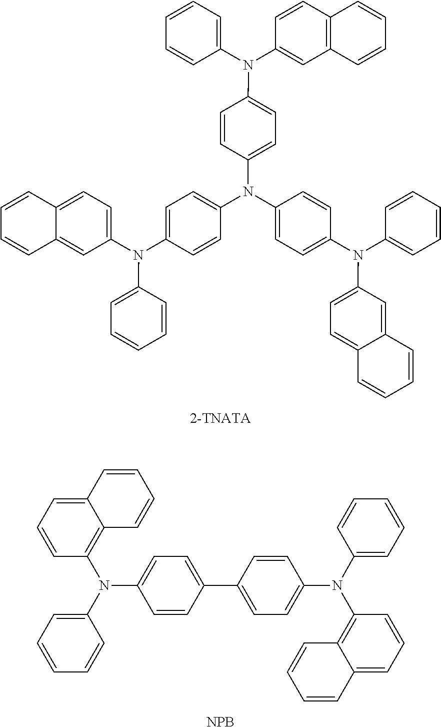 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

On top of that, a light emitting layer was thermally deposited in the vacuum as follows. The light emitting layer was formed by depositing the compound shown in Table 13 below as a red host and doping the host with (piq)₂(Ir) (acac) in an amount of 2 wt. % while using (piq)₂(Ir) (acac) below as the red phosphorescent dopant, thereby depositing with a thickness of 400 Å.

Thereafter, Alq₃ was deposited with a thickness of 120 Å as an electron transport layer, and Bphen below was deposited with a thickness of 120 Å as a charge generating layer thereon, Also, on it, MoO₃ was deposited with a thickness of 100 Å as a charge generating layer, and N,N′-Di(1-naphthyl)-N,N′-diphenyl-(1,1′-biphenyl)-4,4′-diamine (NPB) was formed thereon as a hole transport layer. On it, the light emitting layer was thermally deposited in vacuum as follows. The light emitting layer was formed by depositing the compound shown in Table 13 below as a red host and doping the host with (piq)₂(Ir) (acac) in an amount of 2 wt. % while using the following (piq)₂ (Ir) (acac) as a red phosphorescent dopant, thereby depositing with a thickness of 400 Å.

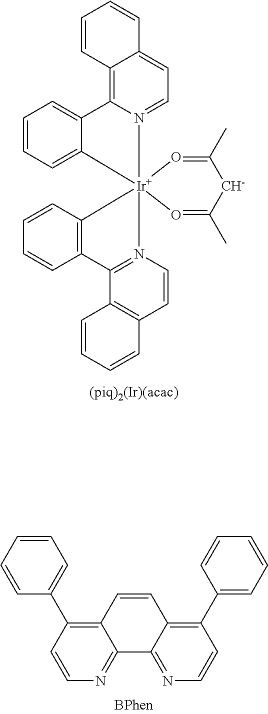

Thereafter, Alq₃ was deposited with a thickness of 300 Å as an electron transport layer. Finally, on the electron transport layer, lithium fluoride (LiF) was deposited with a thickness of 20 Å to form an electron injection layer, and then a negative electrode of aluminum (A1) was deposited with a thickness of 1,200 Å on the injection layer to form a negative electrode, thereby manufacturing an organic electroluminescence device.

Meanwhile, all organic compounds required for the manufacture of OLED devices were purified by vacuum sublimation under 10⁻⁶ to 10⁻⁸ torr for each material, and used for the manufacture of OLED (Organic Light Emitting Device).

(2) Operating Voltage and Luminous Efficiency of Organic Electroluminescence Device

For the organic electroluminescence devices manufactured as described above, electroluminescence (EL) characteristics were measured with M7000 from McScience Co. With the measurement results, T₉₀ was measured at a reference luminance of 6,000 cd/m² through the lifetime measuring device (M6000) manufactured by McScience Co. The characteristics of the organic electroluminescence device of the present invention are shown in Table 13 below. In the above, T₉₀ means a lifetime (unit: hour (h)) that is a time at which the luminance becomes 90% compared to the initial luminance.

The results of measuring the operating voltage, luminous efficiency, color coordinate (CIE), and lifetime of the organic light emitting devices manufactured according to the present invention are shown in Table 13 below. In addition, the results of measuring the operating voltage, luminous efficiency, color coordinate (CIE), and lifetime of the organic light emitting devices manufactured using the following comparative compound as a compound for a light emitting layer are shown in Table 13 below.

TABLE 13

| | | | | Oper- | | Color | |
|---|---|---|---|---|---|---|---|
| | | | | ating | Effi- | coordi- | |
| | First | Second | Ratio | voltage | ciency | nate | Yield |
| | host | host | (N:P) | (V) | (cd/A) | (x, y) | (T₉₀) |
| Example 62 | 67 | 1-3 | 1:2 | 6.33 | 52.3 | (0.685, | 359 |
| | | | | | | 0.315) | |
| Example 63 | | 1-5 | | 6.22 | 55.5 | (0.668, | 390 |
| | | | | | | 0.351) | |
| Example 64 | | 1-8 | | 6.16 | 57.8 | (0.681, | 420 |
| | | | | | | 0.319) | |
| Example 65 | 79 | 1-1 | 1:3 | 6.13 | 45.1 | (0.683, | 408 |
| | | | | | | 0.317) | |
| Example 66 | | 1-6 | | 6.00 | 52.1 | (0.675, | 421 |
| | | | | | | 0.325) | |
| Example 67 | | 1-13 | | 4.77 | 71.1 | (0.684, | 453 |
| | | | | | | 0.316) | |
| Example 68 | 101 | 1-7 | | 6.27 | 49.0 | (0.682, | 399 |
| | | | | | | 0.317) | |
| Example 69 | 118 | 1-16 | 1:2 | 5.82 | 73.3 | (0.675, | 527 |
| | | | | | | 0.325) | |
| Example 70 | 154 | | | 6.19 | 60.9 | (0.682, | 416 |
| | | | | | | 0.317) | |
| Example 71 | 162 | 1-14 | 1:1 | 6.01 | 67.8 | (0.691, | 488 |
| | | | | | | 0.309) | |
| Example 72 | 171 | | | 6.20 | 59.9 | (0.681, | 386 |
| | | | | | | 0.319) | |
| Example 73 | 16 | | | 5.87 | 75.5 | (0.682, | 490 |
| | | | | | | 0.317) | |
| Example 74 | 137 | | | 6.21 | 61.1 | (0.691, | 404 |
| | | | | | | 0.309) | |
| Example 75 | 127 | 1-2 | 2:1 | 6.11 | 59.9 | (0.684, | 416 |
| | | | | | | 0.316) | |
| Example 76 | | 1-7 | 2:1 | 5.90 | 60.7 | (0.683, | 434 |
| | | | | | | 0.317) | |
| Example 77 | | 1-10 | 1:3 | 5.88 | 61.9 | (0.669, | 456 |
| | | | | | | 0.321) | |
| Comparative | A | 1-8 | 1:1 | 6.59 | 24.7 | (0.669, | 141 |
| Example 13 | | | | | | 0.321) | |
| Comparative | D | | | 7.21 | 24.4 | (0.691, | 80 |
| Example 14 | | | | | | 0.309) | |
| Comparative | F | | | 6.60 | 25.3 | (0.681, | 100 |
| Example 15 | | | | | | 0.319) | |
| Example 61 | 4 | | | 5.82 | 81.1 | (0.678, | 530 |
| | | | | | | 0.322) | |

As shown in Table 13 above, it was confirmed that if an organic light emitting device is manufactured by stacking two stacks of light emitting layer comprising both the compound represented by Formula 1 and the compound represented by Formula 2 of the present invention, the light emitting layer is deposited twice, thereby increasing the efficiency compared to a single stack.

Meanwhile, it was confirmed that even in the case of the organic light emitting device including two stacks, the compound represented by Formula 1 of the present invention is incorporated into the organic layer of the organic light emitting device, as in the case of the organic light emitting device manufactured in a single stack, and thus the operating voltage can be significantly improved due to an increase in hole mobility. In addition, it was confirmed that the efficiency is also improved due to the reduction of current leakage and the electron confinement through the electron blocking.

All simple modifications and variations of the present invention fall within the scope of the present invention, and the specific protection scope of the present invention will be clarified by the appended claims.

### Description of Symbol

100 Substrate
200 Positive electrode
300 Organic layer
301 Hole injection layer
302 Hole transport layer
303 Light emitting layer
304 Hole blocking layer
305 Electron transport layer
306 Electron injection layer
400 Negative electrode

1. A heterocyclic compound represented by Formula 1 below: 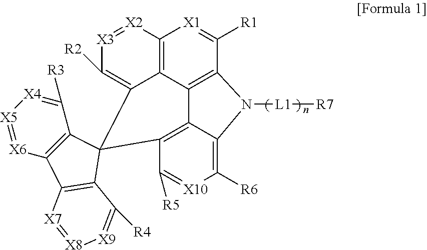
wherein,
X1 is N or CR11, X2 is N or CR12, X3 is N or CR13, X4 is N or CR14, X5 is N or CR15, X6 is N or CR16, X7 is N or CR17, X8 is N or CR18, X9 is N or CR19, and X10 is N or CR20,
R1 to R6 and R11 to R20 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other are combined with each other to form a substituted or unsubstituted C6 to C60 aromatic hydrocarbon ring or a substituted or unsubstituted C2 to C60 heterocyclic ring, wherein R101, R102, and R103 are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
R7 is a substituted or unsubstituted C6 to C60 aryl group; or a substituted or unsubstituted C2 to C60 heteroaryl group,
L1 is a direct bond; a substituted or unsubstituted C6 to C60 arylene group; or a substituted or unsubstituted C2 to C60 heteroarylene group, and
n is an integer from 0 to 5, and when n is 2 or more, L1 is the same as or different from each other.

2. The heterocyclic compound according to claim 1, wherein Formula 1 is represented by any one of Formula 1-1 to Formula 1-3 below: 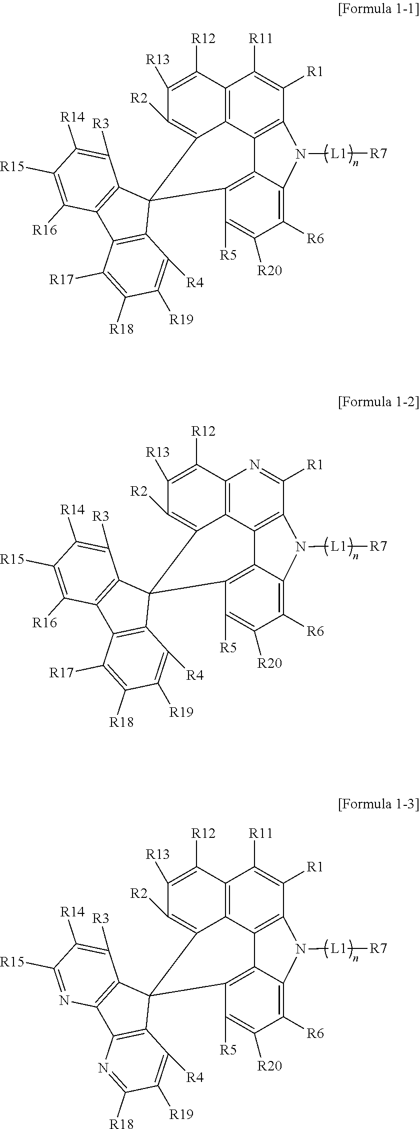
wherein the definitions of R1 to R7, R11 to R20, L1, and n are the same as in Formula 1 above.

3. The heterocyclic compound according to claim 1, wherein at least one of R11 to R20 is represented by Formula 1-4 below: 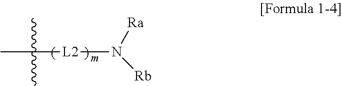
wherein,
Ra and Rb are the same as or different from each other, and are each independently a substituted or unsubstituted C6 to C60 aryl group; or a substituted or unsubstituted C2 to C60 heteroaryl group,
L2 is a direct bond; a substituted or unsubstituted C6 to C60 arylene group; or a substituted or unsubstituted C2 to C60 heteroarylene group, and
m is an integer from 0 to 5, and when m is 2 or more, L2 is the same as or different from each other.

4. The heterocyclic compound according to claim 1, wherein R7 is represented by Formula 1-5 below: 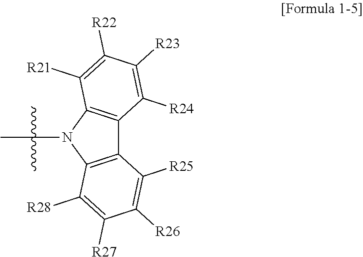
wherein,
R21 to R28 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other are combined with each other to form a substituted or unsubstituted C6 to C60 aromatic hydrocarbon ring or a substituted or unsubstituted C2 to C60 heterocyclic ring, wherein R101, R102, and R103 are the same as or different from each other, and are each independently a substituted or unsubstituted C1 to C60 alkyl group; a substituted or unsubstituted C6 to C60 aryl group; or a substituted or unsubstituted C2 to C60 heteroaryl group.

5. The heterocyclic compound according to claim 1, wherein Formula 1 is represented by any one of Formula 1-6 to Formula 1-9 below: 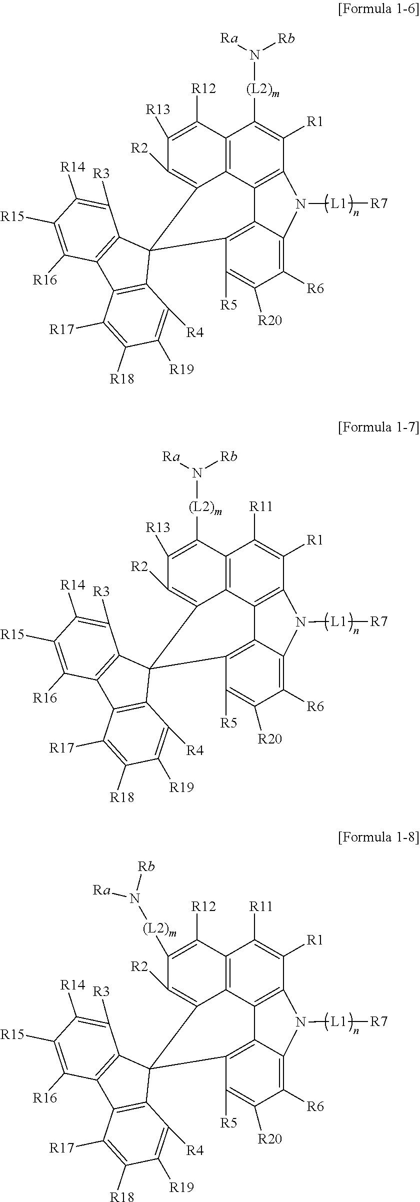 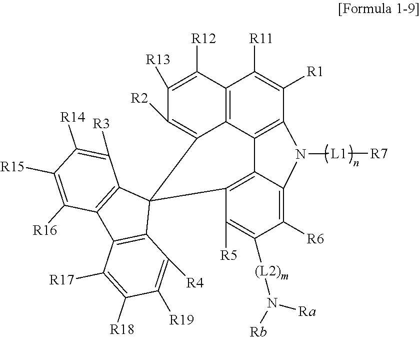
wherein,
R11 to R20 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other are combined with each other to form a substituted or unsubstituted C6 to C60 aromatic hydrocarbon ring or a substituted or unsubstituted C2 to C60 heterocyclic ring,
L2 is a direct bond; a substituted or unsubstituted C6 to C60 arylene group; or a substituted or unsubstituted C2 to C60 heteroarylene group,
m is an integer from 0 to 5, and when m is 2 or more, L2 is the same as or different from each other, and
the definitions of R1 to R7, L1 and n are the same as those in Formula 1 above.

6. The heterocyclic compound according to claim 1, wherein Formula 1 is represented by Formula 1-10 below: 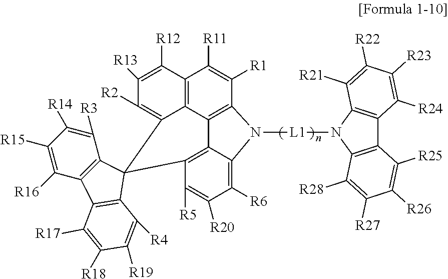
wherein,
R11 to R20 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other are combined with each other to form a substituted or unsubstituted C6 to C60 aromatic hydrocarbon ring or a substituted or unsubstituted C2 to C60 heterocyclic ring,
R21 to R28 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other are combined with each other to form a substituted or unsubstituted C6 to C60 aromatic hydrocarbon ring or a substituted or unsubstituted C2 to C60 heterocyclic ring, wherein R101, R102, and R103 are the same as or different from each other, and are each independently a substituted or unsubstituted C1 to C60 alkyl group; a substituted or unsubstituted C6 to C60 aryl group; or a substituted or unsubstituted C2 to C60 heteroaryl group, and
the definitions of R1 to R6, L1, and n are the same as those in Formula 1 above.

7. The heterocyclic compound according to claim 1, wherein Formula 1 is represented by any one of the compounds below:

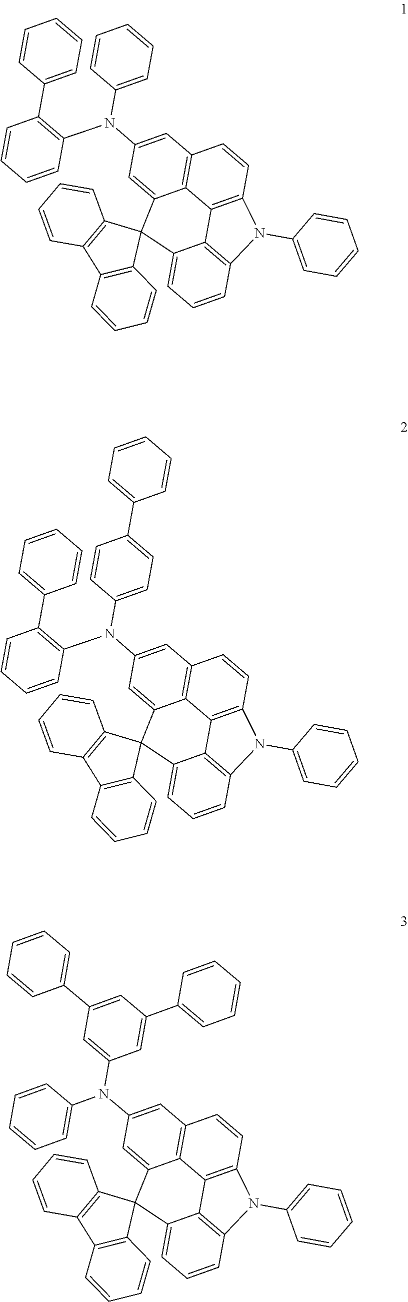 [SMILES: c1ccc(-c2ccccc2N(c2ccccc2)c2cc3c4c(ccc5c4c4c(cccc4n5-c4ccccc4)C34c3ccccc3-c3ccccc34)c2)cc1 ; c1ccc(-c2ccc(N(c3cc4c5c(ccc6c5c5c(cccc5n6-c5ccccc5)C45c4ccccc4-c4ccccc45)c3)c3ccccc3-c3ccccc3)cc2)cc1 ; c1ccc(-c2cc(-c3ccccc3)cc(N(c3ccccc3)c3cc4c5c(ccc6c5c5c(cccc5n6-c5ccccc5)C45c4ccccc4-c4ccccc45)c3)c2)cc1]

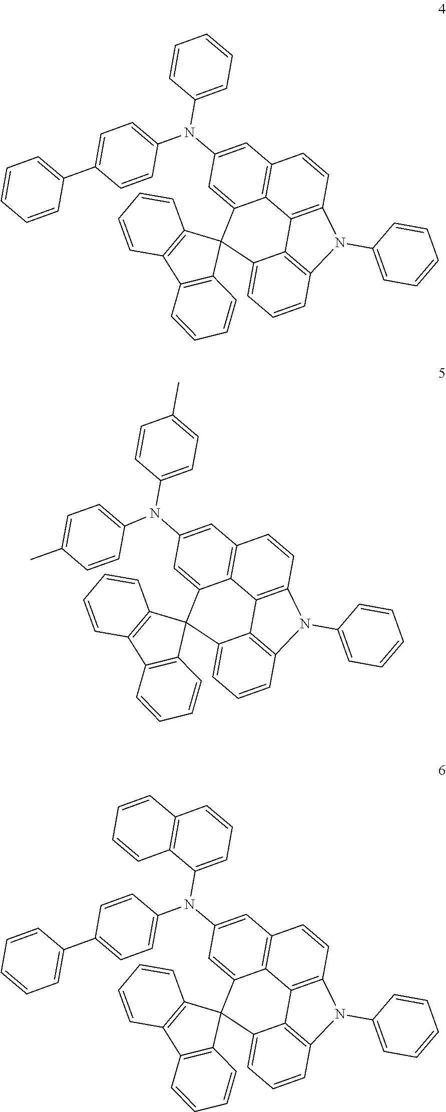

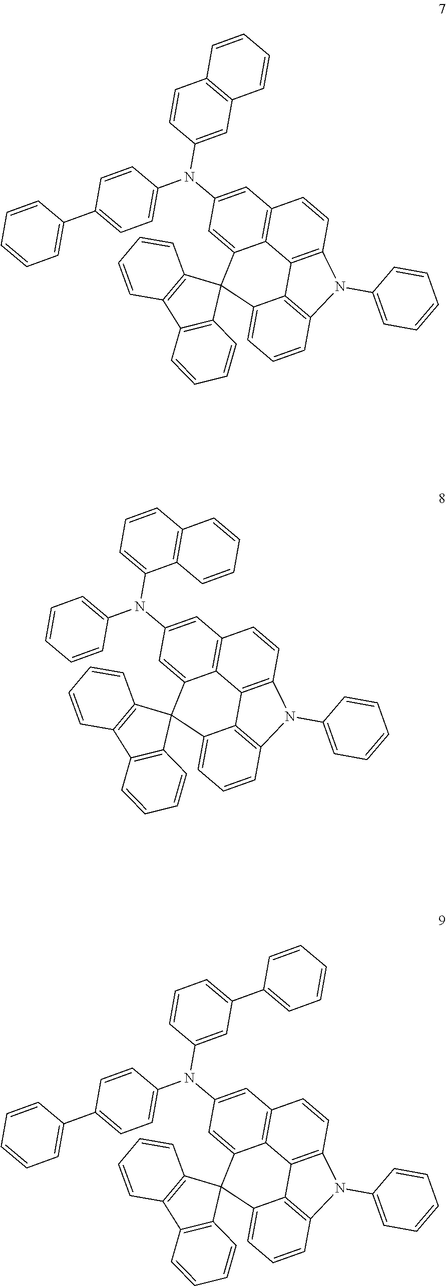 [SMILES: c1ccc(-c2ccc(N(c3ccc4ccccc4c3)c3cc4c5c(ccc6c5c5c(cccc5n6-c5ccccc5)C45c4ccccc4-c4ccccc45)c3)cc2)cc1 ; c1ccc(N(c2cc3c4c(ccc5c4c4c(cccc4n5-c4ccccc4)C34c3ccccc3-c3ccccc34)c2)c2cccc3ccccc23)cc1 ; c1ccc(-c2ccc(N(c3cccc(-c4ccccc4)c3)c3cc4c5c(ccc6c5c5c(cccc5n6-c5ccccc5)C45c4ccccc4-c4ccccc45)c3)cc2)cc1]

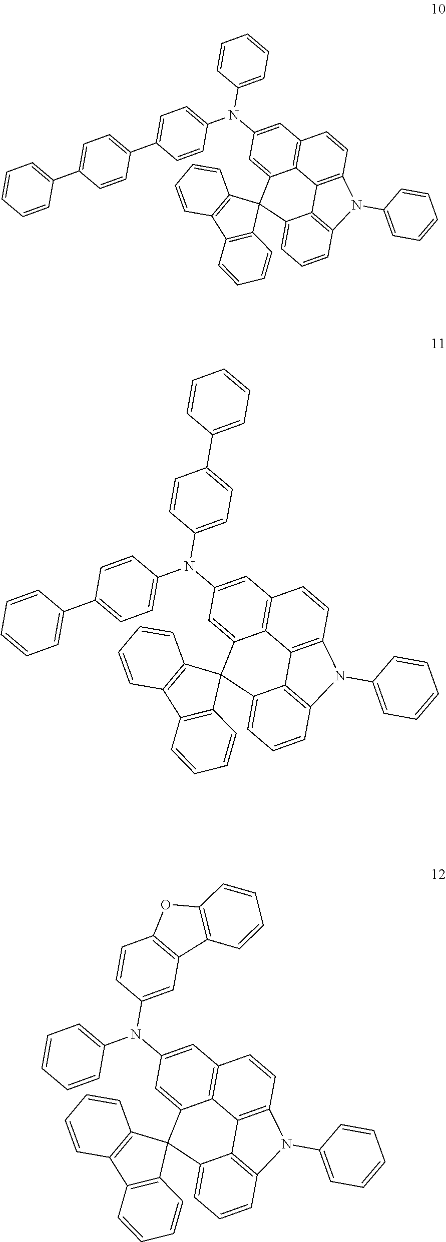 [SMILES: c1ccc(-c2ccc(-c3ccc(N(c4ccccc4)c4cc5c6c(ccc7c6c6c(cccc6n7-c6ccccc6)C56c5ccccc5-c5ccccc56)c4)cc3)cc2)cc1 ; c1ccc(-c2ccc(N(c3ccc(-c4ccccc4)cc3)c3cc4c5c(ccc6c5c5c(cccc5n6-c5ccccc5)C45c4ccccc4-c4ccccc45)c3)cc2)cc1 ; c1ccc(N(c2cc3c4c(ccc5c4c4c(cccc4n5-c4ccccc4)C34c3ccccc3-c3ccccc34)c2)c2ccc3oc4ccccc4c3c2)cc1]

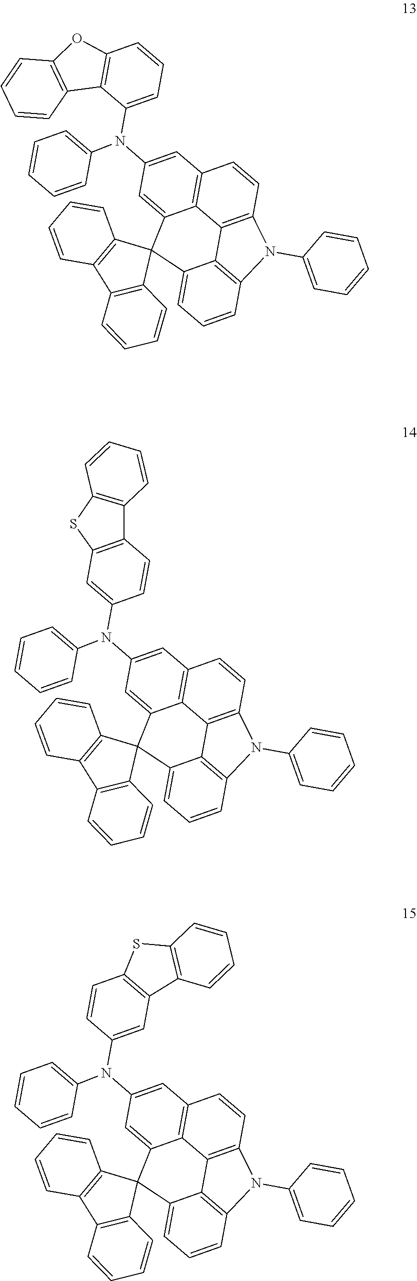 [SMILES: c1ccc(N(c2cc3c4c(ccc5c4c4c(cccc4n5-c4ccccc4)C34c3ccccc3-c3ccccc34)c2)c2cccc3oc4ccccc4c23)cc1 ; c1ccc(N(c2cc3c4c(ccc5c4c4c(cccc4n5-c4ccccc4)C34c3ccccc3-c3ccccc34)c2)c2ccc3c(c2)sc2ccccc23)cc1 ; c1ccc(N(c2cc3c4c(ccc5c4c4c(cccc4n5-c4ccccc4)C34c3ccccc3-c3ccccc34)c2)c2ccc3sc4ccccc4c3c2)cc1]

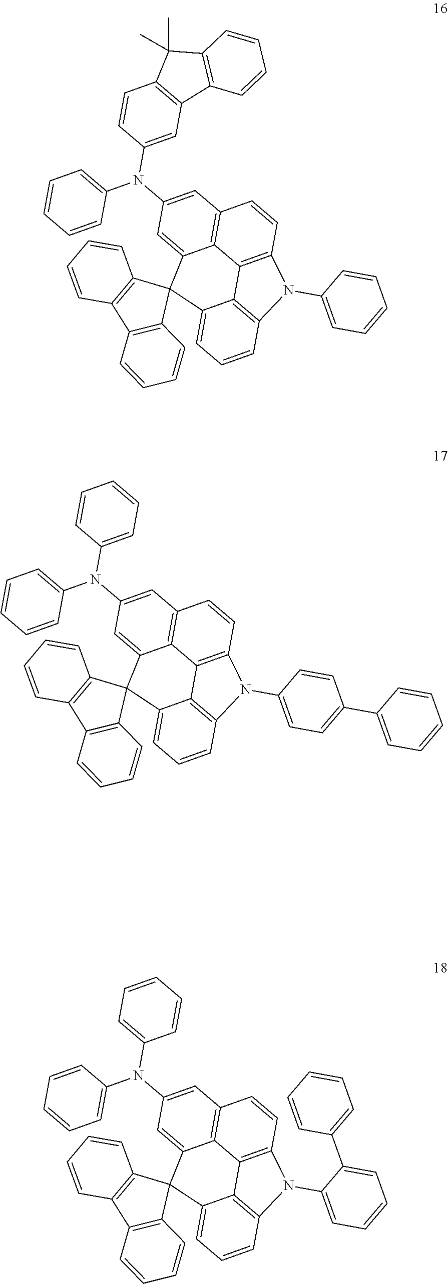 [SMILES: CC1(C)c2ccccc2-c2cc(N(c3ccccc3)c3cc4c5c(ccc6c5c5c(cccc5n6-c5ccccc5)C45c4ccccc4-c4ccccc45)c3)ccc21 ; c1ccc(-c2ccc(-n3c4cccc5c4c4c6c(cc(N(c7ccccc7)c7ccccc7)cc6ccc43)C53c4ccccc4-c4ccccc43)cc2)cc1 ; c1ccc(-c2ccccc2-n2c3cccc4c3c3c5c(cc(N(c6ccccc6)c6ccccc6)cc5ccc32)C42c3ccccc3-c3ccccc32)cc1]

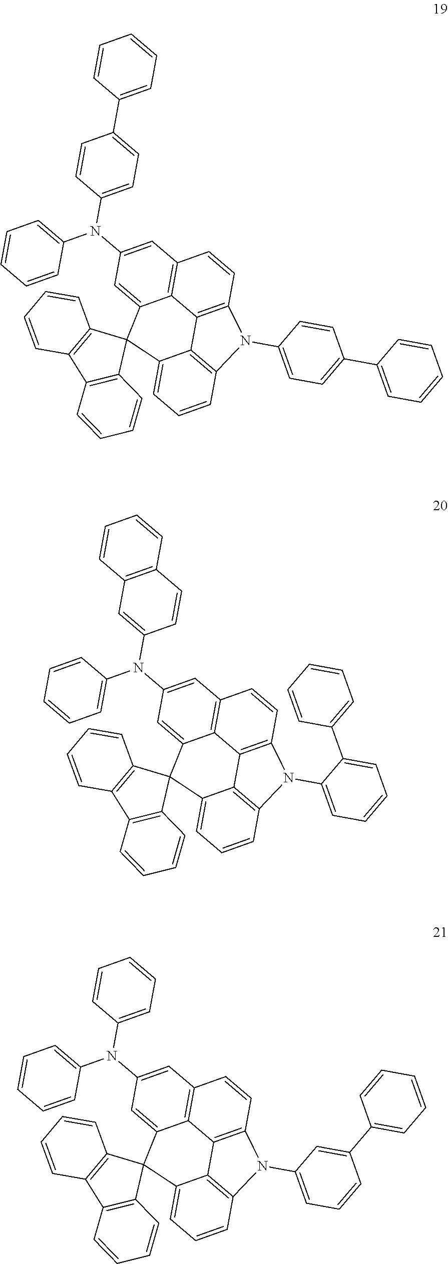 [SMILES: c1ccc(-c2ccc(N(c3ccccc3)c3cc4c5c(ccc6c5c5c(cccc5n6-c5ccc(-c6ccccc6)cc5)C45c4ccccc4-c4ccccc45)c3)cc2)cc1 ; c1ccc(-c2ccccc2-n2c3cccc4c3c3c5c(cc(N(c6ccccc6)c6ccc7ccccc7c6)cc5ccc32)C42c3ccccc3-c3ccccc32)cc1 ; c1ccc(-c2cccc(-n3c4cccc5c4c4c6c(cc(N(c7ccccc7)c7ccccc7)cc6ccc43)C53c4ccccc4-c4ccccc43)c2)cc1]

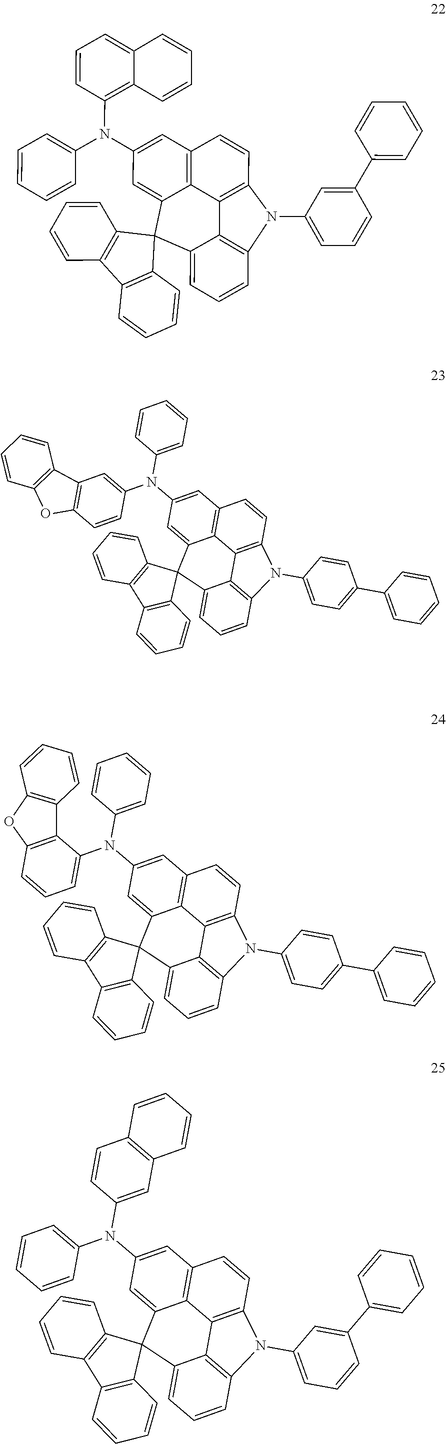 [SMILES: c1ccc(-c2cccc(-n3c4cccc5c4c4c6c(cc(N(c7ccccc7)c7cccc8ccccc78)cc6ccc43)C53c4ccccc4-c4ccccc43)c2)cc1 ; c1ccc(-c2ccc(-n3c4cccc5c4c4c6c(cc(N(c7ccccc7)c7ccc8oc9ccccc9c8c7)cc6ccc43)C53c4ccccc4-c4ccccc43)cc2)cc1 ; c1ccc(-c2ccc(-n3c4cccc5c4c4c6c(cc(N(c7ccccc7)c7cccc8oc9ccccc9c78)cc6ccc43)C53c4ccccc4-c4ccccc43)cc2)cc1 ; c1ccc(-c2cccc(-n3c4cccc5c4c4c6c(cc(N(c7ccccc7)c7ccc8ccccc8c7)cc6ccc43)C53c4ccccc4-c4ccccc43)c2)cc1]

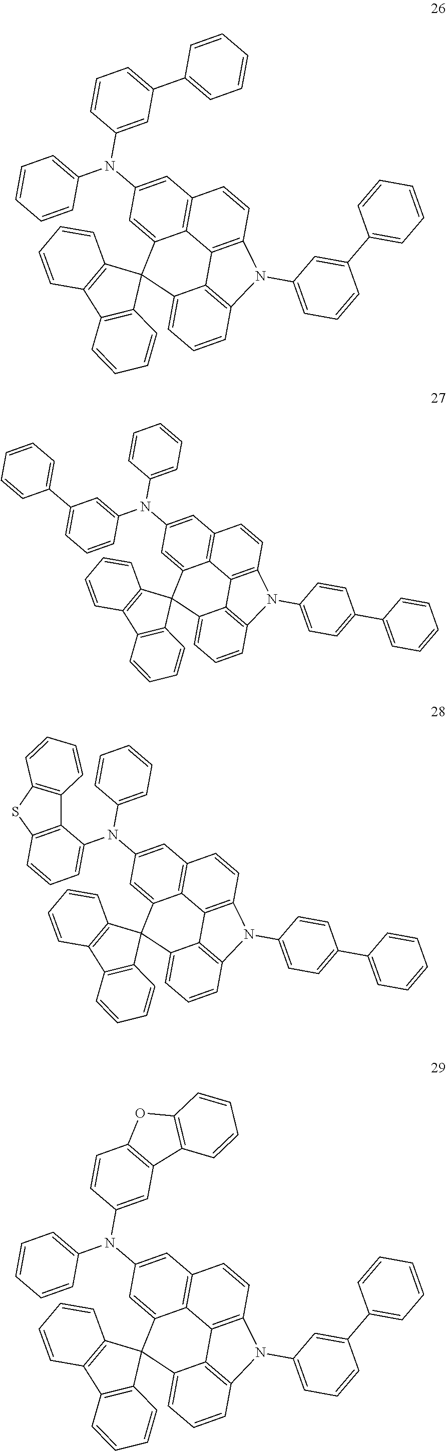 [SMILES: c1ccc(-c2cccc(N(c3ccccc3)c3cc4c5c(ccc6c5c5c(cccc5n6-c5cccc(-c6ccccc6)c5)C45c4ccccc4-c4ccccc45)c3)c2)cc1 ; c1ccc(-c2ccc(-n3c4cccc5c4c4c6c(cc(N(c7ccccc7)c7cccc(-c8ccccc8)c7)cc6ccc43)C53c4ccccc4-c4ccccc43)cc2)cc1 ; c1ccc(-c2ccc(-n3c4cccc5c4c4c6c(cc(N(c7ccccc7)c7cccc8sc9ccccc9c78)cc6ccc43)C53c4ccccc4-c4ccccc43)cc2)cc1 ; c1ccc(-c2cccc(-n3c4cccc5c4c4c6c(cc(N(c7ccccc7)c7ccc8oc9ccccc9c8c7)cc6ccc43)C53c4ccccc4-c4ccccc43)c2)cc1]

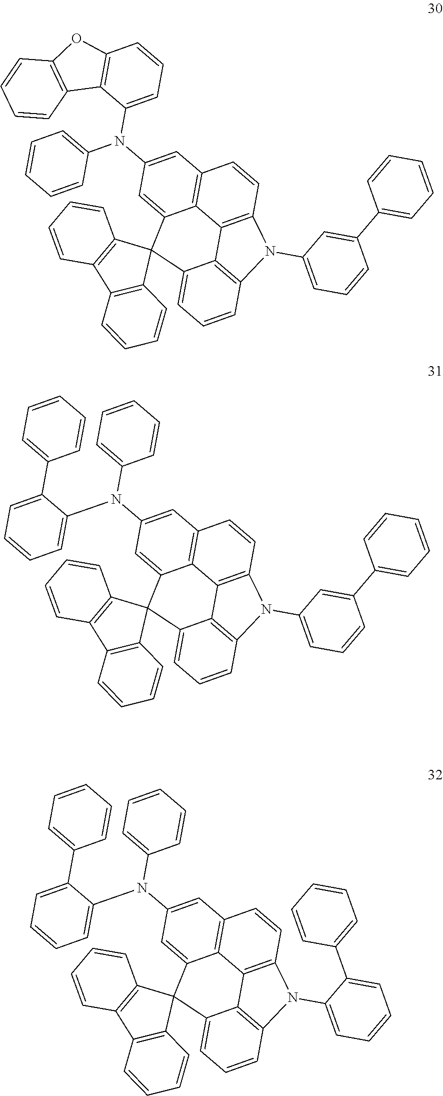 [SMILES: c1ccc(-c2cccc(-n3c4cccc5c4c4c6c(cc(N(c7ccccc7)c7cccc8oc9ccccc9c78)cc6ccc43)C53c4ccccc4-c4ccccc43)c2)cc1 ; c1ccc(-c2cccc(-n3c4cccc5c4c4c6c(cc(N(c7ccccc7)c7ccccc7-c7ccccc7)cc6ccc43)C53c4ccccc4-c4ccccc43)c2)cc1 ; c1ccc(-c2ccccc2N(c2ccccc2)c2cc3c4c(ccc5c4c4c(cccc4n5-c4ccccc4-c4ccccc4)C34c3ccccc3-c3ccccc34)c2)cc1]

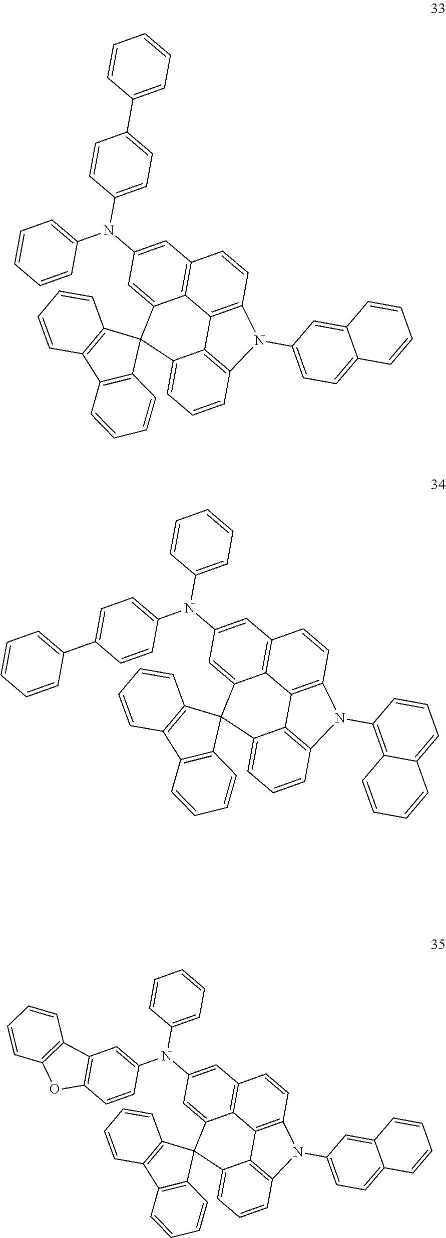 [SMILES: c1ccc(-c2ccc(N(c3ccccc3)c3cc4c5c(ccc6c5c5c(cccc5n6-c5ccc6ccccc6c5)C45c4ccccc4-c4ccccc45)c3)cc2)cc1 ; c1ccc(-c2ccc(N(c3ccccc3)c3cc4c5c(ccc6c5c5c(cccc5n6-c5cccc6ccccc56)C45c4ccccc4-c4ccccc45)c3)cc2)cc1 ; c1ccc(N(c2cc3c4c(ccc5c4c4c(cccc4n5-c4ccc5ccccc5c4)C34c3ccccc3-c3ccccc34)c2)c2ccc3oc4ccccc4c3c2)cc1]

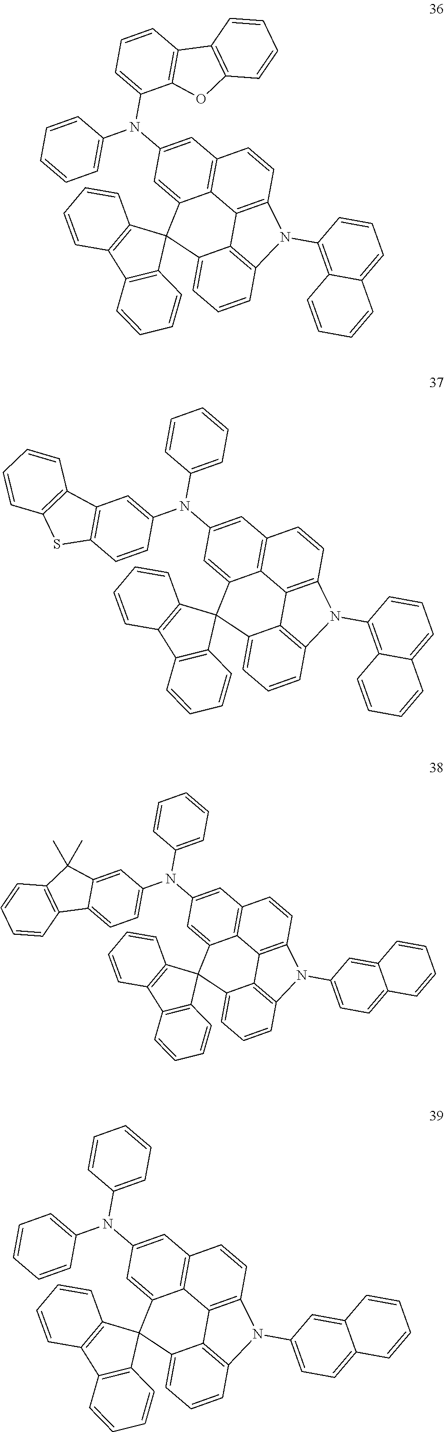 [SMILES: c1ccc(N(c2cc3c4c(ccc5c4c4c(cccc4n5-c4cccc5ccccc45)C34c3ccccc3-c3ccccc34)c2)c2cccc3c2oc2ccccc23)cc1 ; c1ccc(N(c2cc3c4c(ccc5c4c4c(cccc4n5-c4cccc5ccccc45)C34c3ccccc3-c3ccccc34)c2)c2ccc3sc4ccccc4c3c2)cc1 ; CC1(C)c2ccccc2-c2ccc(N(c3ccccc3)c3cc4c5c(ccc6c5c5c(cccc5n6-c5ccc6ccccc6c5)C45c4ccccc4-c4ccccc45)c3)cc21 ; c1ccc(N(c2ccccc2)c2cc3c4c(ccc5c4c4c(cccc4n5-c4ccc5ccccc5c4)C34c3ccccc3-c3ccccc34)c2)cc1]

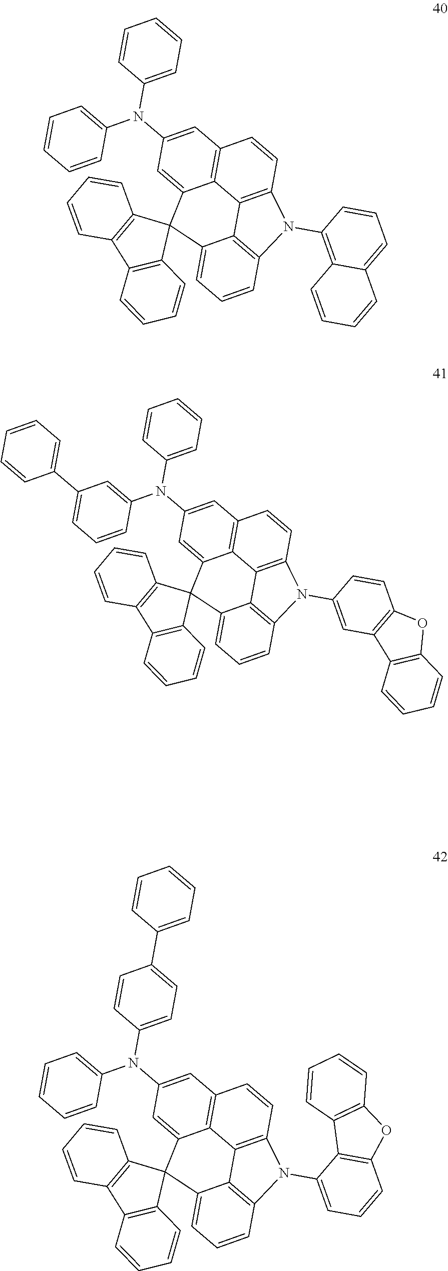 [SMILES: c1ccc(N(c2ccccc2)c2cc3c4c(ccc5c4c4c(cccc4n5-c4cccc5ccccc45)C34c3ccccc3-c3ccccc34)c2)cc1 ; c1ccc(-c2cccc(N(c3ccccc3)c3cc4c5c(ccc6c5c5c(cccc5n6-c5ccc6oc7ccccc7c6c5)C45c4ccccc4-c4ccccc45)c3)c2)cc1 ; c1ccc(-c2ccc(N(c3ccccc3)c3cc4c5c(ccc6c5c5c(cccc5n6-c5cccc6oc7ccccc7c56)C45c4ccccc4-c4ccccc45)c3)cc2)cc1]

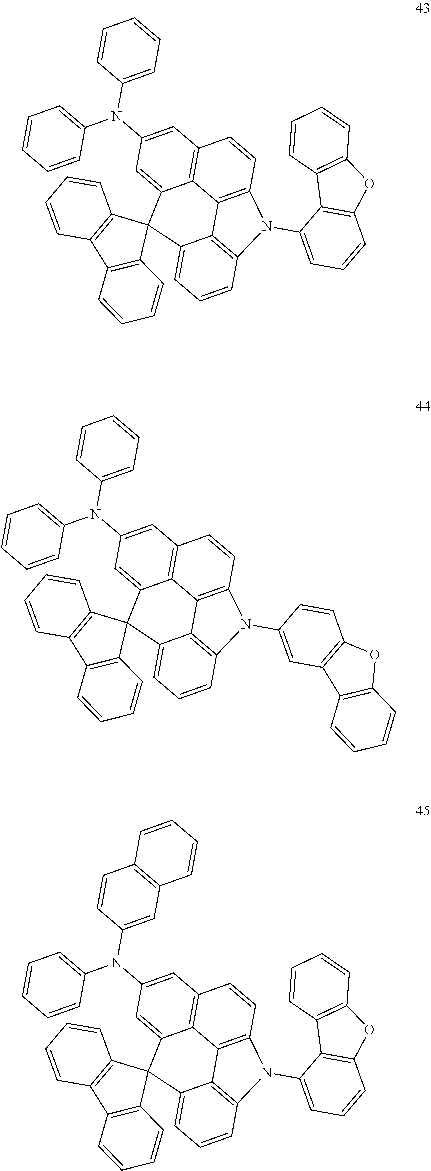 [SMILES: c1ccc(N(c2ccccc2)c2cc3c4c(ccc5c4c4c(cccc4n5-c4cccc5oc6ccccc6c45)C34c3ccccc3-c3ccccc34)c2)cc1 ; c1ccc(N(c2ccccc2)c2cc3c4c(ccc5c4c4c(cccc4n5-c4ccc5oc6ccccc6c5c4)C34c3ccccc3-c3ccccc34)c2)cc1 ; c1ccc(N(c2ccc3ccccc3c2)c2cc3c4c(ccc5c4c4c(cccc4n5-c4cccc5oc6ccccc6c45)C34c3ccccc3-c3ccccc34)c2)cc1]

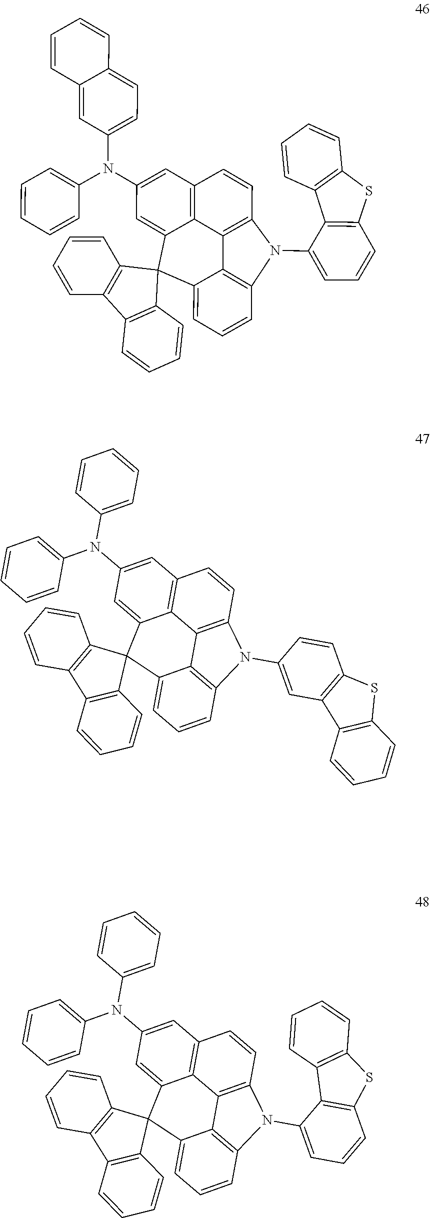 [SMILES: c1ccc(N(c2ccc3ccccc3c2)c2cc3c4c(ccc5c4c4c(cccc4n5-c4cccc5sc6ccccc6c45)C34c3ccccc3-c3ccccc34)c2)cc1 ; c1ccc(N(c2ccccc2)c2cc3c4c(ccc5c4c4c(cccc4n5-c4ccc5sc6ccccc6c5c4)C34c3ccccc3-c3ccccc34)c2)cc1 ; c1ccc(N(c2ccccc2)c2cc3c4c(ccc5c4c4c(cccc4n5-c4cccc5sc6ccccc6c45)C34c3ccccc3-c3ccccc34)c2)cc1]

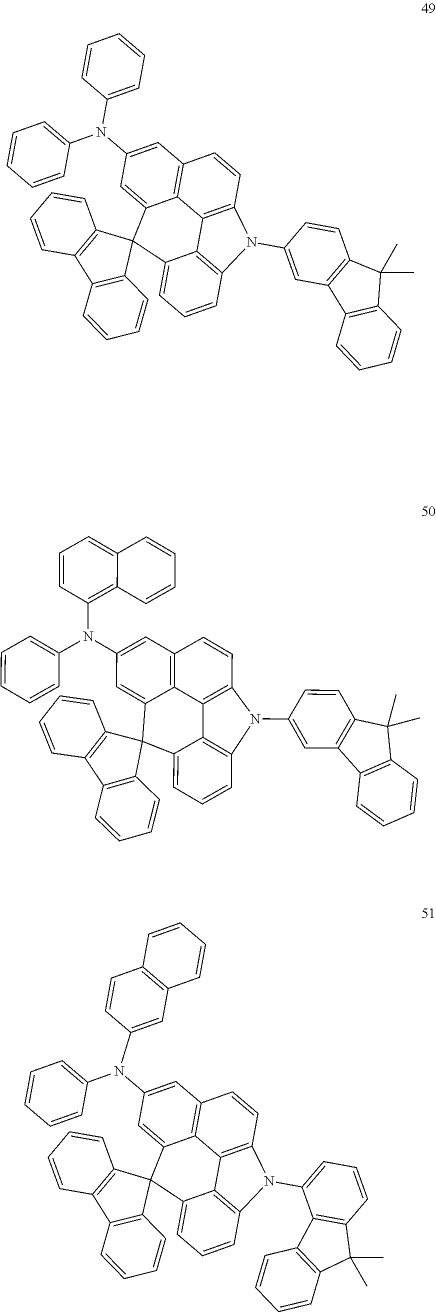 [SMILES: CC1(C)c2ccccc2-c2cc(-n3c4cccc5c4c4c6c(cc(N(c7ccccc7)c7ccccc7)cc6ccc43)C53c4ccccc4-c4ccccc43)ccc21 ; CC1(C)c2ccccc2-c2cc(-n3c4cccc5c4c4c6c(cc(N(c7ccccc7)c7cccc8ccccc78)cc6ccc43)C53c4ccccc4-c4ccccc43)ccc21 ; CC1(C)c2ccccc2-c2c(-n3c4cccc5c4c4c6c(cc(N(c7ccccc7)c7ccc8ccccc8c7)cc6ccc43)C53c4ccccc4-c4ccccc43)cccc21]

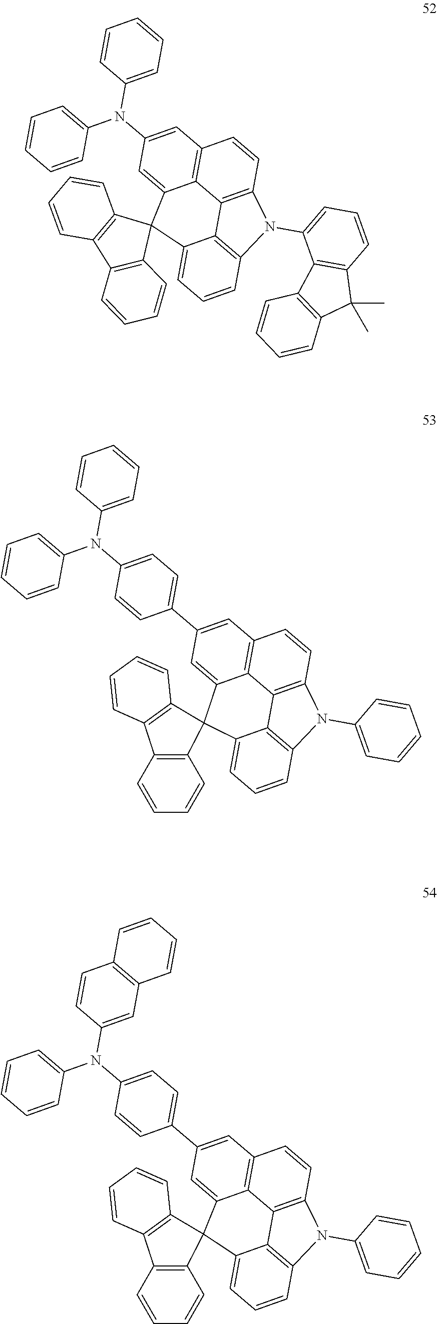 [SMILES: CC1(C)c2ccccc2-c2c(-n3c4cccc5c4c4c6c(cc(N(c7ccccc7)c7ccccc7)cc6ccc43)C53c4ccccc4-c4ccccc43)cccc21 ; c1ccc(N(c2ccccc2)c2ccc(-c3cc4c5c(ccc6c5c5c(cccc5n6-c5ccccc5)C45c4ccccc4-c4ccccc45)c3)cc2)cc1 ; c1ccc(N(c2ccc(-c3cc4c5c(ccc6c5c5c(cccc5n6-c5ccccc5)C45c4ccccc4-c4ccccc45)c3)cc2)c2ccc3ccccc3c2)cc1]

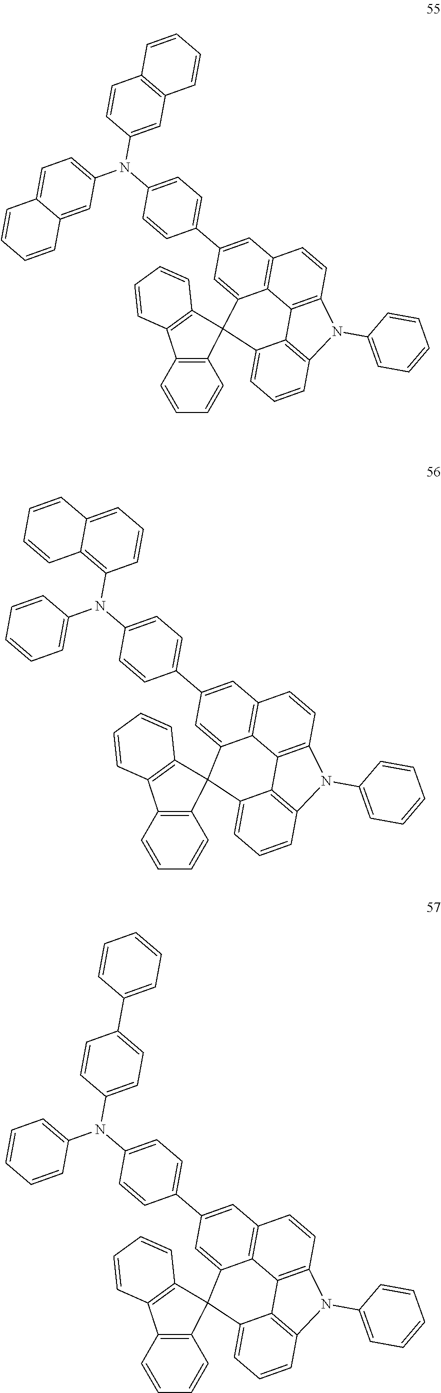 [SMILES: c1ccc(-n2c3cccc4c3c3c5c(cc(-c6ccc(N(c7ccc8ccccc8c7)c7ccc8ccccc8c7)cc6)cc5ccc32)C42c3ccccc3-c3ccccc32)cc1 ; c1ccc(N(c2ccc(-c3cc4c5c(ccc6c5c5c(cccc5n6-c5ccccc5)C45c4ccccc4-c4ccccc45)c3)cc2)c2cccc3ccccc23)cc1 ; c1ccc(-c2ccc(N(c3ccccc3)c3ccc(-c4cc5c6c(ccc7c6c6c(cccc6n7-c6ccccc6)C56c5ccccc5-c5ccccc56)c4)cc3)cc2)cc1]

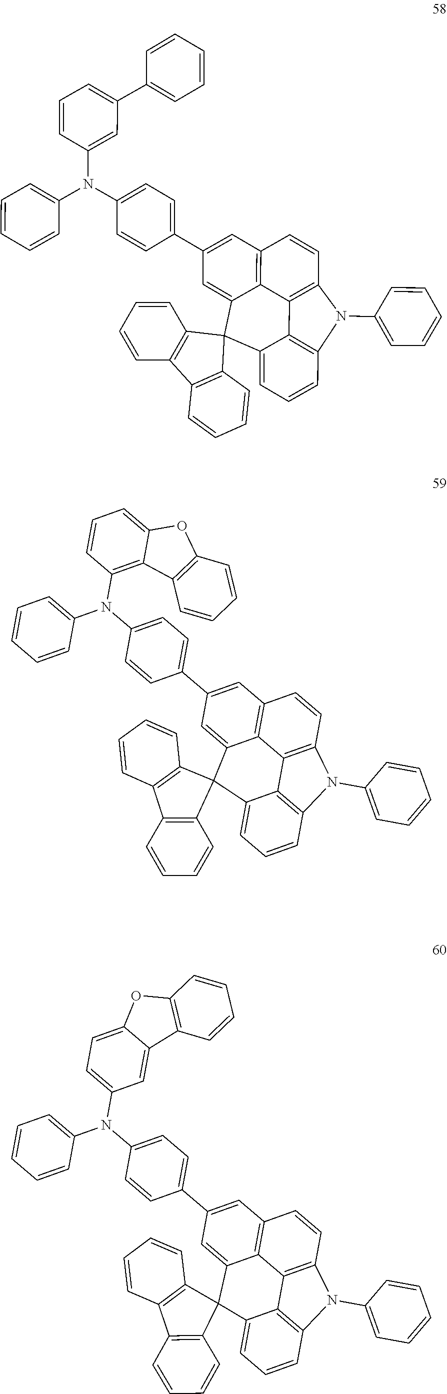 [SMILES: c1ccc(-c2cccc(N(c3ccccc3)c3ccc(-c4cc5c6c(ccc7c6c6c(cccc6n7-c6ccccc6)C56c5ccccc5-c5ccccc56)c4)cc3)c2)cc1 ; c1ccc(N(c2ccc(-c3cc4c5c(ccc6c5c5c(cccc5n6-c5ccccc5)C45c4ccccc4-c4ccccc45)c3)cc2)c2cccc3oc4ccccc4c23)cc1 ; c1ccc(N(c2ccc(-c3cc4c5c(ccc6c5c5c(cccc5n6-c5ccccc5)C45c4ccccc4-c4ccccc45)c3)cc2)c2ccc3oc4ccccc4c3c2)cc1]

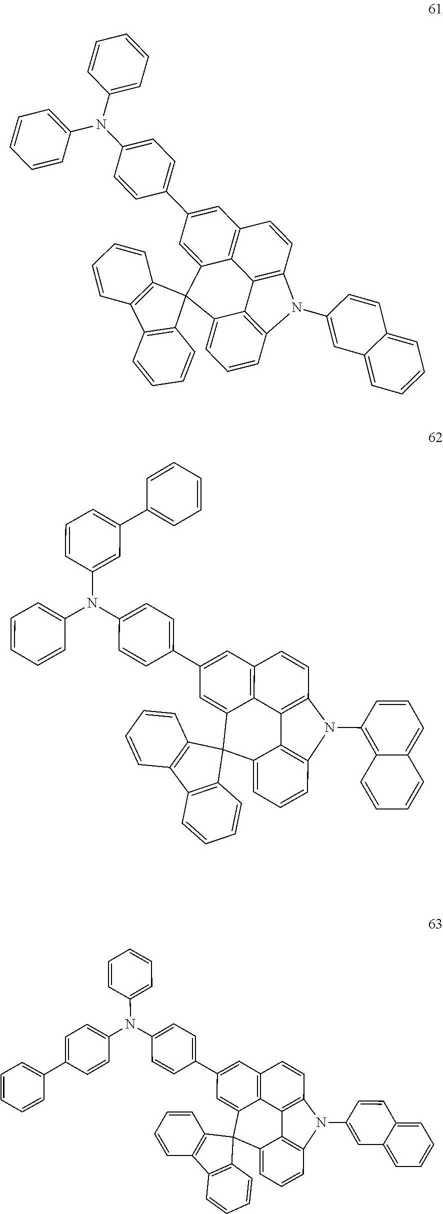 [SMILES: c1ccc(N(c2ccccc2)c2ccc(-c3cc4c5c(ccc6c5c5c(cccc5n6-c5ccc6ccccc6c5)C45c4ccccc4-c4ccccc45)c3)cc2)cc1 ; c1ccc(-c2cccc(N(c3ccccc3)c3ccc(-c4cc5c6c(ccc7c6c6c(cccc6n7-c6cccc7ccccc67)C56c5ccccc5-c5ccccc56)c4)cc3)c2)cc1 ; c1ccc(-c2ccc(N(c3ccccc3)c3ccc(-c4cc5c6c(ccc7c6c6c(cccc6n7-c6ccc7ccccc7c6)C56c5ccccc5-c5ccccc56)c4)cc3)cc2)cc1]

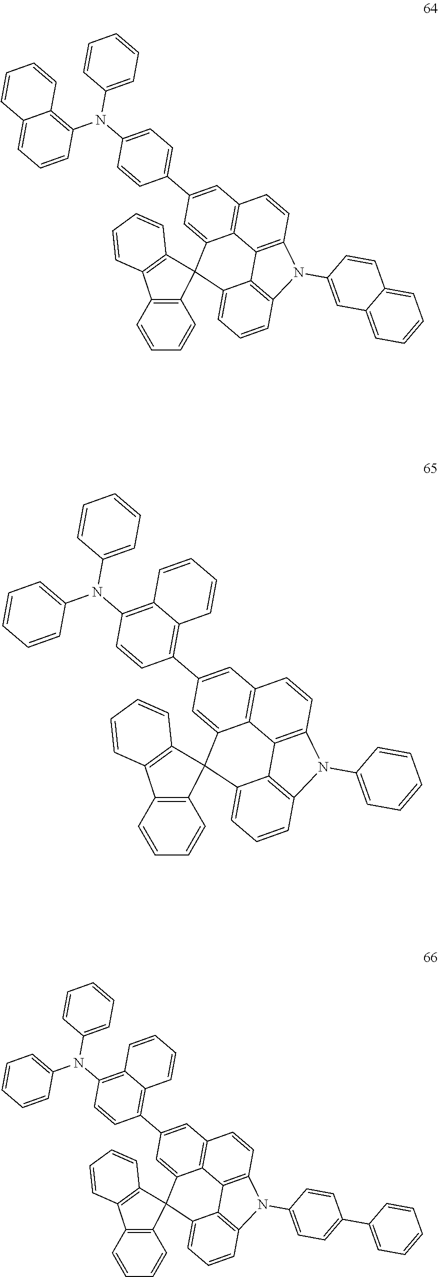 [SMILES: c1ccc(N(c2ccc(-c3cc4c5c(ccc6c5c5c(cccc5n6-c5ccc6ccccc6c5)C45c4ccccc4-c4ccccc45)c3)cc2)c2cccc3ccccc23)cc1 ; c1ccc(N(c2ccccc2)c2ccc(-c3cc4c5c(ccc6c5c5c(cccc5n6-c5ccccc5)C45c4ccccc4-c4ccccc45)c3)c3ccccc23)cc1 ; c1ccc(-c2ccc(-n3c4cccc5c4c4c6c(cc(-c7ccc(N(c8ccccc8)c8ccccc8)c8ccccc78)cc6ccc43)C53c4ccccc4-c4ccccc43)cc2)cc1]

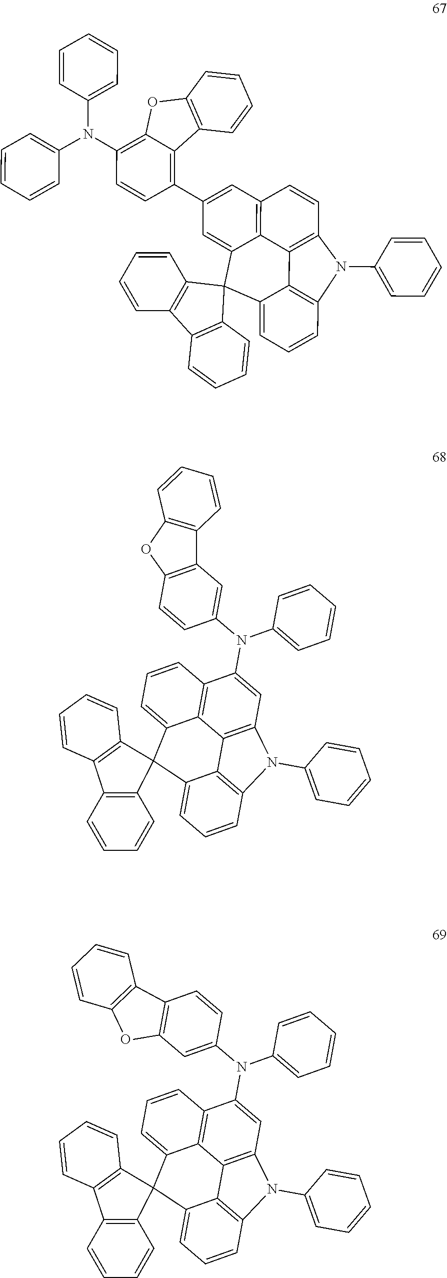 [SMILES: c1ccc(N(c2ccccc2)c2ccc(-c3cc4c5c(ccc6c5c5c(cccc5n6-c5ccccc5)C45c4ccccc4-c4ccccc45)c3)c3c2oc2ccccc23)cc1 ; c1ccc(N(c2ccc3oc4ccccc4c3c2)c2cc3c4c5c(cccc25)C2(c5ccccc5-c5ccccc52)c2cccc(c24)n3-c2ccccc2)cc1 ; c1ccc(N(c2ccc3c(c2)oc2ccccc23)c2cc3c4c5c(cccc25)C2(c5ccccc5-c5ccccc52)c2cccc(c24)n3-c2ccccc2)cc1]

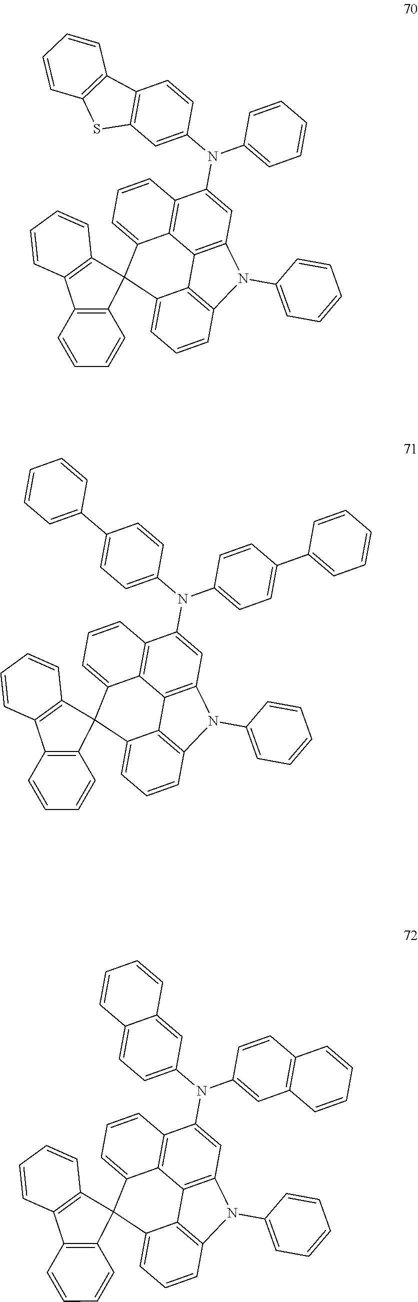 [SMILES: c1ccc(N(c2ccc3c(c2)sc2ccccc23)c2cc3c4c5c(cccc25)C2(c5ccccc5-c5ccccc52)c2cccc(c24)n3-c2ccccc2)cc1 ; c1ccc(-c2ccc(N(c3ccc(-c4ccccc4)cc3)c3cc4c5c6c(cccc36)C3(c6ccccc6-c6ccccc63)c3cccc(c35)n4-c3ccccc3)cc2)cc1 ; c1ccc(-n2c3cccc4c3c3c5c(cccc5c(N(c5ccc6ccccc6c5)c5ccc6ccccc6c5)cc32)C42c3ccccc3-c3ccccc32)cc1]

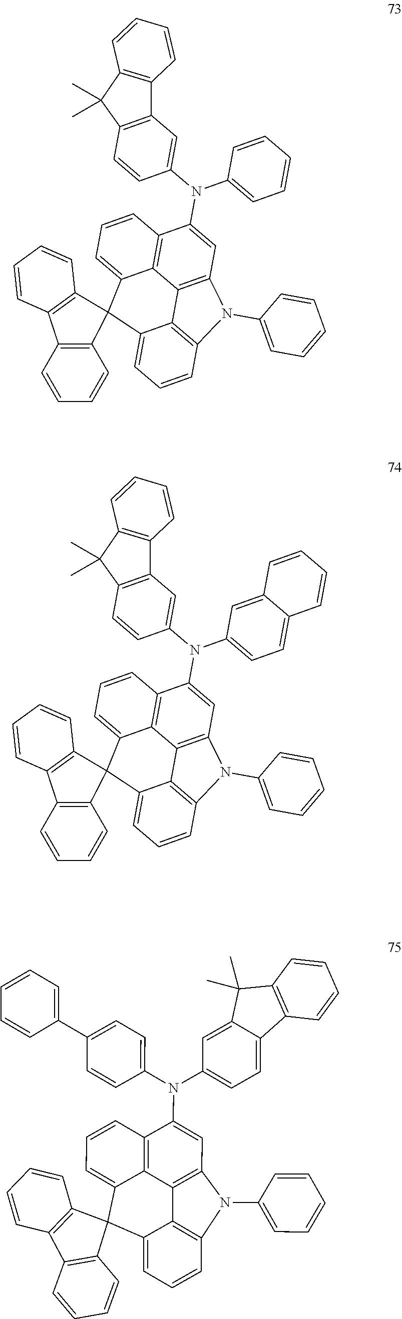 [SMILES: CC1(C)c2ccccc2-c2cc(N(c3ccccc3)c3cc4c5c6c(cccc36)C3(c6ccccc6-c6ccccc63)c3cccc(c35)n4-c3ccccc3)ccc21 ; CC1(C)c2ccccc2-c2cc(N(c3ccc4ccccc4c3)c3cc4c5c6c(cccc36)C3(c6ccccc6-c6ccccc63)c3cccc(c35)n4-c3ccccc3)ccc21 ; CC1(C)c2ccccc2-c2ccc(N(c3ccc(-c4ccccc4)cc3)c3cc4c5c6c(cccc36)C3(c6ccccc6-c6ccccc63)c3cccc(c35)n4-c3ccccc3)cc21]

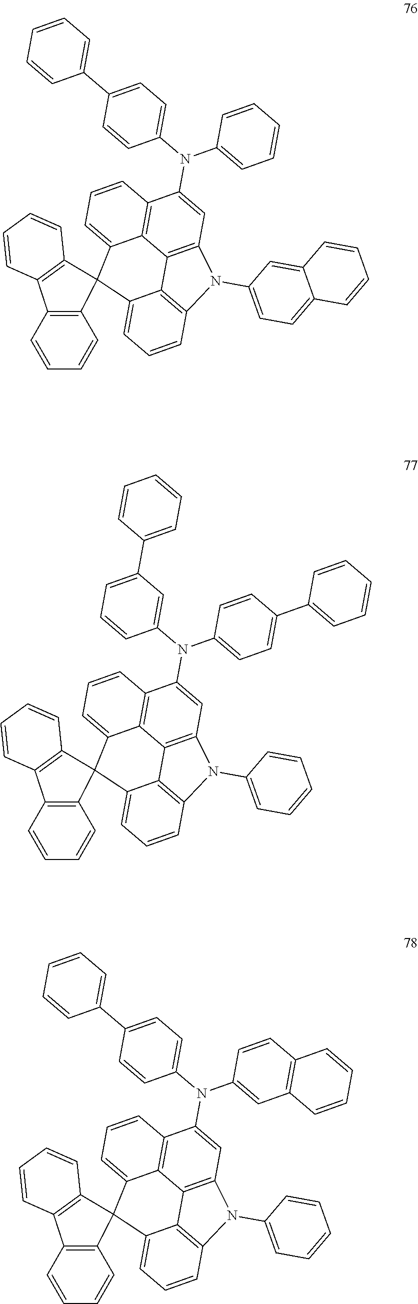 [SMILES: c1ccc(-c2ccc(N(c3ccccc3)c3cc4c5c6c(cccc36)C3(c6ccccc6-c6ccccc63)c3cccc(c35)n4-c3ccc4ccccc4c3)cc2)cc1 ; c1ccc(-c2ccc(N(c3cccc(-c4ccccc4)c3)c3cc4c5c6c(cccc36)C3(c6ccccc6-c6ccccc63)c3cccc(c35)n4-c3ccccc3)cc2)cc1 ; c1ccc(-c2ccc(N(c3ccc4ccccc4c3)c3cc4c5c6c(cccc36)C3(c6ccccc6-c6ccccc63)c3cccc(c35)n4-c3ccccc3)cc2)cc1]

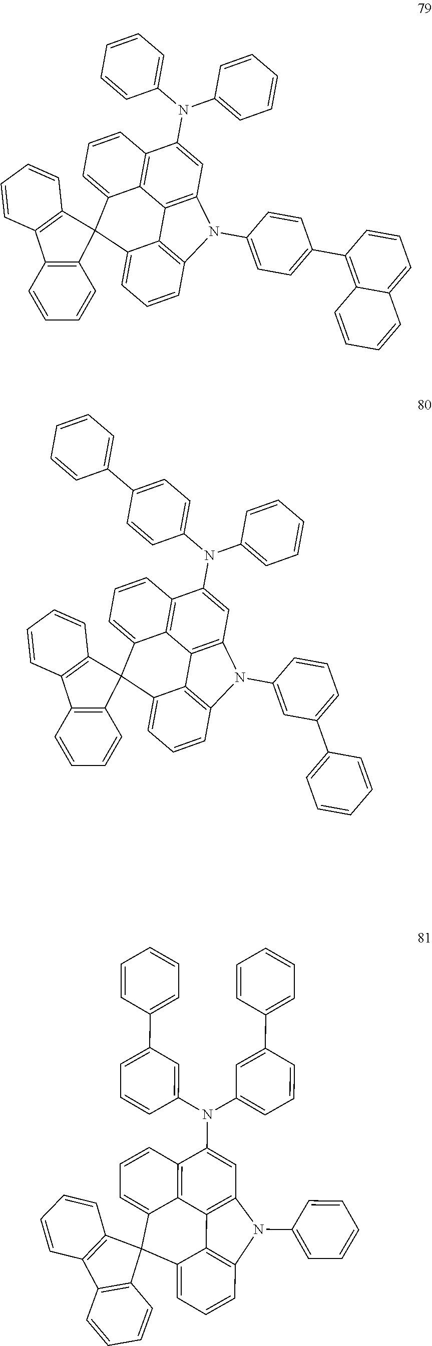 [SMILES: c1ccc(N(c2ccccc2)c2cc3c4c5c(cccc25)C2(c5ccccc5-c5ccccc52)c2cccc(c24)n3-c2ccc(-c3cccc4ccccc34)cc2)cc1 ; c1ccc(-c2ccc(N(c3ccccc3)c3cc4c5c6c(cccc36)C3(c6ccccc6-c6ccccc63)c3cccc(c35)n4-c3cccc(-c4ccccc4)c3)cc2)cc1 ; c1ccc(-c2cccc(N(c3cccc(-c4ccccc4)c3)c3cc4c5c6c(cccc36)C3(c6ccccc6-c6ccccc63)c3cccc(c35)n4-c3ccccc3)c2)cc1]

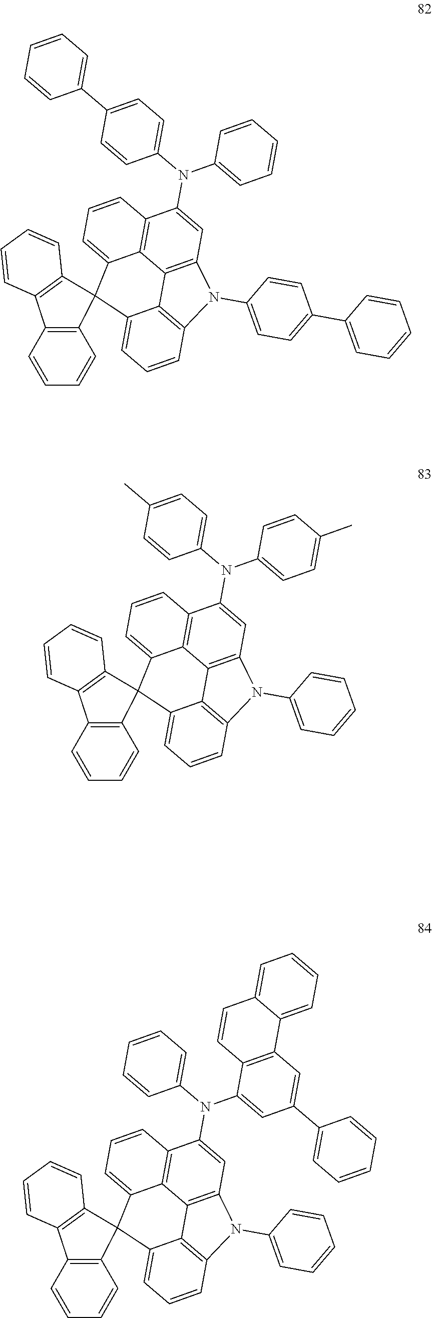 [SMILES: c1ccc(-c2ccc(N(c3ccccc3)c3cc4c5c6c(cccc36)C3(c6ccccc6-c6ccccc63)c3cccc(c35)n4-c3ccc(-c4ccccc4)cc3)cc2)cc1 ; Cc1ccc(N(c2ccc(C)cc2)c2cc3c4c5c(cccc25)C2(c5ccccc5-c5ccccc52)c2cccc(c24)n3-c2ccccc2)cc1 ; c1ccc(-c2cc(N(c3ccccc3)c3cc4c5c6c(cccc36)C3(c6ccccc6-c6ccccc63)c3cccc(c35)n4-c3ccccc3)c3ccc4ccccc4c3c2)cc1]

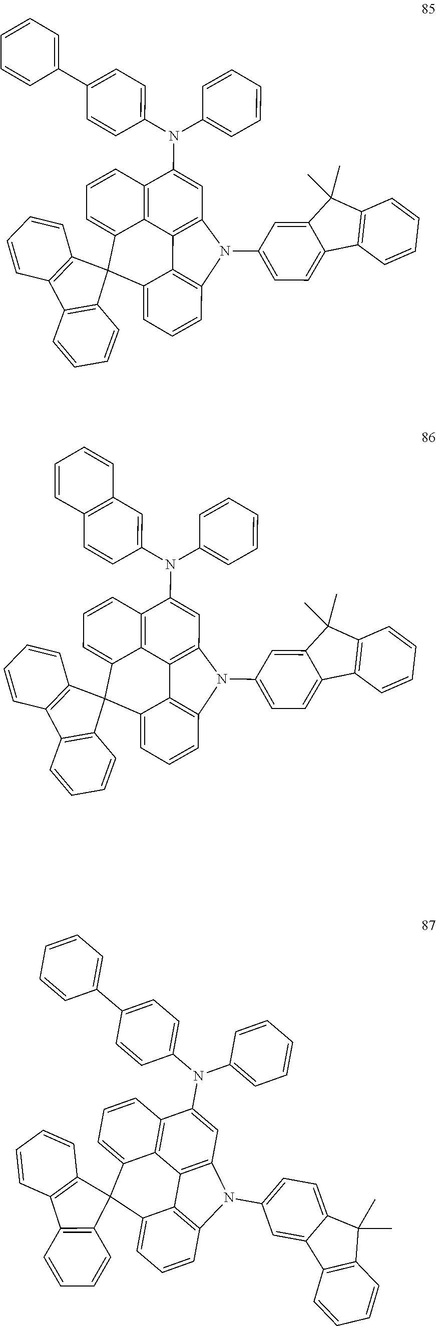 [SMILES: CC1(C)c2ccccc2-c2ccc(-n3c4cccc5c4c4c6c(cccc6c(N(c6ccccc6)c6ccc(-c7ccccc7)cc6)cc43)C53c4ccccc4-c4ccccc43)cc21 ; CC1(C)c2ccccc2-c2ccc(-n3c4cccc5c4c4c6c(cccc6c(N(c6ccccc6)c6ccc7ccccc7c6)cc43)C53c4ccccc4-c4ccccc43)cc21 ; CC1(C)c2ccccc2-c2cc(-n3c4cccc5c4c4c6c(cccc6c(N(c6ccccc6)c6ccc(-c7ccccc7)cc6)cc43)C53c4ccccc4-c4ccccc43)ccc21]

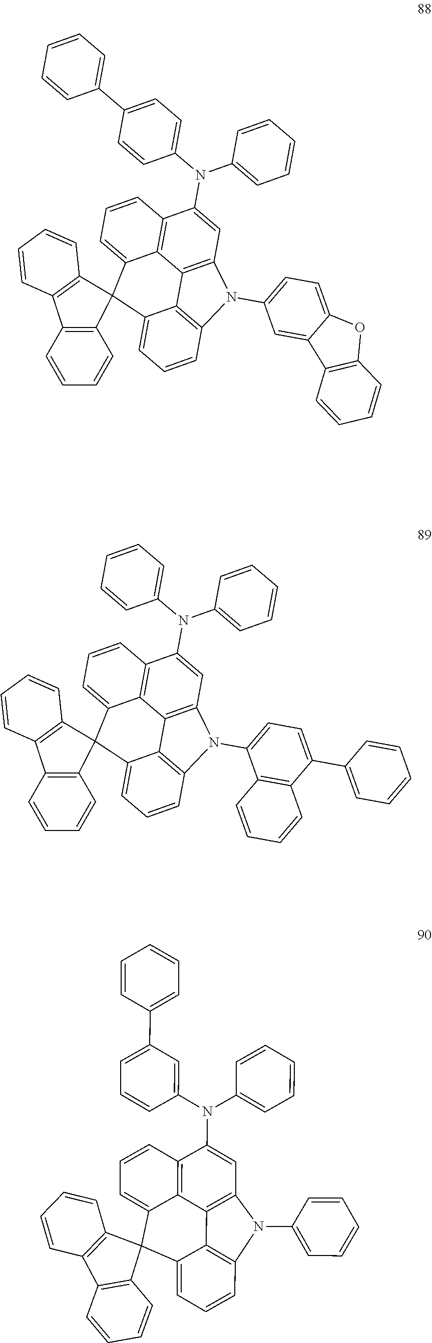 [SMILES: c1ccc(-c2ccc(N(c3ccccc3)c3cc4c5c6c(cccc36)C3(c6ccccc6-c6ccccc63)c3cccc(c35)n4-c3ccc4oc5ccccc5c4c3)cc2)cc1 ; c1ccc(-c2ccc(-n3c4cccc5c4c4c6c(cccc6c(N(c6ccccc6)c6ccccc6)cc43)C53c4ccccc4-c4ccccc43)c3ccccc23)cc1 ; c1ccc(-c2cccc(N(c3ccccc3)c3cc4c5c6c(cccc36)C3(c6ccccc6-c6ccccc63)c3cccc(c35)n4-c3ccccc3)c2)cc1]

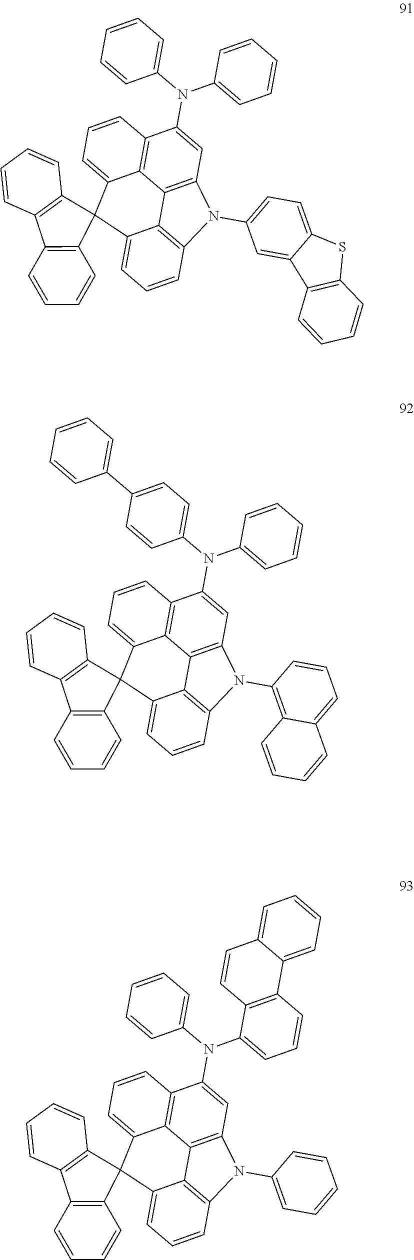 [SMILES: c1ccc(N(c2ccccc2)c2cc3c4c5c(cccc25)C2(c5ccccc5-c5ccccc52)c2cccc(c24)n3-c2ccc3sc4ccccc4c3c2)cc1 ; c1ccc(-c2ccc(N(c3ccccc3)c3cc4c5c6c(cccc36)C3(c6ccccc6-c6ccccc63)c3cccc(c35)n4-c3cccc4ccccc34)cc2)cc1 ; c1ccc(N(c2cccc3c2ccc2ccccc23)c2cc3c4c5c(cccc25)C2(c5ccccc5-c5ccccc52)c2cccc(c24)n3-c2ccccc2)cc1]

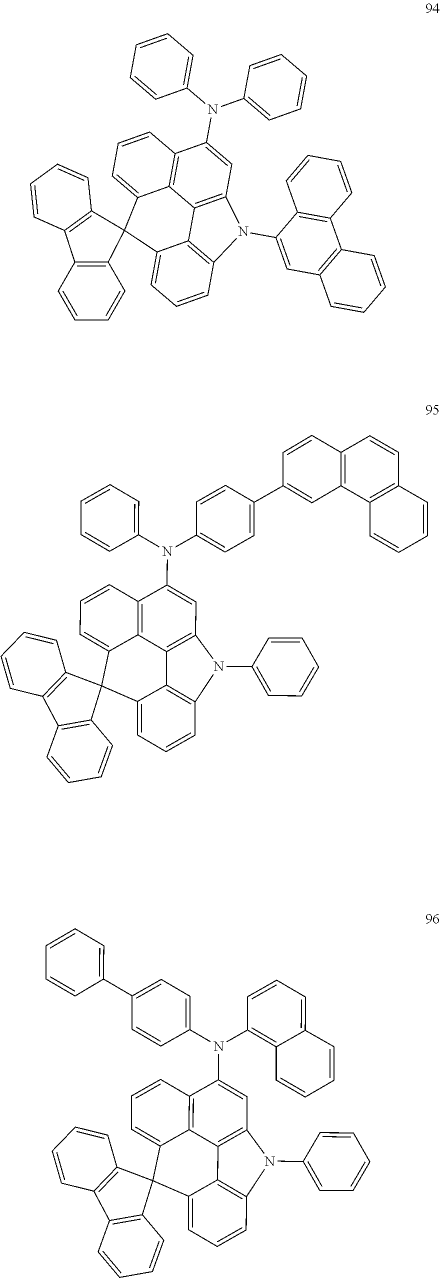 [SMILES: c1ccc(N(c2ccccc2)c2cc3c4c5c(cccc25)C2(c5ccccc5-c5ccccc52)c2cccc(c24)n3-c2cc3ccccc3c3ccccc23)cc1 ; c1ccc(N(c2ccc(-c3ccc4ccc5ccccc5c4c3)cc2)c2cc3c4c5c(cccc25)C2(c5ccccc5-c5ccccc52)c2cccc(c24)n3-c2ccccc2)cc1 ; c1ccc(-c2ccc(N(c3cccc4ccccc34)c3cc4c5c6c(cccc36)C3(c6ccccc6-c6ccccc63)c3cccc(c35)n4-c3ccccc3)cc2)cc1]

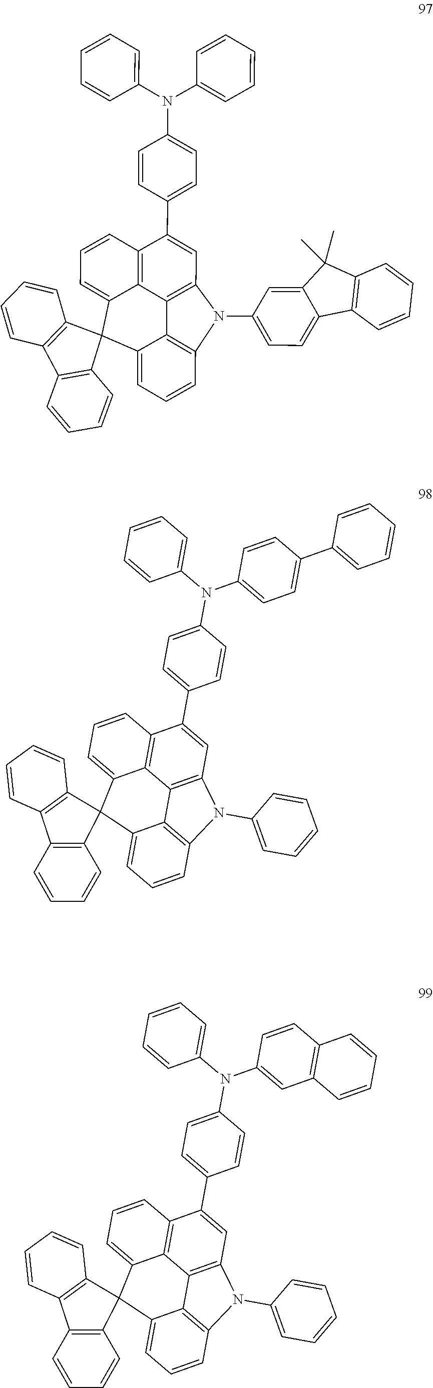 [SMILES: CC1(C)c2ccccc2-c2ccc(-n3c4cccc5c4c4c6c(cccc6c(-c6ccc(N(c7ccccc7)c7ccccc7)cc6)cc43)C53c4ccccc4-c4ccccc43)cc21 ; c1ccc(-c2ccc(N(c3ccccc3)c3ccc(-c4cc5c6c7c(cccc47)C4(c7ccccc7-c7ccccc74)c4cccc(c46)n5-c4ccccc4)cc3)cc2)cc1 ; c1ccc(N(c2ccc(-c3cc4c5c6c(cccc36)C3(c6ccccc6-c6ccccc63)c3cccc(c35)n4-c3ccccc3)cc2)c2ccc3ccccc3c2)cc1]

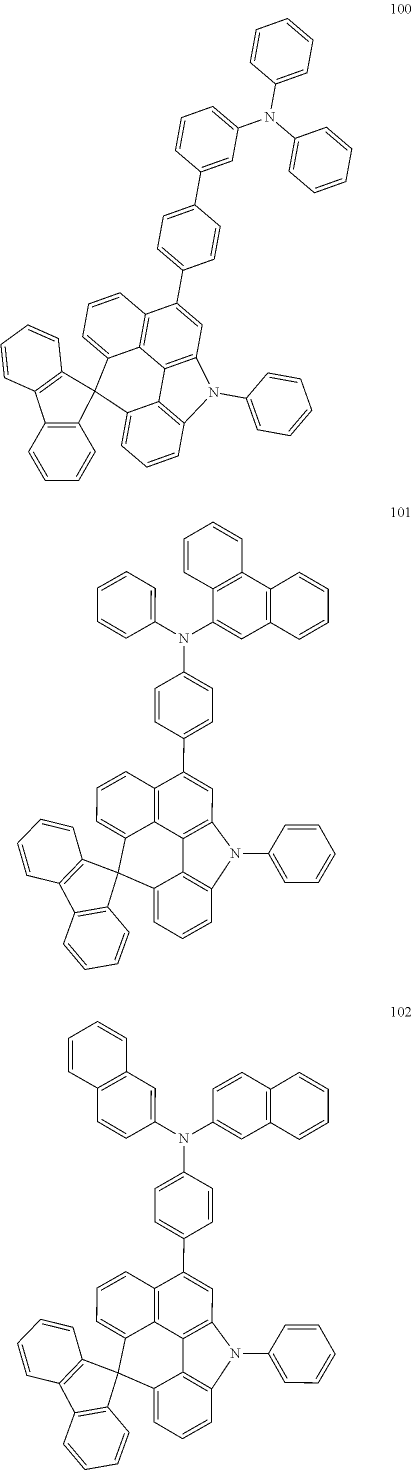 [SMILES: c1ccc(N(c2ccccc2)c2cccc(-c3ccc(-c4cc5c6c7c(cccc47)C4(c7ccccc7-c7ccccc74)c4cccc(c46)n5-c4ccccc4)cc3)c2)cc1 ; c1ccc(N(c2ccc(-c3cc4c5c6c(cccc36)C3(c6ccccc6-c6ccccc63)c3cccc(c35)n4-c3ccccc3)cc2)c2cc3ccccc3c3ccccc23)cc1 ; c1ccc(-n2c3cccc4c3c3c5c(cccc5c(-c5ccc(N(c6ccc7ccccc7c6)c6ccc7ccccc7c6)cc5)cc32)C42c3ccccc3-c3ccccc32)cc1]

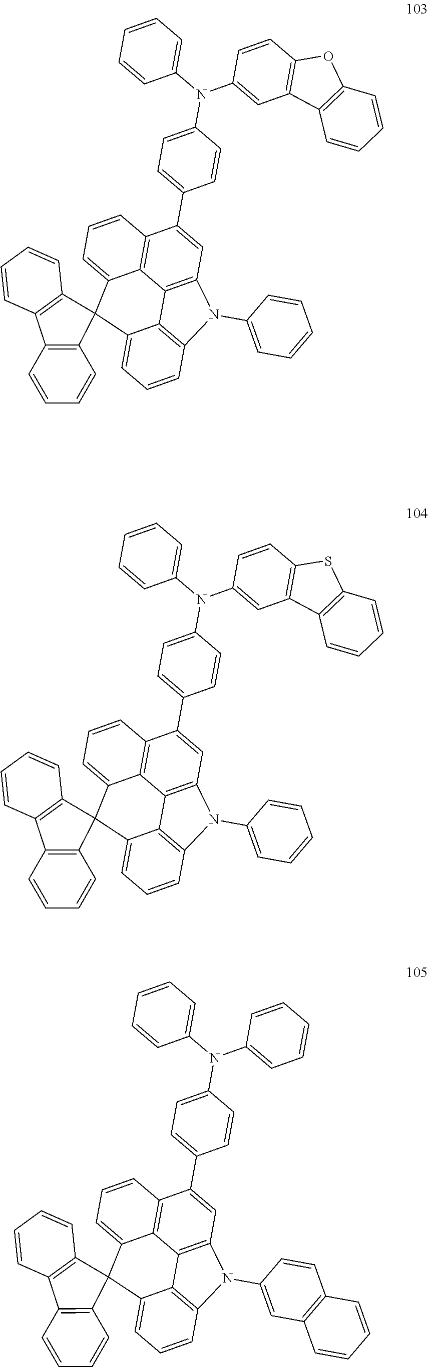 [SMILES: c1ccc(N(c2ccc(-c3cc4c5c6c(cccc36)C3(c6ccccc6-c6ccccc63)c3cccc(c35)n4-c3ccccc3)cc2)c2ccc3oc4ccccc4c3c2)cc1 ; c1ccc(N(c2ccc(-c3cc4c5c6c(cccc36)C3(c6ccccc6-c6ccccc63)c3cccc(c35)n4-c3ccccc3)cc2)c2ccc3sc4ccccc4c3c2)cc1 ; c1ccc(N(c2ccccc2)c2ccc(-c3cc4c5c6c(cccc36)C3(c6ccccc6-c6ccccc63)c3cccc(c35)n4-c3ccc4ccccc4c3)cc2)cc1]

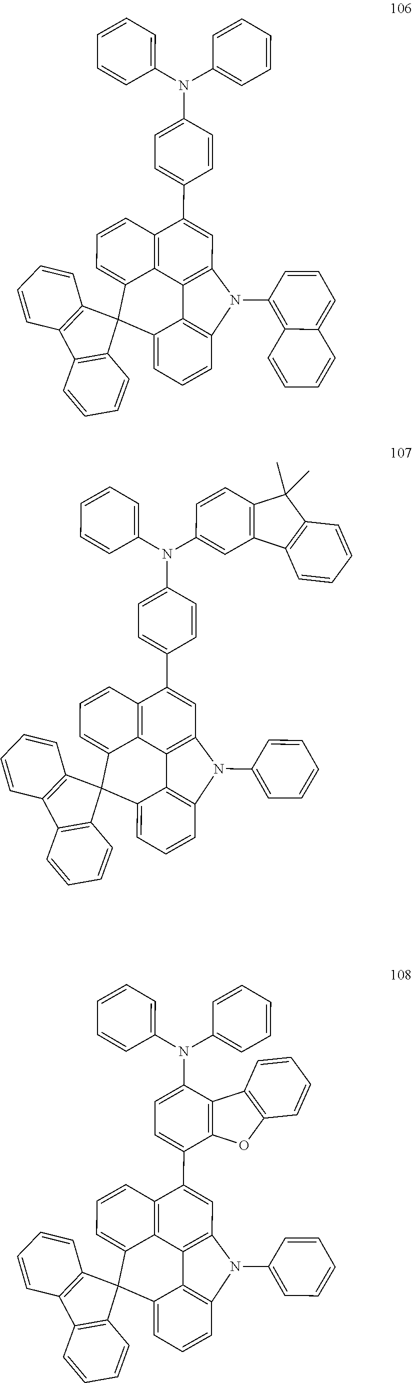 [SMILES: c1ccc(N(c2ccccc2)c2ccc(-c3cc4c5c6c(cccc36)C3(c6ccccc6-c6ccccc63)c3cccc(c35)n4-c3cccc4ccccc34)cc2)cc1 ; CC1(C)c2ccccc2-c2cc(N(c3ccccc3)c3ccc(-c4cc5c6c7c(cccc47)C4(c7ccccc7-c7ccccc74)c4cccc(c46)n5-c4ccccc4)cc3)ccc21 ; c1ccc(N(c2ccccc2)c2ccc(-c3cc4c5c6c(cccc36)C3(c6ccccc6-c6ccccc63)c3cccc(c35)n4-c3ccccc3)c3oc4ccccc4c23)cc1]

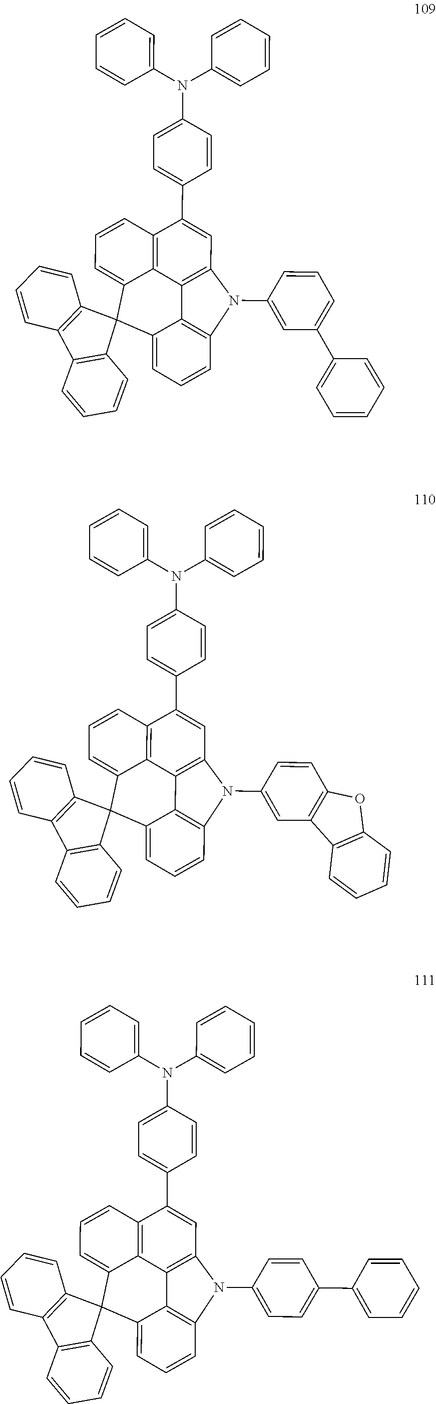 [SMILES: c1ccc(-c2cccc(-n3c4cccc5c4c4c6c(cccc6c(-c6ccc(N(c7ccccc7)c7ccccc7)cc6)cc43)C53c4ccccc4-c4ccccc43)c2)cc1 ; c1ccc(N(c2ccccc2)c2ccc(-c3cc4c5c6c(cccc36)C3(c6ccccc6-c6ccccc63)c3cccc(c35)n4-c3ccc4oc5ccccc5c4c3)cc2)cc1 ; c1ccc(-c2ccc(-n3c4cccc5c4c4c6c(cccc6c(-c6ccc(N(c7ccccc7)c7ccccc7)cc6)cc43)C53c4ccccc4-c4ccccc43)cc2)cc1]

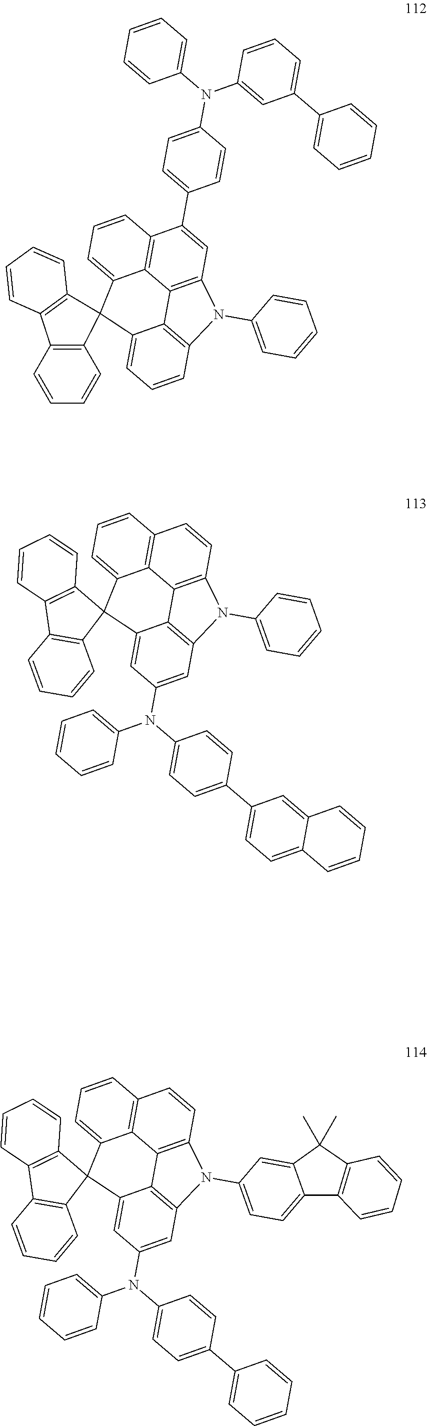 [SMILES: c1ccc(-c2cccc(N(c3ccccc3)c3ccc(-c4cc5c6c7c(cccc47)C4(c7ccccc7-c7ccccc74)c4cccc(c46)n5-c4ccccc4)cc3)c2)cc1 ; c1ccc(N(c2ccc(-c3ccc4ccccc4c3)cc2)c2cc3c4c5c6c(cccc6ccc5n(-c5ccccc5)c4c2)C32c3ccccc3-c3ccccc32)cc1 ; CC1(C)c2ccccc2-c2ccc(-n3c4cc(N(c5ccccc5)c5ccc(-c6ccccc6)cc5)cc5c4c4c6c(cccc6ccc43)C53c4ccccc4-c4ccccc43)cc21]

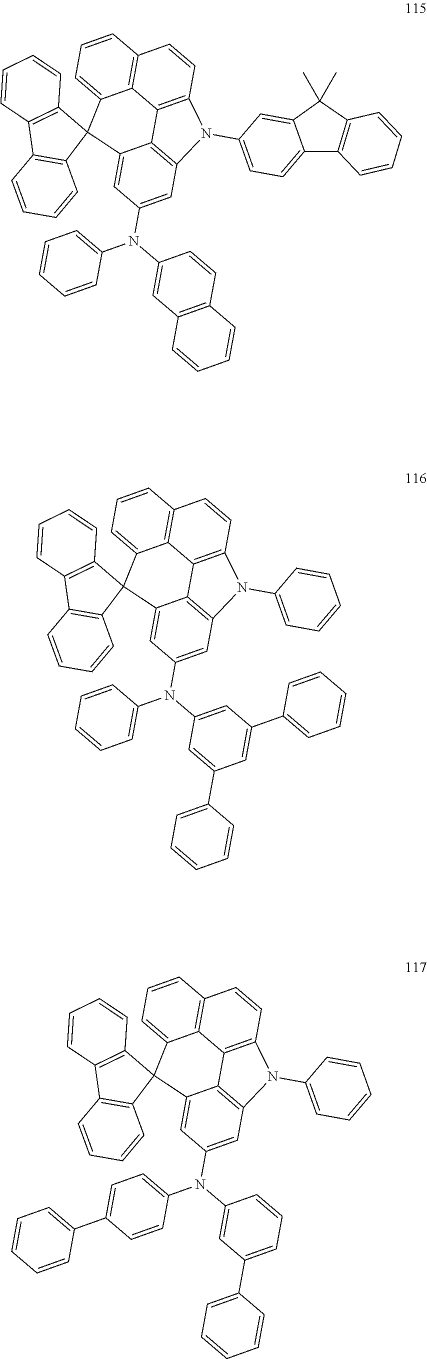 [SMILES: CC1(C)c2ccccc2-c2ccc(-n3c4cc(N(c5ccccc5)c5ccc6ccccc6c5)cc5c4c4c6c(cccc6ccc43)C53c4ccccc4-c4ccccc43)cc21 ; c1ccc(-c2cc(-c3ccccc3)cc(N(c3ccccc3)c3cc4c5c6c7c(cccc7ccc6n(-c6ccccc6)c5c3)C43c4ccccc4-c4ccccc43)c2)cc1 ; c1ccc(-c2ccc(N(c3cccc(-c4ccccc4)c3)c3cc4c5c6c7c(cccc7ccc6n(-c6ccccc6)c5c3)C43c4ccccc4-c4ccccc43)cc2)cc1]

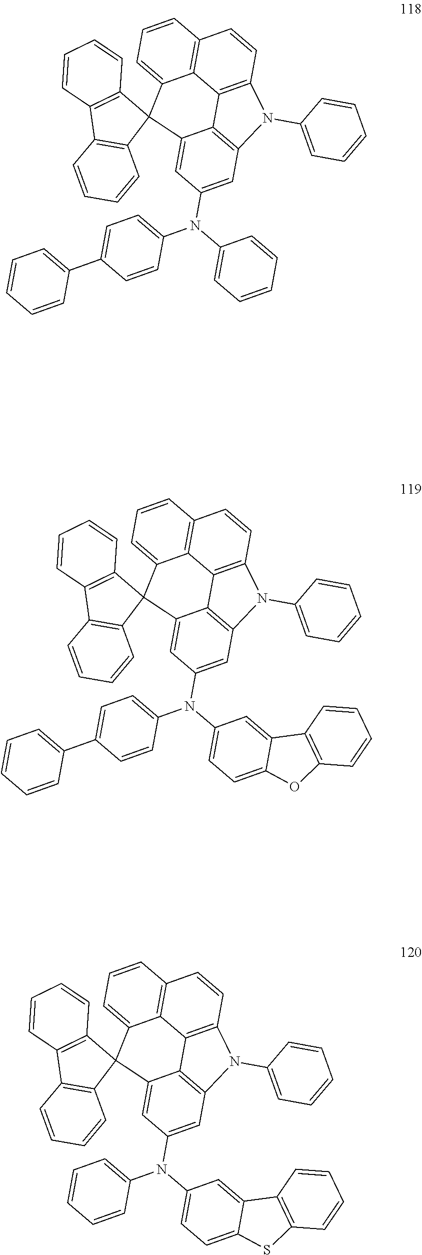 [SMILES: c1ccc(-c2ccc(N(c3ccccc3)c3cc4c5c6c7c(cccc7ccc6n(-c6ccccc6)c5c3)C43c4ccccc4-c4ccccc43)cc2)cc1 ; c1ccc(-c2ccc(N(c3ccc4oc5ccccc5c4c3)c3cc4c5c6c7c(cccc7ccc6n(-c6ccccc6)c5c3)C43c4ccccc4-c4ccccc43)cc2)cc1 ; c1ccc(N(c2ccc3sc4ccccc4c3c2)c2cc3c4c5c6c(cccc6ccc5n(-c5ccccc5)c4c2)C32c3ccccc3-c3ccccc32)cc1]

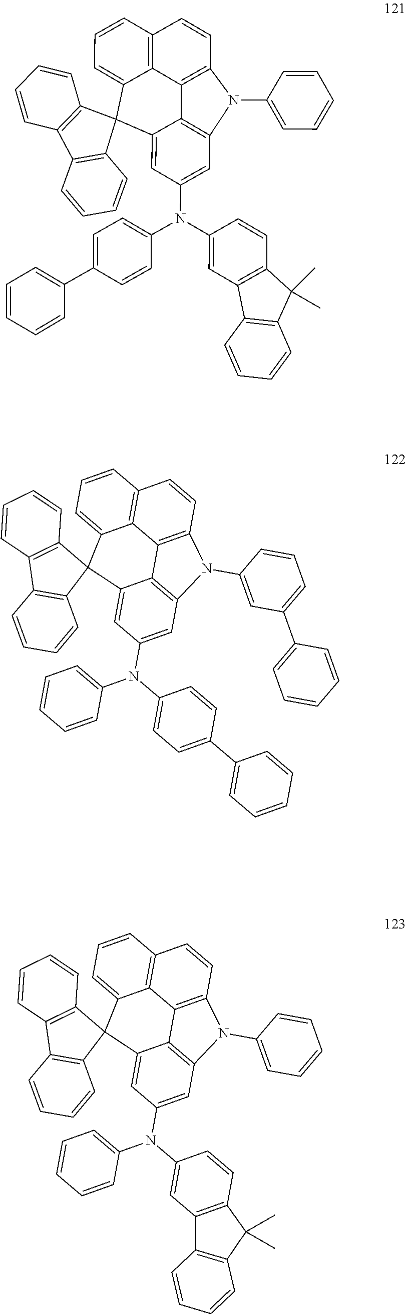 [SMILES: CC1(C)c2ccccc2-c2cc(N(c3ccc(-c4ccccc4)cc3)c3cc4c5c6c7c(cccc7ccc6n(-c6ccccc6)c5c3)C43c4ccccc4-c4ccccc43)ccc21 ; c1ccc(-c2ccc(N(c3ccccc3)c3cc4c5c6c7c(cccc7ccc6n(-c6cccc(-c7ccccc7)c6)c5c3)C43c4ccccc4-c4ccccc43)cc2)cc1 ; CC1(C)c2ccccc2-c2cc(N(c3ccccc3)c3cc4c5c6c7c(cccc7ccc6n(-c6ccccc6)c5c3)C43c4ccccc4-c4ccccc43)ccc21]

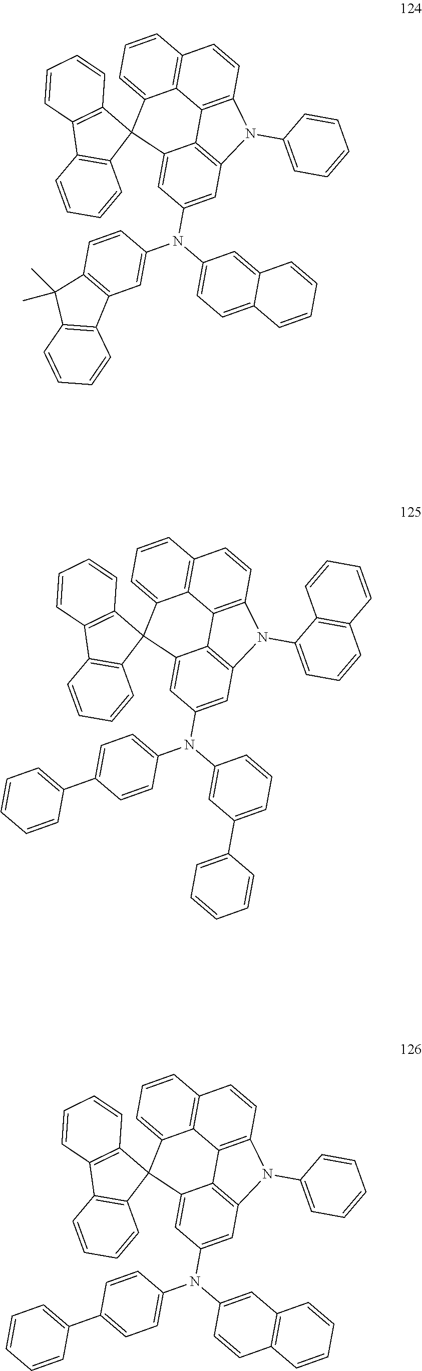 [SMILES: CC1(C)c2ccccc2-c2cc(N(c3ccc4ccccc4c3)c3cc4c5c6c7c(cccc7ccc6n(-c6ccccc6)c5c3)C43c4ccccc4-c4ccccc43)ccc21 ; c1ccc(-c2ccc(N(c3cccc(-c4ccccc4)c3)c3cc4c5c6c7c(cccc7ccc6n(-c6cccc7ccccc67)c5c3)C43c4ccccc4-c4ccccc43)cc2)cc1 ; c1ccc(-c2ccc(N(c3ccc4ccccc4c3)c3cc4c5c6c7c(cccc7ccc6n(-c6ccccc6)c5c3)C43c4ccccc4-c4ccccc43)cc2)cc1]

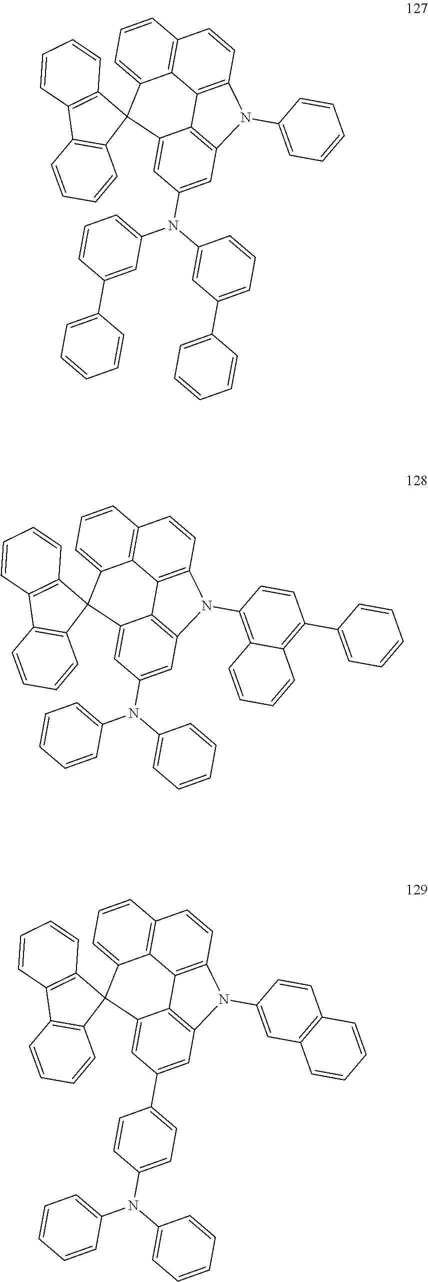 [SMILES: c1ccc(-c2cccc(N(c3cccc(-c4ccccc4)c3)c3cc4c5c6c7c(cccc7ccc6n(-c6ccccc6)c5c3)C43c4ccccc4-c4ccccc43)c2)cc1 ; c1ccc(-c2ccc(-n3c4cc(N(c5ccccc5)c5ccccc5)cc5c4c4c6c(cccc6ccc43)C53c4ccccc4-c4ccccc43)c3ccccc23)cc1 ; c1ccc(N(c2ccccc2)c2ccc(-c3cc4c5c6c7c(cccc7ccc6n(-c6ccc7ccccc7c6)c5c3)C43c4ccccc4-c4ccccc43)cc2)cc1]

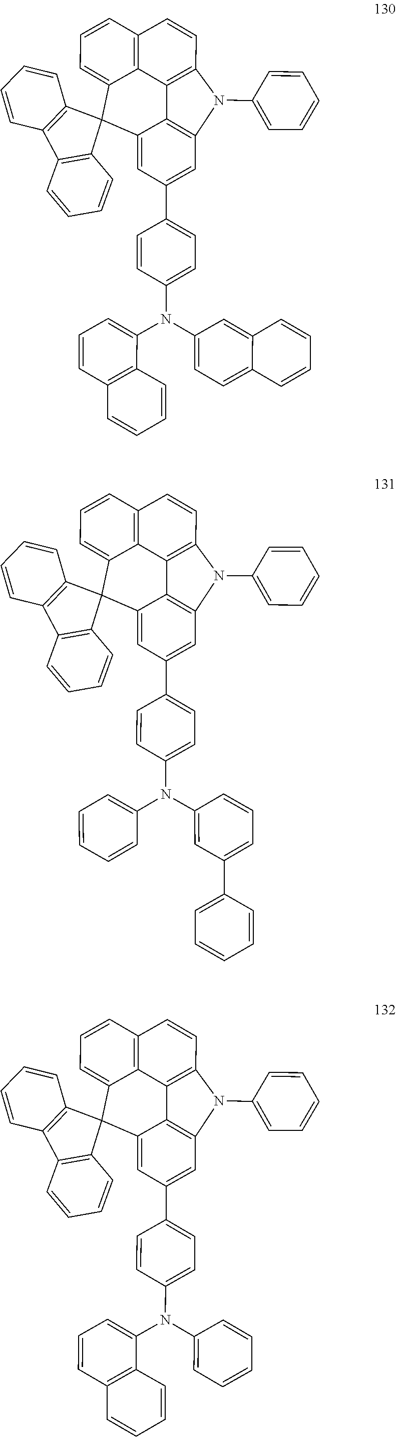 [SMILES: c1ccc(-n2c3cc(-c4ccc(N(c5ccc6ccccc6c5)c5cccc6ccccc56)cc4)cc4c3c3c5c(cccc5ccc32)C42c3ccccc3-c3ccccc32)cc1 ; c1ccc(-c2cccc(N(c3ccccc3)c3ccc(-c4cc5c6c7c8c(cccc8ccc7n(-c7ccccc7)c6c4)C54c5ccccc5-c5ccccc54)cc3)c2)cc1 ; c1ccc(N(c2ccc(-c3cc4c5c6c7c(cccc7ccc6n(-c6ccccc6)c5c3)C43c4ccccc4-c4ccccc43)cc2)c2cccc3ccccc23)cc1]

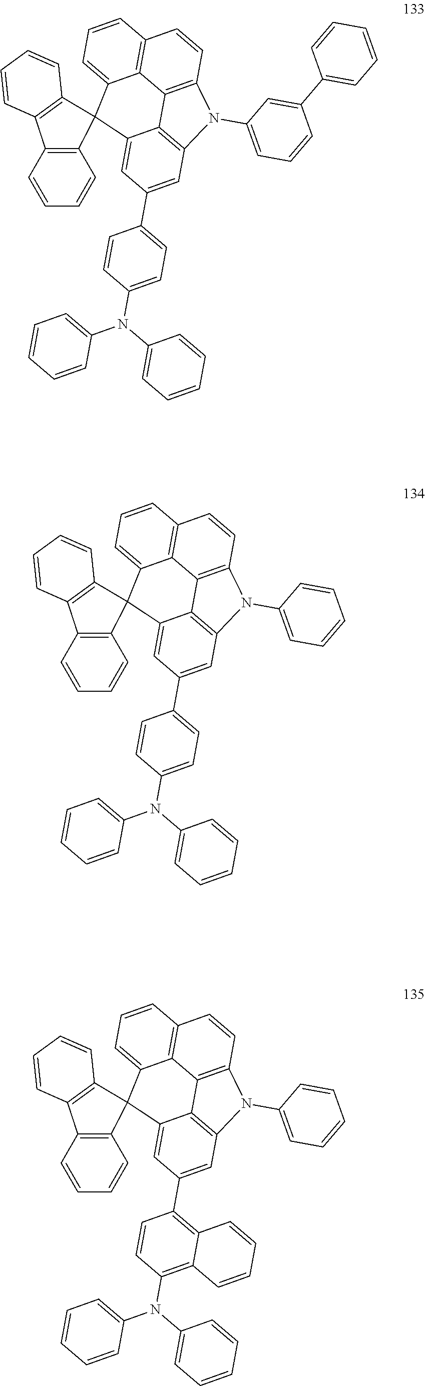 [SMILES: c1ccc(-c2cccc(-n3c4cc(-c5ccc(N(c6ccccc6)c6ccccc6)cc5)cc5c4c4c6c(cccc6ccc43)C53c4ccccc4-c4ccccc43)c2)cc1 ; c1ccc(N(c2ccccc2)c2ccc(-c3cc4c5c6c7c(cccc7ccc6n(-c6ccccc6)c5c3)C43c4ccccc4-c4ccccc43)cc2)cc1 ; c1ccc(N(c2ccccc2)c2ccc(-c3cc4c5c6c7c(cccc7ccc6n(-c6ccccc6)c5c3)C43c4ccccc4-c4ccccc43)c3ccccc23)cc1]

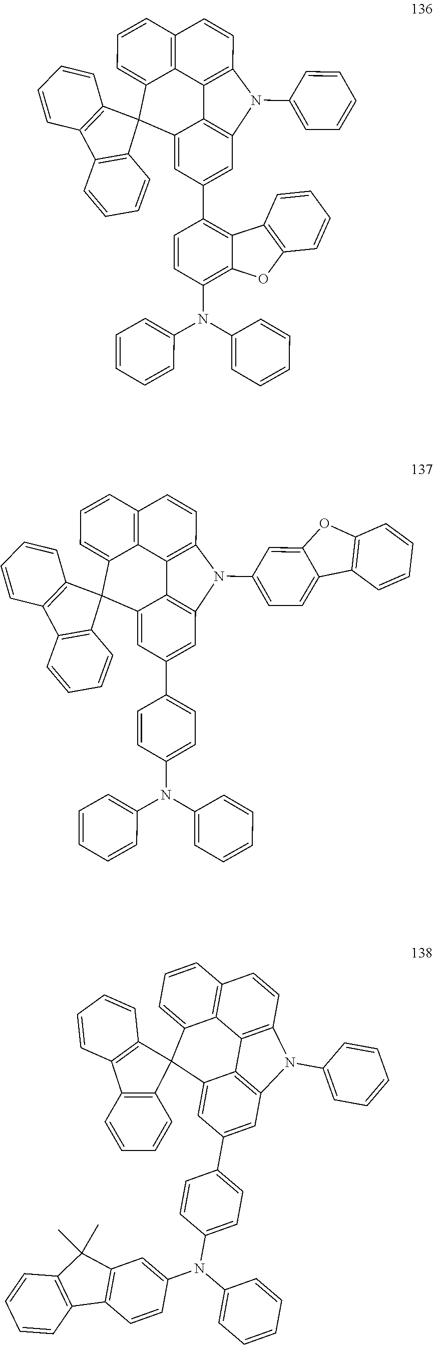 [SMILES: c1ccc(N(c2ccccc2)c2ccc(-c3cc4c5c6c7c(cccc7ccc6n(-c6ccccc6)c5c3)C43c4ccccc4-c4ccccc43)c3c2oc2ccccc23)cc1 ; c1ccc(N(c2ccccc2)c2ccc(-c3cc4c5c6c7c(cccc7ccc6n(-c6ccc7c(c6)oc6ccccc67)c5c3)C43c4ccccc4-c4ccccc43)cc2)cc1 ; CC1(C)c2ccccc2-c2ccc(N(c3ccccc3)c3ccc(-c4cc5c6c7c8c(cccc8ccc7n(-c7ccccc7)c6c4)C54c5ccccc5-c5ccccc54)cc3)cc21]

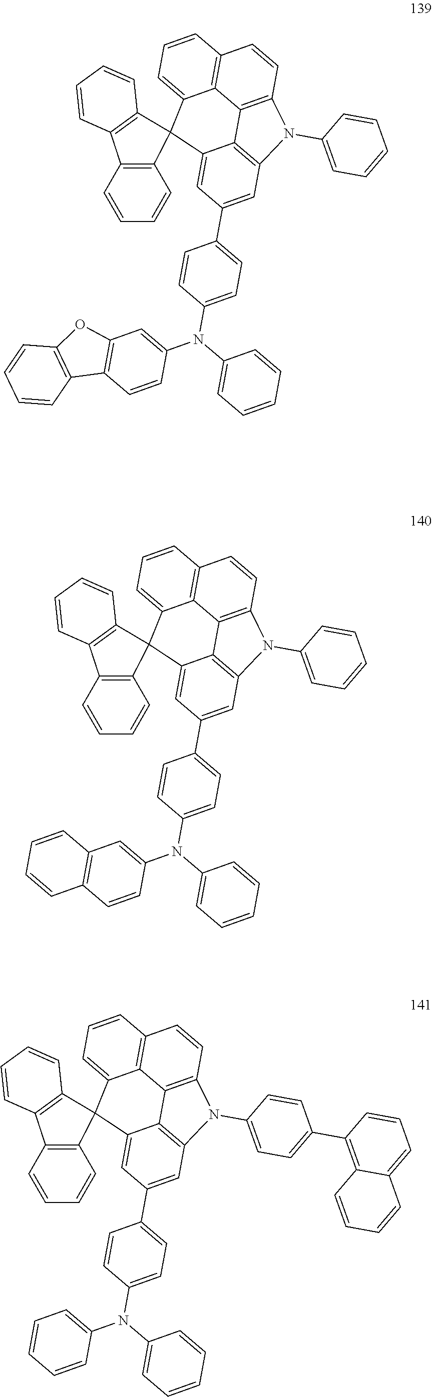 [SMILES: c1ccc(N(c2ccc(-c3cc4c5c6c7c(cccc7ccc6n(-c6ccccc6)c5c3)C43c4ccccc4-c4ccccc43)cc2)c2ccc3c(c2)oc2ccccc23)cc1 ; c1ccc(N(c2ccc(-c3cc4c5c6c7c(cccc7ccc6n(-c6ccccc6)c5c3)C43c4ccccc4-c4ccccc43)cc2)c2ccc3ccccc3c2)cc1 ; c1ccc(N(c2ccccc2)c2ccc(-c3cc4c5c6c7c(cccc7ccc6n(-c6ccc(-c7cccc8ccccc78)cc6)c5c3)C43c4ccccc4-c4ccccc43)cc2)cc1]

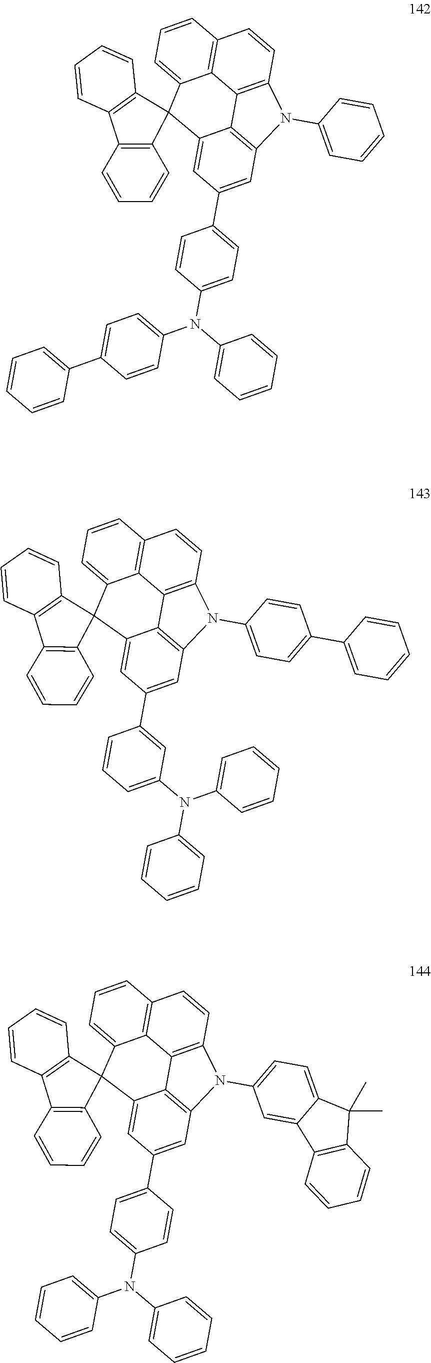 [SMILES: c1ccc(-c2ccc(N(c3ccccc3)c3ccc(-c4cc5c6c7c8c(cccc8ccc7n(-c7ccccc7)c6c4)C54c5ccccc5-c5ccccc54)cc3)cc2)cc1 ; c1ccc(-c2ccc(-n3c4cc(-c5cccc(N(c6ccccc6)c6ccccc6)c5)cc5c4c4c6c(cccc6ccc43)C53c4ccccc4-c4ccccc43)cc2)cc1 ; CC1(C)c2ccccc2-c2cc(-n3c4cc(-c5ccc(N(c6ccccc6)c6ccccc6)cc5)cc5c4c4c6c(cccc6ccc43)C53c4ccccc4-c4ccccc43)ccc21]

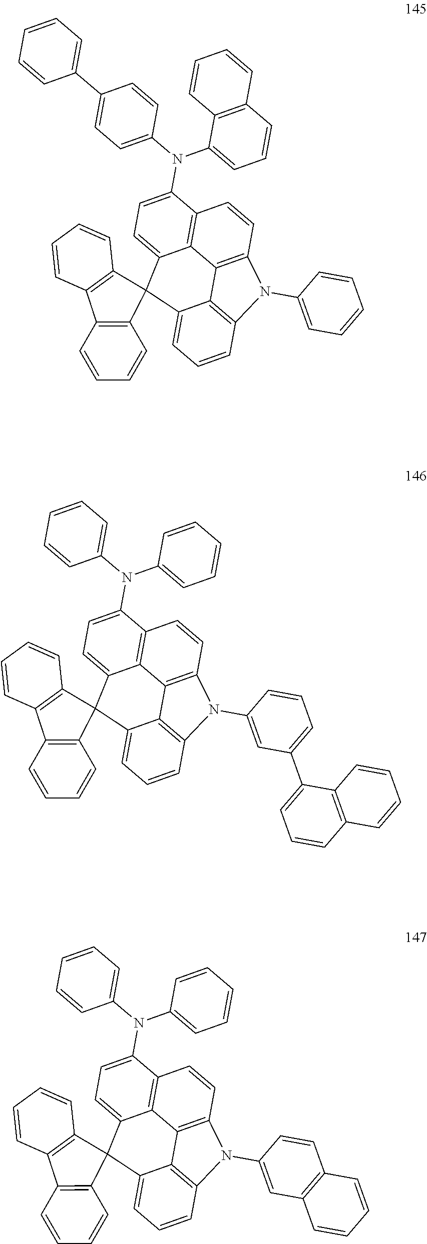 [SMILES: c1ccc(-c2ccc(N(c3cccc4ccccc34)c3ccc4c5c3ccc3c5c5c(cccc5n3-c3ccccc3)C43c4ccccc4-c4ccccc43)cc2)cc1 ; c1ccc(N(c2ccccc2)c2ccc3c4c2ccc2c4c4c(cccc4n2-c2cccc(-c4cccc5ccccc45)c2)C32c3ccccc3-c3ccccc32)cc1 ; c1ccc(N(c2ccccc2)c2ccc3c4c2ccc2c4c4c(cccc4n2-c2ccc4ccccc4c2)C32c3ccccc3-c3ccccc32)cc1]

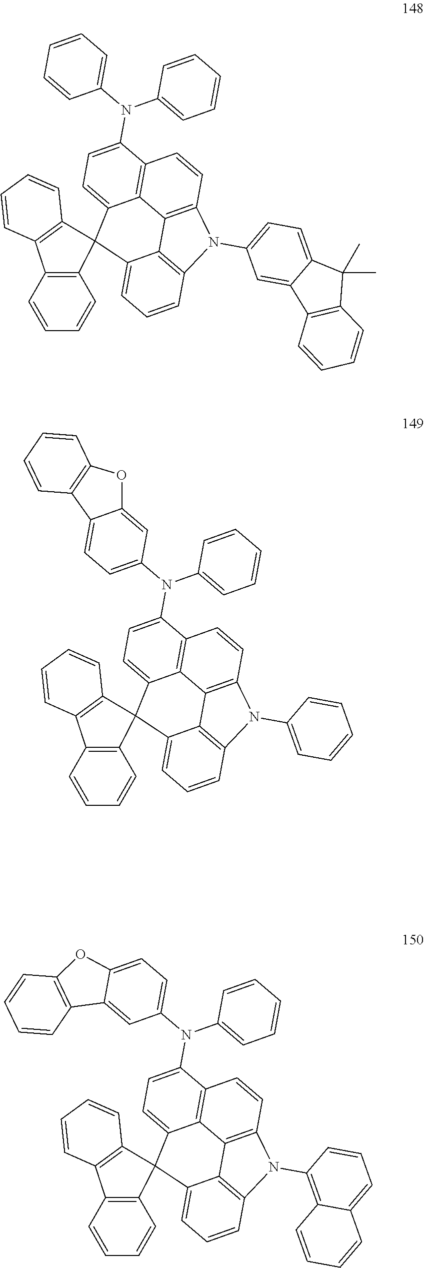 [SMILES: CC1(C)c2ccccc2-c2cc(-n3c4cccc5c4c4c6c(ccc(N(c7ccccc7)c7ccccc7)c6ccc43)C53c4ccccc4-c4ccccc43)ccc21 ; c1ccc(N(c2ccc3c(c2)oc2ccccc23)c2ccc3c4c2ccc2c4c4c(cccc4n2-c2ccccc2)C32c3ccccc3-c3ccccc32)cc1 ; c1ccc(N(c2ccc3oc4ccccc4c3c2)c2ccc3c4c2ccc2c4c4c(cccc4n2-c2cccc4ccccc24)C32c3ccccc3-c3ccccc32)cc1]

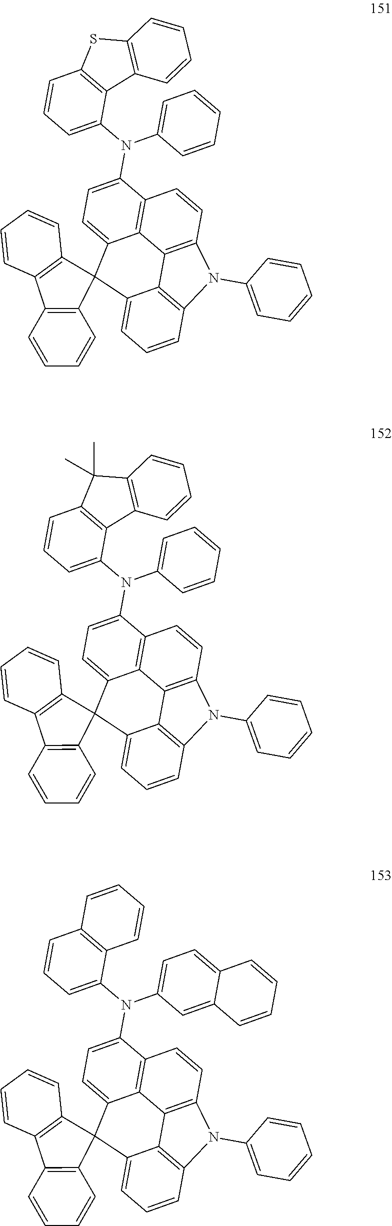 [SMILES: c1ccc(N(c2ccc3c4c2ccc2c4c4c(cccc4n2-c2ccccc2)C32c3ccccc3-c3ccccc32)c2cccc3sc4ccccc4c23)cc1 ; CC1(C)c2ccccc2-c2c(N(c3ccccc3)c3ccc4c5c3ccc3c5c5c(cccc5n3-c3ccccc3)C43c4ccccc4-c4ccccc43)cccc21 ; c1ccc(-n2c3cccc4c3c3c5c(ccc(N(c6ccc7ccccc7c6)c6cccc7ccccc67)c5ccc32)C42c3ccccc3-c3ccccc32)cc1]

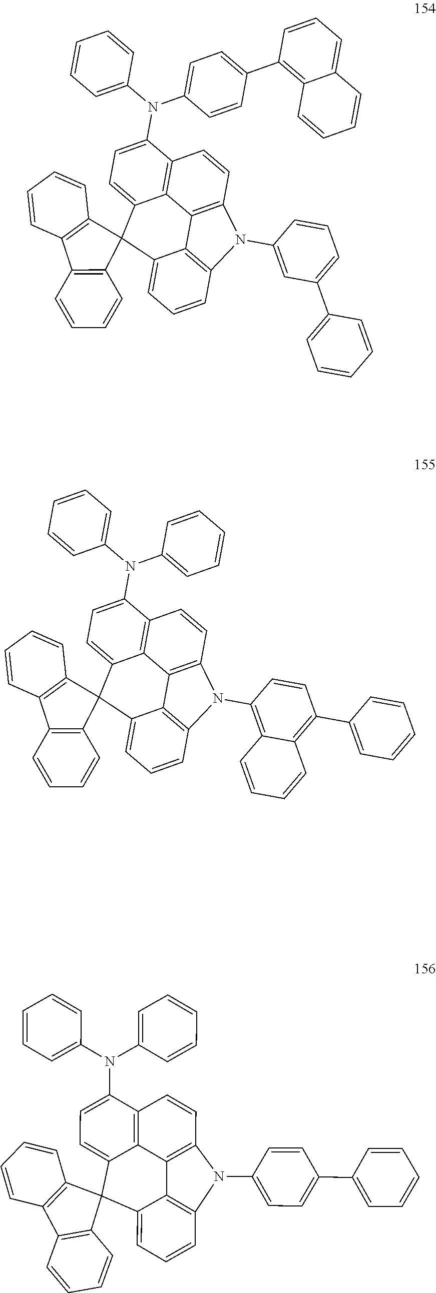 [SMILES: c1ccc(-c2cccc(-n3c4cccc5c4c4c6c(ccc(N(c7ccccc7)c7ccc(-c8cccc9ccccc89)cc7)c6ccc43)C53c4ccccc4-c4ccccc43)c2)cc1 ; c1ccc(-c2ccc(-n3c4cccc5c4c4c6c(ccc(N(c7ccccc7)c7ccccc7)c6ccc43)C53c4ccccc4-c4ccccc43)c3ccccc23)cc1 ; c1ccc(-c2ccc(-n3c4cccc5c4c4c6c(ccc(N(c7ccccc7)c7ccccc7)c6ccc43)C53c4ccccc4-c4ccccc43)cc2)cc1]

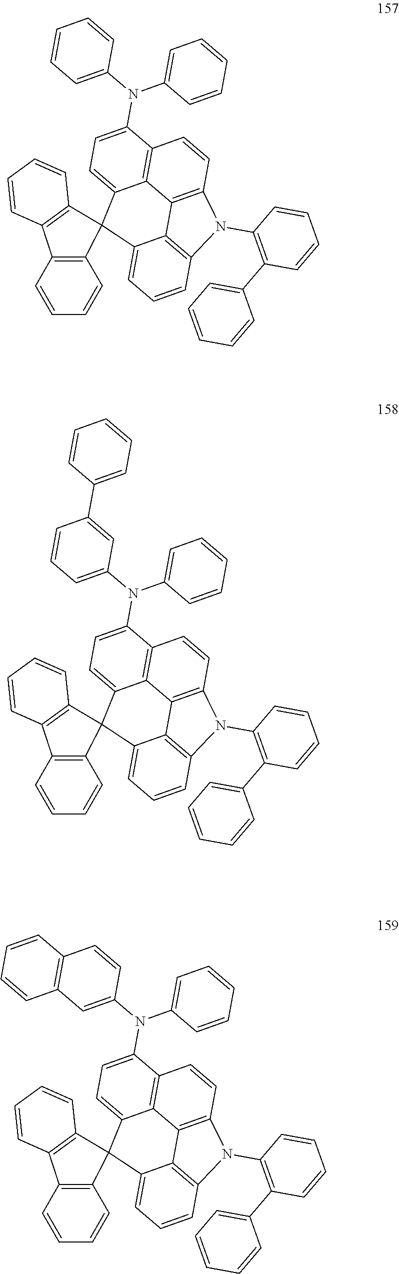 [SMILES: c1ccc(-c2ccccc2-n2c3cccc4c3c3c5c(ccc(N(c6ccccc6)c6ccccc6)c5ccc32)C42c3ccccc3-c3ccccc32)cc1 ; c1ccc(-c2cccc(N(c3ccccc3)c3ccc4c5c3ccc3c5c5c(cccc5n3-c3ccccc3-c3ccccc3)C43c4ccccc4-c4ccccc43)c2)cc1 ; c1ccc(-c2ccccc2-n2c3cccc4c3c3c5c(ccc(N(c6ccccc6)c6ccc7ccccc7c6)c5ccc32)C42c3ccccc3-c3ccccc32)cc1]

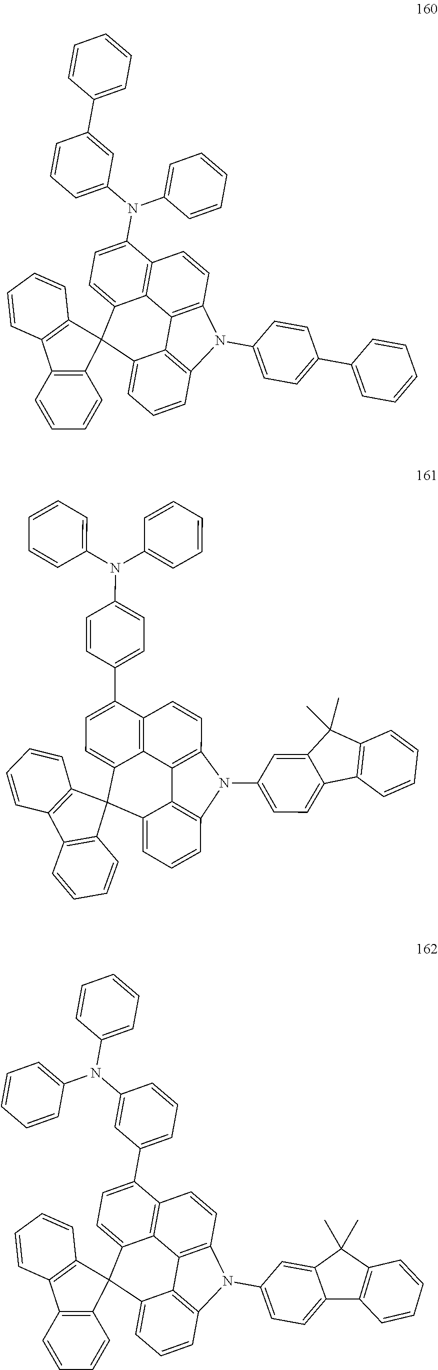 [SMILES: c1ccc(-c2ccc(-n3c4cccc5c4c4c6c(ccc(N(c7ccccc7)c7cccc(-c8ccccc8)c7)c6ccc43)C53c4ccccc4-c4ccccc43)cc2)cc1 ; CC1(C)c2ccccc2-c2ccc(-n3c4cccc5c4c4c6c(ccc(-c7ccc(N(c8ccccc8)c8ccccc8)cc7)c6ccc43)C53c4ccccc4-c4ccccc43)cc21 ; CC1(C)c2ccccc2-c2ccc(-n3c4cccc5c4c4c6c(ccc(-c7cccc(N(c8ccccc8)c8ccccc8)c7)c6ccc43)C53c4ccccc4-c4ccccc43)cc21]

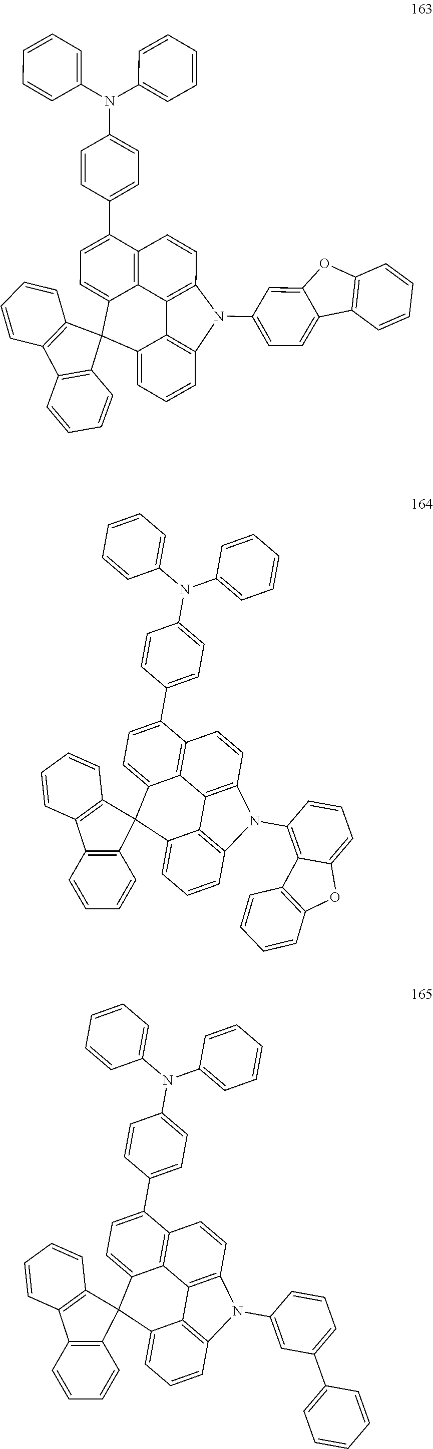 [SMILES: c1ccc(N(c2ccccc2)c2ccc(-c3ccc4c5c3ccc3c5c5c(cccc5n3-c3ccc5c(c3)oc3ccccc35)C43c4ccccc4-c4ccccc43)cc2)cc1 ; c1ccc(N(c2ccccc2)c2ccc(-c3ccc4c5c3ccc3c5c5c(cccc5n3-c3cccc5oc6ccccc6c35)C43c4ccccc4-c4ccccc43)cc2)cc1 ; c1ccc(-c2cccc(-n3c4cccc5c4c4c6c(ccc(-c7ccc(N(c8ccccc8)c8ccccc8)cc7)c6ccc43)C53c4ccccc4-c4ccccc43)c2)cc1]

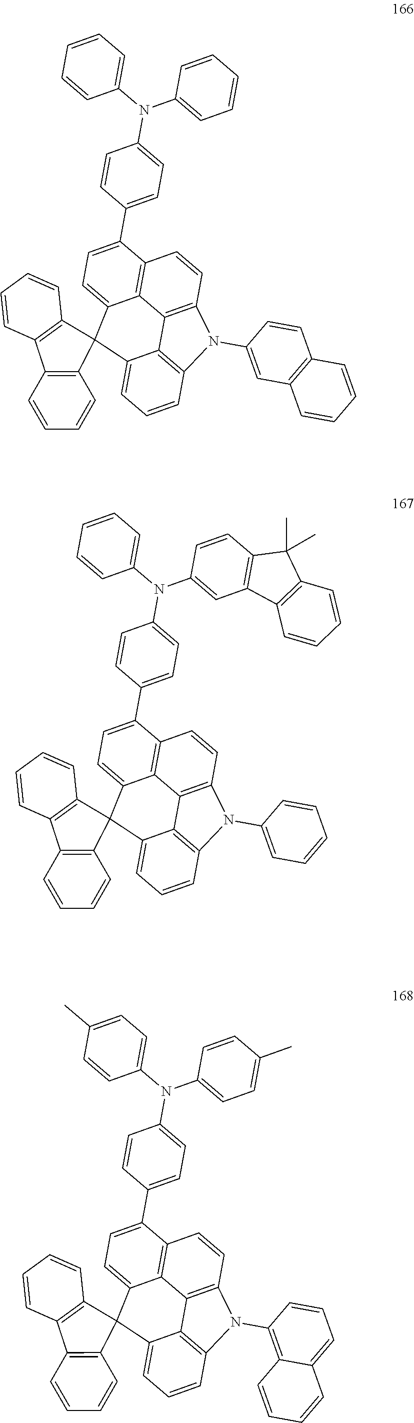 [SMILES: c1ccc(N(c2ccccc2)c2ccc(-c3ccc4c5c3ccc3c5c5c(cccc5n3-c3ccc5ccccc5c3)C43c4ccccc4-c4ccccc43)cc2)cc1 ; CC1(C)c2ccccc2-c2cc(N(c3ccccc3)c3ccc(-c4ccc5c6c4ccc4c6c6c(cccc6n4-c4ccccc4)C54c5ccccc5-c5ccccc54)cc3)ccc21 ; Cc1ccc(N(c2ccc(C)cc2)c2ccc(-c3ccc4c5c3ccc3c5c5c(cccc5n3-c3cccc5ccccc35)C43c4ccccc4-c4ccccc43)cc2)cc1]

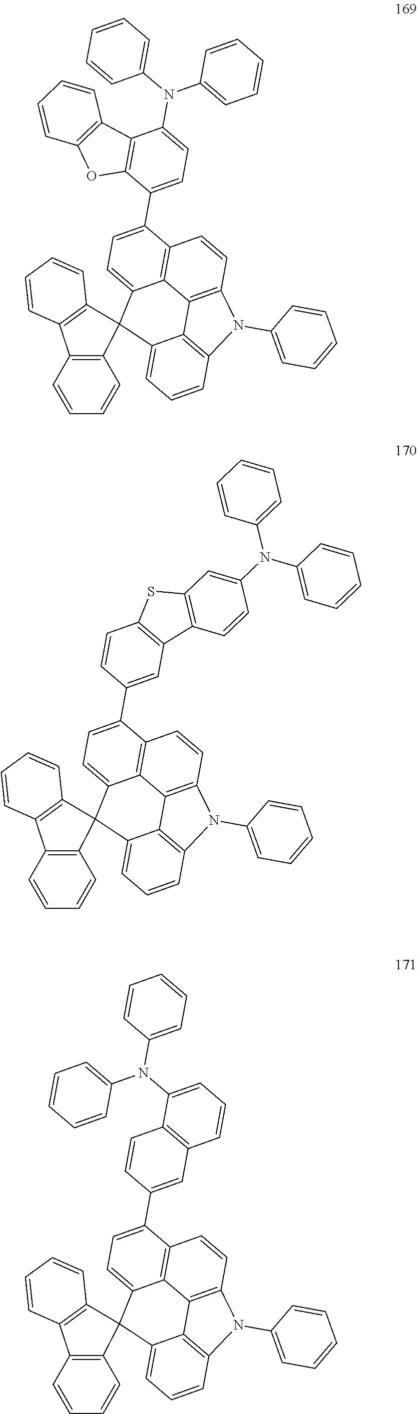 [SMILES: c1ccc(N(c2ccccc2)c2ccc(-c3ccc4c5c3ccc3c5c5c(cccc5n3-c3ccccc3)C43c4ccccc4-c4ccccc43)c3oc4ccccc4c23)cc1 ; c1ccc(N(c2ccccc2)c2ccc3c(c2)sc2ccc(-c4ccc5c6c4ccc4c6c6c(cccc6n4-c4ccccc4)C54c5ccccc5-c5ccccc54)cc23)cc1 ; c1ccc(N(c2ccccc2)c2cccc3cc(-c4ccc5c6c4ccc4c6c6c(cccc6n4-c4ccccc4)C54c5ccccc5-c5ccccc54)ccc23)cc1]

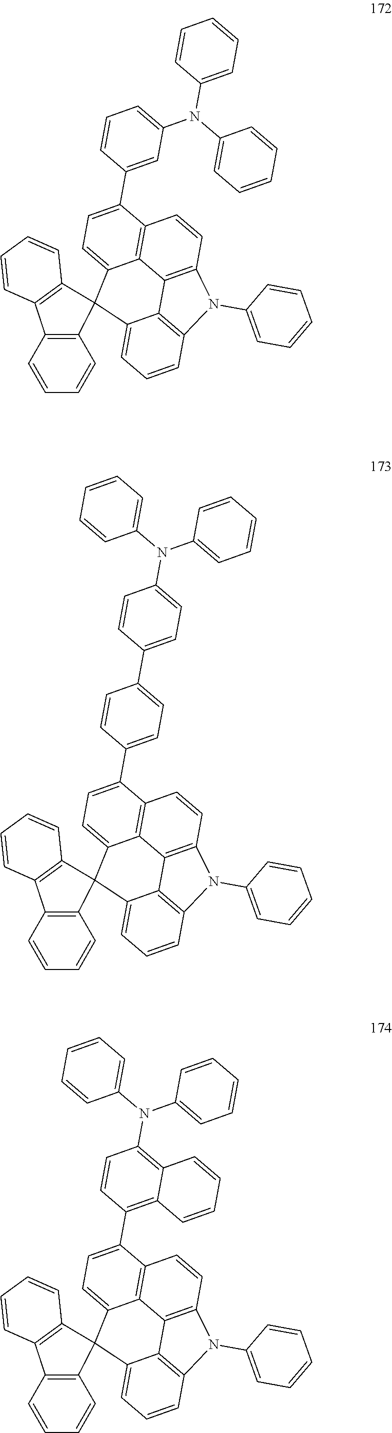 [SMILES: c1ccc(N(c2ccccc2)c2cccc(-c3ccc4c5c3ccc3c5c5c(cccc5n3-c3ccccc3)C43c4ccccc4-c4ccccc43)c2)cc1 ; c1ccc(N(c2ccccc2)c2ccc(-c3ccc(-c4ccc5c6c4ccc4c6c6c(cccc6n4-c4ccccc4)C54c5ccccc5-c5ccccc54)cc3)cc2)cc1 ; c1ccc(N(c2ccccc2)c2ccc(-c3ccc4c5c3ccc3c5c5c(cccc5n3-c3ccccc3)C43c4ccccc4-c4ccccc43)c3ccccc23)cc1]

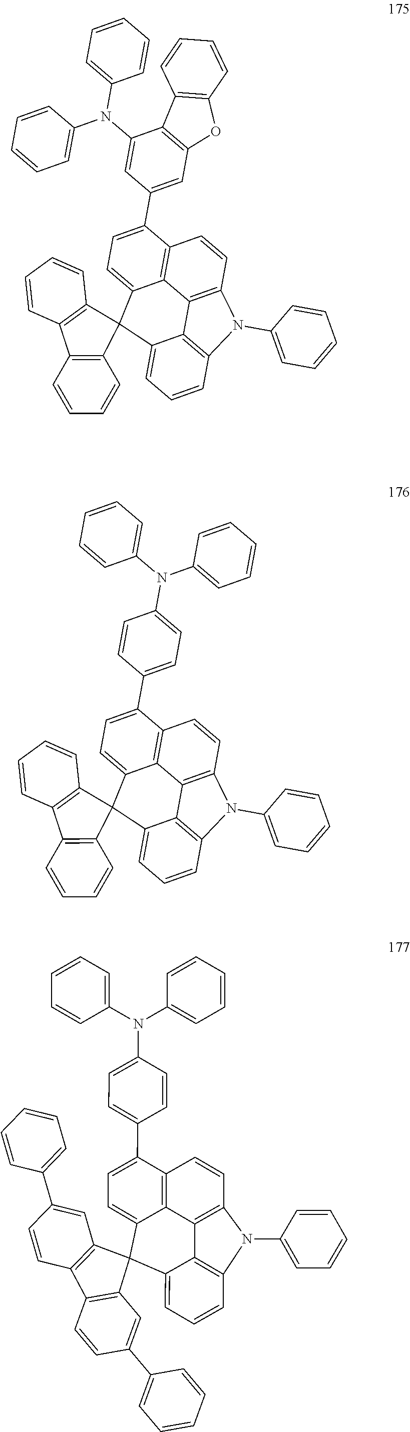 [SMILES: c1ccc(N(c2ccccc2)c2cc(-c3ccc4c5c3ccc3c5c5c(cccc5n3-c3ccccc3)C43c4ccccc4-c4ccccc43)cc3oc4ccccc4c23)cc1 ; c1ccc(N(c2ccccc2)c2ccc(-c3ccc4c5c3ccc3c5c5c(cccc5n3-c3ccccc3)C43c4ccccc4-c4ccccc43)cc2)cc1 ; c1ccc(-c2ccc3c(c2)C2(c4cc(-c5ccccc5)ccc4-3)c3ccc(-c4ccc(N(c5ccccc5)c5ccccc5)cc4)c4ccc5c(c34)c3c2cccc3n5-c2ccccc2)cc1]

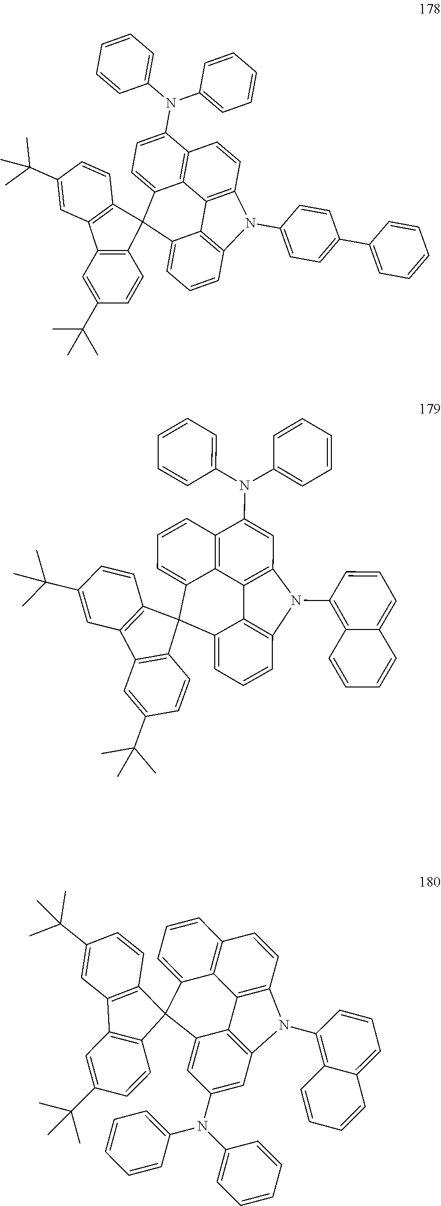 [SMILES: CC(C)(C)c1ccc2c(c1)-c1cc(C(C)(C)C)ccc1C21c2ccc(N(c3ccccc3)c3ccccc3)c3ccc4c(c23)c2c1cccc2n4-c1ccc(-c2ccccc2)cc1 ; CC(C)(C)c1ccc2c(c1)-c1cc(C(C)(C)C)ccc1C21c2cccc3c(N(c4ccccc4)c4ccccc4)cc4c(c23)c2c1cccc2n4-c1cccc2ccccc12 ; CC(C)(C)c1ccc2c(c1)-c1cc(C(C)(C)C)ccc1C21c2cccc3ccc4c(c23)c2c1cc(N(c1ccccc1)c1ccccc1)cc2n4-c1cccc2ccccc12]

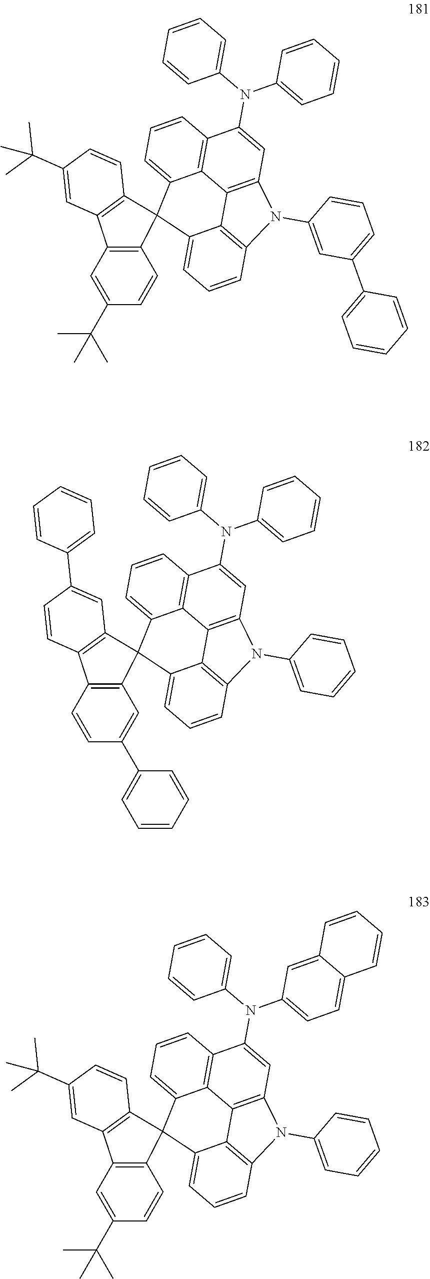 [SMILES: CC(C)(C)c1ccc2c(c1)-c1cc(C(C)(C)C)ccc1C21c2cccc3c(N(c4ccccc4)c4ccccc4)cc4c(c23)c2c1cccc2n4-c1cccc(-c2ccccc2)c1 ; c1ccc(-c2ccc3c(c2)C2(c4cc(-c5ccccc5)ccc4-3)c3cccc4c(N(c5ccccc5)c5ccccc5)cc5c(c34)c3c2cccc3n5-c2ccccc2)cc1 ; CC(C)(C)c1ccc2c(c1)-c1cc(C(C)(C)C)ccc1C21c2cccc3c(N(c4ccccc4)c4ccc5ccccc5c4)cc4c(c23)c2c1cccc2n4-c1ccccc1]

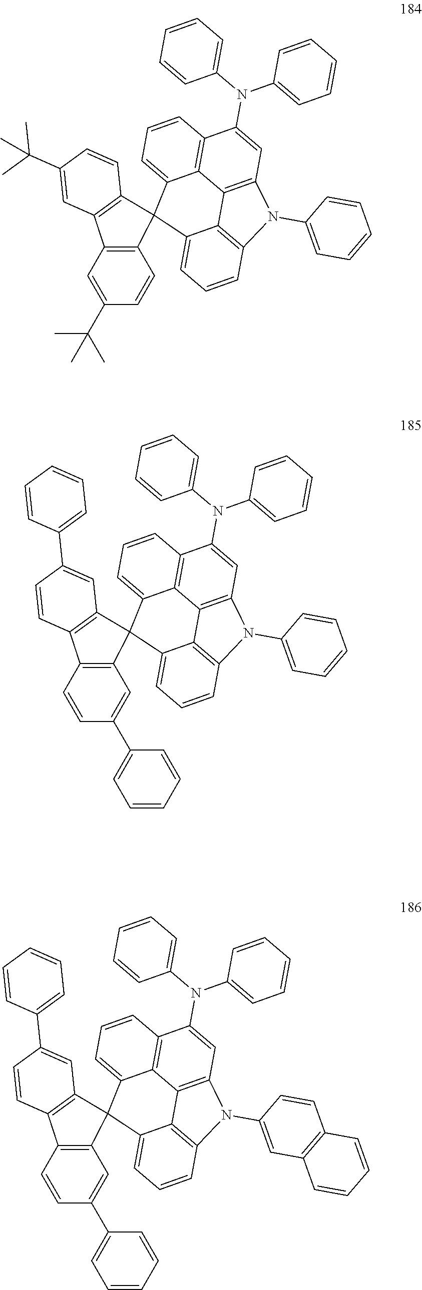 [SMILES: CC(C)(C)c1ccc2c(c1)-c1cc(C(C)(C)C)ccc1C21c2cccc3c(N(c4ccccc4)c4ccccc4)cc4c(c23)c2c1cccc2n4-c1ccccc1 ; c1ccc(-c2ccc3c(c2)C2(c4cc(-c5ccccc5)ccc4-3)c3cccc4c(N(c5ccccc5)c5ccccc5)cc5c(c34)c3c2cccc3n5-c2ccccc2)cc1 ; c1ccc(-c2ccc3c(c2)C2(c4cc(-c5ccccc5)ccc4-3)c3cccc4c(N(c5ccccc5)c5ccccc5)cc5c(c34)c3c2cccc3n5-c2ccc3ccccc3c2)cc1]

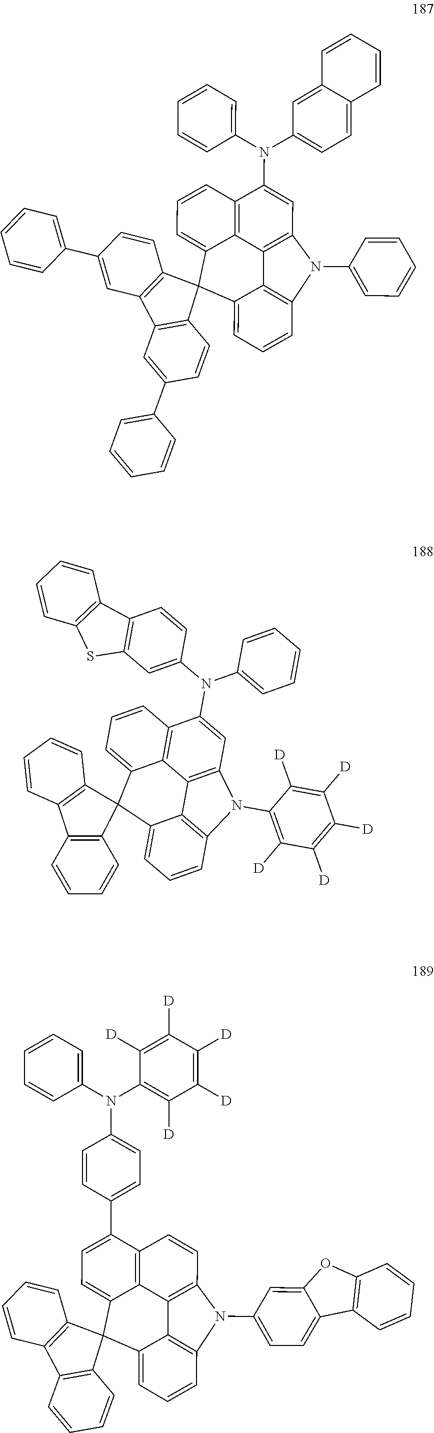 [SMILES: c1ccc(-c2ccc3c(c2)-c2cc(-c4ccccc4)ccc2C32c3cccc4c(N(c5ccccc5)c5ccc6ccccc6c5)cc5c(c34)c3c2cccc3n5-c2ccccc2)cc1 ; [2H]c1c([2H])c([2H])c(-n2c3cccc4c3c3c5c(cccc5c(N(c5ccccc5)c5ccc6c(c5)sc5ccccc56)cc32)C42c3ccccc3-c3ccccc32)c([2H])c1[2H] ; [2H]c1c([2H])c([2H])c(N(c2ccccc2)c2ccc(-c3ccc4c5c3ccc3c5c5c(cccc5n3-c3ccc5c(c3)oc3ccccc35)C43c4ccccc4-c4ccccc43)cc2)c([2H])c1[2H]]

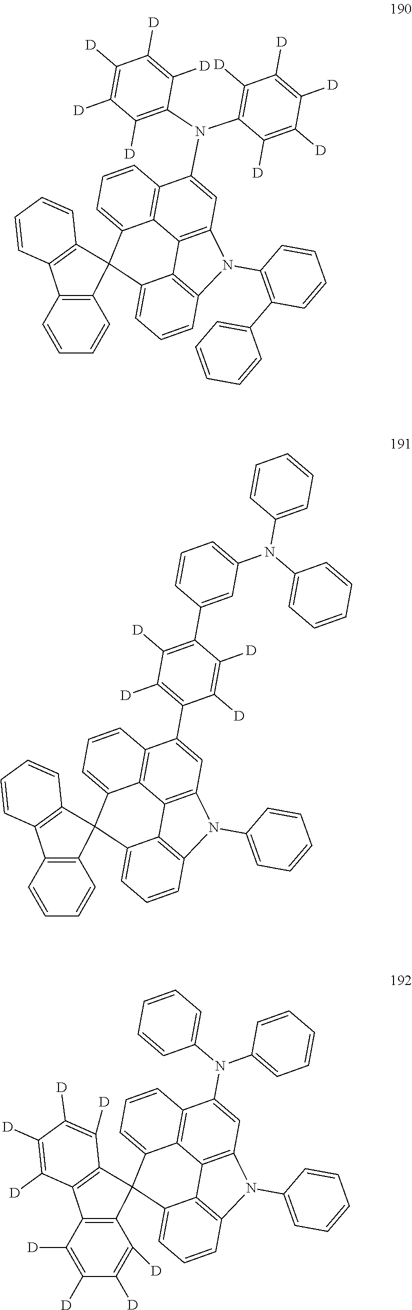 [SMILES: [2H]c1c([2H])c([2H])c(N(c2c([2H])c([2H])c([2H])c([2H])c2[2H])c2cc3c4c5c(cccc25)C2(c5ccccc5-c5ccccc52)c2cccc(c24)n3-c2ccccc2-c2ccccc2)c([2H])c1[2H] ; [2H]c1c([2H])c(-c2cc3c4c5c(cccc25)C2(c5ccccc5-c5ccccc52)c2cccc(c24)n3-c2ccccc2)c([2H])c([2H])c1-c1cccc(N(c2ccccc2)c2ccccc2)c1 ; [2H]c1c([2H])c([2H])c2c(c1[2H])-c1c([2H])c([2H])c([2H])c([2H])c1C21c2cccc3c(N(c4ccccc4)c4ccccc4)cc4c(c23)c2c1cccc2n4-c1ccccc1]

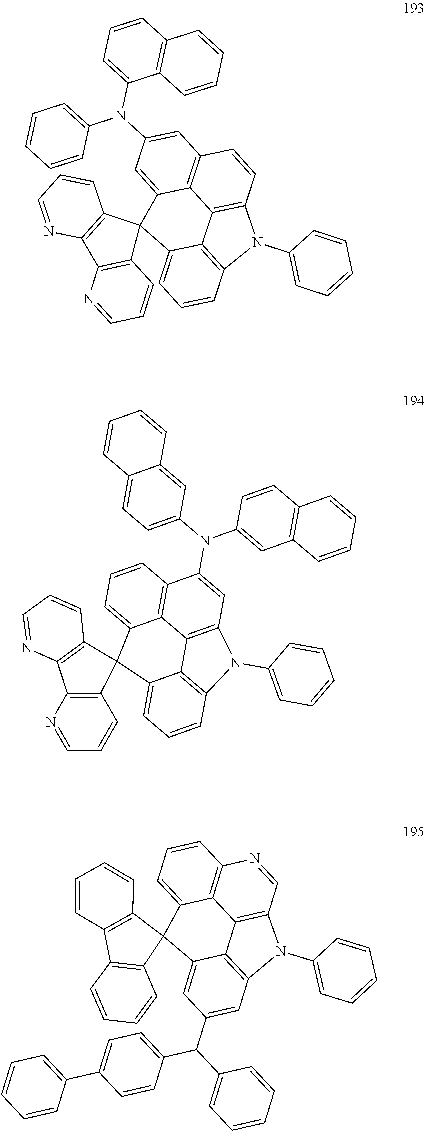 [SMILES: c1ccc(N(c2cc3c4c(ccc5c4c4c(cccc4n5-c4ccccc4)C34c3cccnc3-c3ncccc34)c2)c2cccc3ccccc23)cc1 ; c1ccc(-n2c3cccc4c3c3c5c(cccc5c(N(c5ccc6ccccc6c5)c5ccc6ccccc6c5)cc32)C42c3cccnc3-c3ncccc32)cc1 ; c1ccc(-c2ccc(C(c3ccccc3)c3cc4c5c6c7c(cccc7ncc6n(-c6ccccc6)c5c3)C43c4ccccc4-c4ccccc43)cc2)cc1]

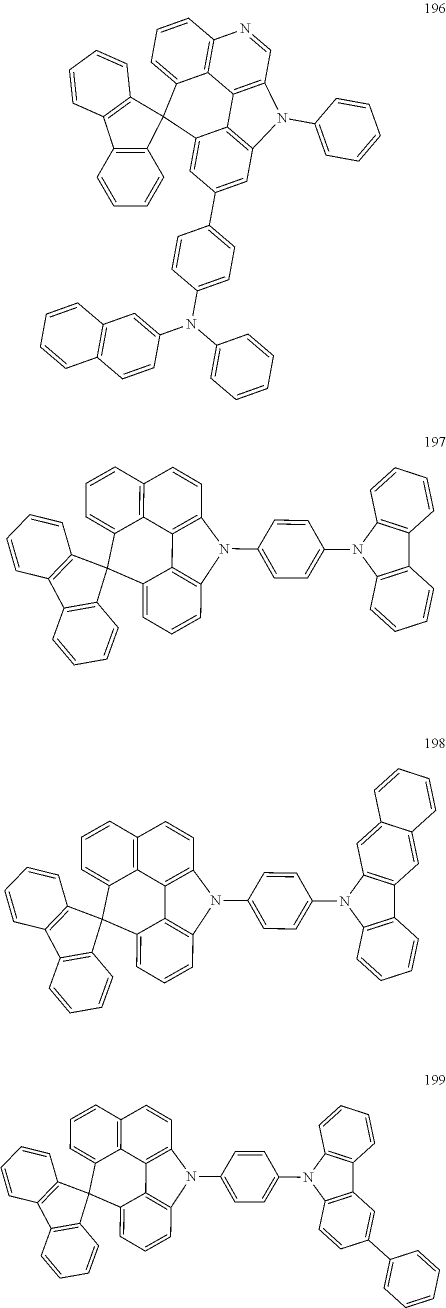 [SMILES: c1ccc(N(c2ccc(-c3cc4c5c6c7c(cccc7ncc6n(-c6ccccc6)c5c3)C43c4ccccc4-c4ccccc43)cc2)c2ccc3ccccc3c2)cc1 ; c1ccc2c(c1)-c1ccccc1C21c2cccc3ccc4c(c23)c2c1cccc2n4-c1ccc(-n2c3ccccc3c3ccccc32)cc1 ; c1ccc2c(c1)-c1ccccc1C21c2cccc3ccc4c(c23)c2c1cccc2n4-c1ccc(-n2c3ccccc3c3cc4ccccc4cc32)cc1 ; c1ccc(-c2ccc3c(c2)c2ccccc2n3-c2ccc(-n3c4cccc5c4c4c6c(cccc6ccc43)C53c4ccccc4-c4ccccc43)cc2)cc1]

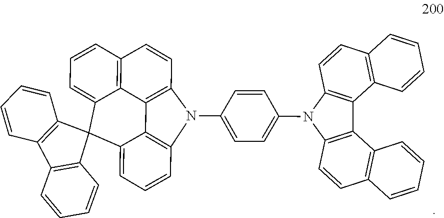 [SMILES: c1ccc2c(c1)-c1ccccc1C21c2cccc3ccc4c(c23)c2c1cccc2n4-c1ccc(-n2c3ccc4ccccc4c3c3c4ccccc4ccc32)cc1]

8. An organic light emitting device comprising,
a first electrode;
a second electrode provided to face the first electrode; and
one or more organic layers provided between the first electrode and the second electrode,
wherein at least one of the one or more organic layers comprises the heterocyclic compound according to claim 1.

9. The organic light emitting device according to claim 8, wherein the one or more organic layers further comprises a heterocyclic compound represented by Formula 2 below: 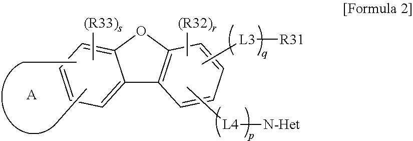
wherein,
N-Het is a substituted or unsubstituted, C2 to C60 monocyclic or polycyclic heterocyclic group containing one or more N,
L3 and L4 are the same as or different from each other, and are each independently a direct bond; a substituted or unsubstituted C6 to C60 arylene group; or a substituted or unsubstituted C2 to C60 heteroarylene group, p is an integer from 0 to 5, and when p is 2 or more, each L3 is the same as or different from each other, q is an integer from 0 to 5, and when q is 2 or more, each L4 is the same as or different from each other,
A is a substituted or unsubstituted C6 to C60 aryl ring; or a substituted or unsubstituted C2 to C60 heteroaryl ring,
R31 to R33 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other are combined with each other to form a substituted or unsubstituted C6 to C60 aromatic hydrocarbon ring or a substituted or unsubstituted C2 to C60 heterocyclic ring, wherein R201, R202, and R203 are the same as or different from each other, and are each independently a substituted or unsubstituted C1 to C60 alkyl group; a substituted or unsubstituted C6 to C60 aryl group; or a substituted or unsubstituted C2 to C60 heteroaryl group, and
r and s are each an integer of 0 to 2, and when r is 2 or more, each R32 is the same as or different from each other, and when s is 2 or more, each R33 is the same as or different from each other.

10. The organic light emitting device according to claim 9, wherein Formula 2 is represented by any one of Formula 2-1 to Formula 2-3 below: 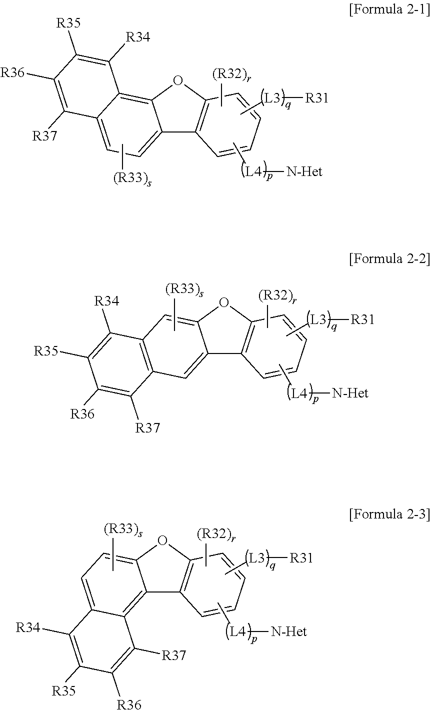
wherein,
R34 to R37 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other are combined with each other to form a substituted or unsubstituted C6 to C60 aromatic hydrocarbon ring or a substituted or unsubstituted C2 to C60 heterocyclic ring, wherein R201, R202, and R203 are the same as or different from each other, and are each independently a substituted or unsubstituted C1 to C60 alkyl group; a substituted or unsubstituted C6 to C60 aryl group; or a substituted or unsubstituted C2 to C60 heteroaryl group, and
the definitions of N-Het, L3, L4, R31 to R33, p, q, r and s are the same as those in Formula 2 above.

11. The organic light emitting device according to claim 9, wherein N-Het is a heterocyclic compound represented by any one of Formula 3-1 to Formula 3-4 below: 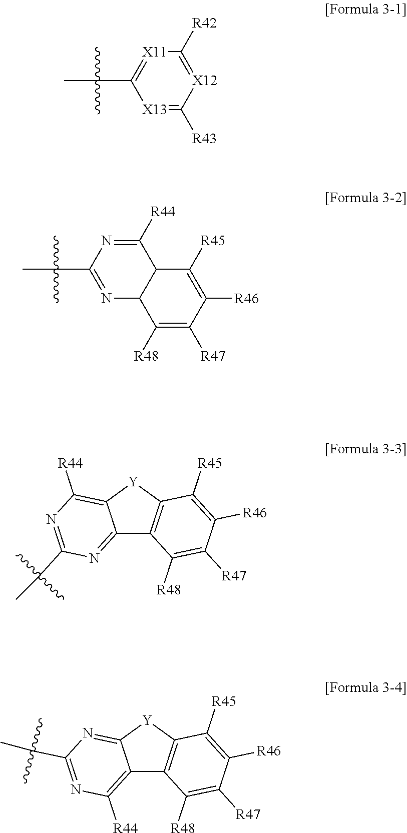
wherein,
X11 to X13 are the same as or different from each other, and each independently are N or CR41, and at least two of X11 to X13 are N,
Y is O; or S,
R42 to R44 are the same as or different from each other, and are each independently a substituted or unsubstituted C6 to C60 aryl group; a substituted or unsubstituted C2 to C60 heteroaryl group,
R41 and R45 to R48 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R301R302; —SiR301R302R303; and —NR301R302, or two or more groups adjacent to each other are combined with each other to form a substituted or unsubstituted C6 to C60 aromatic hydrocarbon ring or a substituted or unsubstituted C2 to C60 heterocyclic ring, wherein R301, R302, and R303 are the same as or different from each other, and are each independently a substituted or unsubstituted C1 to C60 alkyl group; a substituted or unsubstituted C6 to C60 aryl group; or a substituted or unsubstituted C2 to C60 heteroaryl group.

12. The organic light emitting device according to claim 9, wherein the heterocyclic compound represented by Formula 2 is any one selected from compounds below:

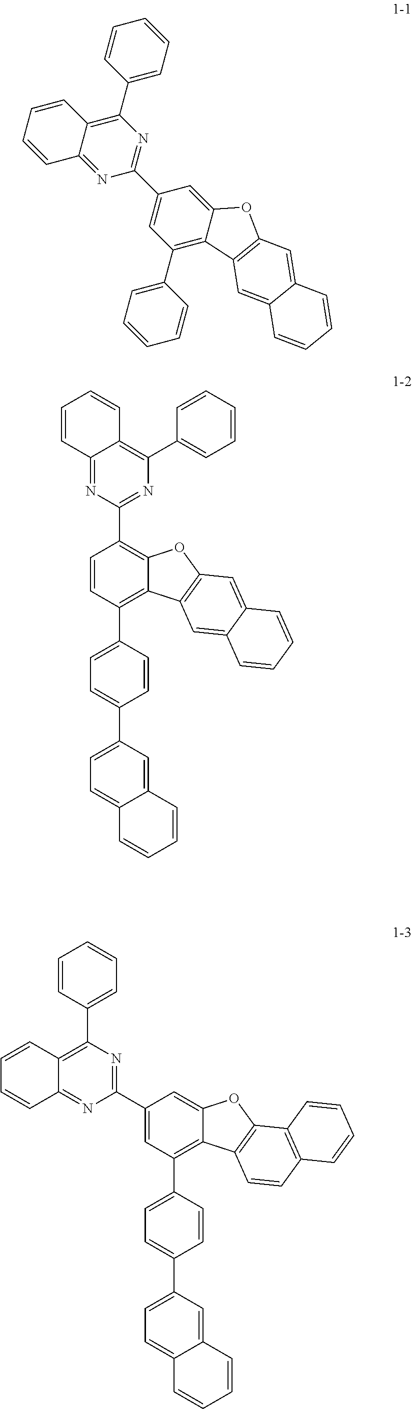 [SMILES: c1ccc(-c2nc(-c3cc(-c4ccccc4)c4c(c3)oc3cc5ccccc5cc34)nc3ccccc23)cc1 ; c1ccc(-c2nc(-c3ccc(-c4ccc(-c5ccc6ccccc6c5)cc4)c4c3oc3cc5ccccc5cc34)nc3ccccc23)cc1 ; c1ccc(-c2nc(-c3cc(-c4ccc(-c5ccc6ccccc6c5)cc4)c4c(c3)oc3c5ccccc5ccc34)nc3ccccc23)cc1]

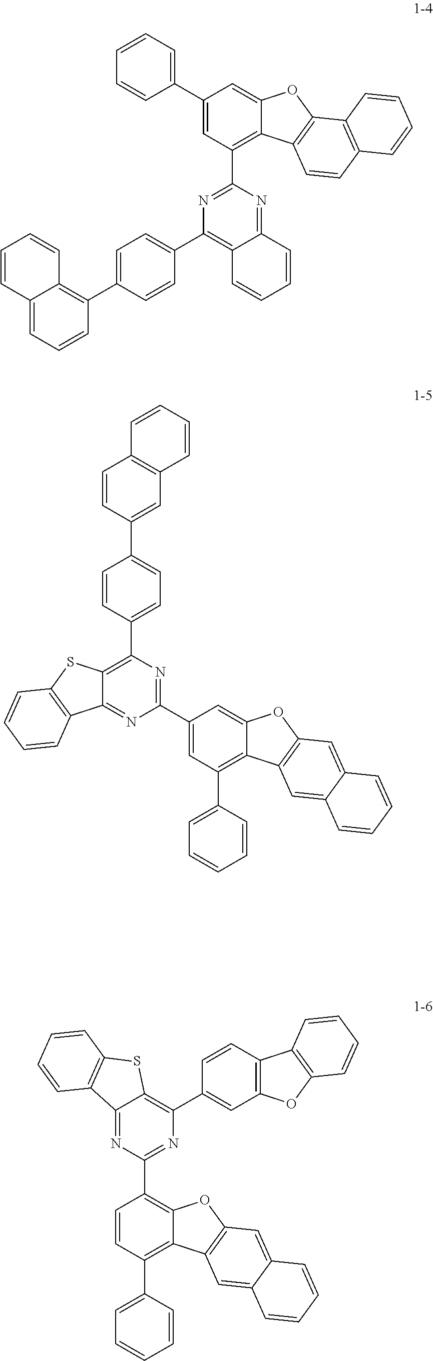 [SMILES: c1ccc(-c2cc(-c3nc(-c4ccc(-c5cccc6ccccc56)cc4)c4ccccc4n3)c3c(c2)oc2c4ccccc4ccc23)cc1 ; c1ccc(-c2cc(-c3nc(-c4ccc(-c5ccc6ccccc6c5)cc4)c4sc5ccccc5c4n3)cc3oc4cc5ccccc5cc4c23)cc1 ; c1ccc(-c2ccc(-c3nc(-c4ccc5c(c4)oc4ccccc45)c4sc5ccccc5c4n3)c3oc4cc5ccccc5cc4c23)cc1]

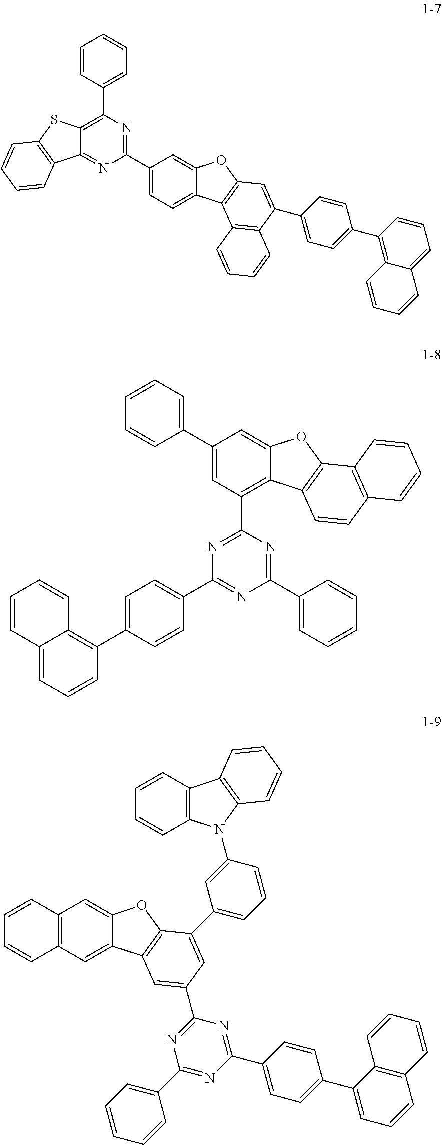 [SMILES: c1ccc(-c2nc(-c3ccc4c(c3)oc3cc(-c5ccc(-c6cccc7ccccc67)cc5)c5ccccc5c34)nc3c2sc2ccccc23)cc1 ; c1ccc(-c2cc(-c3nc(-c4ccccc4)nc(-c4ccc(-c5cccc6ccccc56)cc4)n3)c3c(c2)oc2c4ccccc4ccc23)cc1 ; c1ccc(-c2nc(-c3ccc(-c4cccc5ccccc45)cc3)nc(-c3cc(-c4cccc(-n5c6ccccc6c6ccccc65)c4)c4oc5cc6ccccc6cc5c4c3)n2)cc1]

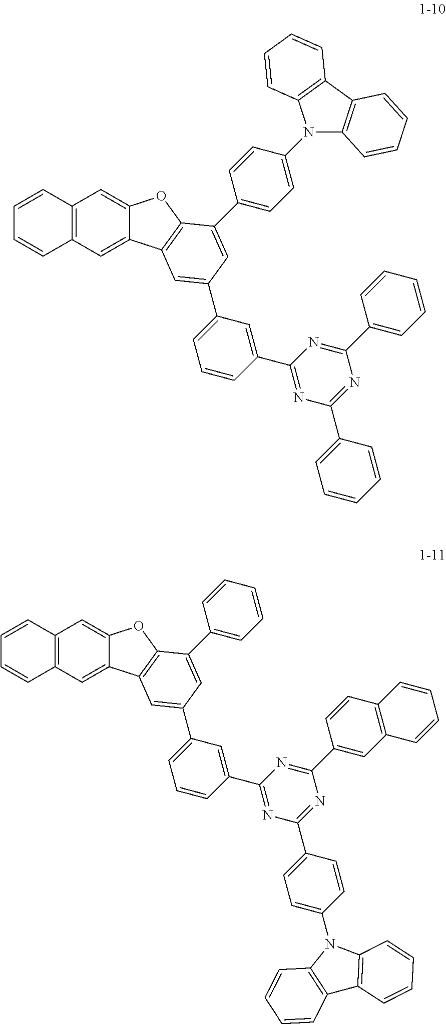 [SMILES: c1ccc(-c2nc(-c3ccccc3)nc(-c3cccc(-c4cc(-c5ccc(-n6c7ccccc7c7ccccc76)cc5)c5oc6cc7ccccc7cc6c5c4)c3)n2)cc1 ; c1ccc(-c2cc(-c3cccc(-c4nc(-c5ccc(-n6c7ccccc7c7ccccc76)cc5)nc(-c5ccc6ccccc6c5)n4)c3)cc3c2oc2cc4ccccc4cc23)cc1]

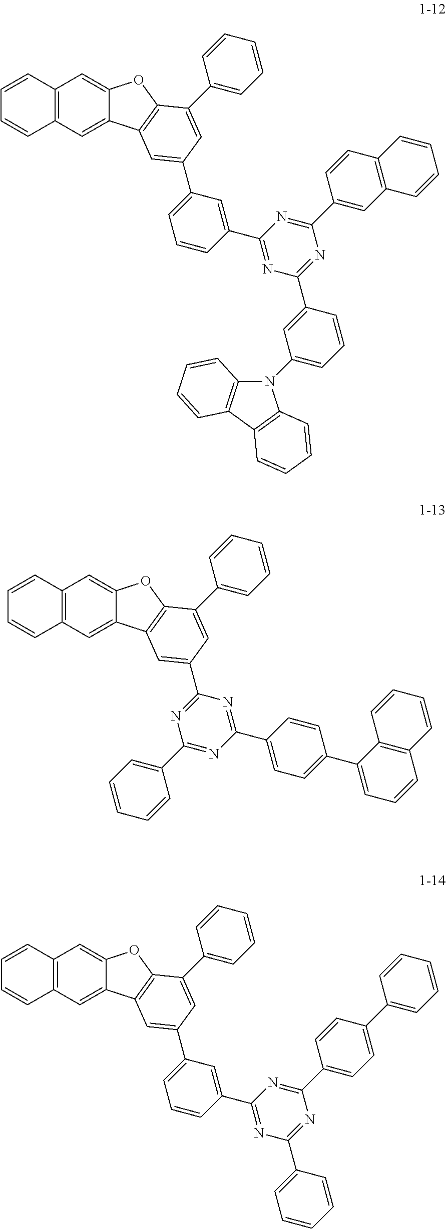 [SMILES: c1ccc(-c2cc(-c3cccc(-c4nc(-c5cccc(-n6c7ccccc7c7ccccc76)c5)nc(-c5ccc6ccccc6c5)n4)c3)cc3c2oc2cc4ccccc4cc23)cc1 ; c1ccc(-c2nc(-c3ccc(-c4cccc5ccccc45)cc3)nc(-c3cc(-c4ccccc4)c4oc5cc6ccccc6cc5c4c3)n2)cc1 ; c1ccc(-c2ccc(-c3nc(-c4ccccc4)nc(-c4cccc(-c5cc(-c6ccccc6)c6oc7cc8ccccc8cc7c6c5)c4)n3)cc2)cc1]

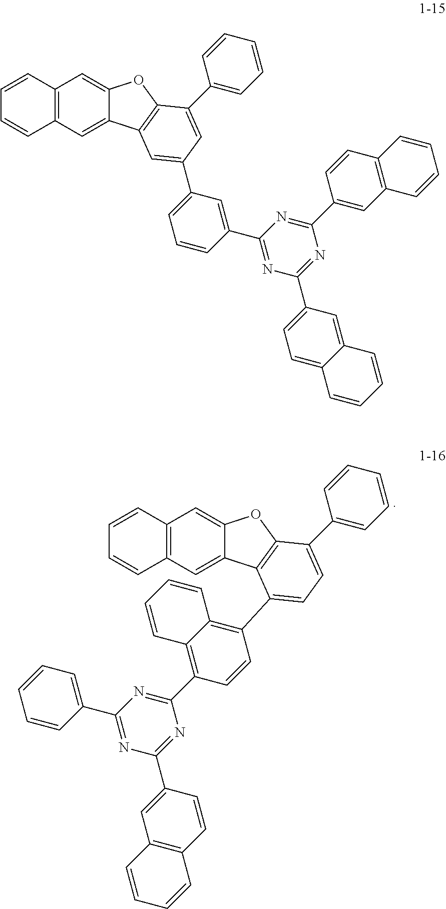 [SMILES: c1ccc(-c2cc(-c3cccc(-c4nc(-c5ccc6ccccc6c5)nc(-c5ccc6ccccc6c5)n4)c3)cc3c2oc2cc4ccccc4cc23)cc1 ; c1ccc(-c2nc(-c3ccc4ccccc4c3)nc(-c3ccc(-c4ccc(-c5ccccc5)c5oc6cc7ccccc7cc6c45)c4ccccc34)n2)cc1 ; "."]

13. The organic light emitting device according to claim 8, wherein the one or more organic layer comprises a light emitting layer, the light emitting layer comprises a host material, and the host material includes the heterocyclic compound represented by Formula 1 above and the heterocyclic compound represented by Formula 2 below. 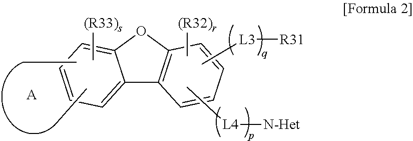
wherein,
N-Het is a substituted or unsubstituted, C2 to C60 monocyclic or polycyclic heterocyclic group containing one or more N,
L3 and L4 are the same as or different from each other, and are each independently a direct bond; a substituted or unsubstituted C6 to C60 arylene group; or a substituted or unsubstituted C2 to C60 heteroarylene group, p is an integer from 0 to 5, and when p is 2 or more, L3 is the same as or different from each other, q is an integer from 0 to 5, and when q is 2 or more, L4 is the same or different from each other,
A is a substituted or unsubstituted C6 to C60 aryl ring; or a substituted or unsubstituted C2 to C60 heteroaryl ring,
R31 to R33 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other are combined with each other to form a substituted or unsubstituted C6 to C60 aromatic hydrocarbon ring or a substituted or unsubstituted C2 to C60 heterocyclic ring, wherein R201, R202, and R203 are the same as or different from each other, and are each independently a substituted or unsubstituted C1 to C60 alkyl group; a substituted or unsubstituted C6 to C60 aryl group; or a substituted or unsubstituted C2 to C60 heteroaryl group, and
r and s are each an integer of 0 to 2, and when r is 2 or more, R32 is the same as or different from each other, and when s is 2 or more, R33 is the same as or different from each other.

14. The organic light emitting device according to claim 8, wherein the organic light emitting device further comprises one or more layers selected from the group consisting of a light emitting layer, a hole injection layer, a hole transport layer, an electron injection layer, an electron transport layer, an electron blocking layer, and a hole blocking layer.

15. A composition for organic layer of an organic light emitting device comprising a heterocyclic compound represented by Formula 1 according to claim 1 and a heterocyclic compound represented by Formula 2 below: 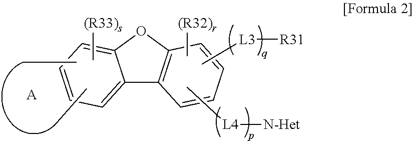
wherein,
N-Het is a substituted or unsubstituted, C2 to C60 monocyclic or polycyclic heterocyclic group containing one or more N,
L3 and L4 are the same as or different from each other, and are each independently a direct bond; a substituted or unsubstituted C6 to C60 arylene group; or a substituted or unsubstituted C2 to C60 heteroarylene group, p is an integer from 0 to 5, and when p is 2 or more, L3 is the same as or different from each other, q is an integer from 0 to 5, and when q is 2 or more, L4 is the same or different from each other,
A is a substituted or unsubstituted C6 to C60 aryl ring; or a substituted or unsubstituted C2 to C60 heteroaryl ring,
R31 to R33 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R201R202; —SiR201R202R203; and —NR201R202, or two or more groups adjacent to each other are combined with each other to form a substituted or unsubstituted C6 to C60 aromatic hydrocarbon ring or a substituted or unsubstituted C2 to C60 heterocyclic ring, wherein R201, R202, and R203 are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
r and s are each an integer of 0 to 2, and when r is 2 or more, R32 is the same as or different from each other, and when s is 2 or more, R33 is the same as or different from each other.

16. The composition for organic layer of the organic light emitting device according to claim 15, wherein the weight ratio of the heterocyclic compound represented by Formula 1:the heterocyclic compound represented by Formula 2 is 1:10 to 10:1.

17-18. (canceled)
